(19) United States
(12) Patent Application Publication
(10) Pub. No.: US 2007/0238669 A1
(43) Pub. Date: Oct. 11, 2007

(54) HUMAN GLUCAGON-LIKE-PEPTIDE-1 MODULATORS AND THEIR USE IN THE TREATMENT OF DIABETES RELATED CONDITIONS

(75) Inventors: Tasir Shamsul Haque, Yardley, PA (US); William R. Ewing, Yardley, PA (US); Claudio Mapelli, Plainsboro, NJ (US); Ving G. Lee, Hamilton, NJ (US); Richard B. Sulsky, West Trenton, NJ (US); Douglas James Riexinger, Flemington, NJ (US); Rogelio L. Martinez, Monmouth Junction, NJ (US); Yeheng Zhu, Stockton, NJ (US); Zheming Ruan, Dayton, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/622,142

(22) Filed: Jan. 11, 2007

Related U.S. Application Data
(60) Provisional application No. 60/758,165, filed on Jan. 11, 2006.
(60) Provisional application No. 60/758,164, filed on Jan. 11, 2006.
(60) Provisional application No. 60/758,096, filed on Jan. 11, 2006.
(60) Provisional application No. 60/758,107, filed on Jan. 11, 2006.

(51) Int. Cl. A61K 38/00; C07C 233/00; C07C 321/00; C07K 7/00

(52) U.S. Cl. 514/15; 530/327; 564/162; 564/163

(57) ABSTRACT

The present invention provides novel human glucagon-like peptide-1 (GLP-1)-receptor modulators that have biological activity similar or superior to native GLP-1 peptide and thus are useful for the treatment or prevention of diseases or disorders associated with GLP activity. Further, the present invention provides novel, chemically modified compounds that not only stimulate insulin secretion in type II diabetics, but also produce other beneficial insulinotropic responses. These synthetic peptide GLP-1 receptor modulators exhibit increased stability to proteolytic cleavage making them ideal therapeutic candidates for oral or parenteral administration. The compounds of this invention show desirable pharmacokinetic properties and desirable potency in efficacy models of diabetes.

This application claims priority from U.S. Application Ser. No. 60/758,165, filed Jan. 11, 2006; U.S. Application Ser. No. 60/758,164, filed Jan. 11, 2006; U.S. Application Ser. No. 60/758,096, filed Jan. 11, 2006; and U.S. Application Ser. No. 60/758,107, filed Jan. 11, 2006. Each application is incorporated herein by reference in their entirety.

### FIELD OF THE INVENTION

The subject matter described and claimed herein provides novel human glucagon-like peptide-1 (GLP-1) peptide receptor modulators, agonists or partial agonists, which exhibit superior biological properties relative to the native peptide, GLP-1. These compounds exhibit increased stability to proteolytic cleavage and thus are useful for the treatment and amelioration of the diabetic condition.

### BACKGROUND OF THE INVENTION

GLP-1 is an important gut hormone with regulatory function in glucose metabolism and gastrointestinal secretion and metabolism. Human GLP-1 is a 30 amino acid peptide originating from preproglucagon, which is synthesized, for example, in the L-cells in the distal ileum, in the pancreas, and in the brain. Processing of preproglucagon to yield GLP-1 (7-36) amide and GLP-2 occurs mainly in the L-cells and the brainstem. GLP-1 is normally secreted in response to food intake; carbohydrates and lipids in particular stimulate GLP-1 secretion. GLP-1 has been identified as a very potent and efficacious stimulator of glucose-dependent insulin release with a reduced risk to induce hypoglycemia. GLP-1 lowers plasma glucagon concentrations, slows gastric emptying, stimulates insulin biosynthesis and enhances insulin sensitivity (Nauck, Horm. Metab. Res., 29:9 (411-416) 1997). GLP-1 also enhances the ability of the pancreatic beta-cells to sense and respond to glucose in subjects with impaired glucose tolerance (Byrne, M. M. et al., Eur. J. Clin. Invest., 28(1):72-78 (1998)). The insulinotropic effect of GLP-1 in humans increases the rate of glucose metabolism, partly due to increased insulin levels and partly due to enhanced insulin sensitivity (D'Alessio, Eur. J. Clin. Invest., Vol. 15, No. 12 2005). Inhibition of glucagon release is thought to be an additional mechanism which contributes to the improvements in glucose homeostasis observed following treatment of type II diabetic patients with GLP-1 (Nauck, M. A. et al., Diabetologia, 36(8):741-744 (1993)). The above stated pharmacological properties of GLP-1 make it a highly desirable therapeutic agent for the treatment of type-II diabetes.

Additionally, recent studies have shown that infusions of slightly supraphysiological amounts of GLP-1 significantly enhance satiety and reduce food intake in normal subjects (Flint, A. et al., J. Clin. Invest., 101(3):515-520 (1998); Gutzwiller, J. P. et al., Gut, 44(1):81-86 (1999)). The effect on food intake and satiety has also been reported to be preserved in obese subjects (Naslund, E. et al., Int. J. Obes. Relat. Metab. Disord., 23(3):304-311 (1999)).

In the above-cited studies a pronounced effect of GLP-1 on gastric emptying was also suspected to occur. Gastric emptying results in post-prandial glucose excursions. It has also been shown that in addition to stimulation of insulin secretion, GLP-1 stimulates the expression of the transcription factor islet-duodenal homeobox-1 (IDX-1), while stimulating B-cell neogenesis, and may thereby be an effective treatment and/or preventive agent for diabetes (Stoffers, D. A. et al., Diabetes, 49(5):741-748 (2000)). GLP-1 has also been shown to inhibit gastric acid secretion (Wettergren, A. et al., Dig. Dis. Sci., 38(4):665-673 (1993)), which may provide protection against gastric ulcers.

It has recently been reported that GLP-1 has a number of additional extra-pancreatic effects that could, for example, result in cardioprotection, neuroprotection, and induction of learning and memory (reviewed in Ahren, B., Horm. Metab. Res., 36(11-12):842-845 (2004)). Therefore, it has also been proposed that GLP-1 could be used in the treatment of heart failure (Nikolaidis, L. A. et al., Circulation, 110(8):955-961 (2004)), ischemia/reperfusion injury (Nikolaidis, L. A. et al., Circulation, 109(8):962-965 (2004)), and Alzheimer's Disease (Perry, T. et al., J. Alzheimers Dis., 4(6):487-496 (2002)).

GLP-1 is an incretin hormone, for example, an intestinal hormone that enhances meal-induced insulin secretion (Holst, J. J., Curr. Med. Chem., 6(11): 1005-1017 (1999)). It is a product of the glucagon gene encoding proglucagon. This gene is expressed not only in the A-cells of the pancreas but also in the endocrine L-cells of the intestinal mucosa. Proglucagon is a peptide (protein) containing 160 amino acids. Further processing of proglucagon results in the generation of: a) glucagon, b) an N-terminal, presumably inactive fragment, and c) a large C-terminal fragment commonly referred as “the major proglucagon fragment”. This fragment is considered to be biologically inactive. Even though this fragment is present in both the pancreas and in the L-cells of the gut, it is only in the intestines that the breakdown products of the “the major proglucagon fragment” resulting in two highly homologous compounds commonly referred as GLP-1 and GLP-2 are observed. These two compounds have important biological activities. As such, the amino acid sequence of GLP-1, which is present in the L-cells, is identical to amino acids 78-107 of proglucagon.

Presently, therapy involving the use of GLP-1-type molecules presents a significant challenge because the serum half-life of such compounds is quite short. For example, GLP-1 (7-37) has a serum half-life of less than five minutes. Thus, there exists a critical need for biologically active GLP-1 receptor modulators, agonists or partial agonists, that possess extended pharmacodynamic profiles. The present invention is directed to this and other needs.

The present invention provides novel compounds that act as GLP-1 receptor modulators, agonists, or partial agonists, which exhibit similar or superior biological properties of the native peptide, GLP-1, and thus are useful for the treatment and amelioration of the diabetic and related conditions.

### SUMMARY OF THE INVENTION

The synthetic isolated compounds described herein are capable of modulating the GLP-1 receptor, desirably as agonists or partial agonists. These synthetic compounds exhibit superior in vivo efficacy and pharmacokinetic properties relative to GLP-1, including postprandial plasma glucose lowering and concomitant increase in plasma insulin levels, thus making them ideal therapeutic candidates. The candidates may be administered via a number of routes including subcutaneous, pulmonary, nasal, buccal or sustained release formulations.

The GLP-1 analogs of the present invention may comprise amino acids in positions that are not in the native GLP-1 molecule, and typically comprise at least eleven contiguous amino acids.

One embodiment described herein is an isolated polypeptide comprising a sequence of Formula I:
Xₐₐ₁-Xₐₐ₂-Xₐₐ₃-Xₐₐ₄-Xₐₐ₅-Xₐₐ₆-Xₐₐ₇-Xₐₐ₈-Xₐₐ₉-Xₐₐ₁₀-Xₐₐ₁₁ I

wherein,

Xₐₐ₁ is a naturally or non-naturally occurring amino acid comprising an imidazole, such as histidine; wherein any of the carbon atoms of said amino acid are optionally substituted with hydrogen, or with one or more alkyl groups, wherein the free amino group of said amino acid is optionally substituted with hydrogen, alkyl, acyl, benzoyl, L-lactyl, alkyloxycarbonyl, aryloxycarbonyl, arylalkyloxycarbonyl, heterocyclyloxycarbonyl, heteroarylalkyloxycarbonyl, alkylcarbamoyl, arylcarbamoyl, arylalkylcarbamoyl, heterocyclylsulfonyl, alkylsulfonyl, arylsulfonyl, arylalkylsulfonyl, heteroarylalkylsulfonyl or heteroarylsulfonyl; and wherein the amino group of Xₐₐ1 is optionally absent, such that Xₐₐ1 is the des-amino acid of histidine in which any of the carbon atoms are optionally substituted with hydrogen or one or more alkyl groups; and wherein the amino group of Xₐₐ1 is optionally replaced with a hydroxyl group;

Xₐₐ₂ is a naturally or non-naturally occurring amino acid selected from the group consisting of alanine, α-amino-isobutyric acid (Aib), N-methyl-D-alanine, N-ethyl-D-alanine, 2-methyl-azetidine-2-carboxylic acid, alpha-methyl-(L)-proline, 2-methylpiperidine-2-carboxylic acid and isovaline;

Xₐₐ₃ is a naturally or non-naturally occurring amino acid comprising an amino acid side chain which contains a carboxylic acid, for example aspartic acid or glutamic acid; or wherein Xₐₐ₃ is a naturally or non-naturally occurring amino acid containing an imidazole side chain, for example histidine, and wherein any of the carbon atoms of said amino acid are optionally substituted with one or more alkyl groups;

Xₐₐ₄ is glycine;

Xₐₐ₅ is a naturally or non-naturally occurring amino acid selected from the group consisting of (L)-threonine, and (L)-norvaline; and wherein any of the carbon atoms of said amino acid are optionally substituted with one or more alkyl groups;

Xₐₐ₆ is a naturally or non-naturally occurring amino acid comprising an alpha carbon which is di-substituted; wherein one of the side chains of said amino acid contains an aromatic ring, for example alpha-methyl-phenylalanine, alpha-methyl-2-fluorophenylalanine, and alpha-methyl-2,6-difluorophenylalanine; wherein any of the carbon atoms of said amino acid are optionally substituted with one or more alkyl groups or one or more halo groups;

Xₐₐ₇ is a naturally or non-naturally occurring amino acid comprising an amino acid side chain which is substituted with a hydroxyl group, for example L-threonine; wherein any of the carbon atoms of said amino acid are optionally substituted with one or more alkyl groups;

Xₐₐ₈ is a naturally or non-naturally occurring amino acid selected from the group consisting of L-serine, and L-histidine; wherein one or more of the carbon atoms of said amino acid is optionally substituted with one or more alkyl groups;

Xₐₐ₉ is a naturally or non-naturally occurring amino acid comprising an amino acid side chain which contains a carboxylic acid, for example L-aspartic acid or L-glutamic acid; wherein one or more of the carbon atoms of said amino acid is optionally substituted with one or more alkyl groups;

Xₐₐ₁₀ is a naturally or non-naturally occurring amino acid of Formula II:

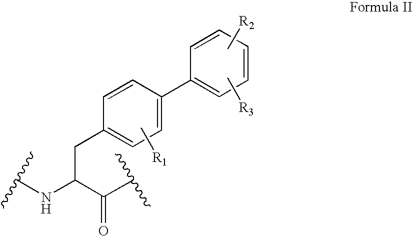

wherein R₁ is selected from the group consisting of hydrogen, alkyl, and halo;

wherein R₂ and R₃ are each independently selected from the group consisting of hydrogen, halo, methyl, ethyl, alkyl, hydroxyl, methoxy, and alkoxy;

the amino acid of Formula II may further comprise at least one R₁, R₂ or R₃ groups, which may or may not be equivalent; and

Xₐₐ₁₁ may be a naturally or non-naturally occurring amino acid of Formula III:

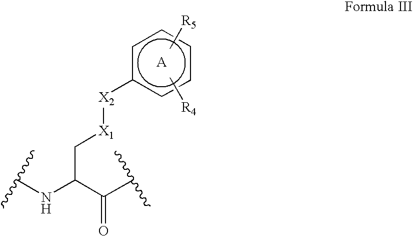

wherein the C-terminal carbonyl carbon of said amino acid is attached to a nitrogen to form a carboxamide (NH₂);

wherein ring A is selected from the group consisting of aryl and heteroaryl;

wherein R₄ and R₅ are each independently selected from the group consisting of hydrogen, halo, methyl, ethyl, alkyl, hydroxyl, methoxy, alkoxy, aryl, heteroaryl; and

wherein X₁ and X₂ are each CH-alkyl, CH₂, NH, S or O.

The amino acid of Formula III may further comprise at least one R₄ or R₅ groups, and, if more than one are present, may or may not be equivalent.

Xₐₐ₁₁ may be a naturally or non-naturally occurring amino acid of Formula IV:

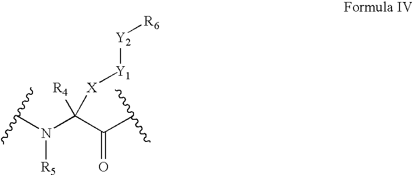

wherein the C-terminal carbonyl carbon of said amino acid is attached to a nitrogen to form a carboxamide (NH₂);

wherein R₄ is selected from the group consisting of hydrogen, hydroxyl, methyl, ethyl, alkyl, methoxy, alkoxy, aryl, heteroaryl;

wherein R₅ is selected from the group consisting of hydrogen, methyl, ethyl, alkyl, cycloalkyl, aryl, heteroaryl, alkylaryl, or heteroalkylaryl;

wherein X is selected from the group consisting of CH₂, CH₂CH₂, or CHCH₃;

wherein Y₁ is selected from the group consisting of —NH—, —O—, and —C═O—;

wherein Y₂ is selected from the group consisting of —C═O—, —O═C—O— and —SO₂— when Y₁ is NH or O;

wherein Y₂ is selected from the group consisting of —NH—, —N—, or —O— when Y₁ is C═O; and

wherein R₆ is selected from the group consisting of hydrogen, methyl, ethyl, alkyl, cycloalkyl, aryl, heteroaryl, alkylaryl, or alkylheteroaryl.

The amino acid of Formula IV comprises at least one R₆ group and, if more than one are present, may or may not, be equivalent.

Xₐₐ₁₁ may also be a naturally or non-naturally occurring amino acid of Formula V:

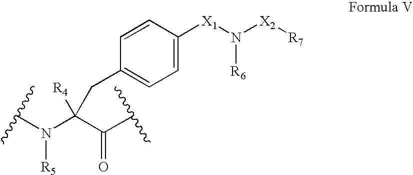

wherein the C-terminal carbonyl carbon of said amino acid is attached to a nitrogen to form a carboxamide (NH₂);

wherein R₄ is selected from the group consisting of hydrogen, hydroxyl, methyl, ethyl, alkyl, methoxy, alkoxy, aryl, heteroaryl;

wherein R₅ is selected from the group consisting of hydrogen, methyl, ethyl, alkyl, cycloalkyl, aryl, heteroaryl, alkylaryl, or heteroalkylaryl;

wherein X₁ is either absent or consists of CH₂;

wherein X₂ is selected from the group consisting of —CO—, CO—N(−)₂, —CO—O—, —SO—, and —SO₂—;

wherein R₆ and R₇ are independently selected from the group consisting of hydrogen, alkyl, alkenyl, alkynyl, cycloalkyl, aryl, heteroaryl, alkylaryl, or alkylheteroaryl;

The amino acid of Formula V comprises at least one R₇ group, and, if more than one are present, may or may not be equivalent.

Xₐₐ₁₁ may also be a naturally or non-naturally occurring amino acid of Formula VI:

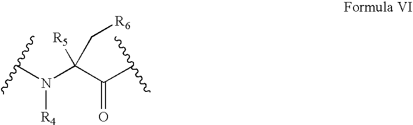

wherein the C-terminal carbonyl carbon of said amino acid is attached to a nitrogen to form a carboxamide (NH₂);

wherein R₄ is selected from the group consisting of hydrogen, methyl, ethyl, alkyl, cycloalkyl, aryl, heteroaryl, alkylaryl, or heteroalkylaryl;

wherein R₅ is selected from the group consisting of hydrogen, hydroxyl, methyl, ethyl, alkyl, methoxy, and alkoxy;

wherein R₆ is selected from the group consisting of hydrogen, methyl, ethyl, alkyl, cycloalkyl, heterocycloalkyl, hydroxyl, methoxy, and alkoxy.

The molecule of Formula VI may further comprise at least one R₆ group, and, if more than one are present, may or may not be equivalent.

The molecule of Formula VI may further comprise of R₅ and R₆ groups which together form a cycloalkyl, heterocycloalkyl, cycloalkylaryl, or cycloalkylheteroaryl group.

Another embodiment is an isolated polypeptide of Formula VII,

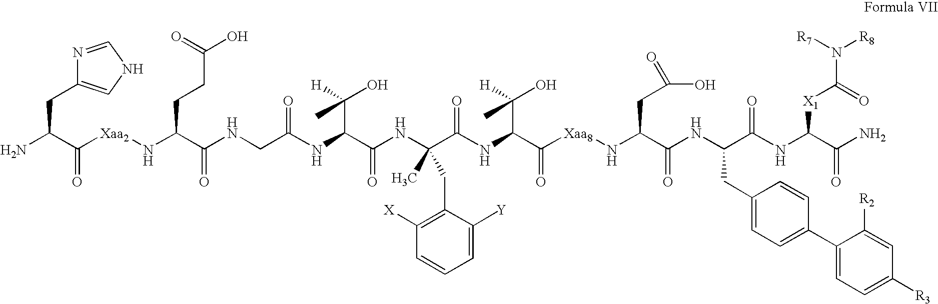

wherein:

Xₐₐ₂ is an amino acid selected from the group consisting of N-methyl-D-Ala, α-methyl-L-Pro, 2-methyl-azetidine-2-carboxylic acid, 2-methylpiperidine-2-carboxylic acid and aminoisobutyric (Aib);

X and Y are each independently selected from the group consisting of hydrogen and fluoro;

Xₐₐ₈ is an amino acid selected from the group consisting of L-Ser and L-His;

R₂ is selected from the group consisting of hydrogen, methyl and ethyl;

R₃ is selected from the group consisting of hydrogen, hydroxy, methoxy and ethoxy;

X₁ is selected from the group consisting of CH₂ and CH₂CH₂;

R₇ is selected from the group consisting of hydrogen, methyl, ethyl, alkyl, hydroxyl, methoxy, alkoxy, aryl, heteroaryl, alkylaryl, alkylheteroaryl; and

R₈ is selected from the group consisting of consisting of hydrogen, methyl, ethyl, alkyl, hydroxyl, methoxy, alkoxy, aryl, heteroaryl, alkylaryl, alkylheteroaryl

Another embodiment is an isolated polypeptide of Formula VIII,

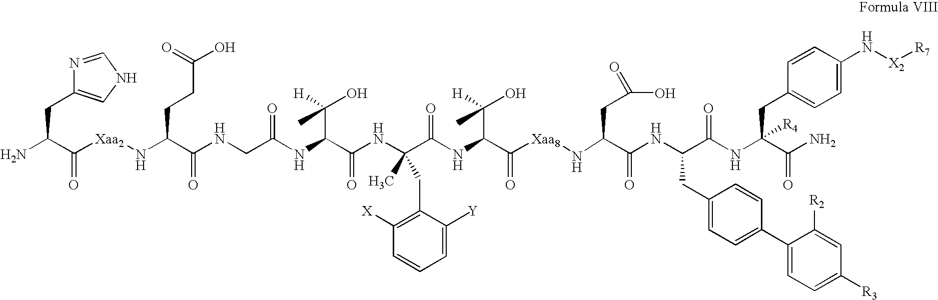

wherein:

Xₐₐ₂ is an amino acid selected from the group consisting of N-methyl-D-Ala, α-methyl-L-Pro, 2-methyl-azetidine-2-carboxylic acid, 2-methylpiperidine-2-carboxylic acid and aminoisobutyric (Aib);

X and Y are each independently selected from the group consisting of hydrogen and fluoro;

Xₐₐ₈ is an amino acid selected from the group consisting of L-Ser and L-His;

R₂ is selected from the group of hydrogen, methyl and ethyl;

R₃ is selected from the group of hydrogen, hydroxy, methoxy and ethoxy;

R₄ is selected from the group consisting of hydrogen and methyl;

X₂ is selected from the group consisting of —CO— and —SO₂—;

and R₇ is selected from the group consisting of alkyl, cycloalkyl, aryl, heteroaryl, alkylaryl, or alkylheteroaryl.

The peptide of Formula VIII comprises at least one R₇ group, and, if more than one are present, may or may not be equivalent.

Another embodiment is an isolated polypeptide of Formula IX

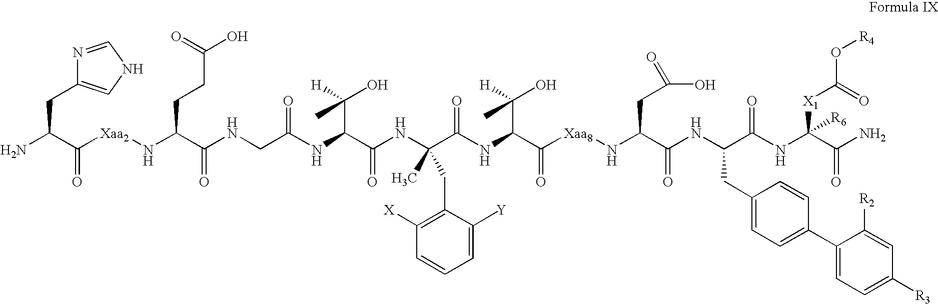

wherein:

Xₐₐ₂ is an amino acid selected from the group consisting of N-methyl-D-Ala, α-methyl-L-Pro, 2-methyl-azetidine-2-carboxylic acid, 2-methylpiperidine-2-carboxylic acid and aminoisobutyric (Aib);

X and Y are each independently selected from the group consisting of hydrogen and fluoro;

Xₐₐ₈ is an amino acid selected from the group consisting of L-Ser and L-His;

X₁ is selected from the group consisting of CH₂ and CH₂CH₂;

R₂ is selected from the group consisting of hydrogen, methyl and ethyl;

R₃ is selected from the group consisting of hydrogen, hydroxy, methoxy and ethoxy;

R₄ is selected from the group consisting of methyl, ethyl, alkyl, aryl, heteroaryl, alkylaryl, alkylheteroaryl; and

R₆ is hydrogen.

Another embodiment is an isolated polypeptide of Formula X:

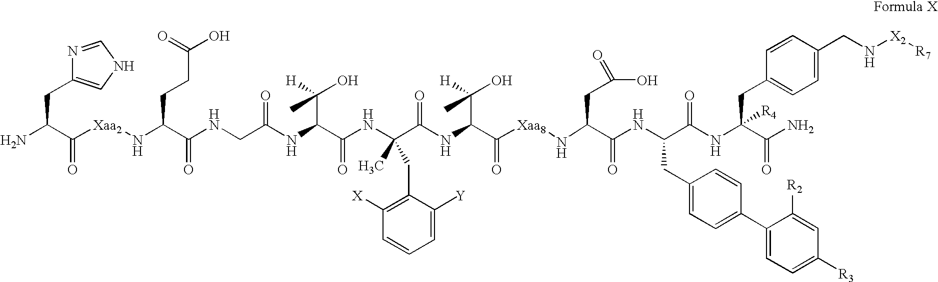

wherein:

Xₐₐ₂ is an amino acid selected from the group consisting of D-Ala, N-methyl-D-Ala, α-methyl-L-Pro, α-aminoisobutyric (Aib), 2-methyl-azetidine-2-carboxylic acid, and 2-methylpiperidine-2-carboxylic acid;

X and Y are each independently selected from the group consisting of hydrogen and fluoro;

Xₐₐ₈ is an amino acid selected from the group consisting of L-Ser and L-His;

R₂ is methyl or ethyl;

R₃ is selected from the group of hydrogen, methyl, ethyl, and methoxy;

R₄ is selected from the group consisting of hydrogen and methyl;

wherein X₂ is selected from the group consisting of —CO— and —SO₂—;

and wherein R₇ is selected from the group consisting of alkyl, cycloalkyl, aryl, heteroaryl, alkylaryl, or alkylheteroaryl.

The peptide of Formula X comprises at least one R₇ group, and, if more than one are present, may or may not be equivalent.

Another embodiment is an isolated polypeptide of Formula I, wherein Xₐₐ₁ is L-Histidine and wherein the terminal amino group is optionally substituted with hydrogen, alkyl, dialkyl, acyl, benzoyl, L-lactyl, alkyloxycarbonyl, aryloxycarbonyl, arylalkyloxycarbonyl, heterocyclyloxycarbonyl, heteroarylalkyloxycarbonyl, alkylcarbamoyl, arylcarbamoyl, aralkylcarbamoyl, heterocyclylsulfonyl, alkylsulfonyl, arylsulfonyl, arylalkylsulfonyl, heteroarylalkylsulfonyl or heteroarylsulfonyl.

Another embodiment is an isolated polypeptide of Formula I, wherein Xₐₐ₁ is selected from the group consisting of L-His, L-N-methyl-His, L-α-methyl-His, des-amino-His, 3-(1H-imidazol-4-yl)-2-methylpropanoyl, and (S)-3-(1H-imidazol-4-yl)-2-hydroxypropanoyl(L-β-imidazolelactyl).

Another embodiment is an isolated polypeptide of Formula I, wherein Xₐₐ₂ is selected from the group consisting of α-amino-isobutyric acid (Aib), D-alanine, N-methyl-D-alanine, alpha-methyl-(L)-proline, 2-methyl-azetidine-2-carboxylic acid and 2-methylpiperidine-2-carboxylic acid.

Another embodiment is an isolated polypeptide of Formula I, wherein Xₐₐ₃ is selected from the group consisting of L-glutamic acid and L-aspartic acid.

Another embodiment is an isolated polypeptide of Formula I, wherein Xₐₐ₄ is Gly.

Another embodiment is an isolated polypeptide of Formula I, wherein Xₐₐ₅ is selected from the group consisting of L-Thr, and L-Nva.

Another embodiment is an isolated polypeptide of Formula I, wherein Xₐₐ₆ is selected from the group consisting of L-α-Me-Phe, L-α-Me-2-fluoro-Phe, and L-α-Me-2,6-difluoro-Phe.

Another embodiment is an isolated polypeptide of Formula I, wherein Xₐₐ₇ is L-Thr.

Another embodiment is an isolated polypeptide of Formula I, wherein said Xₐₐ₈ is selected from the group consisting of L-Ser, and L-His.

Another embodiment is an isolated polypeptide of Formula I, wherein said Xₐₐ₉ is L-Asp.

Another embodiment is an isolated polypeptide of Formula I, wherein Xₐₐ₁₀, as Formula II, is selected from the group consisting of 4-phenyl-phenylalanine, 4-[(4′-methoxy-2′-ethyl)phenyl]phenylalanine, 4-[(4′-ethoxy-2′-ethyl)phenyl]phenylalanine, 4-[(4′-methoxy-2′-methyl)phenyl]phenylalanine, 4-[(4′-ethoxy-2′-methyl)phenyl]phenylalanine, 4-(2′-ethylphenyl)phenylalanine, 4-(2′-methylphenyl)phenylalanine, 4-[(3′,5′-dimethyl)phenyl]phenylalanine and 4-[(3′,4′-dimethoxy)phenyl]phenylalanine.

Another embodiment is an isolated polypeptide of Formula I, wherein Xₐₐ₁₁ is an amino acid selected from the group consisting of (S)-2-amino-5-phenylpentanoic acid, (S)-2-amino-4-phenoxybutanoic acid, (S)-2-amino-5-(4-chlorophenyl)pentanoic acid, (S)-2-amino-5-(quinolin-5-yl)pentanoic acid, and (S)-2-amino-4-(2-chlorophenoxy)butanoic acid; wherein the C-terminal carbonyl carbon of said amino acid is attached to a nitrogen to form a carboxamide (NH₂); and wherein R₆ is chosen from the group consisting of hydrogen and methyl.

A preferred embodiment is an isolated polypeptide Formula XI:

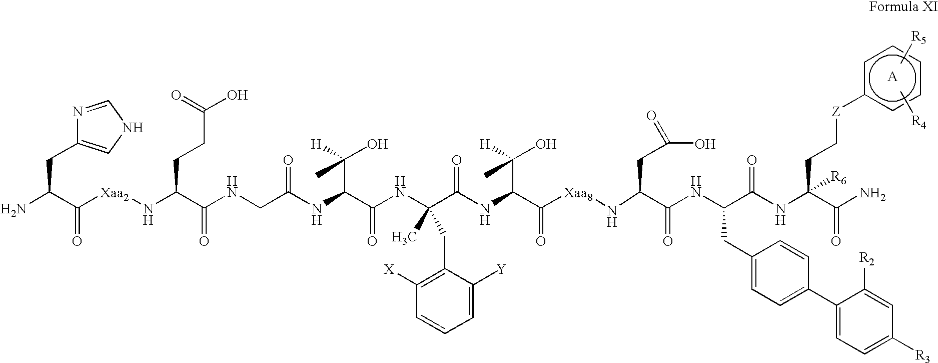

wherein Xₐₐ₂ is an amino acid selected from the group consisting of D-Ala, N-methyl-D-Ala, α-methyl-L-Pro, 2-methyl-azetidine-2-carboxylic acid, 2-methylpiperidine-2-carboxylic acid and α-aminoisobutyric (Aib);

X and Y are each independently selected from the group consisting of hydrogen and fluoro;

Xₐₐ₈ is an amino acid selected from the group consisting of L-Ser and L-His;

R₂ is selected from the group consisting of hydrogen, methyl and ethyl;

R₃ is selected from the group consisting of hydrogen, hydroxy, methoxy and ethoxy;

Z is selected from the group consisting of CH₂ and O;

wherein ring A is selected from the group consisting of aryl and heteroaryl;

R₄ is selected from the group consisting of hydrogen, fluoro, methyl and ethyl;

R₅ is selected from the group consisting of hydrogen, methyl and methoxy; and

R₆ is selected from the group consisting of hydrogen and methyl.

A more preferred embodiment is an isolated polypeptide of Formula XII, wherein:

Xₐₐ₂ is an amino acid selected from the group consisting of N-methyl-D-Ala, α-methyl-L-Pro and α-aminoisobutyric (Aib);

X is fluoro;

Y is hydrogen;

Z is selected from the group consisting of CH₂ and O;

Xₐₐ₈ is an amino acid selected from the group consisting of L-Ser and L-His;

R₂ is ethyl;

R₃ is methoxy;

R₄ is selected from the group consisting of hydrogen, methyl and ethyl;

R₅ is selected from the group consisting of hydrogen, methyl and ethyl; and

R₇ is selected from the group consisting of hydrogen.

Another preferred embodiment is an isolated polypeptide of Formula XII,

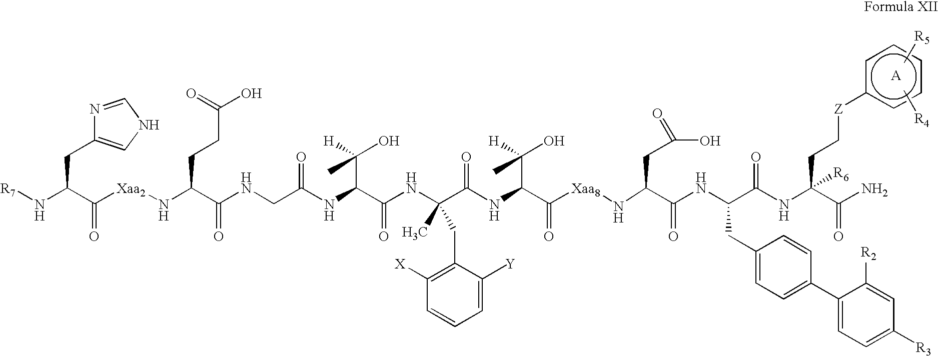

wherein:

R₇ is selected from the group consisting of methyl, ethyl,

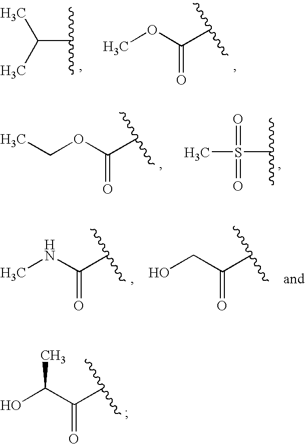

Xₐₐ₂ is an amino acid selected from the group consisting of D-Ala, N-methyl-D-Ala, α-methyl-L-Pro, α-aminoisobutyric (Aib), 2-methyl-azetidine-2-carboxylic acid, and 2-methylpiperidine-2-carboxylic acid;

X and Y are each independently selected from the group consisting of hydrogen and fluoro;

ring A is selected from the group consisting of aryl and heteroaryl;

Z is from the group of CH₂ and O;

Xₐₐ₈ is an amino acid selected from the group consisting of L-Ser and L-His;

R₂ is methyl or ethyl;

R₃ is selected from the group consisting of hydrogen, methyl, ethyl, and methoxy;

R₄ and R₅ are selected from the group consisting of hydrogen, methyl, ethyl, aryl, halo, or alkoxy; and

R₆ is selected from the group consisting of hydrogen, and methyl.

Another embodiment is an isolated polypeptide of Formula XIII,

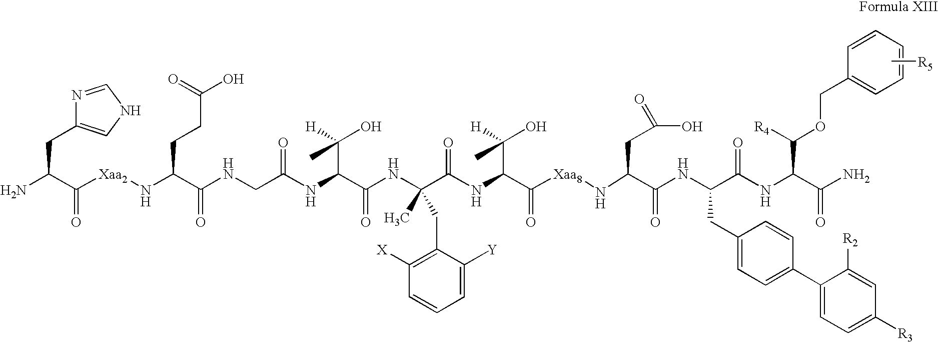

wherein:

Xₐₐ₂ is an amino acid selected from the group consisting of N-methyl-D-Ala, α-methyl-L-Pro, 2-methyl-azetidine-2-carboxylic acid, 2-methylpiperidine-2-carboxylic acid and aminoisobutyric (Aib);

X and Y are each independently selected from the group consisting of hydrogen and fluoro;

Xₐₐ₈ is an amino acid selected from the group consisting of L-Ser and L-His;

R₂ is selected from the group consisting of hydrogen, methyl and ethyl;

R₃ is selected from the group consisting of hydrogen, hydroxy, methoxy and ethoxy;

R₄ is chosen from the group of hydrogen or methyl;

R₅ is selected from the group consisting of hydrogen, halo, methyl, ethyl, alkyl, hydroxyl, methoxy, alkoxy, aryl, heteroaryl, alkylaryl, alkylheteroaryl; and

the compound may contain at least one R₅ group, and, if more than one are present, may or may not be identical.

Another embodiment is an isolated polypeptide of Formula XIV:

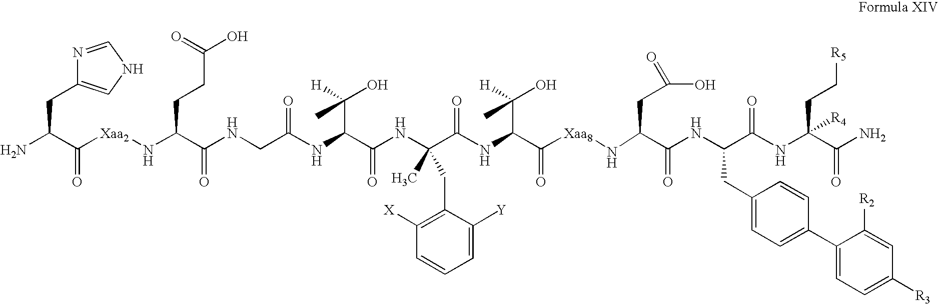

wherein:

Xₐₐ₂ is an amino acid selected from the group consisting of D-Ala, N-methyl-D-Ala, α-methyl-L-Pro, 2-methyl-azetidine-2-carboxylic acid, 2-methylpiperidine-2-carboxylic acid and α-aminoisobutyric (Aib);

X and Y are each independently selected from the group consisting of hydrogen and fluoro;

Xₐₐ₈ is an amino acid selected from the group consisting of L-Ser and L-His;

R₂ is selected from the group of hydrogen, methyl and ethyl;

R₃ is selected from the group of hydrogen, hydroxy, methoxy and ethoxy;

R₄ is selected from the group of hydrogen and methyl;

R₅ is selected from the group consisting of alkyl, heteroalkyl, cycloalkyl, or heterocycloalkyl.

A more preferred embodiment is an isolated polypeptide is chosen from polypeptides of Formula XIV, wherein:

Xₐₐ₂ is an amino acid selected from the group consisting of N-methyl-D-Ala, α-methyl-L-Pro and α-aminoisobutyric (Aib);

X is fluoro;

Y is hydrogen;

Xₐₐ₈ is an amino acid selected from the group consisting of L-Ser and L-His;

R₂ is ethyl;

R₃ is methoxy;

R₄ is selected from the group of hydrogen and methyl;

R₅ is selected from the group of methyl, ethyl, propyl, butyl, hexyl, cyclohexyl, and methylcyclohexyl;

R₄ and R₅ together comprise a cyclic moiety, including (but not limited to) cyclopentane and cyclohexane.

Another preferred embodiment is an isolated polypeptide of Formula XV:

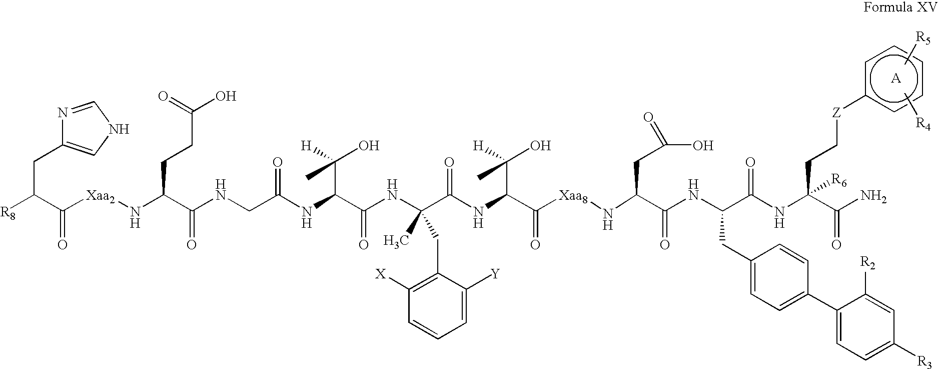

wherein:

R₈ is selected from the group consisting of hydrogen, hydroxyl, methyl and alkyl;

Xₐₐ₂ is an amino acid selected from the group consisting of D-Ala, N-methyl-D-Ala, α-methyl-L-Pro, α-aminoisobutyric acid (Aib), 2-methyl-azetidine-2-carboxylic acid and 2-methylpiperidine-2-carboxylic acid;

X and Y are each independently selected from the group consisting of hydrogen and fluoro;

Xₐₐ₈ is an amino acid selected from the group consisting of L-Ser and L-His;

Z is chosen from the group consisting of CH₂ and O;

ring A is selected from the group consisting of aryl and heteroaryl;

R₂ is methyl or ethyl;

R₃ is selected from the group consisting of hydrogen, methyl, methoxy and ethyl;

R₄ and R₅ are selected from the group consisting of hydrogen, methyl, ethyl, aryl, halo, or alkoxy; and

R₆ is selected from the group consisting of hydrogen and methyl.

Another preferred embodiment is an isolated polypeptide of Formula XV, wherein:

R₈ is selected from the group consisting of hydrogen and methyl;

Xₐₐ₂ is an amino acid selected from the group consisting of N-methyl-D-Ala, α-methyl-L-Pro, and α-aminoisobutyric acid (Aib);

X is fluoro;

Y is hydrogen;

Xₐₐ₈ is an amino acid selected from the group consisting of L-Ser and L-His;

R₂ is ethyl;

R₃ is methoxy;

R₄ is selected from the group consisting of methyl and ethyl;

R₅ is hydrogen; and

R₆ is selected from the group consisting of hydrogen and methyl.

Another embodiment is an isolated polypeptide of Formula XVI,

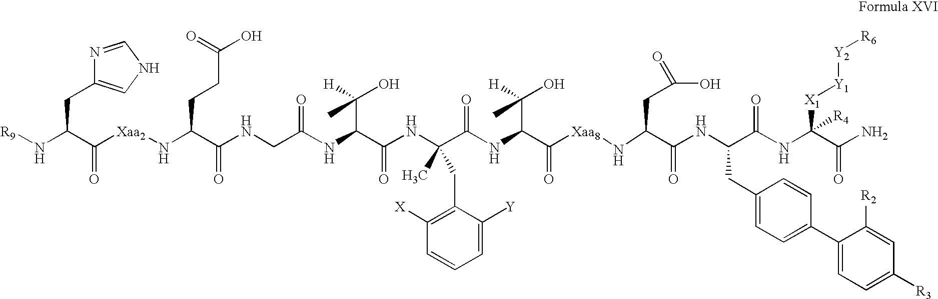

wherein:

R₉ is selected from the group consisting of methyl, ethyl,

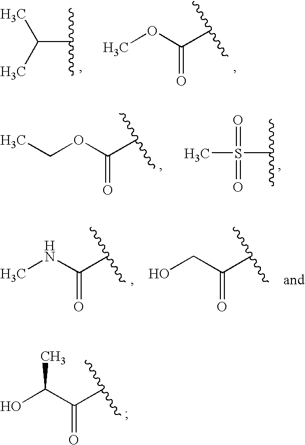

Xₐₐ₂ is an amino acid selected from the group consisting of D-Ala, N-methyl-D-Ala, α-methyl-L-Pro, α-aminoisobutyric (Aib), 2-methyl-azetidine-2-carboxylic acid, and 2-methylpiperidine-2-carboxylic acid;

X and Y are each independently selected from the group consisting of hydrogen and fluoro;

Xₐₐ₈ is an amino acid selected from the group consisting of L-Ser and L-His;

R₂ is methyl or ethyl;

R₃ is selected from the group consisting of hydrogen, methyl, ethyl, and methoxy;

R₄ is selected from the group consisting of hydrogen, methyl, ethyl, alkyl, aryl, or alkoxy;

wherein X₁ is selected from the group consisting of CH₂, CH₂CH₂, or CHCH₃;

wherein Y₁ is selected from the group consisting of —NH—, —O—, and —C═O—;

wherein Y₂ is selected from the group consisting of —C═O—, —O═C—O— and —SO₂— when Y₁ is NH or O;

wherein Y₂ is selected from the group consisting of —NH—, —N—, or —O— when Y₁ is C═O; and

wherein R₆ is selected from the group consisting of hydrogen, methyl, ethyl, alkyl, cycloalkyl, aryl, heteroaryl, alkylaryl, or alkylheteroaryl.

The peptide of Formula XVI comprises at least one R₆ group, and, if more than one are present, may or may not be equivalent.

Another embodiment is an isolated polypeptide of Formula XVII,

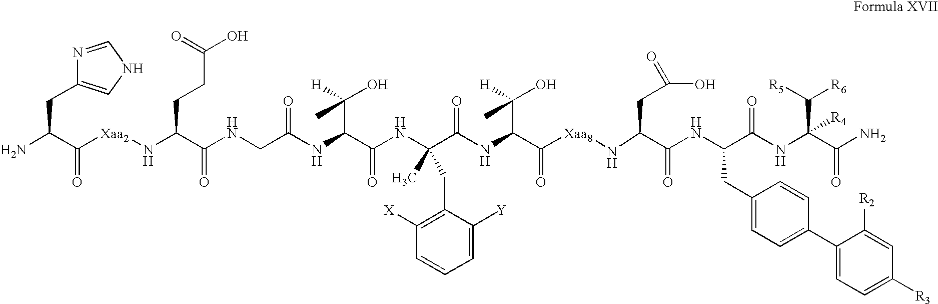

wherein:

Xₐₐ₂ is an amino acid selected from the group consisting of D-Ala, N-methyl-D-Ala, α-methyl-L-Pro, α-aminoisobutyric (Aib), 2-methyl-azetidine-2-carboxylic acid, and 2-methylpiperidine-2-carboxylic acid;

X and Y are each independently selected from the group consisting of hydrogen and fluoro;

Xₐₐ₈ is an amino acid selected from the group consisting of L-Ser and L-His;

R₂ is ethyl;

R₃ is methoxy;

R₄ is selected from the group of hydrogen and methyl;

R₅ is methyl;

R₆ is selected from the group of alkyl, heteroalkyl, cycloalkyl, and heterocycloalkyl.

Another embodiment is an isolated polypeptide of Formula XVIII,

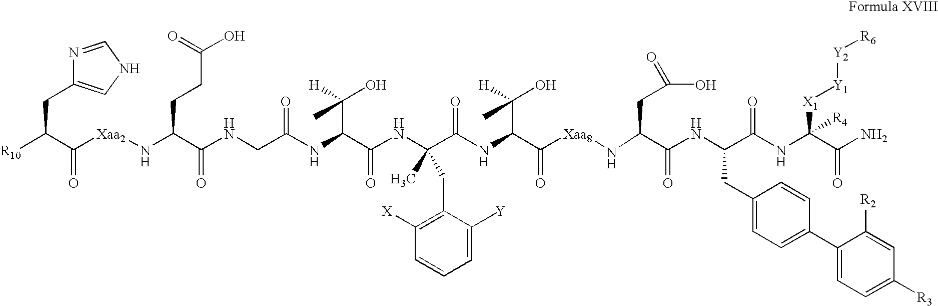

wherein:

R₁₀ is selected from the group consisting of hydrogen, hydroxyl, methyl and alkyl;

Xₐₐ₂ is an amino acid selected from the group consisting of D-Ala, N-methyl-D-Ala, α-methyl-L-Pro, α-aminoisobutyric acid (Aib), 2-methyl-azetidine-2-carboxylic acid and 2-methylpiperidine-2-carboxylic acid;

X and Y are each independently selected from the group consisting of hydrogen and fluoro;

Xₐₐ₈ is an amino acid selected from the group consisting of L-Ser and L-His;

R₂ is methyl or ethyl;

R₃ is selected from the group consisting of hydrogen, methyl, methoxy and ethyl;

R₄ is selected from the group consisting of hydrogen, methyl, ethyl, alkyl, aryl, or alkoxy;

wherein X₁ is selected from the group consisting of CH₂, CH₂CH₂, or CHCH₃;

wherein Y₁ is selected from the group consisting of —NH—, —O—, and —C═O—;

wherein Y₂ is selected from the group consisting of —C═O—, —O═C—O— and —SO₂— when Y₁ is NH or O;

wherein Y₂ is selected from the group consisting of —NH—, —N—, or —O— when Y₁ is C═O; and

wherein R₆ is selected from the group consisting of hydrogen, methyl, ethyl, alkyl, cycloalkyl, aryl, heteroaryl, alkylaryl, or alkylheteroaryl.

The peptide of Formula XVIII may further comprise at least one R₆ group and, if more than one are present, may, or may not, be equivalent.

Another embodiment is an isolated polypeptide of Formula XIX

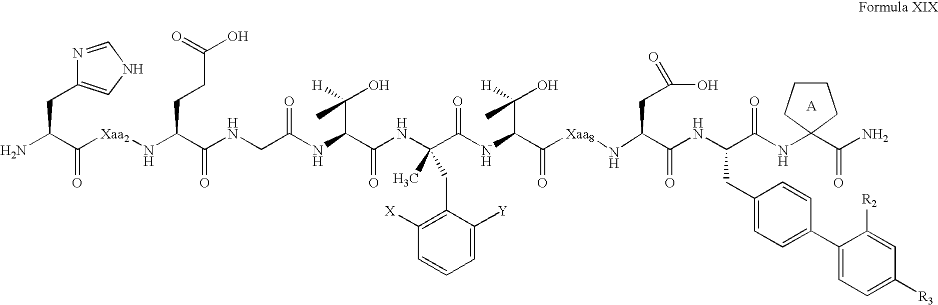

wherein:

Xₐₐ₂ is an amino acid selected from the group consisting of D-Ala, N-methyl-D-Ala, α-methyl-L-Pro, α-aminoisobutyric acid (Aib), 2-methyl-azetidine-2-carboxylic acid and 2-methylpiperidine-2-carboxylic acid;

X and Y are each independently selected from the group consisting of hydrogen and fluoro;

Xₐₐ₈ is an amino acid selected from the group consisting of L-Ser and L-His;

R₂ is methyl or ethyl;

R₃ is selected from the group of hydrogen, methyl, methoxy and ethyl;

R₄ is hydrogen or methyl;

Ring A is selected from the group of a cycloalkyl, cycloalkylaryl, heterocycloalkyl or cycloalkylheteroaryl.

Another embodiment is a compound of Formula XX:

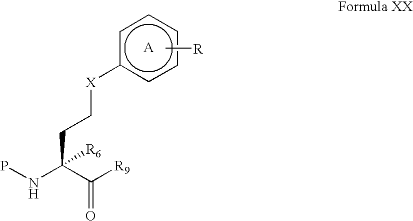

wherein:

P is hydrogen or fluorenylmethyloxycarbonyl (Fmoc) or t-butyloxycarbonyl (t-Boc);

Ring A is selected from the group consisting of aryl and heteroaryl;

R is selected from the group consisting of methyl, ethyl, chloro and fluoro;

R₆ is chosen from the group consisting of hydrogen and methyl;

R₉ is chosen from the group consisting of OH and NH₂;

X is chosen from the group consisting of CH₂ and O and NH and S; and

the peptide of Formula XX may further comprise at least one R group, and, if more than one are present, may or may not be equivalent.

Another embodiment is a compound of Formula XXI:

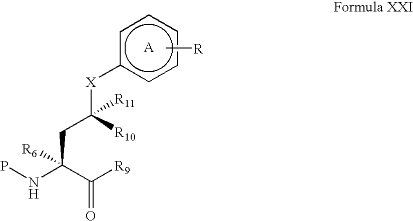

wherein:

P is hydrogen or fluorenylmethyloxycarbonyl (Fmoc) or t-butyloxycarbonyl (t-Boc);

R is selected from the group consisting of methyl, ethyl, chloro, and fluoro;

R₆ is chosen from the group consisting of hydrogen and methyl;

R₉ is chosen from the group consisting of OH and NH₂;

R₁₀ and R₁₁ are each chosen from the group consisting of hydrogen or ethyl or methyl;

X is chosen from the group consisting of CH₂ and O and NH and S; and

the molecule of Formula XXI may further comprise at least one R group, and, if more than one are present, may or may not be equivalent.

Preferably embodiments are include 11-mer to 15-mer peptides and such polypeptides bind to and activates the GLP-1 receptor.

Described herein are methods for making a polypeptide that mimics the activity of a GLP-1 receptor agonist.

The synthetic compounds described herein possess the ability to mimic the biological activity of GLP-1 peptides, with a preference for mimicking native GLP-1 activity. These synthetic GLP-1 mimics exhibit desirable in vivo properties, thus making them ideal therapeutic candidates for oral or parenteral administration.

Further described herein is an isolated polypeptide according to Formula I, wherein the polypeptide is a Glucagon-Like-Peptide derivative, preferably a Glucagon-Like-Peptide-1 derivative.

Further described are isolated peptides comprising a core sequence selected from the group consisting of: Thr-Ser-Asp-Bip-Xaa, wherein Xaa is an amino acid comprising a 2-amino-pentamide; Thr-Ser-Asp-Bip-Xaa, wherein Xaa is an amino acid comprising a 2-amino-butanamide; Thr-His-Asp-Bip-Xaa, wherein Xaa is an amino acid comprising a 2-amino-butanamide; Thr-Ser-Asp-Bip-Xaa, wherein Xaa is an amino acid comprising urea; Thr-Ser-Asp-Bip-Xaa, wherein Xaa comprises Glu; Thr-Ser-Asp-Bip-Xaa, wherein Xaa is an amino acid comprising 2-amino-propanoic acid; Thr-Ser-Asp-Bip-Xaa, wherein Xaa is an amino acid comprising a 3-amino-succinamide; Thr-Ser-Asp-Bip-Xaa, wherein Xaa is an amino acid comprising a 2-amino-propanamide; Thr-Ser-Asp-Bip-Xaa, wherein Xaa is an amino acid comprising an ocopropylcarbamate; Thr-Ser-Asp-Bip-Xaa, wherein Xaa is an amino acid comprising an isonicotinamide; Thr-Ser-Asp-Bip-Xaa, wherein Xaa is an amino acid comprising a methylpicolinamide; Thr-Ser-Asp-Bip-Xaa, wherein Xaa is an amino acid further comprising a 1- or 2-amino hexanoic, carboxylic, octanoic, decanoic, butanoic, pentanoic, and enoic acid; and Thr-Ser-Asp-Bip-Xaa, wherein Xaa comprises at least one amino acid coupled to a benzyl group; wherein said isolated peptide comprising said core sequence binds and activates a GLP-1 receptor.

Further described herein are compounds of Formula I, pharmaceutical compositions employing such compounds, and methods of using such compounds and compositions. In particular, the present invention provides a pharmaceutical composition comprising a therapeutically effective amount of a compound of Formula I, alone, or in combination, with a pharmaceutically acceptable carrier.

Further provided is a method for treating or delaying the progression or onset of diabetes, especially type II diabetes, including complications of diabetes, such as retinopathy, neuropathy, nephropathy, and delayed wound healing, and related diseases such as insulin resistance (impaired glucose homeostasis), hyperglycemia, hyperinsulinemia, elevated blood levels of fatty acids or glycerol, obesity, hyperlipidemia, including hypertriglyceridemia, Syndrome X, atherosclerosis, and hypertension, and for increasing high density lipoprotein levels, wherein a therapeutically effective amount of a compound of Formula I is administered to a mammalian, e.g. human, patient in need of treatment.

The compounds can be used alone, in combination with other compounds of the present invention, or in combination with one or more other agent(s) active in the therapeutic areas described herein.

In addition, a method is provided for treating diabetes and related diseases as defined above and hereinafter, wherein a therapeutically effective amount of a combination of a compound of Formula I and at least one other type of therapeutic agent, such as an antidiabetic agent, a hypolipidemic agent or anti-obesity agent, is administered to a human patient in need of treatment.

### DETAILED DESCRIPTION OF THE INVENTION

### Figures

FIG. 1 illustrates the effects of subcutaneous injection of a peptide of SEQ ID NO: 1 on plasma glucose in an ipGTT model in ob/ob mice.

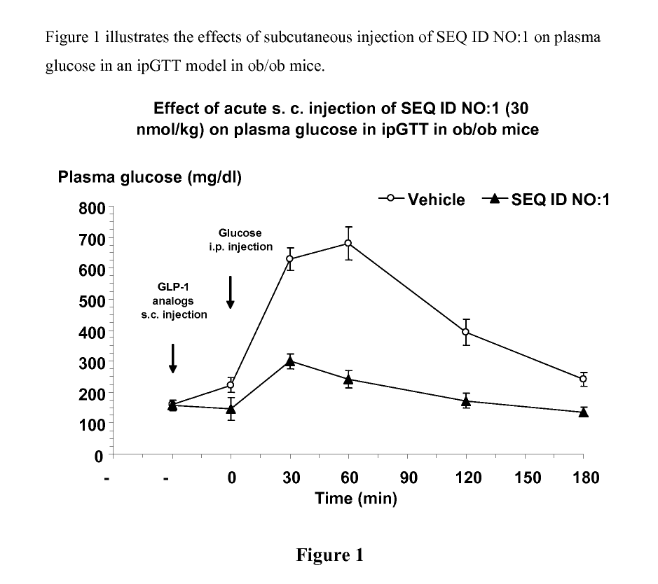

### DEFINITIONS

The definitions provided herein apply, without limitation, to the terms as used throughout this specification, unless otherwise limited in specific instances.

Those skilled in the art of amino acid and peptide chemistry are aware that an amino acid includes a compound represented by the general structure:

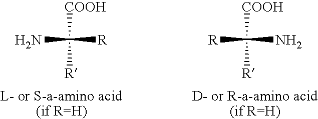

Where R and R′ are as discussed herein. Unless otherwise indicated, the term “amino acid” as employed herein alone, or as part of another group includes, without limitation, an amino group and a carboxyl group linked to the same carbon, referred to as “α” carbon, where R and/or R′ can be a natural or an un-natural side chain, including hydrogen. The absolute “S” configuration at the “α” carbon is commonly referred to as the “L” or “natural” configuration. In the case where both the “R” and the “R′” substituents equal hydrogen, the amino acid is glycine and is not chiral.

Unless otherwise indicated, the term “amino-alcohol” as employed herein alone, or as part of another group, includes, without limitation, a natural or un-natural amino acid in which the carboxy group is replaced (reduced) to a methyl alcohol such as valinol, glycinol, alaminol, arylalaminol, heteroarylalaminol.

Unless otherwise indicated, the term “alkyl” as employed herein alone, or as part of another group, includes, without limitation, both straight and branched chain hydrocarbons, containing 1 to 40 carbons, preferably 1 to 20 carbons, more preferably 1 to 8 carbons, in the normal chain, such as methyl, ethyl, propyl, isopropyl, butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, the various branched chain isomers thereof, and the like. Further, alkyl groups, as defined herein, may optionally be substituted on any available carbon atom with one or more functional groups commonly attached to such chains, such as, but not limited to alkyl, aryl, alkenyl, alkynyl, hydroxy, arylalkyl, cycloalkyl, cycloalkylalkyl, alkoxy, arylalkyloxy, heteroaryloxy, heteroarylalkyloxy, alkanoyl, halo, hydroxyl, thio, nitro, cyano, carboxyl, carbonyl 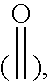
carboxamido, amino, alkylamino, dialkylamino, amido, alkylamino, arylamido, heterarylamido, azido, guanidino, amidino, phosphonic, phosphinic, sulfonic, sulfonamido, haloaryl, CF₃, OCF₂, OCF₃, aryloxy, heteroaryl, cycloalkylalkoxyalkyl, cycloheteroalkyl and the like to form alkyl groups such as trifluoro methyl, 3-hydroxyhexyl, 2-carboxypropyl, 2-fluoroethyl, carboxymethyl, cyanobutyl and the like.

Unless otherwise indicated, the term “alkenyl” as employed herein alone, or as part of another group, includes, without limitation, both straight and branched chain hydrocarbons, containing 2 to 40 carbons with one or more double bonds, preferably 2 to 20 carbons with one to three double bonds, more preferably 2 to 8 carbons with one to two double bonds, in the normal chain, such that any carbon may be optionally substituted as described above for “alkyl”.

Unless otherwise indicated, the term “alkynyl” as employed herein alone, or as part of another group, includes, without limitation, both straight and branched chain hydrocarbons, containing 2 to 40 carbons with one or more triple bonds, preferably 2 to 20 carbons with one to three triple bonds, more preferably 2 to 8 carbons with one to two triple bonds, in the normal chain, such that any carbon may be optionally substituted as described above for “alkyl”.

Unless otherwise indicated, the term “cycloalkyl” as employed herein alone, or as part of another group, includes, without limitation, saturated or partially unsaturated (containing 1 or 2 double bonds) cyclic hydrocarbon groups containing 1 to 3 rings, appended or fused, including monocyclic alkyl, bicyclic alkyl and tricyclic alkyl, containing a total of 3 to 20 carbons forming the rings, preferably 4 to 7 carbons, forming each ring; which may be fused to 1 aromatic ring as described for aryl, which include cyclopropyl, cyclobutyl, cyclopentyl, cyclohexyl, cycloheptyl, cyclooctyl, cyclodecyl, cyclododecyl, cyclohexenyl, 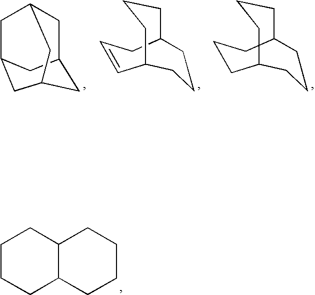
any of which groups may be optionally substituted through any available carbon atoms with 1 or more groups selected from hydrogen, halo, haloalkyl, alkyl, haloalkyl, alkoxy, haloalkoxy, alkenyl, trifluoromethyl, trifluoromethoxy, alkynyl, cycloalkylalkyl, fluorenyl, heterocycloalkyl, heterocycloalkylalkyl, aryl, heteroaryl, arylalkyl, aryloxy, aryloxyalkyl, arylalkoxy, arylthio, arylazo, heteroarylalkyl, heteroarylalkenyl, heteroarylheteroaryl, heteroaryloxy, hydroxy, nitro, oxo, cyano, carboxyl, carbonyl 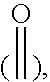
carboxamido, amino, substituted amino wherein the amino includes 1 or 2 substituents (which are alkyl, aryl or any of the other aryl compounds mentioned in the definitions), amido, azido, guanidino, amidino, phosphonic, phosphinic, sulfonic, sulfonamido, thiol, alkylthio, arylthio, heteroarylthio, arylthioalkyl, alkoxyarylthio, alkylcarbonyl, arylcarbonyl, alkylaminocarbonyl, arylaminocarbonyl, alkoxycarbonyl, aminocarbonyl, alkylcarbonyloxy, arylcarbonyloxy, alkylcarbonylamino, arylcarbonylamino, arylsulfinyl, arylsulfinylalkyl, arylsulfonylamino or arylsulfonaminocarbonyl, or any of alkyl substituents as set out above.

The term “aryl” as employed herein alone or as part of another group refers, without limitation, to monocyclic and bicyclic aromatic groups containing 6 to 10 carbons in the ring portion (such as phenyl or naphthyl) and may optionally include one to three additional rings fused to “aryl” (such as aryl, cycloalkyl, heteroaryl or heterocycloalkyl rings) and may be optionally substituted through any available carbon atoms with 1 or more groups selected from hydrogen, alkyl, halo, haloalkyl, alkoxy, haloalkoxy, alkenyl, trifluoromethyl, trifluoromethoxy, alkynyl, cycloalkylalkyl, fluorenyl, heterocycloalkyl, heterocycloalkylalkyl, aryl, heteroaryl, arylalkyl, aryloxy, aryloxyalkyl, arylalkoxy, arylthio, arylazo, heteroarylalkyl, heteroarylalkenyl, heteroaryloxy, hetroarylalkyloxy, hetroarylalkyloxyalkyl, hydroxy, nitro, oxo, cyano, amino, substituted amino wherein the amino includes 1 or 2 substituents (which are alkyl, cycloalkyl, heterocycloalkyl, heteroaryl, or aryl or any of the other aryl compounds mentioned in the definitions), thiol, alkylthio, arylthio, heteroarylthio, arylthioalkyl, alkoxyarylthio, alkylcarbonyl, arylcarbonyl, alkylaminocarbonyl, cycloalkylaminocarbonyl, arylaminocarbonyl, heteroarylaminocarbonyl, heteroarylalkylaminocarbonyl, alkoxycarbonyl, aminocarbonyl, alkylcarbonyloxy, arylcarbonyloxy, alkylcarbonylamino, arylcarbonylamino, arylsulfinyl, arylsulfinylalkyl, arylsulfonylamino or arylsulfonaminocarbonyl, or any of alkyl substituents as set out above.

The term “arylalkyl” as used herein alone or as part of another group refers, without limitation, to alkyl groups as defined above having an aryl substituent, such as benzyl, phenethyl or naphthylpropyl, wherein said aryl and/or alkyl groups may optionally be substituted as defined above.

The term “alkoxy”, “aryloxy”, “heteroaryloxy”, “arylalkyloxy” or “heteroarylalkyloxy” as employed herein alone, or as part of another group, includes, without limitation, an alkyl or aryl group as defined above linked through an oxygen atom.

The term “heterocyclo”, “heterocycle”, “heterocyclyl” or “heterocyclic”, as used herein, represents, without limitation, an unsubstituted or substituted stable 4-, 5-, 6- or 7-membered monocyclic ring system which may be saturated or unsaturated, and which consists of carbon atoms and from one to four heteroatoms selected from nitrogen, sulfur, oxygen and/or a SO or SO₂ group, wherein the nitrogen and sulfur heteroatoms may optionally be oxidized, and the nitrogen heteroatom may optionally be quaternized. The heterocyclic ring may be attached at any heteroatom or carbon atom which results in the creation of a stable structure. Examples of such heterocyclic groups include, but is not limited to, tetrahydrofuranyl, tetrahydrothiophenyl pyrrolidinyl, piperidinyl, piperazinyl, oxopyrrolidinyl, oxopiperazinyl, oxopiperidinyl and oxadiazolyl. Optionally a heterocyclo group may be substituted with one or more functional groups, such as those described for “alkyl” or “aryl”.

The term “heterocycloalkyl” as used herein alone or as part of another group refers, without limitation, to alkyl groups as defined above having a heterocycloalkyl substituent, wherein said “heterocyclo” and/or alkyl groups may optionally be substituted as defined above.

The term “heteroaryl” as used herein refers, without limitation, to a 5-, 6- or 7-membered aromatic heterocyclic ring which contains one or more heteroatoms selected from nitrogen, sulfur, oxygen and/or a SO or SO₂ group. Such rings may be fused to another aryl or heteroaryl ring and include possible N-oxides. Examples of such heteroaryl groups include, but are not limited to, furan, pyrrole, thiophene, pyridine, pyrimidine, pyrazine, pyridazine, isoxazole, oxazole, imidazole and the like. Optionally a heteroaryl group may be substituted with one or more functional groups commonly attached to such chains, such as those described for “alkyl” or “aryl”.

The term “heteroarylalkyl” as used herein alone or as part of another group refers, without limitation, to alkyl groups as defined above having a heteroaryl substituent, wherein said heteroaryl and/or alkyl groups may optionally be substituted as defined above.

The term “alkyloxycarbonyl” as used herein alone or as part of another group refers, without limitation, to alkyl groups as defined above attached to the oxygen of an —OC(O)— group, for example CH₃OC(O)—, CH₃CH₂OC(O)— or CH₂(OH)CH₂OC(O)—.

The term “aryloxycarbonyl” as used herein alone or as part of another group refers, without limitation, to aryl groups as defined above attached to the oxygen of an —OC(O)— group.

The term “arylalkyloxycarbonyl” as used herein alone or as part of another group refers, without limitation, to aralkyl groups as defined above attached to the oxygen of an —OC(O)— group.

The term “heterocyclyloxycarbonyl” as used herein alone or as part of another group refers, without limitation, to heterocyclyl groups as defined above attached by any carbon atom of the heterocyclyl group to the oxygen of an —OC(O)— group.

The term “heteroarylalkyloxycarbonyl” as used herein alone or as part of another group refers, without limitation, to heteroarylalkyl groups as defined above attached by any carbon atom of the heterocyclyl group to the oxygen of an —OC(O)— group.

The term “alkylcarbamoyl” as used herein alone or as part of another group refers, without limitation, to alkyl groups as defined above attached to the nitrogen of a —NC(O)— group, for example CH₃NHC(O)—, CH₃CH₂NHC(O)— or (CH₃)₂NHC(O)— and wherein 2 alkyl groups are present, the alkyl groups can optionally be attached to form a 4, 5, 6 or 7 membered ring, for example,

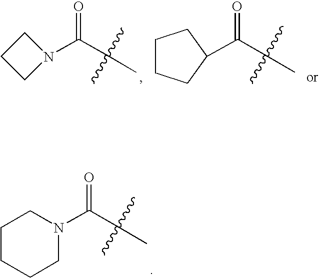

The term “arylalkylcarbamoyl” as used herein alone or as part of another group refers, without limitation, to arylalkyl groups as defined above attached to the nitrogen of a —NC(O)— group.

The term “heterocyclylcarbamoyl” as used herein alone or as part of another group refers, without limitation, to heterocyclyl groups as defined above attached to the nitrogen of an —NC(O)— group.

The term “alkylsulfonyl” as used herein alone or as part of another group refers, without limitation, to alkyl groups as defined above attached to the sulfur of an —S(O)₂— group for example CH₃S(O)₂—, CH₃CH₂S(O)₂— or (CH₃)₂CH₂S(O)₂—.

The term “arylsulfonyl” as used herein alone or as part of another group refers, without limitation, to aryl groups as defined above attached to the sulfur of an —S(O)₂— group.

The term “arylalkylsulfonyl” as used herein alone or as part of another group refers, without limitation, to arylalkyl groups as defined above attached to the sulfur of an —S(O)₂— group.

The term “heteroarylsulfonyl” as used herein alone or as part of another group refers, without limitation, to heteroaryl groups as defined above attached to the sulfur of an —S(O)₂— group.

The term “heteroarylalkylsulfonyl” as used herein alone or as part of another group refers, without limitation, to heteroarylalkyl groups as defined above attached to the sulfur of an —S(O)₂— group.

The term “receptor modulator” refers to a compound that acts at the GLP-1 receptor to alter its ability to regulate downstream signaling events. Examples of receptor modulators include agonists, antagonists, partial agonists, inverse agonists, allosteric antagonists and allosteric potentiators as defined in standard pharmacology textbooks (e.g., E. M. Ross and T. P. Kenakin in Goodman and Gilman's The Pharmacological Basis of Therapeutics, 10th Ed., Chapter 2, pp. 31-43, McGraw Hill, New York (2001)).

One of skill in the art will readily appreciate the meaning of such terms as provided in the present case and in the art.

The term “diabetes and related diseases or related conditions” refers, without limitation, to Type II diabetes, Type I diabetes, impaired glucose tolerance, obesity, hyperglycemia, Syndrome X, dysmetabolic syndrome, diabetic complications, and hyperinsulinemia.

The term “lipid-modulating” or “lipid lowering” agent as employed herein refers, without limitation, to agents that lower LDL and/or raise HDL and/or lower triglycerides and/or lower total cholesterol and/or other known mechanisms for therapeutically treating lipid disorders.

An administration of a therapeutic agent of the invention includes, without limitation, administration of a therapeutically effective amount of the agent of the invention. The term “therapeutically effective amount” as used herein refers, without limitation, to an amount of a therapeutic agent to treat or prevent a condition treatable by administration of a composition of the invention. That amount is the amount sufficient to exhibit a detectable therapeutic or preventative or ameliorative effect. The effect may include, for example and without limitation, treatment or prevention of the conditions listed herein. The precise effective amount for a subject will depend upon the subject's size and health, the nature and extent of the condition being treated, recommendations of the treating physician, and the therapeutics or combination of therapeutics selected for administration. Thus, it is not useful to specify an exact effective amount in advance.

The compounds and analogs thereof described herein may be produced by chemical synthesis using various solid-phase techniques such as those described in G. Barany and R. B. Merrifield, The Peptides: Analysis, Synthesis, Biology, Vol. 2, “Special Methods in Peptide Synthesis, Part A”, pp. 3-284, E. Gross and J. Meienhofer, eds., Academic Press, New York (1980); and in J. M. Stewart and J. D. Young, Solid Phase Peptide Synthesis, 2nd Ed., Pierce Chemical Co., Rockford, Ill., (1984). The desired strategy for use in this invention is based on the Fmoc (9-Fluorenylmethyloxycarbonyl) group for temporary protection of the α-amino group, in combination with the tert-butyl group for temporary protection of the amino acid side chains (see for example E. Atherton and R. C. Sheppard, “The Fluorenylmethoxycarbonyl Amino Protecting Group”, in The Peptides: Analysis, Synthesis, Biology, Vol. 9, “Special Methods in Peptide Synthesis, Part C”, pp. 1-38, S. Undenfriend and J. Meienhofer, eds., Academic Press, San Diego (1987).

The compounds can be synthesized in a stepwise manner on an insoluble polymer support (also referred to as “resin”) starting from the C-terminus of the compound. A synthesis is begun by appending the C-terminal amino acid of the peptide to the resin through formation of an amide or ester linkage. This allows the eventual release of the resulting peptide as a C-terminal amide or carboxylic acid, respectively.

The C-terminal amino acid and all other amino acids used in the synthesis are required to have their α-amino groups and side chain functionalities (if present) differentially protected such that the α-amino protecting group may be selectively removed during the synthesis. The coupling of an amino acid is performed by activation of its carboxyl group as an active ester and reaction thereof with the unblocked α-amino group of the N-terminal amino acid appended to the resin. The sequence of α-amino group deprotection and coupling is repeated until the entire peptide sequence is assembled. The peptide is then released from the resin with concomitant deprotection of the side chain functionalities, usually in the presence of appropriate scavengers to limit side reactions. The resulting peptide is finally purified by reverse phase HPLC.

The synthesis of the peptidyl-resins required as precursors to the final compounds utilizes commercially available cross-linked polystyrene polymer resins (Novabiochem, San Diego, Calif.; Applied Biosystems, Foster City, Calif.). Preferred solid supports for use in this invention are: 4-(2′,4′-dimethoxyphenyl-Fmoc-aminomethyl)-phenoxyacetyl-p-methyl benzhydrylamine resin (Rink amide MBHA resin); 9-Fmoc-amino-xanthen-3-yloxy-Merrifield resin (Sieber amide resin); 4-(9-Fmoc)aminomethyl-3,5-dimethoxyphenoxy)valeryl-aminomethyl-Merrifield resin (PAL resin), for C-terminal carboxamides. Coupling of first and subsequent amino acids can be accomplished using HOBT or HOAT active esters produced from DIC/HOBT, HBTU/HOBT, BOP, PyBOP, or from DIC/HOAT, HATU/HOAT, respectively. Preferred solid supports for use in this invention are: 2-Chlorotrityl chloride resin and 9-Fmoc-amino-xanthen-3-yloxy-Merrifield resin (Sieber amide resin) for protected peptide fragments. Loading of the first amino acid onto the 2-chlorotrityl chloride resin is best achieved by reacting the Fmoc-protected amino acid with the resin in dichloromethane and DIEA. If necessary, a small amount of DMF may be added to facilitate dissolution of the amino acid.

The syntheses of the GLP-1 peptide analogs described herein can be carried out by using a peptide synthesizer, such as an Advanced Chemtech Multiple Peptide Synthesizer (MPS396) or an Applied Biosystems Inc. peptide synthesizer (ABI 433A). If the MPS396 was used, up to 96 compounds were simultaneously synthesized. If the ABI 433A synthesizer was used, individual compounds were synthesized sequentially. In both cases the stepwise solid phase peptide synthesis was carried out utilizing the Fmoc/t-butyl protection strategy described herein.

The non-natural non-commercial amino acids present at position-Xₐₐ₁₁ were incorporated into the peptide chain in one of two methods. In the first approach the required non-natural amino acid was built on the resin directly using synthetic organic chemistry procedures. Alternatively, a Boc- or Fmoc-protected non-natural amino acid was prepared in solution using appropriate organic synthetic procedures. The resulting derivative was then used in the step-wise synthesis of the peptide, or in a fragment condensation approach to assemble the final peptide. When a non-natural non-commercial amino acid was needed for incorporation at position Xₐₐ₆, Xₐₐ₁₀, or at any other Xₐₐ position, the required Fmoc-protected non-natural amino acid was synthesized in solution. Such a derivative was then used in stepwise solid phase peptide synthesis.

Desired for use in the present invention are the Fmoc amino acids derivatives shown below.

### Orthogonally Protected Amino Acids Used in Solid Phase Synthesis

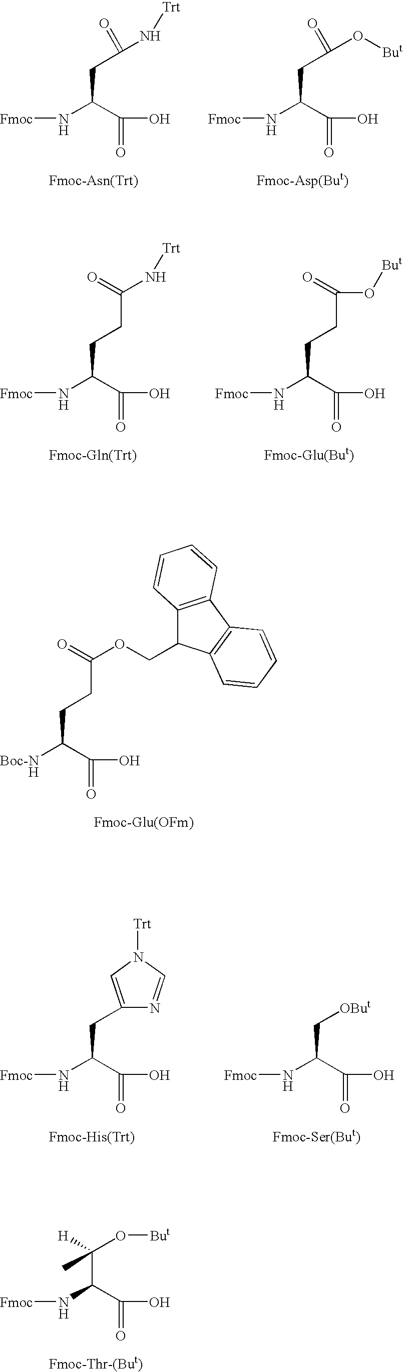

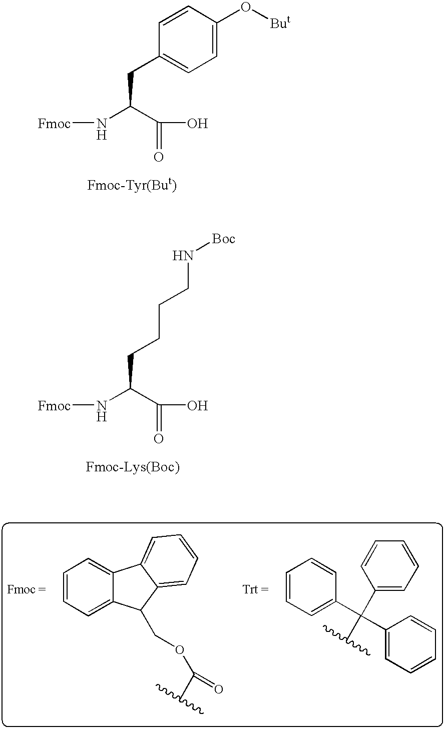

### Protected Amino Acids Used in Solid Phase Synthesis

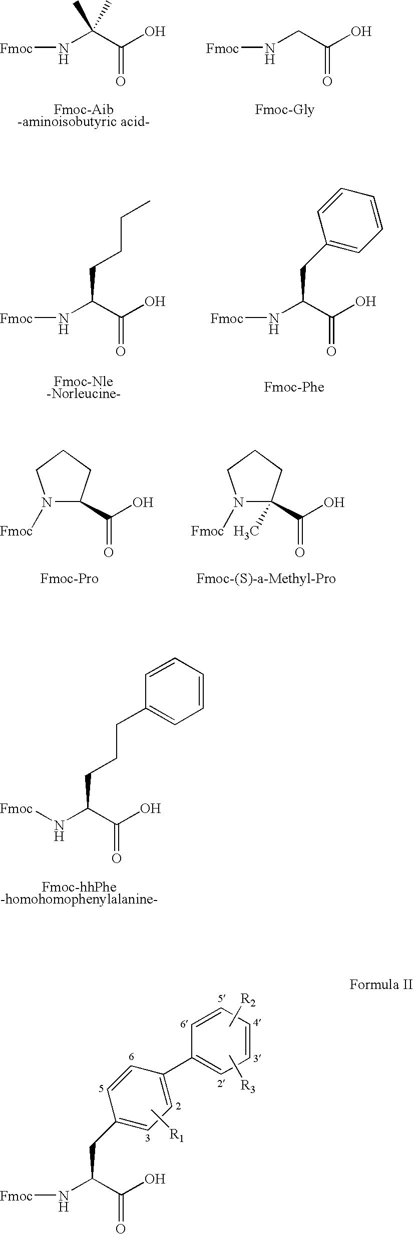

The peptidyl-resin precursors for their respective peptides may be cleaved and deprotected using any standard procedure (see, for example, D. S. King et al. Int. J. Pept. Protein Res., 36(3):255-266 (1990)). A desired method is the use of TFA in the presence of water and TIS as scavengers. Typically, the peptidyl-resin is stirred in TFA/water/TIS (94:3:3, v:v:v; 1 mL/100 mg of peptidyl resin) for 2-6 hours at room temperature. The spent resin is then filtered off and the TFA solution is concentrated or dried under reduced pressure. The resulting crude peptide is either precipitated and washed with Et₂O or is redissolved directly into DMSO or 50% aqueous acetic acid for purification by preparative HPLC.

Compounds with the desired purity can be obtained by purification using preparative HPLC, for example, on a Waters Model 4000 or a Shimadzu Model LC-8A liquid chromatograph. The solution of crude is injected into a YMC S5 ODS (20×100 mm) column and eluted with a linear gradient of MeCN in water, both buffered with 0.1% TFA, using a flow rate of 14-20 mL/min with effluent monitoring by UV absorbance at 220 nm. The structures of the purified compounds can be confirmed by electro-spray MS analysis.

Abbreviations

The following abbreviations are employed in the Examples and elsewhere herein:

| | |
|---|---|
| Ph = phenyl | DMA = N,N-dimethylacetamide |
| Bn = benzyl | DMAP = 4-(dimethylamino)pyridine |
| i-Bu = iso-butyl | DMF = N,N-dimethylformamide |
| i-Pr = iso-propyl | EDAC = 3-ethyl-3′-(dimethylamino)propyl- |
| Me = methyl | carbodiimide hydrochloride (or 1-[(3- |
| Et = ethyl | (dimethyl)amino)propyl])-3-ethylcarbodiimide |
| Pr = n-propyl | hydrochloride) |
| Bu = n-butyl | Fmoc or FMOC = fluorenylmethyloxycarbonyl |
| O-Bu^{t} or OtBu = tert-butyl | GTT = glucose tolerance test |
| TMS = trimethylsilyl | HATU = O-(7-Azabenzotriazol-1-yl)-1,1,3,3- |
| TIS or TIPS = Triisopropylsilane | tetramethyluronium hexafluorophosphate |
| Et₂O = diethyl ether | HBTU = 2-(1H-Benzotriazol-1-yl)-1,1,3,3- |
| HOAc or AcOH = acetic acid | tetramethyluronium hexafluorophosphate |
| AcCN or MeCN or CH₃CN = acetonitrile | HCTU = 2-(6-Chloro-1-H-benzotriazol-1-yl)- |
| EtOAc = ethyl acetate | 1,1,3,3-tetramethyluronium |
| THF = tetrahydrofuran | hexafluorophosphate |
| TFA = trifluoroacetic acid | HOAT = 1-hydroxy-7-azabenzotriazole |
| TFE = α,α,α-trifluoroethanol | HOBT or HOBT.H₂O = 1- |
| Et₂NH = diethylamine | hydroxybenzotriazole hydrate |
| NMM = N-methylmorpholine | HPLC = high performance liquid |
| DCM = dichloromethane | chromatography |
| n-BuLi = n-butyllithium | IP or ip = intra-peritoneal |
| Pd/C = palladium on carbon | LC/MS = high performance liquid |
| PtO₂ = platinum oxide | chromatography/mass spectrometry |
| TEA = triethylamine | LiBH₄ = lithium borohydride |
| min = minute(s) | MS or Mass Spec = mass spectrometry |
| h or hr = hour(s) | NMP = N-methylpyrrolidinone = 1-methyl-2- |
| L = liter | pyrrolidinone |
| mL or ml = milliliter | NMR = nuclear magnetic resonance |
| μL = microliter | PyAOP reagent = (7-Azabenzotriazol-1- |
| g = gram(s) | yloxy)tris(pyrrolidino) phosphonium |
| mg = milligram(s) | Hexafluorophosphate |
| mol = mole(s) | PyBOP reagent = benzotriazol-1-yloxy- |
| mmol = millimole(s) | tripyrrolidino phosphonium |
| meq = milliequivalent | hexafluorophosphate |
| rt or RT = room temperature | Sc or SC = subcutaneous |
| sat or sat'd = saturated | TLC = thin layer chromatography |
| aq. = aqueous | Cbz = carbobenzyloxy or carbobenzoxy or |
| mp = melting point | benzyloxycarbonyl |
| Bip = biphenylalanine | Cl-HOBt = 6-Chloro-benzotriazole |
| Trt = trityl | DIC = N,N′-diisopropylcarbodiimide |
| 9-BBN = 9-Borabicyclo[3.3.1]nonane | DIEA = Diisopropylethylamine |
| Boc or BOC = tert-butyloxycarbonyl | BOP reagent = benzotriazol-1-yloxy-tris- |
| DIAD = diisopropyl azodicarboxylate | dimethylamino-phosphonium |
| | hexafluorophosphate (Castro's reagent) |

Characterization by Mass Spectrometry

Each compound was characterized by electrospray mass spectrometry (ES-MS) either in flow injection or LC/MS mode. Finnigan SSQ7000 single quadrupole mass spectrometers (ThermoFinnigan, San Jose, Calif.) were used in all analyses in positive and negative ion electrospray mode. Full scan data was acquired over the mass range of 300 to 2200 amu for a scan time of 1.0 second. The quadrupole was operated at unit resolution. For flow injection analyses, the mass spectrometer was interfaced to a Waters 616 HPLC pump (Waters Corp., Milford, Mass.) and equipped with an HTS PAL autosampler (CTC Analytics, Zwingen, Switzerland). Samples were injected into a mobile phase containing 50:50 water:acetonitrile with 0.1% ammonium hydroxide. The flow rate for the analyses was 0.42 mL/min. and the injection volume 6 μl. In all cases, the experimentally measured molecular weight was within 0.5 Daltons of the calculated mono-isotopic molecular weight.

### EXAMPLE 1

### Solid Phase Synthesis of 11-Mer Peptide Analogs Using an Applied Biosystems Model 433A Peptide Synthesizer

The following is a general description of the solid phase synthesis of peptide analogs described herein, using an upgraded Applied Biosystems Model 433A peptide synthesizer. The upgraded hardware and software of the synthesizer enabled conductivity monitoring of the Fmoc deprotection step with feedback control of coupling. The protocols allowed a range of synthesis scale from 0.05 to 1.0 mmol.

The incorporation of the two non-natural C-terminal amino acids can be achieved using the procedures described in Examples 2-5. Such an Fmoc-protected dipeptidyl resin was used in this ABI synthesis. The Fmoc-protected dipeptidyl-resin (0.1 mmol) was added to a vessel of appropriate size on the instrument, washed six times with NMP, and deprotected using two treatments with 22% piperidine/NMP (2 and 8 minutes each). One or two additional monitored deprotection steps were performed until the conditions of the monitoring option were satisfied (i.e., <10% difference between the last two conductivity-based deprotection peaks). The total deprotection time was 10-12 minutes The deprotected dipeptidyl-resin washed six times with NMP and then coupled with the next amino acid.

Fmoc-Asp(OtBu)-OH was coupled using the following method: Fmoc-Asp(OtBu)-OH (1 mmol, 10 eq.) was dissolved in 2 mL of NMP and activated by subsequent addition of 0.45 M HBTU/HOBt in DMF (2.2 mL) and 2 M DIEA/NMP (1 mL). The solution of the activated Fmoc-protected amino acid was then transferred to the reaction vessel and the coupling proceeded for 30 to 60 minutes, depending on the feedback from the deprotection steps. The resin was then washed six times with NMP, and subjected to eight additional deprotection/coupling cycles, as described above, in order to complete the assembly of the desired sequence. The Fmoc-amino acids sequentially used were: Fmoc-Ser(tBu)-OH, Fmoc-Thr(tBu)-OH, Fmoc-α-methyl-Phe(2-Fluoro)-OH or analog thereof, Fmoc-Thr(tBu)-OH, Fmoc-Gly-OH, Fmoc-Glu(OtBu)-OH, Fmoc-Aib-OH and Fmoc-His(Trt)-OH. The Fmoc group was removed with 22% piperidine in NMP as described above, and the peptidyl-resin washed six times with NMP and DCM, and dried in vacuo.

Alternatively, a modified coupling protocol was used in which the Fmoc-protected amino acid (0.26 mmol) was activated by subsequent addition of 0.5 M HOAt in DMF (0.52 mL) and DIC (40 μL), transferred to the reaction vessel and allowed to couple for 14-18 hours.

Cleavage/Deprotection

The desired peptide was cleaved/deprotected from its respective peptidyl-resin by treatment with a solution of TFA/water/tri-isopropylsilane (96:2:2) (3.0 mL) for two hours. The resin was filtered off, rinsed with TFA (1.0 mL), and the combined TFA filtrates were added to 35 mL of Et₂O. The resulting precipitate was collected by centrifugation and finally dried, to yield 100-300 mg of crude peptide product as a white solid. The product was purified by preparative HPLC. The gradient used was from 15% to 45%, 0.1% TFA/MeCN in 0.1% TFA/water over 40 minutes. The fractions containing pure product were pooled and lyophilized, to yield 10-30 mg of pure product.

### EXAMPLE 2

### Synthesis of Biphenylalanine Analogs at Position Xₐₐ₁₀ and Homohomophenylalanine Analogs at Position Xₐₐ₁₁ Represented by Formulas II And III

For those analogs wherein position Xₐₐ₁₀ and position Xₐₐ₁₁ residues were represented by substituted amino acid analogs of Formulas II and III, i.e. biphenylalanine analogs (Bip analogs) or hetero-biphenylalanine analogs, or Homohomophenylalanine analogs (hhPhe analogs), their incorporation into the peptide chain was carried out using one of the following approaches.
1. General Procedure for Preparation of Rink Amide MBHA Resin Containing Amino Acids Represented by Formula III at Position Xₐₐ₁₁ (Hydroboration-Suzuki Couplings) (Scheme 1) 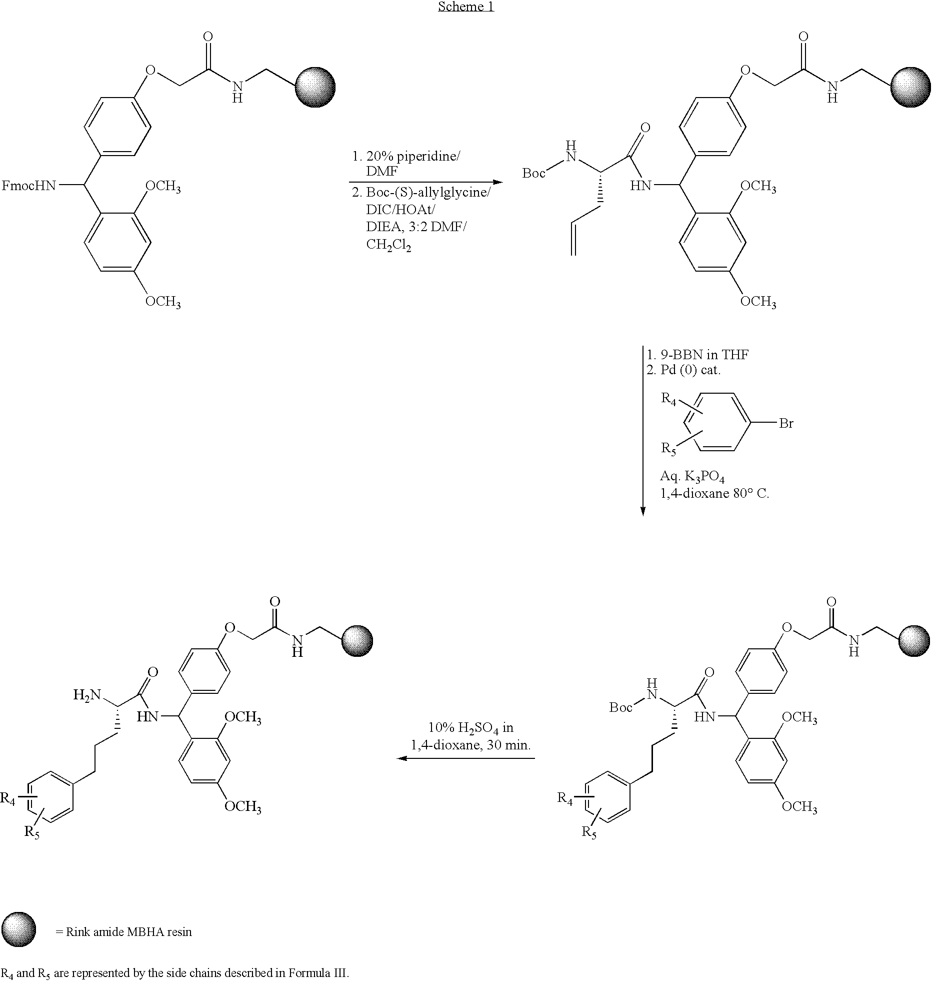
A. General Procedure for X1=X2=C in Formula III

Polystyrene-Rink amide MBHA resin (800 mg, 512 μmol, loading level of 640 μmol/g) was swelled in CH₂Cl₂ (8.0 mL) in a filter tube for ten minutes. The resin was drained and transferred to a 20 mL scintillation vial. Following transfer, 8:2 DMF/piperidine (9.00 mL) was added to the resin. The vial was capped and the contents agitated for 90 minutes. The resin was then transferred to a filter tube, drained, and washed with DMA (3×8 mL), MeOH (3×8 mL), and CH₂Cl₂ (3×8 mL). Meanwhile, (S)-2-(tert-butoxycarbonyl)pent-4-enoic acid (165 mg, 768 μmol) was added to a fresh 20 mL scintillation vial. 1-hydroxy-7-azabenzotriazole (1.37 mL, 0.6 M solution in THF, 819 μmol) was added, followed by addition of 7 mL 3:2 DMF/CH₂Cl₂. Next, 1,3-diisopropylcarbodiimide (0.128 mL, 819 μmol) was added and reacted for five minutes. The deprotected resin was added to the reaction solution. N,N-diisopropylethylamine (0.357 mL, 2048 μmol) was then added to the resin slurry. The vial was capped and placed on an orbital shaker (140 rpm) overnight (18 hours). After 18 hours, the resin was transferred to a filter tube, drained, and washed with DMA (3×8 mL), MeOH (3×8 mL), and CH₂Cl₂ (3×8 mL).

A capped 20 mL scintillation vial was cooled to 0° C. in an ice bath. 9-BBN (0.5 M in THF, 1.60 mL, 800 μmol) was added to the vial, followed by dry Rink-AllylGly-Boc resin (125 mg, 80 μmol). The resin was reacted at 0° C. for five minutes The vial was then removed from the ice bath and agitated for two hours. The vial was vented several times over the course of the reaction to avoid pressure build-up.

The scintillation vial was uncapped and as much solution as possible was pipetted from the vial (without removing resin). Then 1,4-dioxane (2.0 mL) was added to the scintillation vial containing the resin. K₃PO₄ solution (0.400 mL; 2 M aqueous solution, 800 μmol) was added to the vial. Aryl bromide (400 μmol) was then added to the vial, which was then placed in a N₂ atmosphere glove bag. Tetrakis(triphenylphosphine)palladium(0) catalyst (9.2 mg, 8.0 μmol) was added to the vial while in the glove bag, and sealed with a Teflon-lined screw-cap. The vial was heated to 80° C. with agitation for 18 hours. The vial was cooled to room temperature, and the resin was then transferred to a filter tube. The resin was drained, and washed with 1:1 DMA/H₂O (1×2 mL), DMA (3×2 mL), THF (1×2 mL), MeOH (3×2 mL), and CH₂Cl₂ (3×2 mL). The resulting resin was pale yellow in color. The resin was then directly used in the Boc deprotection (see general procedure below).
B. General Procedure for Removal of N-Terminal α-Amine Boc Protecting Group (Scheme 2)

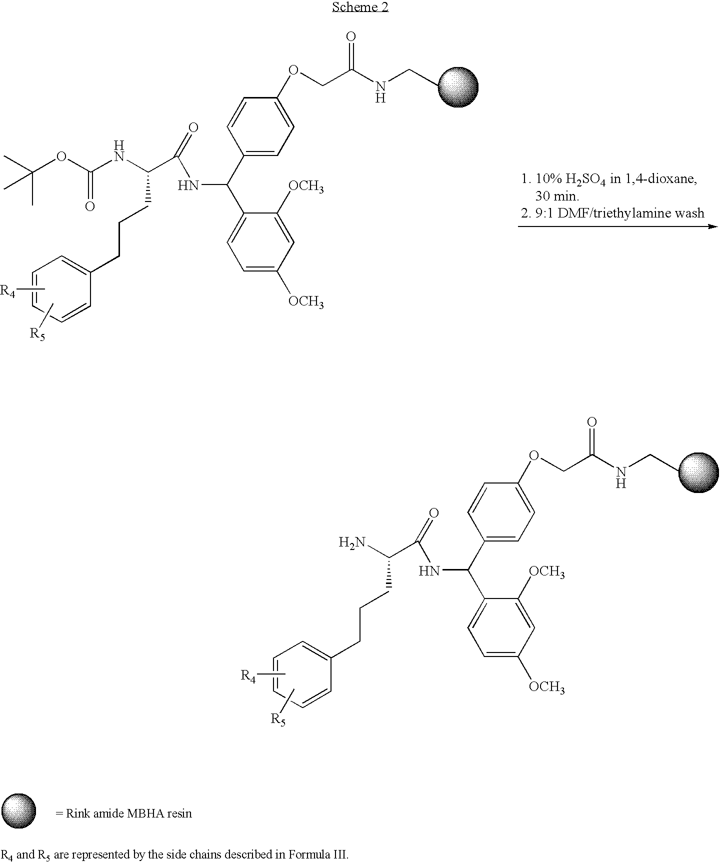

Polystyrene-Rink-amino acid-Boc resin (50 mg, 32 μmol) was swelled in CH₂Cl₂ (0.50 mL) in a plastic tube for ten minutes. The resin was drained followed by addition of 10% H₂SO₄ in 1,4-dioxane (0.50 mL), and reacted for 30 minutes with occasional agitation. The resin was drained and washed with 1,4-dioxane (2×0.5 mL), 9:1 DMF/Et₃N (2×0.5 mL), DMF (3×0.5 mL), MeOH (3×0.5 mL), and CH₂Cl₂ (3×0.5 mL). This procedure provided the free N-terminal α-amine on the polystyrene-Rink resin.
C. General Procedure for Coupling Position 10 Amino Acid to Position 11 Hhphe Analog on Rink Amide MBHA Resin (Scheme 3)

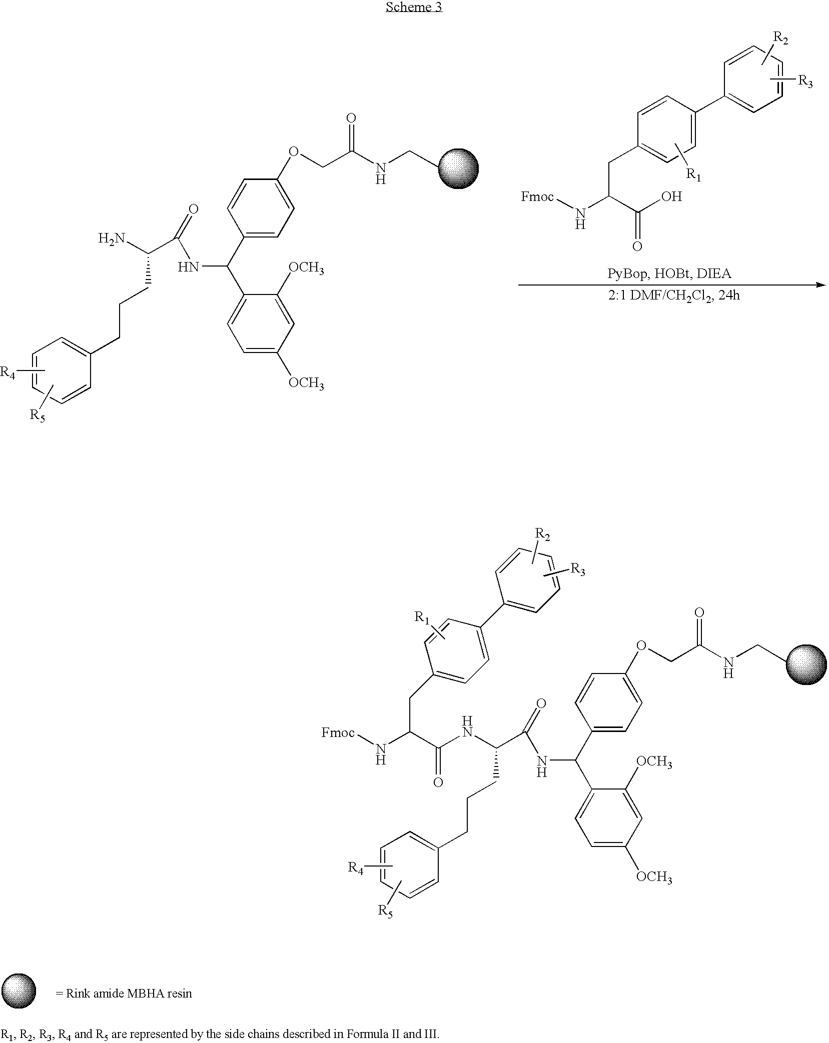

Fmoc-L-(4′-methoxy-2′-ethyl)biphenylalanine (213 mg, 409 μmol) was added to a 20 mL scintillation vial. 1-hydroxybenzotriazole (75 mg, 558 μmol) was added to the vial and dissolved in 2:1 DMF/CH₂Cl₂ (5.8 mL). PyBOP (232 mg, 446 μmol) was added to the vial and reacted for five minutes. N,N-diisopropylethylamine (0.192 mL, 1116 μmol) was added to the reaction solution followed by addition of α-amine deprotected hhPhe resin (600 mg, 372 μmol). The vial was capped and agitated for 24 hours. After 24 hours the resin was transferred to a filter tube, drained, and washed with DMA (3×6 mL), MeOH (3×6 mL), and CH₂Cl₂ (3×6 mL).

### EXAMPLE 3

### Synthesis of Biphenylalanine Analogs at Position Xₐₐ₁₀ and Unnatural Amino Acid Analogs at Position Xₐₐ₁₁ Represented by Formulas II and IV

For those analogs having position Xₐₐ₁₀ and Xₐₐ₁₁ residues as substituted amino acid analogs of Formulas II and IV, i.e. biphenylalanine analogs (Bip analogs) hetero-biphenylalanine analogs at position 10, and aspartic, or glutamic amide, ester, sulfonamide, or reverse amide or serine or threonine ether or ester analogs at position 11, their incorporation into the peptide chain was carried out using the following approach.
1. General Procedure for Preparation of Rink Amide MBHA Resin Containing Aspartic or Glutamic Acid Derivatives Represented by Formula IV at Position Xₐₐ11 (Scheme 4) 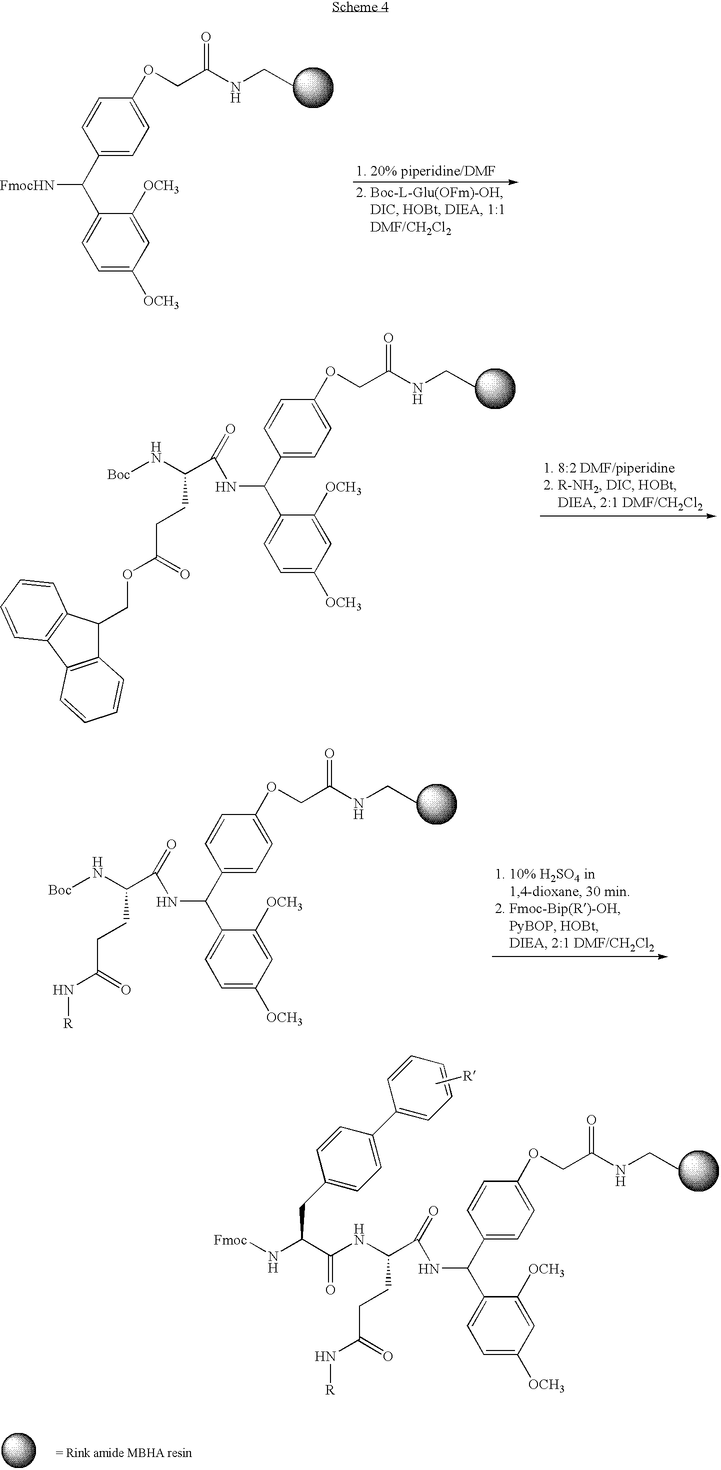
A. General Procedure for Loading MBHA Resin

Polystyrene-Rink amide MBHA resin (400 mg, 256 μmol, loading level of 640 μmol/g) was added to a 20 mL scintillation vial, followed by addition of 8:2 DMF/piperidine (5.00 mL). The vial was capped and the contents agitated for 45 minutes. The resin was transferred to a filter tube, drained, and washed with DMF (3×5 mL), MeOH (3×5 mL), and CH₂Cl₂ (3×5 mL). Boc-L-Glu(OFm)-OH (218 mg, 512 μmol) was added to a fresh 20 mL scintillation vial. 1-hydroxybenzotriazole (88.2 mg, 576 μmol), was added followed by 4 mL of 1:1 DMF/CH₂Cl₂. 1,3-diisopropylcarbodiimide (0.090 mL, 576 μmol) was added and reacted for five minutes. The deprotected resin was added to the resulting reaction solution. N,N-diisopropylethylamine (0.178 mL, 1020 μmol) was added to the resin slurry, the vial was capped, and placed on an orbital shaker (125 rpm) overnight (18 hours). After 18 hours, the resin was transferred to a filter tube, drained, and washed with DMF (3×5 mL), MeOH (3×5 mL), and CH₂Cl₂ (3×5 mL).

B. General Procedure for Deprotection of Position 11 Protected Carboxylate Side Chain and Acylation Procedure (Scheme 5)

PS-Rink-L-Glu(OFm)-Boc resin (25 mg, 16 μmol) was added to a filter tube and swelled in 0.50 mL CH₂Cl₂ for five minutes. The resin was then drained, and 8:2 DMF/piperidine (0.50 mL) was added to the resin. The resin reacted for 45 minutes with occasional agitation. The resin was then drained and rinsed with DMF (3×0.5 mL), MeOH (3×0.5 mL), and CH₂Cl₂ (3×0.5 mL). 1-hydroxybenzotriazole (12.2 mg, 80 μmol) was added to a 1-dram vial, and dissolved in 0.6 mL 2:1 DMF/CH₂Cl₂. 1,3-diisopropylcarbodiimide (0.013 mL, 80 μmol) was added to the solution, followed by addition of 25 mg of deprotected resin. N,N-diisopropylethylamine (0.017 mL, 96 μmol) was added, and the resulting slurry reacted for five minutes. An amine (80 μmol) was added directly to the vial, which was then capped and reacted with mild agitation for 18 hours. The resin was then transferred to a filter tube, drained, and washed with DMF (3×0.5 mL), MeOH (3×0.5 mL), and CH₂Cl₂ (3×0.5 mL). 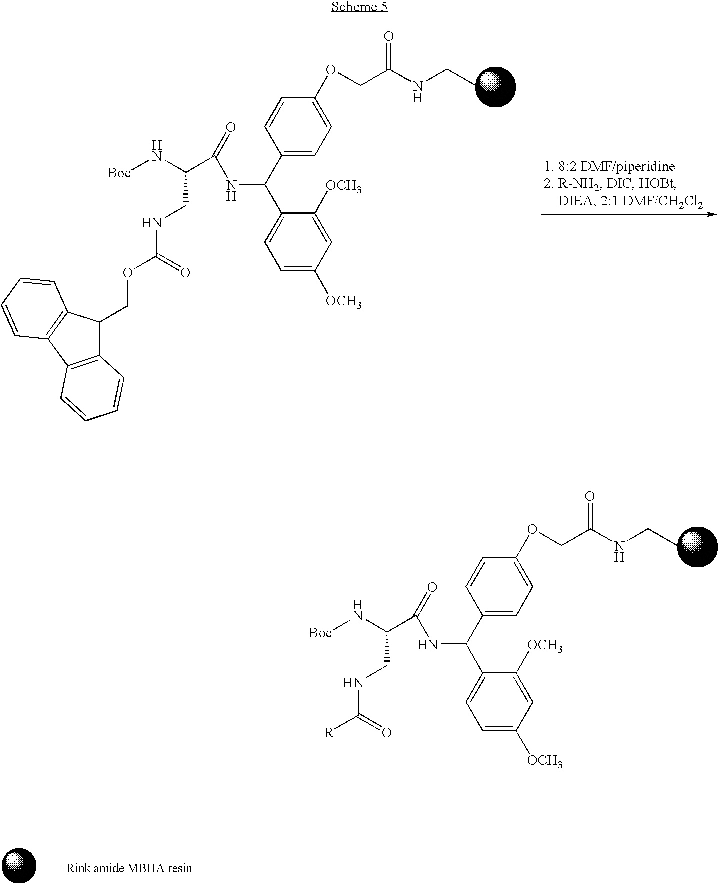
C. General Procedure for Deprotection of Position 11 Protected Amine Side Chain and Acylation Procedure

Boc-3-Fmoc-L-2,3-diaminopropanoic acid (Boc-L-Dap(Fmoc)-OH) was loaded into polystyrene-Rink resin using a procedure analogous to that described above in General Procedure A. PS-Rink-L-Dap(Fmoc)-Boc resin (25 mg, 16 μmol) was added to a filter tube and swelled in 0.50 mL CH₂Cl₂ for five minutes. The resin was then drained, and 8:2 DMF/piperidine (0.50 mL) was added to the resin. The resin reacted for 45 minutes with occasional agitation. The resin was then drained and rinsed with DMF (3×0.5 mL), MeOH (3×0.5 mL), and CH₂Cl₂ (3×0.5 mL).

A carboxylic acid (80 μmol) and 1-hydroxybenzotriazole (12.2 mg, 80 μmol) were added to a 1-dram vial, and dissolved in 0.6 mL 2:1 DMF/CH₂Cl₂. 1,3-diisopropylcarbodiimide (0.013 mL, 80 μmol) was added to this solution and the reaction proceeded for ten minutes. The deprotected resin was added to the resulting coupling solution, followed by N,N-diisopropylethylamine (0.017 mL, 96 μmol). The vial was capped and reacted with mild agitation for 18 hours. The resin was then transferred to a filter tube, drained, and washed with DMF (3×0.5 mL), MeOH (3×0.5 mL), and CH₂Cl₂ (3×0.5 mL).

Alternatively, in place of the carboxylic acid coupling solution, an analogous solution of a chloroformate (80 μmol) and pyridine (160 μmol) was used to generate the corresponding carbamate.

Alternatively, in place of the carboxylic acid coupling solution an analogous solution of an isocyanate (80 μmol) was used to generate the corresponding urea.

D. General Procedure for Removal of N-Terminal α-Amine Boc Protecting Group

PS-Rink-amino acid-Boc resin (25 mg, 16 μmol) was swelled in CH₂Cl₂ (0.50 mL) in a plastic tube for ten minutes. The resin was drained and 10% H₂SO₄ in 1,4-dioxane (0.50 mL) was added and reacted for 30 minutes with occasional agitation. The resin was drained and washed with 1,4-dioxane (2×0.5 mL), 9:1 DMF/Et₃N (2×0.5 mL), DMF (3×0.5 mL), MeOH (3×0.5 mL), and CH₂Cl₂ (3×0.5 mL). This procedure provided the free N-terminal α-amine on the PS-Rink resin.
2. General Procedure for Coupling Position 10 Amino Acid to Position 11 Glutamic Amide Analog on Rink Amide MBHA Resin (Scheme 6)

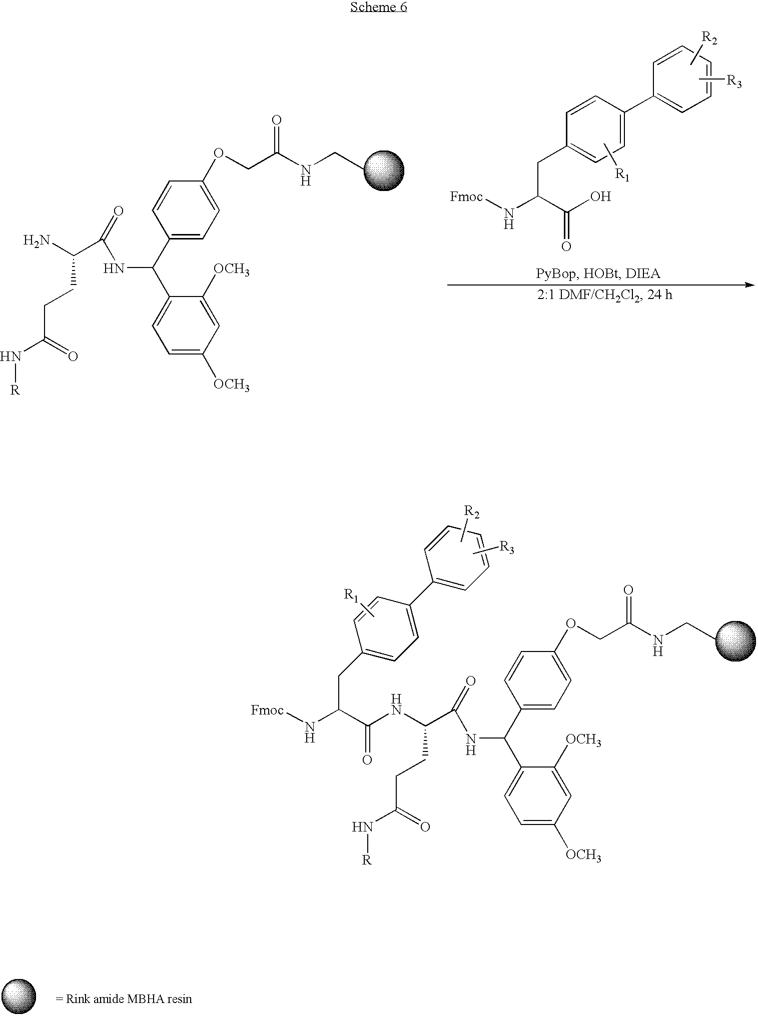

Fmoc-L-Bip(R)-OH (409 μmol) was added to a 20 mL scintillation vial. 1-hydroxybenzotriazole (75 mg, 558 μmol) was added and dissolved in 2:1 DMF/CH₂Cl₂ (5.8 mL). PyBOP (232 mg, 446 μmol) was added and reacted for five minutes. N,N-diisopropylethylamine (0.192 mL, 1116 μmol) was added to the reaction solution. Then the α-amine deprotected Pos. 11 resin (600 mg, 372 μmol) was added to the scintillation vial containing the reaction solution. The vial was then capped and agitated for 24 hours. After 24 hours the resin was transferred to a filter tube, drained, and washed with DMF (3×6 mL), MeOH (3×6 mL), and CH₂Cl₂ (3×6 mL).

### EXAMPLE 4

### Synthesis of Biphenylalanine Analogs at Position Xₐₐ₁₀ and Unnatural Amino Acid Analogs at Position Xₐₐ₁₁ Represented by Formulas II and V

For those analogs having position Xₐₐ₁₀ and Xₐₐ₁₁ residues as substituted amino acid analogs of Formulas II and V, e.g. biphenylalanine analogs (Bip analogs) or hetero-biphenylalanine analogs at position 10, and 4-aminomethylphenylalanine(4-aminomethylPhe) or 4-aminophenylalanine(4-aminoPhe) Analogs at position 11, their incorporation into the peptide chain was carried out using the following approach.
1. General Procedure for Preparation of Rink Amide MBHA Resin Containing 4-AminomethylPhe or 4-AminoPhe Derivatives Represented by Formulas V and VIII at Position Xₐₐ₁₁ (see Scheme 7 for 4-AminomethylPhe Example; Synthesis of 4-AminoPhe Analogs is Analogous) 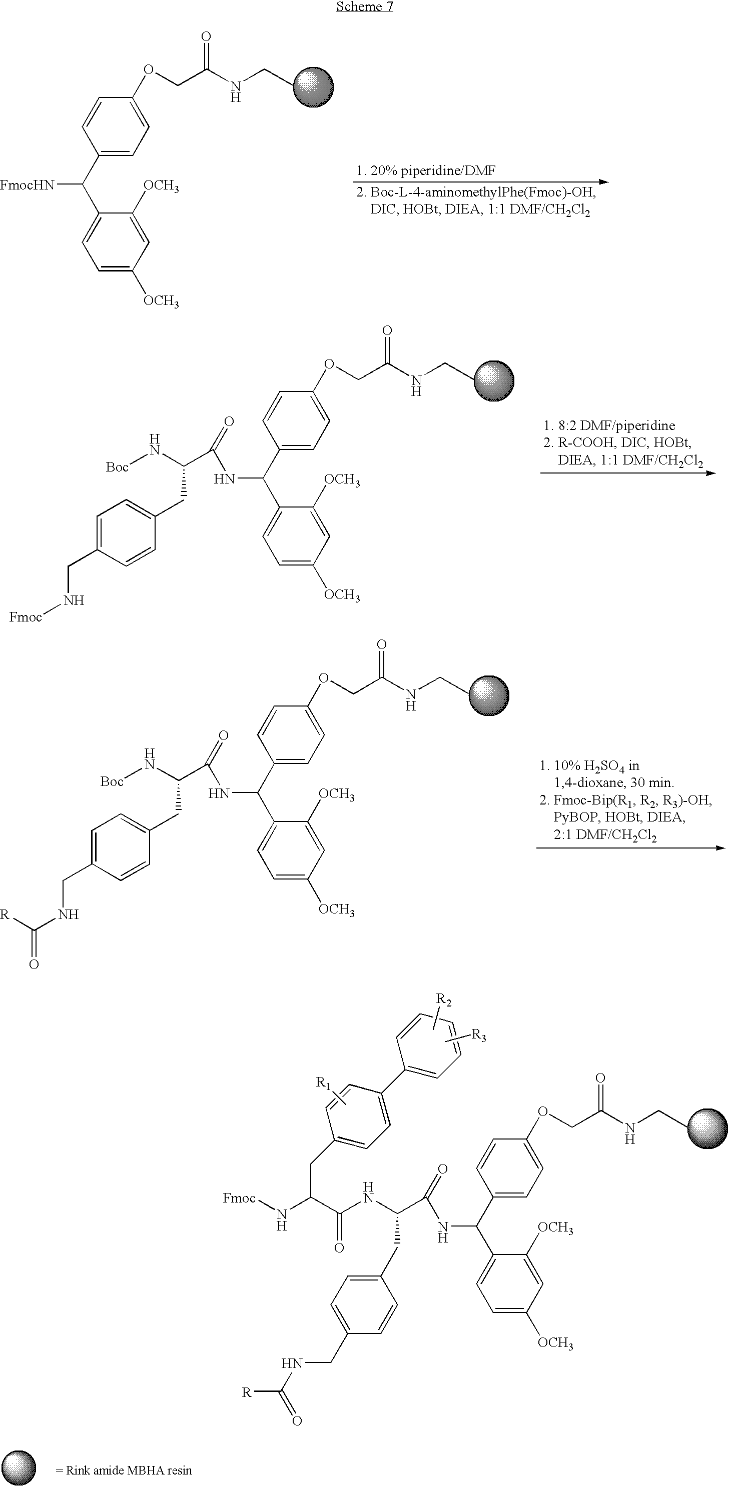
A. General Procedure for Loading MBHA Resin

Polystyrene-Rink amide MBHA resin (1.10 g, 0.704 mmol, loading level of 0.640 mmol/g) was added to a filter tube, then swelled in CH₂Cl₂ (11.0 mL) for ten minutes. The resin was drained and a solution of 8:2 DMF/piperidine (11.0 mL) was added to the filter tube containing the resin. The Fmoc deprotection reaction proceeded for one hour with occasional agitation. The tube was drained, fresh 8:2 DMF/piperidine (11.0 mL) was added to the filter tube, and the resin was deprotected for an additional 30 minutes. The tube was drained, and the resin washed with DMF (3×15 mL), MeOH (3×15 mL), and CH₂Cl₂ (3×15 mL).

Boc-L-4-aminomethylPhe(Fmoc)-OH (0.546 g, 1.06 mmol) was then added to a fresh 20 mL scintillation vial. 1-hydroxybenzotriazole (0.189 g, 1.23 mmol) was added to the vial, followed by 10.0 mL 1:1 DMF/CH₂Cl₂. 1,3-diisopropylcarbodiimide (0.193 mL, 1.23 mmol) was added to the vial containing the amino acid and reacted for five minutes. The deprotected Rink resin was added to the resulting reaction solution. N,N-diisopropylethylamine (0.490 mL, 2.82 mmol) was added to the resin slurry, the vial was capped and placed on an orbital shaker (150 rpm) overnight (22 hours). After 22 hours, the resin was transferred to a filter tube, drained, and washed with DMF (3×15 mL), MeOH (3×15 mL), and CH₂Cl₂ (3×15 mL). 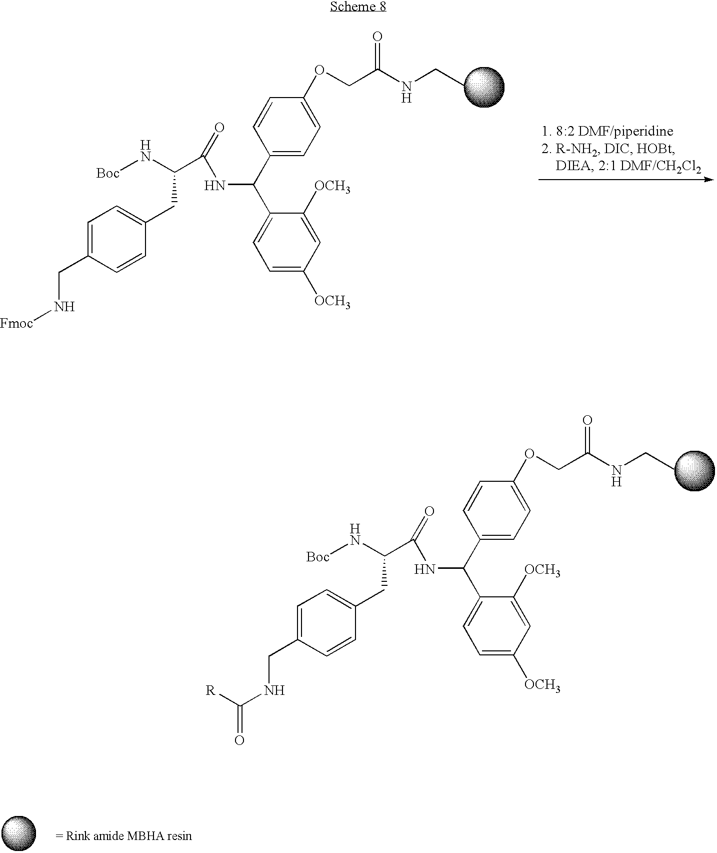
B. General Procedure for Deprotection of Position 11 Protected Amine Side Chain and Acylation Procedure (Scheme 8)

PS-Rink-L-4-aminomethylPhe(Fmoc)-Boc resin (50 mg, 32 μmol) was added to a filter tube and swelled in 0.50 mL CH₂Cl₂ for five minutes. The resin was then drained, and 8:2 DMF/piperidine (0.50 mL) was added to the resin. The resin was reacted for 45 minutes with occasional agitation. The resin was then drained and rinsed with DMF (3×0.5 mL), MeOH (3×0.5 mL), and CH₂Cl₂ (3×0.5 mL). A solution of 1:1 DMF/CH₂Cl₂ (0.5 mL) was added to the resin, followed by N,N-diisopropylethylamine (0.045 mL, 256 μmol).

The selected carboxylic acid (256 μmol) was added to a 1-dram vial. 1-hydroxybenzotriazole (39.2 mg, 256 μmol) was added to the vial, followed by 0.5 mL 1:1 DMF/CH₂Cl₂. 1,3-diisopropylcarbodiimide (0.040 mL, 256 μmol) was added to the resulting solution. The slurry of the deprotected resin prepared above was added directly to the activated carboxylic acid solution, and the 1-dram vial was capped. The reaction proceeded with agitation for 22 hours. The resin was then transferred to a filter tube, drained, and washed with DMF (3×0.5 mL), MeOH (3×0.5 mL), and CH₂Cl₂ (3×0.5 mL). 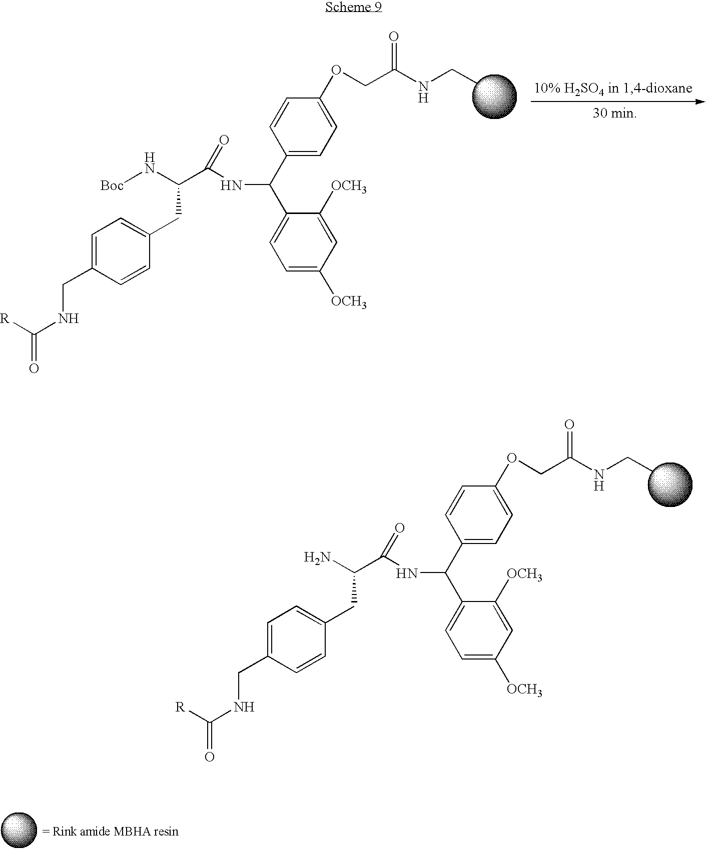
C. General Procedure for Removal of N-Terminal α-Amine Boc Protecting Group (Scheme 9)

PS-Rink-amino acid-Boc resin (50 mg, 32 μmol) was swelled in CH₂Cl₂ (0.50 mL) in a plastic tube for ten minutes. The resin was drained and 10% H₂SO₄ in 1,4-dioxane (0.50 mL) was added and reacted for 30 minutes with occasional agitation. The resin was drained and washed with 1,4-dioxane (2×0.5 mL), 9:1 DMF/Et₃N (2×0.5 mL), DMF (3×0.5 mL), MeOH (3×0.5 mL), and CH₂Cl₂ (3×0.5 mL). This procedure provided the free N-terminal α-amine on the PS-Rink resin. 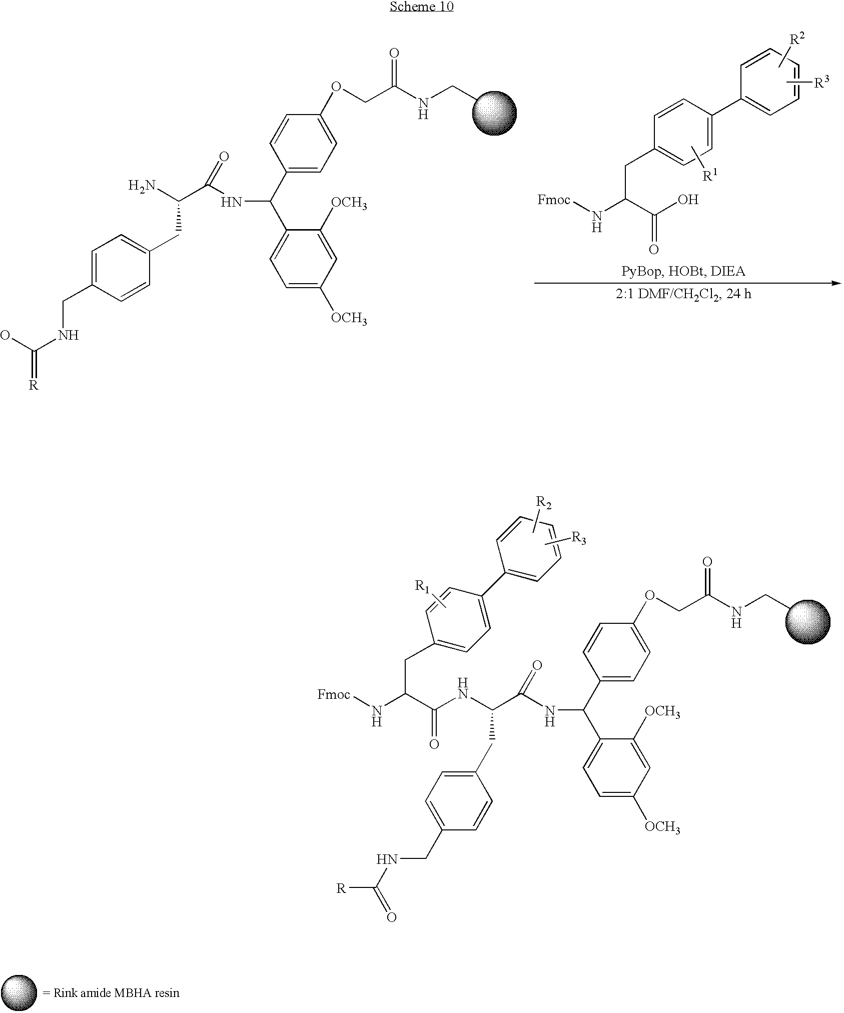
2. General Procedure for Coupling Position 10 Amino Acid to Position 11 4-AminomethylPhe Analog on Rink Amide MBHA Resin (Scheme 10)

Fmoc-L-Bip(R)-OH (704 μmol) was added to a 20 mL scintillation vial. 1-hydroxybenzotriazole (122 mg, 800 μmol) was added to the vial and dissolved in 2:1 DMF/CH₂Cl₂ (10.0 mL). PyBOP (416 mg, 800 μmol) was added to the solution and reacted for five minutes. N,N-diisopropylethylamine (0.334 mL, 1920 μmol) was added to the reaction solution. The resulting solution was evenly distributed (˜0.50 mL/vial) into 20 1-dram vials containing 50 mg PS-Rink-amino acid/vial (0.64 mmol/g loading, 32 μmol/vial, total of 640 μmol resin). The vials were capped and reacted for 20 hours with agitation. The resins were transferred to 1 mL filter tubes, drained, and each tube washed with DMF (3×0.5 mL), MeOH (3×0.5 mL), and CH₂Cl₂ (3×0.5 mL).

### EXAMPLE 5

### Synthesis of Biphenylalanine Analogs at Position Xₐₐ₁₀ and Unnatural Amino Acid Analogs at Position Xₐₐ₁₁ Represented by Formulas II and VI

For those analogs having position Xₐₐ₁₀ and Xₐₐ₁₁ residues represented by substituted amino acid analogs of Formulas II and VI, e.g. biphenylalanine analogs (Bip analogs) or hetero-biphenylalanine analogs at position 10, and (for example) L-2-aminooctanoic acid Analogs at position 11, their incorporation into the peptide chain was carried out using the following approach.
1. General Procedure for Preparation of Sieber Resin-Amino Acid Dimers Containing Amino Acid Derivatives Represented by Formula VI at Position Xₐₐ₁₁ (Scheme 11) 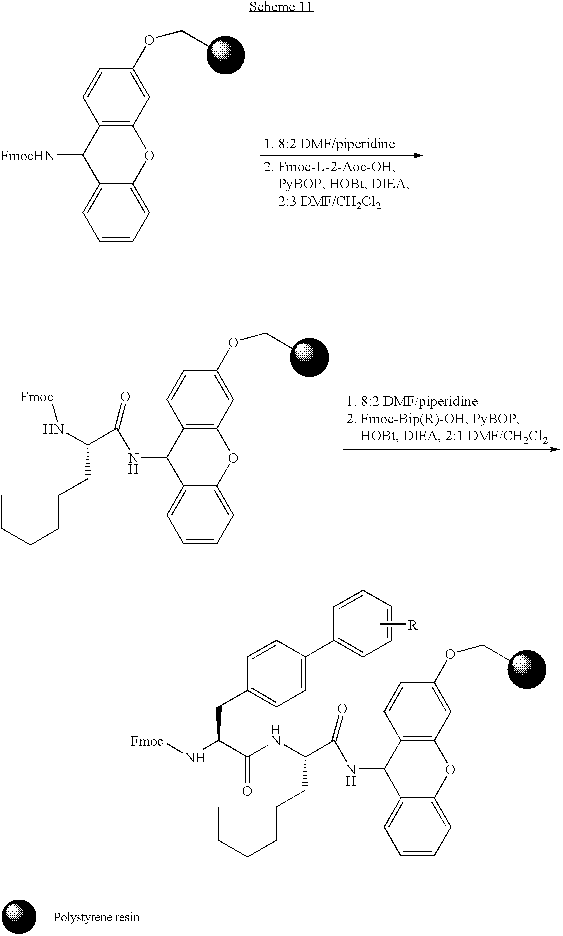
A. General Procedure for Loading Sieber Resin with Position 11 Amino Acid

Polystyrene-Sieber Amide resin (48 mg, 25 μmol, loading level of 520 μmol/g) was added to a 1-dram vial. 8:2 DMF/piperidine (0.500 mL) was added to the vial. The vial was then capped and the contents agitated for 45 minutes. The resin was transferred to a filter tube, drained, and washed with DMF (3×0.5 mL), MeOH (3×0.5 mL), and CH₂Cl₂ (3×0.5 mL). Fmoc-L-2-aminooctanoic acid (38 mg, 100 μmol) was added to a fresh 1-dram vial. 1-hydroxybenzotriazole (16 mg, 100 μmol) was added to the vial containing the amino acid, and the contents of the vial were dissolved in 0.50 mL 2:3 DMF/CH₂Cl₂. PyBOP (52 mg, 100 μmol) was added to the vial containing the amino acid solution, followed by N,N-diisopropylethylamine (0.0.087 mL, 499 μmol), and reacted for five minutes. The deprotected resin from above was added to this solution, the vial capped, and placed on an orbital shaker (125 rpm) overnight (18 hours). After 18 hours, the resin was transferred to a filter tube, drained, and washed with DMF (3×0.5 mL), MeOH (3×0.5 mL), and CH₂Cl₂ (3×0.5 mL).

B. General Procedure for Removal of N-Terminal α-Amine Fmoc Protecting Group

PS-Sieber-amino acid-Boc resin (48 mg, 25 μmol) was swelled in CH₂Cl₂ (0.50 mL) in a plastic tube for ten minutes. The resin was drained and 8:2 DMF/piperidine (0.50 mL) was added to the resin. The resulting slurry was reacted for 40 minutes with occasional agitation. The resin was drained and washed with DMF (3×0.5 mL), MeOH (3×0.5 mL), and CH₂Cl₂ (3×0.5 mL). This reaction provided the free N-terminal α-amine on the PS-Sieber resin.
C. General Procedure for Coupling Position 10 Amino Acid to Position 11 Amino Acid on Rink Amide Sieber Resin (Scheme 12)

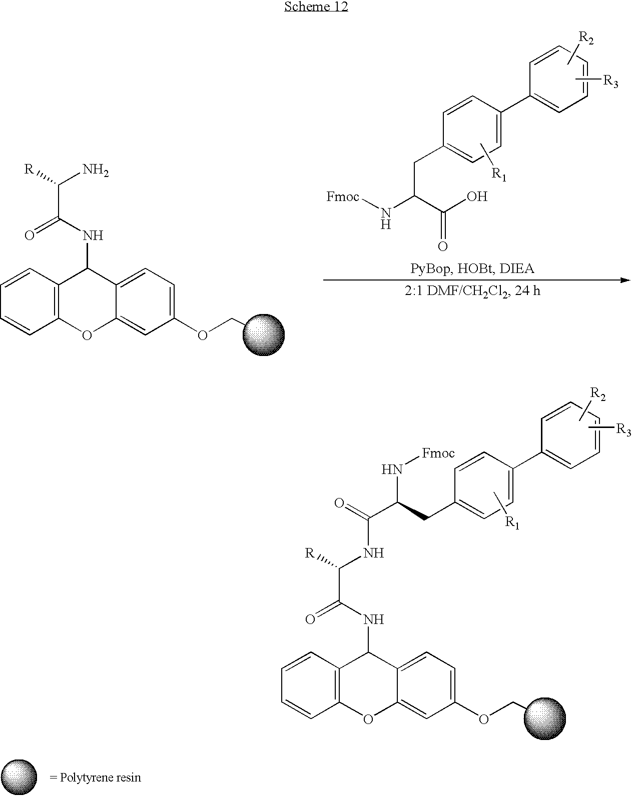

Fmoc-L-Bip(R₁,R₂,R₃)-OH (409 μmol) was added to a 20 mL scintillation vial. 1-hydroxybenzotriazole (75 mg, 558 μmol) was added and dissolved in 2:1 DMF/CH₂Cl₂ (5.8 mL). PyBOP (232 mg, 446 μmol) was added and reacted for five minutes. N,N-diisopropylethylamine (0.192 mL, 1116 μmol) was added to the reaction solution. Alpha-amine deprotected Pos. 11 PS-Sieber resin (600 mg, 372 μmol) was added to the scintillation vial containing the reaction solution. The vial was capped and agitated for 24 hours. After 24 hours the resin was transferred to a filter tube, drained, and washed with DMF (3×6 mL), MeOH (3×6 mL), and CH₂Cl₂ (3×6 mL).

### EXAMPLE 6

### General Procedure for Preparation of Peptides Via Fragment Condensation

Solid phase Suzuki condensation and standard amino acid coupling procedures were practiced to prepare the required amino acids represented by Formula II and Formula III at positions Xₐₐ₁₀ and Xₐₐ₁₁, as described in Example 2. The dipeptide was cleaved from the support, with either prior (see Scheme 13) or simultaneous (see Scheme 14) removal of the N-terminal α-amine protecting group. The dipeptide was then coupled to a fully side chain-protected 9 amino acid peptide (vide infra). Subsequent deprotection of side chains and purification resulted in the desired 11-mer peptide products.

### Solution Phase Fragment Condensation

In this approach, solid phase Suzuki condensations and acylations were performed (as described in Example 2) to prepare the desired dipeptides bound to polystyrene-Rink amide resin, with the N-terminal α-amine either Fmoc-protected or Boc-protected. The dipeptides were either first deprotected then cleaved or directly cleaved from the resin under acidic conditions. The dipeptides containing Fmoc-protected N-terminal G-amines were first deprotected on resin using an 8:2 DMF/piperidine solution, then cleaved from the resin under acidic conditions, as shown in Scheme 13. In the case of Boc-protected N-terminal α-amines, the acidic cleavage afforded simultaneous deprotection of the α-amine as shown in Scheme 14, and these were typically purified before being carried into the fragment coupling sequence.
1. Procedures for Cleavage of Dipeptides from Rink Amide MBHA Resin 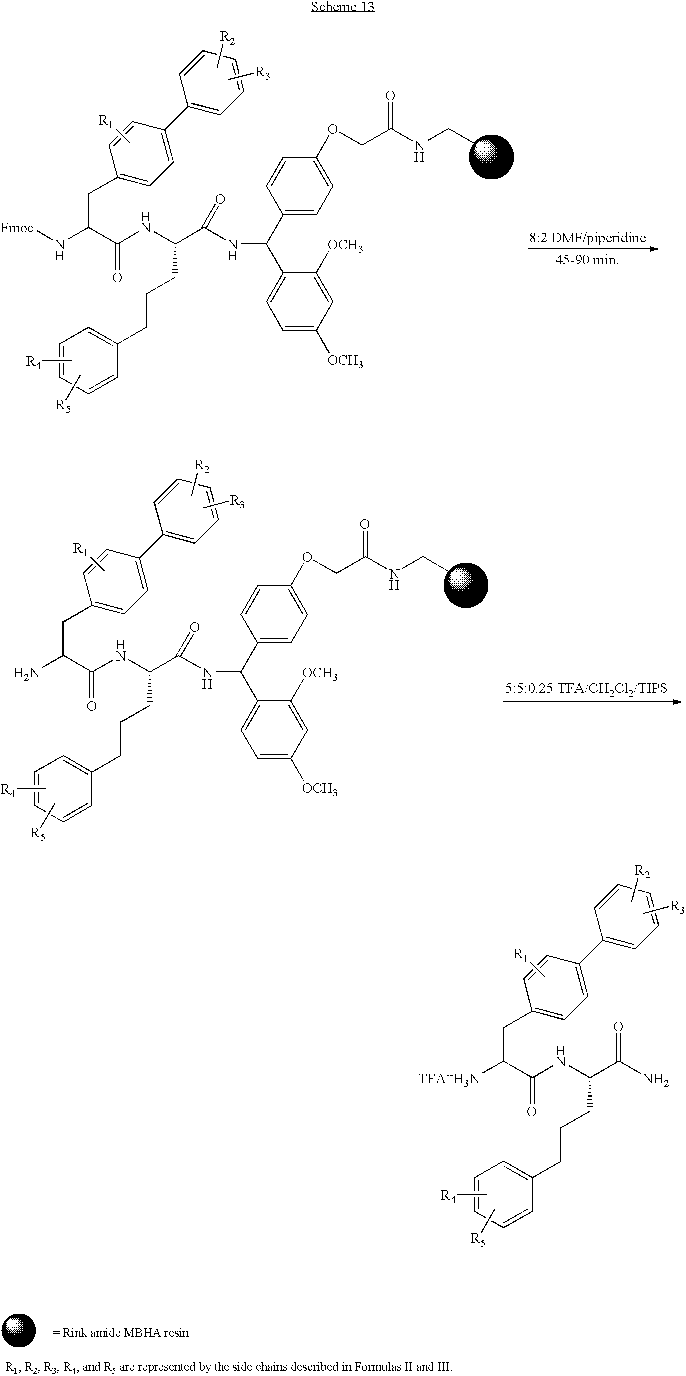
A. Procedure for Fmoc-Protected Dipeptides (Scheme 13)

The Fmoc-protected dipeptide-Rink amide resin (100 mg, 64 μmol) was soaked in dichloromethane (1.5 mL) for ten minutes. The resin was drained, transferred to a 1-dram vial, and a solution of 8:2 DMF/piperidine (1.5 mL) was added to the resin. The vial was capped and agitated for 45-90 minutes. The resin was then transferred to a filter tube, drained, and washed with DMA (3×2 mL), MeOH (3×2 mL), and CH₂Cl₂ (3×2 mL). The resin was transferred to a 1-dram glass vial and a solution of 5:5:0.25 trifluoroacetic acid/CH₂Cl₂/triisopropylsilane (1.5 mL) was added. The vial was capped and the resin cleaved for two hours. After two hours the solution was filtered into a clean vial, and rinsed with MeOH (1×1 mL), which was added to the cleavage solution. Fresh TFA/CH₂Cl₂/TIPS solution was added to the resin and the cleavage reaction was repeated. The cleavage solutions were combined and solvent evaporated. The resulting product was purified by HPLC, using a C-18 column and CH₃CN/H₂O/TFA or MeOH/H₂O/TFA solvent system with either UV or mass directed fraction collection to yield (after evaporation of solvent) the dipeptide as the trifluoroacetic acid salt of the α-amine. 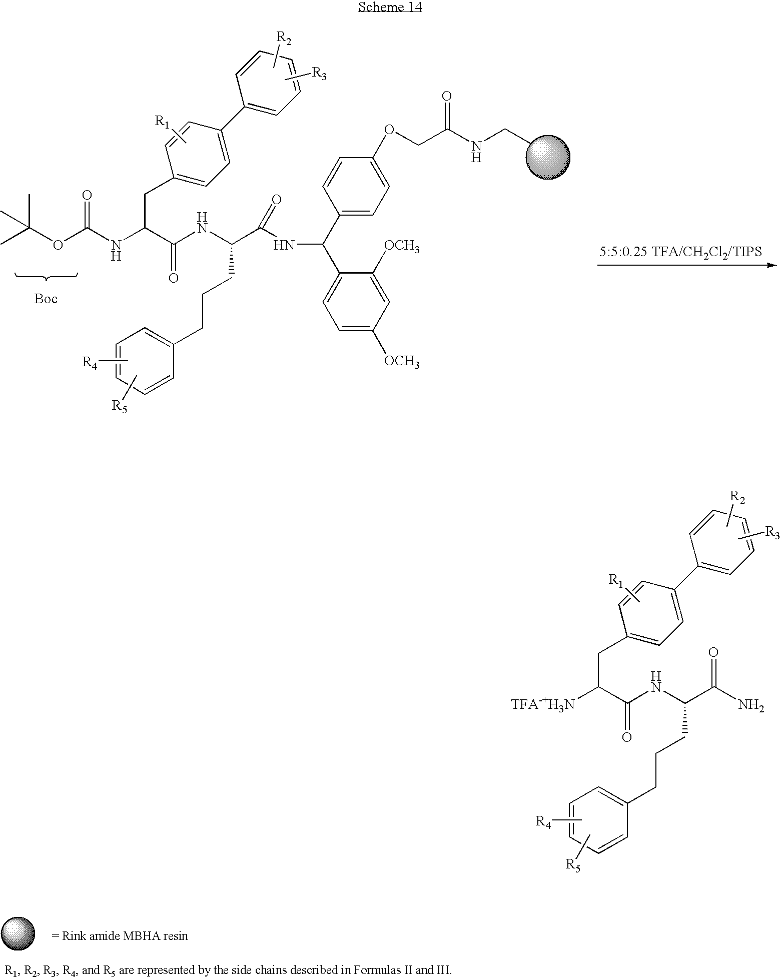
B. Procedure for Boc-Protected Dipeptides (Scheme 14)

The Boc-protected dipeptide-Rink resin (100 mg, 64 μmol) was added to a 1-dram glass vial and a solution of 5:5:0.25 trifluoroacetic acid/CH₂Cl₂/triisopropylsilane (1.5 mL) was added. The vial was capped and the resin cleaved for two hours. After two hours the solution was filtered into a clean vial, and rinsed with MeOH (1×1 mL), which was added to the cleavage solution. Fresh TFA/CH₂Cl₂/TIPS solution was added to the resin and the cleavage reaction was repeated. The cleavage solutions were combined, and the solvent evaporated. The resulting product was purified by HPLC, using a C-18 column and CH₃CN/H₂O/TFA or MeOH/H₂O/TFA solvent system with either UV or mass directed fraction collection to yield (after evaporation of solvent) the dipeptide as the trifluoroacetic acid salt of the α-amine. 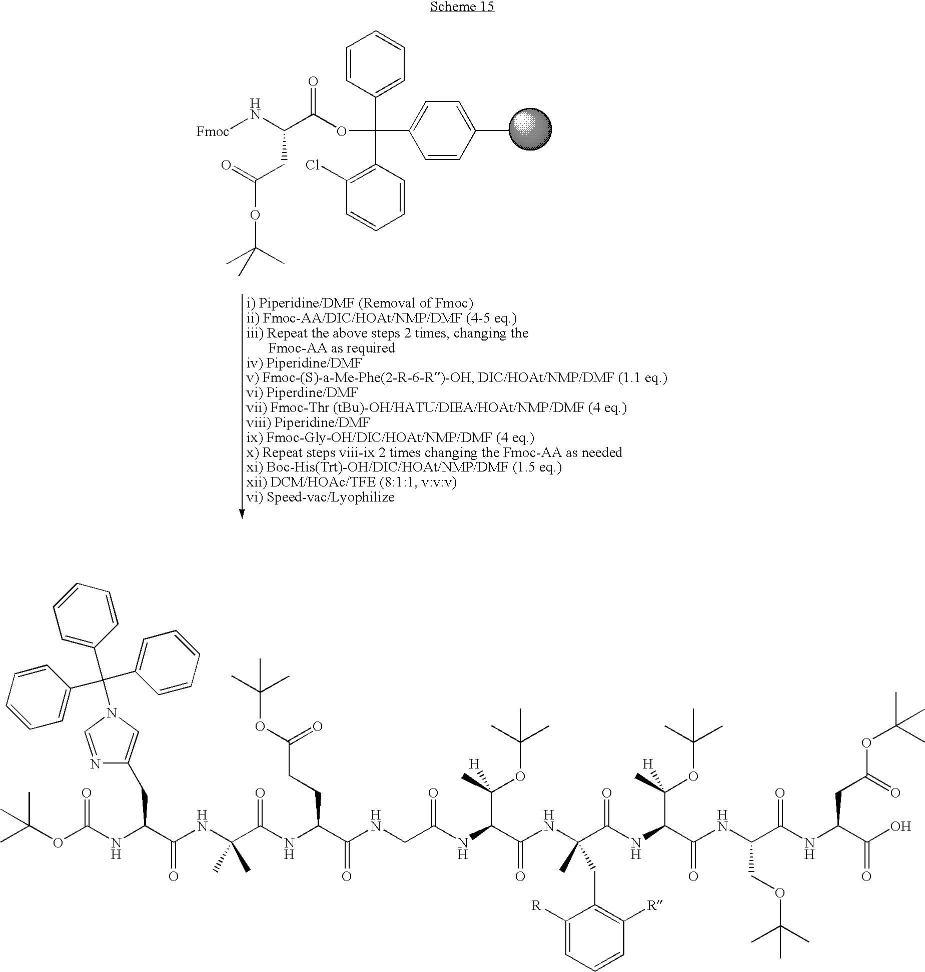
2. Procedure for Solid Phase Synthesis of Side Chain Protected 9-Mer Peptide C-Terminal Carboxylic Acid (Scheme 15)

A solution of Fmoc-(L)-Ser(tBu)-OH (5 eq.), 0.5 M HOAt/DMF (5 eq.) and DIC (5 eq.) in NMP (5 mL) was vortexed with (L)-Asp (OtBu)-2-chloro chlorotrityl resin (3.0 g, 2.16 mmol) for 18 hours at room temperature. After several washes with NMP, the Fmoc group was removed by treatment with 1.5 M piperidine/DMF twice (5 min and 10 min). These coupling and deprotection steps were repeated seven times to assemble the desired sequence, except that 1.1 eq. and 1.5 eq. of Fmoc-α-Me-Phe (2-R-6-R″)-OH and Boc-(L)-His(Trt)-OH were used, respectively, for their couplings, and that HATU/HOAt and DIEA (4 eq.) were used for coupling Fmoc-Thr(tBu)-OH onto (S)-α-Me-Phe(2-R-6-R″)-peptidyl-resin.

Upon assembly completion, the peptidyl-resin washed with DCM and the protected 9-mer peptide C-terminal carboxylic acid was released from the resin by treatment with DCM/AcOH/TFE (8:1:1, v:v:v) for one hour at room temperature. The resin was filtered off and the filtrate evaporated to dryness, redissolved in AcCN/water (2:1) and lyophilized twice, to yield 2.777 g of 81% pure product, which was used in the subsequent fragment coupling step with no further purification. 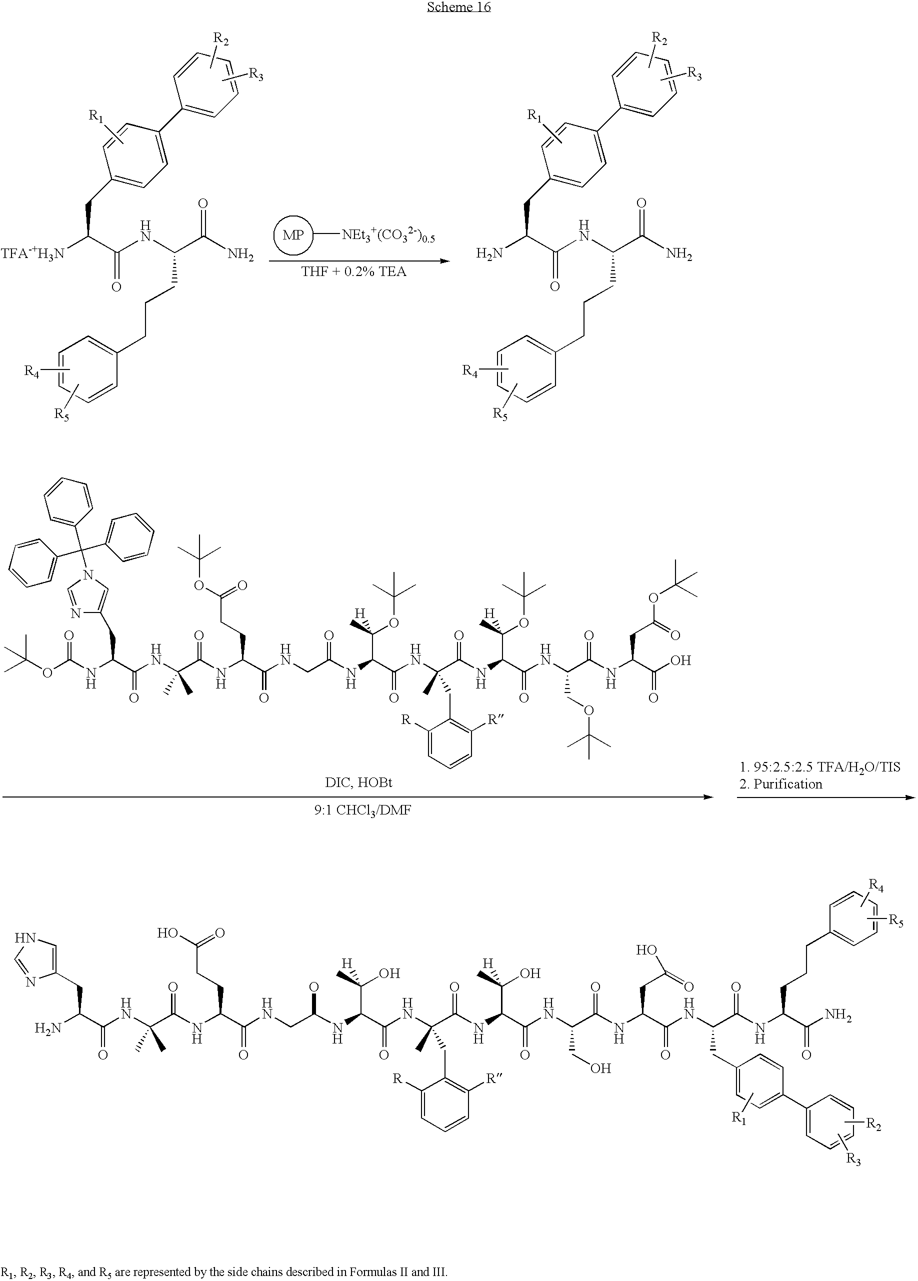
3. Procedure for Solution Phase Fragment Coupling Reaction (Scheme 16)

The reactions were performed in a single-compound format in 1 dram vials as well as in a parallel array of compounds in a 2 ml 96-well plate. The following description (shown in Scheme 16) applies to the single-compound case, but may be readily applied in a 96-well plate.

The TFA-salt of the dipeptide (0.01 mmol) was dissolved in 0.25 ml THF containing 0.2% triethylamine in a 1.5 ml glass vial. Macroporous carbonate resin (MP-carbonate, 0.03 mmol, Argonaut Technologies) was added to the vial. The vial was capped and agitated for two hours at room temperature. The solution was filtered and excess solvent was removed by evaporation.

A solution of 0.15 ml of 9:1 chloroform/N,N-dimethylformamide containing the side chain protected 9-mer peptide C-terminal carboxylic acid (0.008 mmol) and N-hydroxybenzotriazole (HOBt, 0.008 mmol) was added to the vial containing the dipeptide amine. Diisopropylcarbodiimide (DIC, 0.008 mmol) was added in a solution of 0.05 ml 9:1 chloroform/N,N-dimethylformamide. The vial was capped, and the reaction stirred on an orbital shaker at room temperature for 16 hours. The remaining solvent was evaporated from the vial.

The 11-mer peptide side chains and N-terminal α-amine were deprotected with 0.40 ml 95:2.5:2.5 trifluoroacetic acid/water/triisopropylsilane (TFA/H₂O/TIS) for one hour. The remaining solvent was evaporated, and the 11-mer peptide products were purified by HPLC, using a CH₃CN/H₂O/TFA solvent system, and triggering effluent collection by the detection of desired product mass, by the detection of the desired product [(M+2H⁺)/2]⁺ ion, or by UV detection of peaks.

### EXAMPLE 7

### General Procedure for Preparation of Peptides Via Fragment Condensation

In this approach, solid phase acylations were performed (as described in Example 3) to prepare the required amino acids represented by Formula II and Formula IV at positions Xₐₐ₁₀ and Xₐₐ₁₁. The dipeptide was cleaved from a support, with either prior (see Scheme 17) or simultaneous (see Scheme 18) removal of the N-terminal α-amine protecting group. The dipeptide was then coupled to a fully side chain-protected 9 amino acid peptide (Scheme 15). Subsequent deprotection of side chains and purification resulted in the desired 11-mer peptide products.
1. Procedures for Cleavage of Dipeptides from Rink Amide MBHA Resin 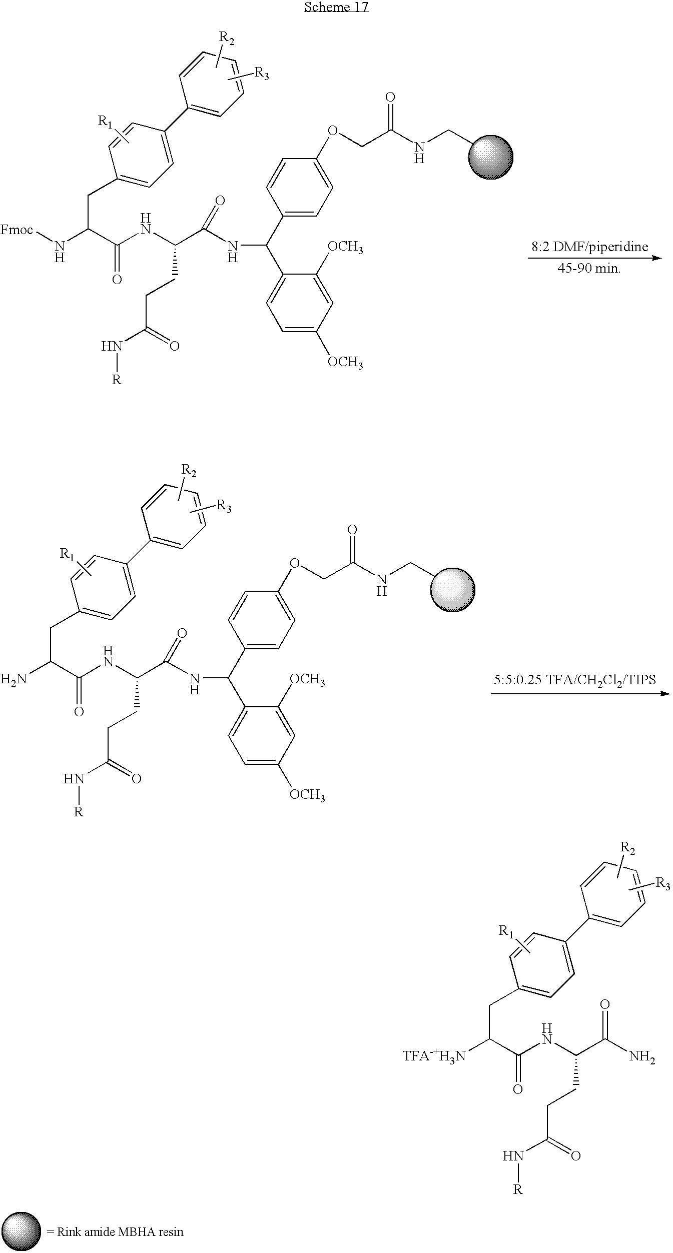
A. Procedure for Fmoc-Protected Dipeptides (Scheme 17)

The Fmoc-protected dipeptide-Rink amide resin (100 mg, 64 μmol) was soaked in dichloromethane (1.5 mL) for ten minutes. The resin was drained, transferred to a 1-dram vial, and a solution of 8:2 DMF/piperidine (1.5 mL) was added to the resin. The vial was capped and agitated for 45-90 minutes. The resin was then transferred back to a filter tube, drained, and washed with DMA (3×2 mL), MeOH (3×2 mL), and CH₂Cl₂ (3×2 mL). The resin was transferred to a 1-dram glass vial and a solution of 5:5:0.25 trifluoroacetic acid/CH₂Cl₂/triisopropylsilane (1.5 mL) was added. The vial was capped and the resin cleaved for two hours. After two hours the solution was filtered into a clean vial, and rinsed with MeOH (1×1 mL), which was added to the cleavage solution. Fresh TFA/CH₂Cl₂/TIPS solution was added to the resin and the cleavage reaction was repeated. The cleavage solutions were combined and solvent evaporated. The resulting product was purified by HPLC, using a C-18 column and CH₃CN/H₂O/TFA or MeOH/H₂O/TFA solvent system with either UV or mass directed fraction collection to yield (after evaporation of solvent) the dipeptide as the trifluoroacetic acid salt of the α-amine. 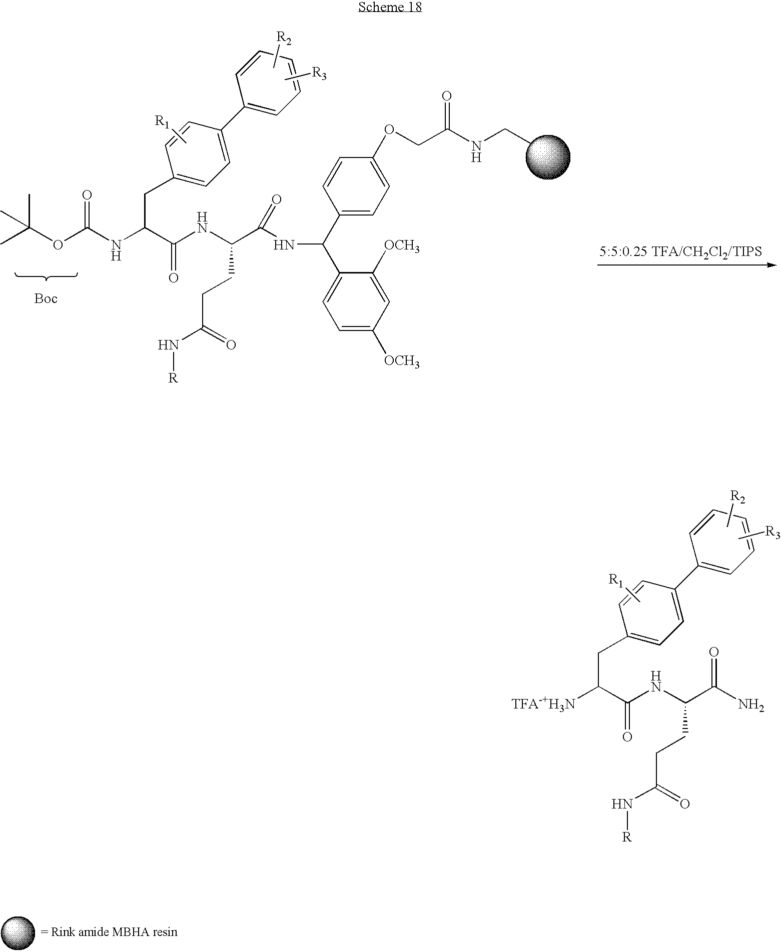
B. Procedure for Boc-Protected Dipeptides (Scheme 18)

The Boc-protected dipeptide-Rink resin (100 mg, 64 μmol) was added to a 1-dram glass vial and a solution of 5:5:0.25 trifluoroacetic acid/CH₂Cl₂/triisopropylsilane (1.5 mL) was added. The vial was capped and the resin cleaved for two hours. After two hours the solution was filtered into a clean vial, and rinsed with MeOH (1×1 mL), which was added to the cleavage solution. Fresh TFA/CH₂Cl₂/TIPS solution was added to the resin and the cleavage reaction was repeated. The cleavage solutions were combined and solvent evaporated. The resulting product was purified by HPLC, using a C-18 column and CH₃CN/H₂O/TFA or MeOH/H₂O/TFA solvent system with either UV or mass directed fraction collection to yield (after evaporation of solvent) the dipeptide as the trifluoroacetic acid salt of the α-amine. 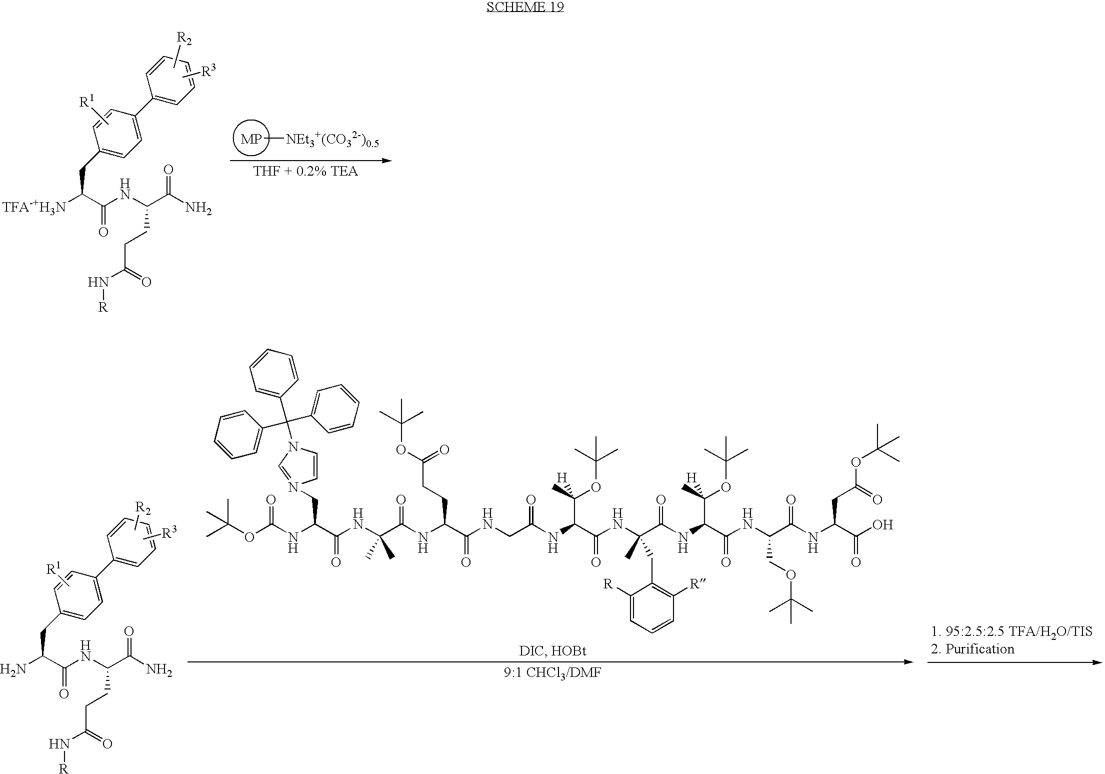 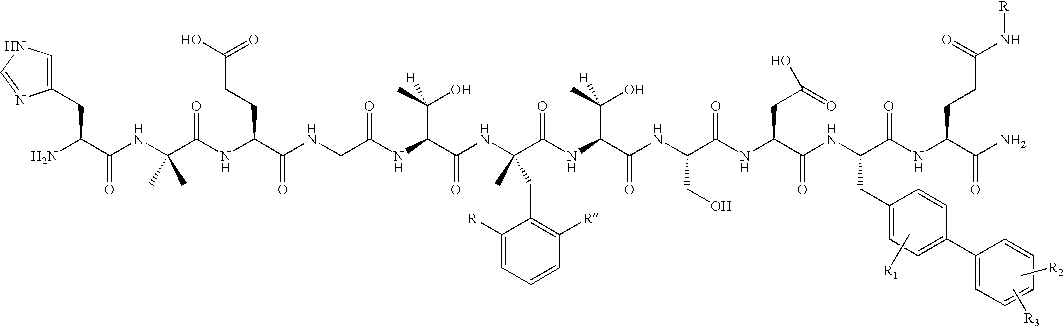
2. Procedure for Solution Phase Fragment Coupling Reaction (Scheme 19)

These reactions were performed in a single-compound format in 1 dram vials as well as in a parallel array of compounds in a 2 ml 96-well plate. The following description (shown in Scheme 19) applies to the single-compound case, but may be readily applied in a 96-well plate.

The TFA-salt of the dipeptide (0.01 mmol) was dissolved in 0.25 ml THF containing 0.2% triethylamine in a 1.5 ml glass vial. Macroporous carbonate resin (MP-carbonate, 0.03 mmol, Argonaut Technologies) was added to the vial. The vial was capped and agitated for two hours at room temperature. The solution was filtered, and excess solvent was removed by evaporation.

A solution of 0.15 ml of 9:1 chloroform/N,N-dimethylformamide containing the side chain protected 9-mer peptide C-terminal carboxylic acid (0.008 mmol) and N-hydroxybenzotriazole (HOBt, 0.008 mmol) was added to the vial containing the dipeptide amine. Diisopropylcarbodiimide (DIC, 0.008 mmol) was added in a solution of 0.05 ml 9:1 chloroform/N,N-dimethylformamide. The vial was capped, and the reaction stirred on an orbital shaker at room temperature for 16 hours. Remaining solvent was evaporated from the vial.

The 11-mer peptide side chains and N-terminal α-amine were deprotected with 0.40 ml 95:2.5:2.5 trifluoroacetic acid/water/triisopropylsilane (TFA/H₂O/TIS) for one hour. The remaining solvent was evaporated, and the 11-mer peptide products were purified by HPLC, using a CH₃CN/H₂O/TFA solvent system, and triggering effluent collection by the detection of desired product mass, by the detection of the desired product [(M+2H⁺)/2]⁺ ion, or by UV detection of peaks.

### EXAMPLE 8

### General Procedure for Preparation of Peptides Via Fragment Condensation

In this approach, solid phase acylations were performed (as described in Example 4) to prepare the required amino acids represented by Formula II and Formula V at positions Xₐₐ10 and Xₐₐ11. The dipeptide was cleaved from the support, with either prior (see Scheme 20) or simultaneous (see Scheme 21) removal of the N-terminal α-amine protecting group. The dipeptide was then coupled to a fully side chain-protected 9 amino acid peptide (Scheme 15). Subsequent deprotection of side chains and purification resulted in the desired 11-mer peptide products.
1. Procedures for Cleavage of Dipeptides from Rink Amide MBHA Resin 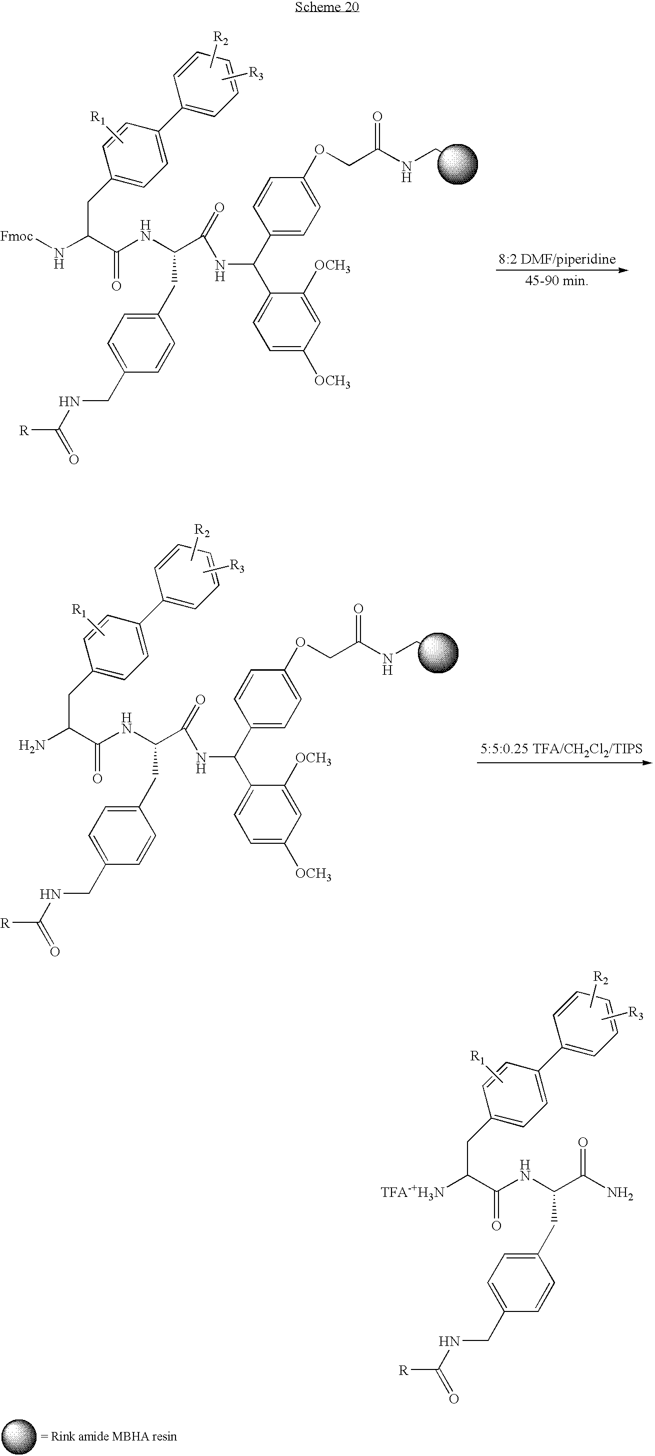
A. Procedure for Fmoc-Protected Dipeptides (Scheme 20)

The Fmoc-protected dipeptide-Rink amide resin (100 mg, 64 μmol) was soaked in dichloromethane (1.5 mL) for ten minutes. The resin was drained, transferred to a 1-dram vial, and a solution of 8:2 DMF/piperidine (1.5 mL) was added to the resin. The vial was capped and agitated for 45-90 minutes The resin was transferred back to a filter tube, drained, and washed with DMA (3×2 mL), MeOH (3×2 mL), and CH₂Cl₂ (3×2 mL). The resin was transferred to a 1-dram glass vial and a solution of 5:5:0.25 trifluoroacetic acid/CH₂Cl₂/triisopropylsilane (1.5 mL) was added. The vial was capped and the resin cleaved for two hours. After two hours the solution was filtered into a clean vial, and rinsed with MeOH (1×1 mL), which was added to the cleavage solution. Fresh TFA/CH₂Cl₂/TIPS solution was added to the resin and the cleavage reaction was repeated. The cleavage solutions were combined and solvent evaporated. The resulting product was purified by HPLC, using a C-18 column and CH₃CN/H₂O/TFA or MeOH/H₂O/TFA solvent system with either UV or mass directed fraction collection to yield (after evaporation of solvent) the dipeptide as the trifluoroacetic acid salt of the α-amine. 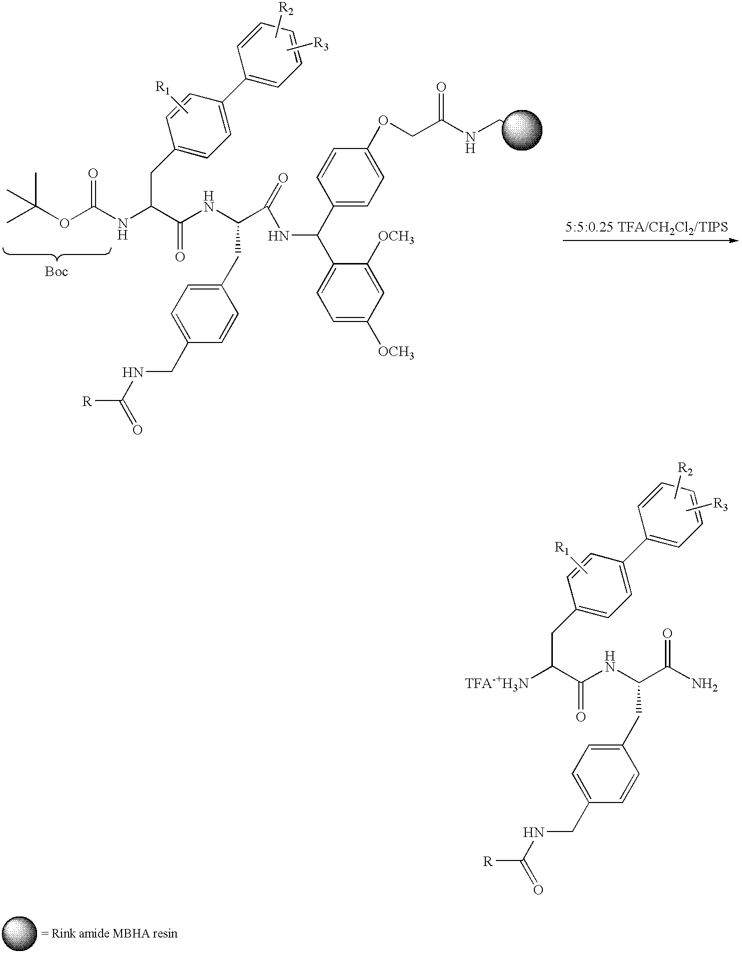
B. Procedure for Boc-Protected Dipeptides (Scheme 21)

The Boc-protected dipeptide-Rink resin (100 mg, 64 μmol) was added to a 1-dram glass vial and a solution of 5:5:0.25 trifluoroacetic acid/CH₂Cl₂/triisopropylsilane (1.5 mL) was added. The vial was capped and the resin cleaved for two hours. After two hours the solution was filtered into a clean vial, and rinsed with MeOH (1×1 mL), which was added to the cleavage solution. Fresh TFA/CH₂Cl₂/TIPS solution was added to the resin and the cleavage reaction was repeated. The cleavage solutions were combined and solvent evaporated. The resulting product was purified by HPLC, using a C-18 column and CH₃CN/H₂O/TFA or MeOH/H₂O/TFA solvent system with either UV or mass directed fraction collection to yield (after evaporation of solvent) the dipeptide as the trifluoroacetic acid salt of the α-amine. 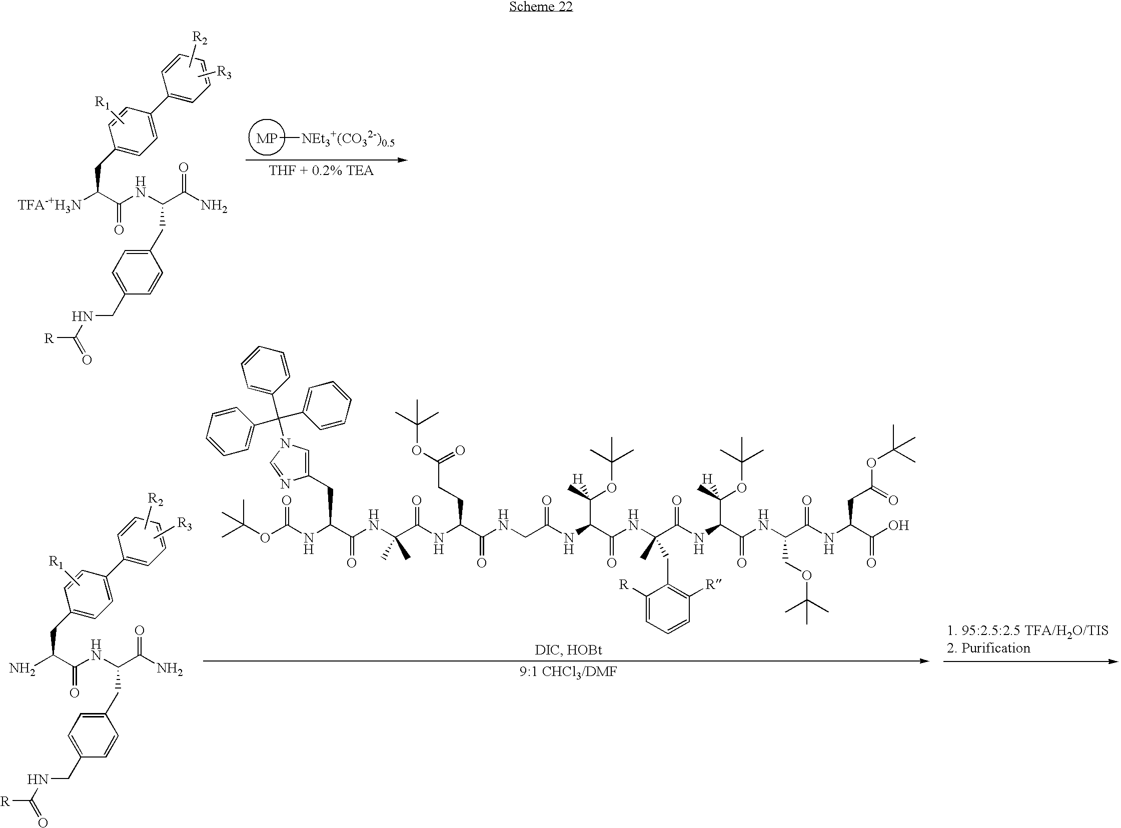 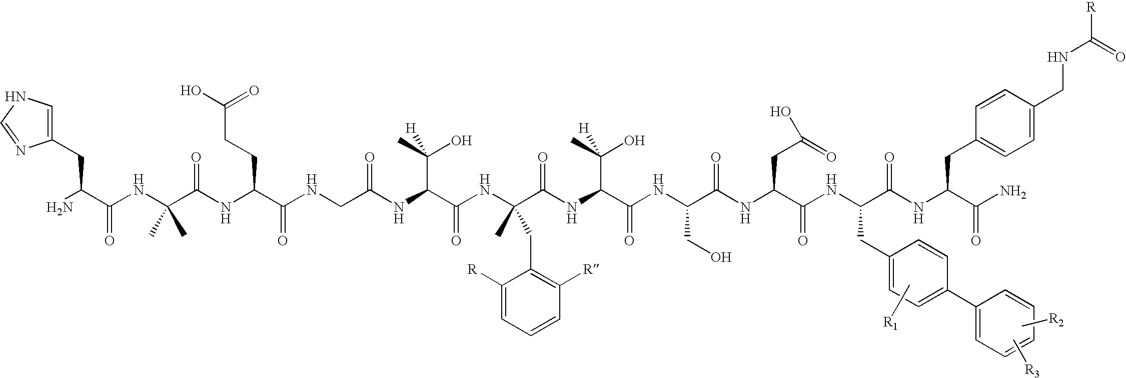
3. Procedure for Solution Phase Fragment Coupling Reaction (Scheme 22)

These reactions were performed in a single-compound format in 1 dram vials as well as in a parallel array of compounds in a 2 ml 96-well plate. The following description (shown in Scheme 24) applies to the single-compound case, but may be readily applied in a 96-well plate.

The TFA-salt of the dipeptide (0.01 mmol) was dissolved in 0.25 ml THF containing 0.2% triethylamine in a 1.5 ml glass vial. Macroporous carbonate resin (MP-carbonate, 0.03 mmol, Argonaut Technologies) was added to the vial. The vial was capped and agitated for two hours at room temperature. The solution was filtered, and excess solvent was removed by evaporation.

A solution of 0.15 ml of 9:1 chloroform/N,N-dimethylformamide containing the side chain protected 9-mer peptide C-terminal carboxylic acid (0.008 mmol) and N-hydroxybenzotriazole (HOBt, 1.22 mg, 0.008 mmol) was added to the vial containing the dipeptide amine. 1,3-diisopropylcarbodiimide (DIC, 1.25 μL, 0.008 mmol) was added in a solution of 0.05 ml 9:1 chloroform/N,N-dimethylformamide. The vial was capped, and the reaction stirred on an orbital shaker at room temperature for 16 hours. The remaining solvent was then evaporated from the vial.

The 11-mer peptide side chains and N-terminal α-amine were deprotected with a solution of 0.40 ml 95:2.5:2.5 trifluoroacetic acid/water/triisopropylsilane (TFA/H₂O/TIS) for one hour. The remaining solvent was evaporated, and the 11-mer peptide products were purified by HPLC, using a CH₃CN/H₂O/TFA solvent system, and triggering effluent collection by the detection of desired product mass, by the detection of the desired product [(M+2H⁺)/2]⁺ ion, or by UV detection of peaks.

### EXAMPLE 9

### General Procedure for Preparation of Peptides Via Fragment Condensation

In this approach, solid phase acylations were performed (as described in Example 5) to prepare the required amino acids of Formula II and Formula VI at positions Xₐₐ₁₀ and Xₐₐ₁₁. The dipeptide was cleaved from the support, with either prior (see Scheme 23) or simultaneous (see Scheme 24) removal of the N-terminal α-amine protecting group. The dipeptide was then coupled to a fully side chain-protected 9 amino acid peptide (Scheme 15). Subsequent deprotection of side chains and purification resulted in the desired 11-mer peptide products.
1. Procedures for Cleavage of Dipeptides from Sieber Amide Resin 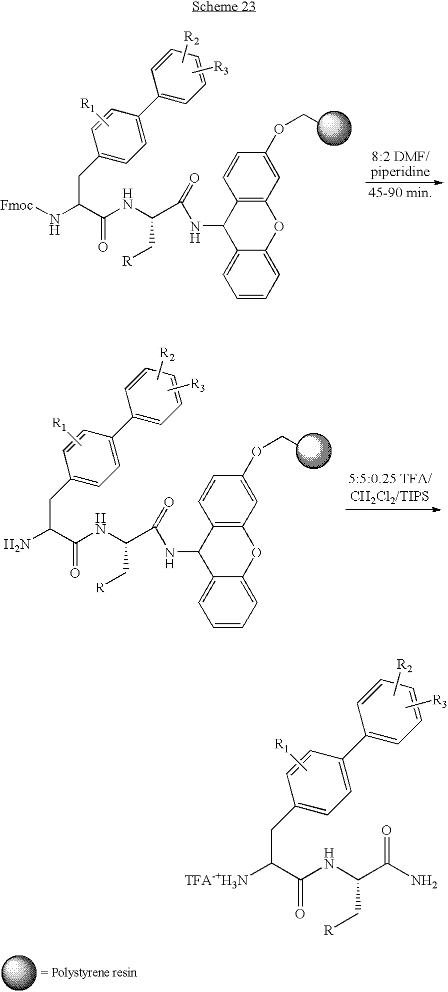
A. Procedure for Fmoc-Protected Dipeptides (Scheme 23)

The Fmoc-protected dipeptide-Sieber amide resin (100 mg, 52 μmol) was soaked in dichloromethane (1.5 mL) for ten minutes. The resin was drained, transferred to a 1-dram vial, and a solution of 8:2 DMF/piperidine (1.5 mL) was added to the resin. The vial was capped and agitated for 45-90 minutes. The resin was then transferred back to a filter tube, drained, and washed with DMA (3×2 mL), MeOH (3×2 mL), and CH₂Cl₂ (3×2 mL). The resin was transferred to a 1-dram glass vial and a solution of 5:5:0.25 trifluoroacetic acid/CH₂Cl₂/triisopropylsilane (1.5 mL) was added. The vial was capped and the resin cleaved for two hours. After two hours the solution was filtered into a clean vial, and rinsed with MeOH (1×1 mL), which was added to the cleavage solution. Fresh TFA/CH₂Cl₂/TIPS solution was added to the resin and the cleavage reaction was repeated. The cleavage solutions were combined and solvent evaporated. The resulting product was purified by HPLC, using a C-18 column and CH₃CN/H₂O/TFA or MeOH/H₂O/TFA solvent system with either UV or mass directed fraction collection to yield (after evaporation of solvent) the dipeptide as the trifluoroacetic acid salt of the α-amine. 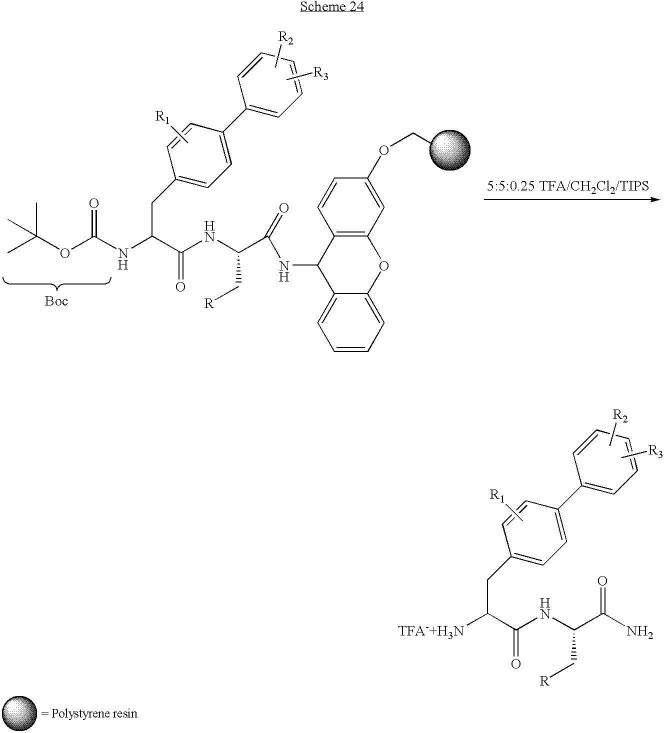
B. Procedure for Boc-Protected Dipeptides (Scheme 24)

The Boc-protected dipeptide-Sieber amide resin (100 mg, 52 μmol) was added to a 1-dram glass vial and a solution of 5:5:0.25 trifluoroacetic acid/CH₂Cl₂/triisopropylsilane (1.5 mL) was added. The vial was capped and the resin cleaved for two hours. After two hours the solution was filtered into a clean vial, and rinsed with MeOH (1×1 mL), which was added to the cleavage solution. Fresh TFA/CH₂Cl₂/TIPS solution was added to the resin and the cleavage reaction was repeated. The cleavage solutions were combined and solvent evaporated. The resulting product was purified by HPLC, using a C-18 column and CH₃CN/H₂O/TFA or MeOH/H₂O/TFA solvent system with either UV or mass directed fraction collection to yield (after evaporation of solvent) the dipeptide as the trifluoroacetic acid salt of the α-amine. 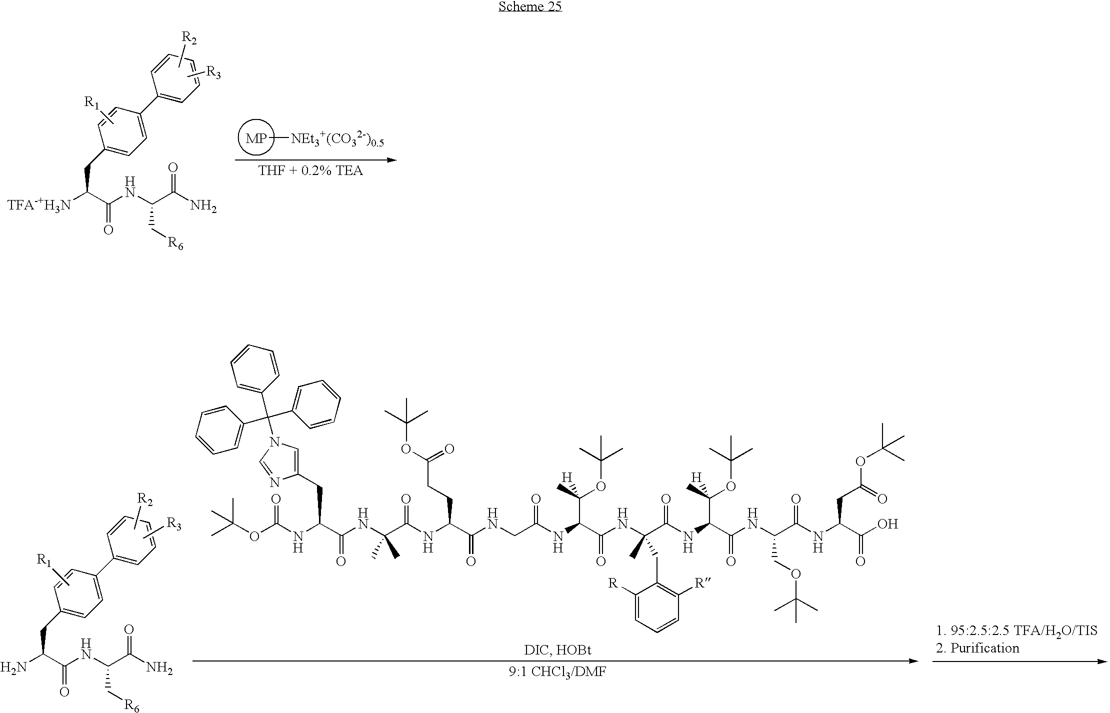 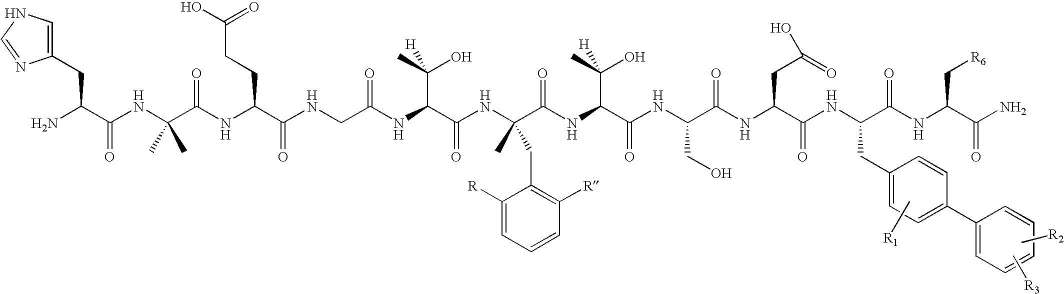
3. Procedure for Solution Phase Fragment Coupling Reaction (Scheme 25)

These reactions were performed in a single-compound format in 1 dram vials as well as in a parallel array of compounds in a 2 ml 96-well plate. The following description (shown in Scheme 25) applies to the single-compound case, but may be readily applied in a 96-well plate.

The TFA-salt of the dipeptide (0.01 mmol) was dissolved in 0.25 ml THF containing 0.2% triethylamine in a 1.5 ml glass vial. Macroporous carbonate resin (MP-carbonate, 0.03 mmol, Argonaut Technologies) was added to the vial. The vial was capped and agitated for two hours at room temperature. The solution was filtered, and excess solvent was removed by evaporation.

A solution of 0.15 ml of 9:1 chloroform/N,N-dimethylformamide containing the side chain protected 9-mer peptide C-terminal carboxylic acid (0.008 mmol) and N-hydroxybenzotriazole (HOBt, 0.008 mmol) was added to the vial containing the dipeptide amine. Diisopropylcarbodiimide (DIC, 0.008 mmol) was added in a solution of 0.05 ml 9:1 chloroform/N,N-dimethylformamide. The vial was capped, and the reaction stirred on an orbital shaker at room temperature for 16 hours. Remaining solvent was evaporated from the vial.

The 11-mer peptide side chains and N-terminal α-amine were deprotected with 0.40 ml 95:2.5:2.5 trifluoroacetic acid/water/triisopropylsilane (TFA/H₂O/TIS) for one hour. The remaining solvent was evaporated, and the 11-mer peptide products were purified by HPLC, using a CH₃CN/H₂O/TFA solvent system, and triggering effluent collection by the detection of desired product mass, by the detection of the desired product [(M+2H⁺)/2]⁺ ion, or by UV detection of peaks.

### EXAMPLE 10

### Synthesis of Biphenylalanine Analogs at Position Xₐₐ₁₀ and Phenoxy HomoSer Analogs at Position Xₐₐ₁₁ Represented by Formula XI

Analogs with position Xₐₐ₁₀ and Xₐₐ₁₁ residues represented by substituted amino acid analogs of Formula XI, i.e. biphenylalanine analogs (Bip analogs) or hetero-biphenylalanine analogs, and phenoxy homoSer analogs, were incorporated into the peptide chain via one of the following approaches. Synthesis of peptides of SEQ ID NOs: 64 and 77 are exemplified.

### EXAMPLE 11

### (Approach A, Synthesis of a Peptide of SEQ ID NO:64)

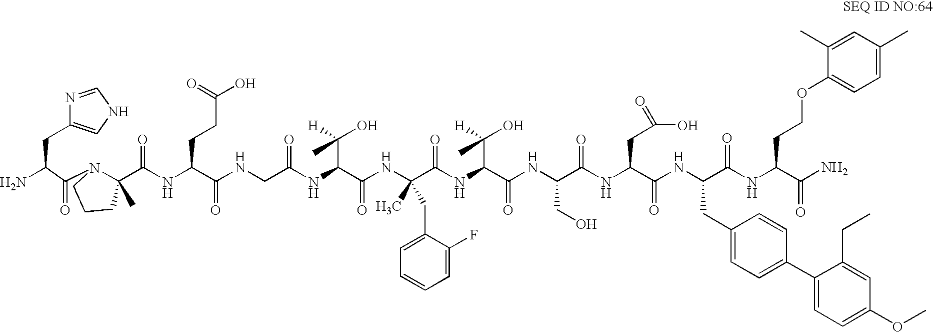

### EXAMPLE 11a

### Procedure for Synthesis of (S)-Benzyl 2-(Tert-Butoxycarbonylamino)-4-(2,4-Dimethylphenoxy)Butanoate

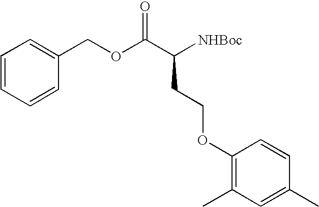

DIAD (55 ul, 0.275 mmol was added at room temperature to the mixture of Boc-Hse-Obzl (77.3 mg, 0.25 mmol), 2,4-dimethyl phenol (36.7 mg, 0.3 mmol) and PPh₃ (72.2 mg, 0.275 mmol) in 1.5 mL of THF. The solution was stirred for four hours under nitrogen. The solvent was removed by evaporation under vacuum. The crude product (S)-benzyl 2-(tert-butoxycarbonyl)-4-(2,4-dimethylphenoxy)butanoate was purified by Prep-HPLC-MS and analyzed by LC-MS. It yielded about 93.44 mg of the desired product, which has 95% of purity with (M+H)⁺ (413.15) in LC-MS.

### EXAMPLE 11b

### Procedure for Synthesis of (S)-tert-butyl 1-Amino-4-(2,4-dimethylphenoxy)-1-oxobutan-2-ylcarbamate

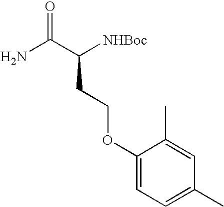

(S)-benzyl 2-(tert-butoxycarbonyl)-4-(2,4-dimethylphenoxy)butanoate (93 mg, 0.23 mmol) was treated with 4 mL of ammonia ˜7N solution in methyl alcohol in a sealed tube at 90° C. for 24 hours. The solution was cooled to room temperature and solvent was removed evaporation under vacuum. The crude product (S)-tert-butyl 1-amino-4-(2,4-dimethylphenoxy)-1-oxobutan-2-ylcarbamate was formed with high purity, which was directly used for next step (˜100% yield). The identity of the product was confirmed by analytical LC-MS.

### EXAMPLE 11c

### Procedure for Synthesis of (S)-2-amino-4-(2,4-dimethylphenoxy)butanamide

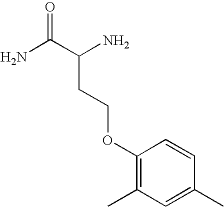

Triethylsilane (100 μl, 0.62 mmol) and TFA (500 μl) was added to the crude (S)-tert-butyl 1-amino-4-(2,4-dimethylphenoxy)-1-oxobutan-2-ylcarbamate (˜0.23 mmol) in 500 μl of DCM. The reaction proceeded for two hours with stirring. The reaction was dried under vacuum. The TFA salt of the crude (S)-2-amino-4-(2,4-dimethylphenoxy)butanamide was formed with high purity, and was directly used for the next step (˜100% yield). The structures were confirmed by analytical LC-MS.

### EXAMPLE 11d

### Procedure for Synthesis and Separation of 1D-1 and 1D-2

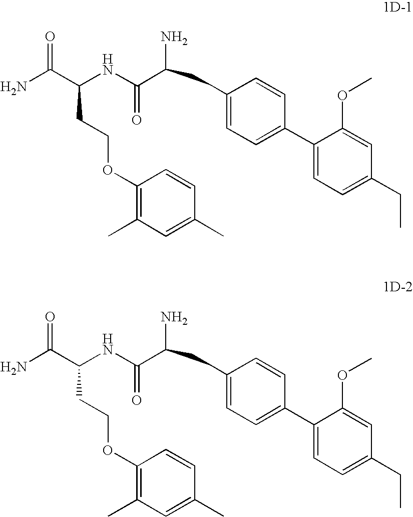

About 0.22 mmol of crude amine(R,S)-2-amino-4-(2,4-dimethylphenoxy)butanamide was added to the reaction vessel along with about 2 mL of DMF solution of Fmoc-L-4′-methoxy-2′-ethylbiphenylalanine (114.8 mg, 0.22 mmol), PyAOP (114.5 mg, 0.22 mmol) and HOBt (33.6 mg, 0.22 mmol) mixture, followed by addition of DIEA (76.5 μl, 0.44 mmol). The reaction was stirred vigorously for 20 hours. The reaction was monitored by analytical LC-MS. Next, 650 μl of piperidine was added into the reaction. The Fmoc-protecting group was removed after stirring for two hours. The reaction mixture was evaporated to dryness under vacuum. The mixture of crude products 1D-1 and 1D-2 were purified and separated by reverse phase Prep-HPLC-MS to provide pure TFA salts 1D-1 (47.5 mg, fast-moving) and 1D-2 (32.3 mg, slow-moving). Compounds were analyzed by LC-MS. The NMR spectra characteristics of 1D-1 was as follows: 1H NMR (500 MHz, MeOH): δ 0.978 (t, 3H), 2.08 (s, 3H), 2.11 (s, 3H), 2.25 (m, 1H), 2.47 (q, 2H), 3.04 (m, 2H), 3.24 (m, 3H), 3.72 (s, 3H), 3.93 (m, 2H), 4.10 (t, 1H), 4.58 (m, 1H), 6.65 (d, 1H), 6.69 (d, 1H), 6.76 (d, 1H), 6.80 (m, 2H), 6.96 (d, 1H), 7.17 (d, 2H), 7.25 (d, 2H).

### EXAMPLE 11e

### Procedure for Generation of a Peptide of SEQ ID NO:64 Via Fragment Coupling

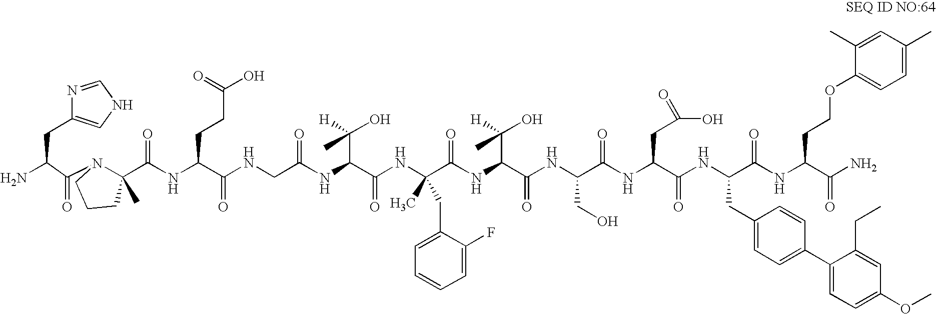

The TFA-salt of the dipeptide 1D-1 (0.015 mmol) was dissolved in 0.5 ml of 9:1 chloroform/N,N-dimethylformamide containing 0.015 mmol DIEA in a 10 mL of glass vial. Then, a solution of 0.5 ml of 9:1 chloroform/N,N-dimethylformamide containing the appropriate side chain protected 9 amino acid peptide (0.015 mmol), N-hydroxybenzotriazole (HOBt, 0.015 mmol) and diisopropylcarbodiimide (DIC, 0.015 mmol) was added into the solution with 1D-1. The vial was capped, and the reaction stirred at room temperature for 16 hours. The remaining solvent was evaporated from the vial.

The resulting 11-mer peptide side chains and N-terminal α-amine were deprotected with 1 ml 95:5:5 trifluoroacetic acid/water/triisopropylsilane (TFA/H₂O/TIS) for two hours. The remaining solvent was evaporated, and the 11-mer peptide product was purified by Prep-HPLC-MS, using a CH₃CN/H₂O/TFA solvent system, and triggering effluent collection by the detection of desired product mass, by the detection of the desired product [(M+2H⁺)/2]⁺ ion, or by UV detection of peaks. Purification provided the TFA salt of the peptide of SEQ ID NO: 64 (5.2 umol, 35% yield). LC-MS analysis indicates 97% purity and observation of the [(M+2H⁺)/2]⁺ ion, 761.93.

### EXAMPLE 12

### (Approach B, Synthesis of A Peptide of SEQ ID NO:77)

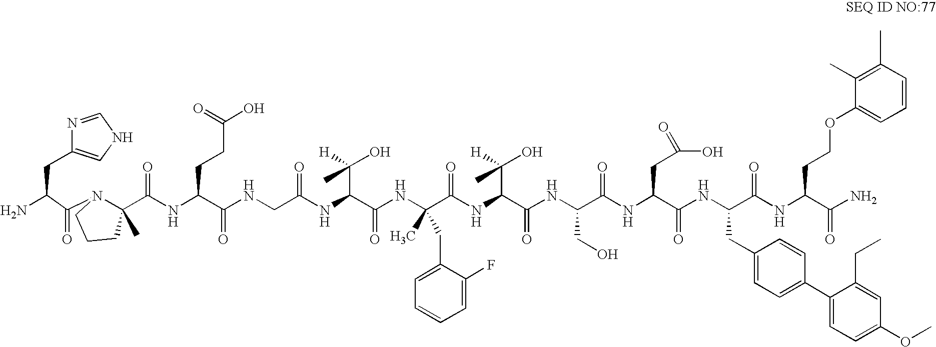

### EXAMPLE 12a

Procedure for Synthesis of (S)-methyl 4-bromo-2-(tert-butoxycarbonylamino)butanoate

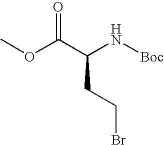

Triethylamine (1 mL, 7.2 mmol) was added to a solution of methyl(S)-2-amino-4-bromobutyrate HBr in 10 ml of dioxane/H₂O. The reaction flask was cooled to 0° C., and di-tert-butyl dicarbonate (944 mg, 4.33 mmol) was added in one batch. After 30 minutes, the cold bath was removed and the reaction mixture was stirred at room temperature overnight. The reaction mixture was concentrated by evaporation under vacuum. The residue was diluted with H₂O and EtOAc. The aqueous layer was extracted with EtOAc (2×). The combined organic layer washed by saturated NaCl solution, and dried over MgSO₄. After filtration, the solvent was removed under vacuum to provide crude (S)-methyl 4-bromo-2-(tert-butoxycarbonyl)butanoate (900 mg) with high purity. This product was directly used in the next step of the reaction.

### EXAMPLE 12b

### Procedure for Synthesis of (S)-methyl 2-(tert-butoxycarbonylamino)-4-(2,3-dimethylphenoxy)butanoate

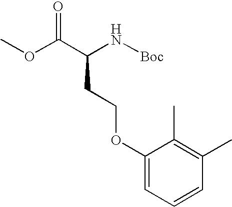

2,3-dimethyl phenol (48.87 mg, 0.375 mmol) and K₂CO₃ (0.6 mmol) was added to a solution of (S)-methyl 4-bromo-2-(tert-butoxycarbonyl)butanoate (44.4 mg, 0.15 mmol) in 1.5 mL DMF. The reaction mixture was heated to 75° C. and stirred for 20 hours. The solution was cooled to room temperature and solvent was removed under vacuum. The crude product (S)-methyl 2-(tert-butoxycarbonyl)-4-(2,3-dimethylphenoxy)butanoate was purified by Prep-HPLC-MS, using a CH₃CN/H₂O/TFA solvent system, and triggering effluent collection by detection of the desired product mass, providing purified product (45 mg).

### EXAMPLE 12c

### Procedure for Synthesis of (S)-tert-butyl 1-amino-4-(2,3-dimethylphenoxy)-1-oxobutan-2-ylcarbamate

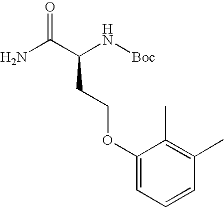

(S)-methyl 2-(tert-butoxycarbonyl)-4-(2,3-dimethylphenoxy)butanoate (45 mg) was treated with 4 mL of ammonia ˜7N solution in methyl alcohol in a sealed tube at 90° C. for 24 hours. The solution was cooled to room temperature and solvent was removed under vacuum. The crude product (S)-tert-butyl 1-amino-4-(2,3-dimethylphenoxy)-1-oxobutan-2-ylcarbamate was formed in high purity, which was then directly used for next step (˜100% yield). The identity of the product was confirmed by LC-MS.

### EXAMPLE 12d

### Procedure for Synthesis of (S)-2-amino-4-(2,3-dimethylphenoxy)butanamide

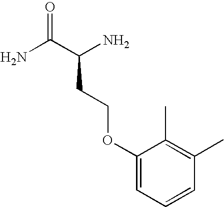

Triethylsilane (100 μl, 0.62 mmol) and TFA (500 μl) was added to the crude product (S)-tert-butyl 1-amino-4-(2,3-dimethylphenoxy)-1-oxobutan-2-ylcarbamate (˜0.135 mmol) in 500 μl of DCM. The reaction proceeded for two hours with stirring. The reaction was dried under vacuum. The TFA salt of crude product (S)-2-amino-4-(2,3-dimethylphenoxy)butanamide was formed with high purity, which was directly used for next step (˜100% yield). The identity of the product was confirmed by LC-MS.

### EXAMPLE 12e

### Procedure for Synthesis and Separation of 2J-1 and 2J-2

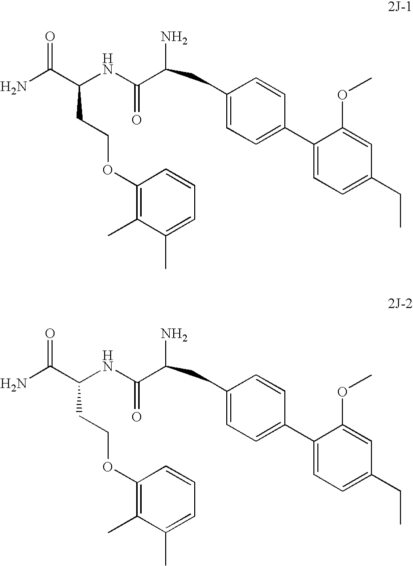

This reaction and separation of resulting diastereomers was conducted in a manner analogous to that described in Example 11d. The NMR characteristics of 2J-1 are the following: ¹H NMR (400 MHz, MeOH): δ 1.09 (t, 3H), 2.17 (s, 3H), 2.25 (s, 3H), 2.39 (m, 1H), 2.59 (q, 2H), 3.12 (m, 1H), 3.34 (m, 3H), 3.83 (s, 3H), 4.07 (m, 2H), 4.19 (dd, 1H), 4.58 (dd, 1H), 6.74 (dd, 1H), 6.80 (dd, 1H), 6.87 (d, 1H), 7.00 (t, 1H), 7.07 (d, 1H), 7.28 (d, 2H), 7.35 (d, 2H).

### EXAMPLE 12f

### Procedure for Generation of a Peptide of SEQ ID NO:77 Via Fragment Coupling

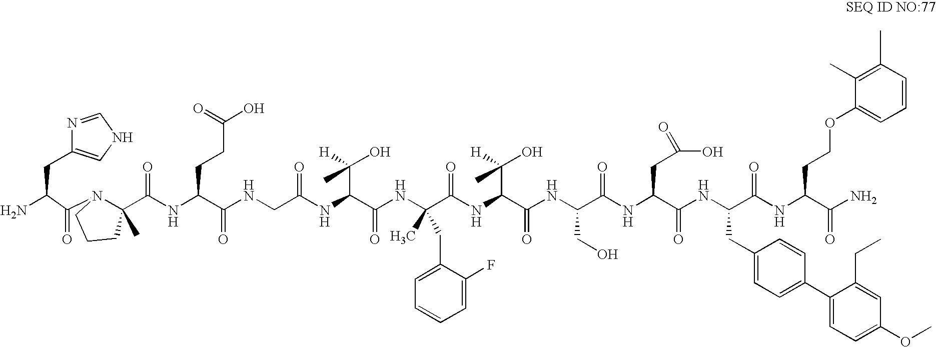

A peptide of SEQ ID NO:77 was prepared using a similar procedure as that used in the preparation of the peptide of SEQ ID NO:64, described in Example 11E, using the starting material 2J-1 (0.015 mmol). The resulting reaction and purification provided the TFA salt of compound 77 (5.1 umol, 35% yield). LC-MS analysis reveals 100% purity and observation of the [(M+2H⁺)/2]⁺ ion, 761.94.

### EXAMPLE 13

### Synthesis of a Peptide of SEQ ID NO:145

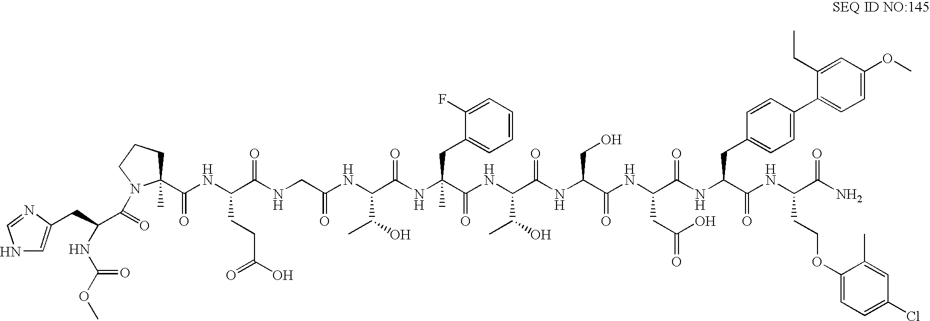

### EXAMPLE 13a

### Procedure for Synthesis of 2-(S)-Fluorenylmethoxycarbonylamino-4-(2-methyl-4-chloro)phenoxybutanoic acid

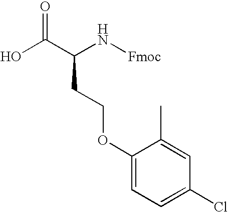

This amino acid can be prepared starting from (S)-methyl 2-(tert-butoxycarbonylamino)-4-((2-methyl-4-chloro)phenoxy)butanoate, which can be prepared using procedures similar to those described in Examples 12a-b. After removal of the methyl ester by saponification and t-Boc removal using TFA, the resulting amino acid can be converted to its Fmoc-protected derivative using standard procedures such as reaction with 9-Fluorenylmethoxycarbonyl chloride (Fmco-Cl) in a solution of aqueous sodium carbonate (Na₂CO₃) and THF or with N-(9-Fluorenylmethoxycarbonyl-oxy)succinimide (Fmoc-OSu) in an aqueous sodium bicarbonate (NaHCO₃) solution and THF.

### EXAMPLE 13b

### Procedure for Synthesis of a Peptide of SEQ ID NO:145 via Stepwise Elongation

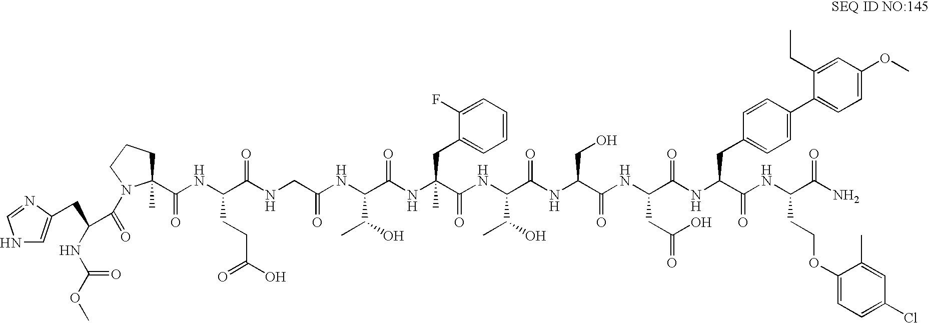 A. Synthesis of Fmoc-Xₐₐ₁₀-Xₐₐ₁₁-Dipeptidyl-Sieber Resin

An amount of 9-Fmoc-aminoxanthen-3-yloxy-Merrifield resin (Sieber amide resin; loading: 0.65 mmol/g) sufficient to synthesize several 11 amino acid analogs was swelled by washing with DMF (1×10 mL, 20 minutes). The Fmoc group was then removed using two treatments, 5 and 15 minutes each respectively, with 20% piperidine in DMF (10 mL/g). The resin washed with DMF (7×10 mL). A solution of 2-(S)-Fluorenylmethoxycarbonylamino-4-(2-methyl-4-chloro)phenoxybutanoic acid (1.1 eq) dissolved in 0.546M HOAt in DMF (1.1 eq) was added to the resin, followed by the addition of DIC (1.1 eq). The resin was then shaken or vortexed for 3.5 days. Coupling completion was monitored using a qualitative ninhydrin test. The resin was drained and washed with DMF (4×10 mL).

A second manual coupling cycle using DIC/HOAt was then performed, starting with the removal of the Fmoc group with 20% piperidine in DMF, as described previously. A solution of Fmoc-Biphenylalanine (2′-Et-4′-OMe)-OH (1.5 eq.) dissolved in 0.546M HOAt in DMF (1.5 eq) was added to the resin, followed by a rinse with DMF (1 mL), and addition of DIC (1.5 eq). The resin was then shaken or vortexed for 16 hours. Coupling completion was monitored using a qualitative ninhydrin test. The resin was drained and washed with DMF (4×10 mL), to yield the desired Fmoc-protected dipeptidyl-Sieber amide resin.

An aliquot of the Fmoc-protected dipeptidyl-resin (0.05 mmol) was added to a vessel of appropriate size on the instrument, washed six times with NMP and deprotected using two treatments with 20% piperidine/NMP (2 and 8 minutes each). One additional monitored deprotection step was performed until the conditions of the monitoring option were satisfied. The total deprotection time was 10-12 minutes. The deprotected dipeptidyl-resin washed six times with NMP and then coupled with Fmoc-L-Asp(OtBu)-OH as follows: Fmoc-L-Asp(OtBu)-OH (1 mmol, 20 eq.) was dissolved in 2 mL of NMP and activated by subsequent addition of 0.45 M HBTU/HOBt in DMF (2.2 mL) and 2 M DIEA/NMP (1 mL).

The solution of the activated Fmoc-protected amino acid was then transferred to the reaction vessel and the coupling proceeded for 30 to 60 minutes, depending on the feedback from the deprotection steps. The resin was then washed six times with NMP and the coupling protocol was repeated. This was subjected to two additional deprotection/coupling cycles, as described above, to complete the assembly of the desired Xₐₐ₇-Xₐₐ₁₁ sequence. The Fmoc-amino acids sequentially coupled were: Fmoc-(L)-Ser(tBu)-OH and Fmoc-(L)-Thr(tBu)-OH.

Fmoc-(S)-2-fluoro-α-Me-Phe-OH was then coupled as follows: Fmoc-(S)-2-fluoro-α-Me-Phe-OH (3.0 eq.) was dissolved in 0.546 M HOAt in DMF (3.0 eq.). The solution was transferred to the reaction vessel followed by two NMP rinses (2×2 mL) and the addition of DIC (3.0 eq.). The coupling proceeded for 16 hours. The resin washed with NMP and the Fmoc group was removed as described previously.

Fmoc-(L)-Thr(tBu)-OH was coupled as follows: Fmoc-(L)-Thr(tBu)-OH (10 eq.) was dissolved in 0.546 M HOAt in DMF (10 eq.). The solution was transferred to the reaction vessel and the vial was rinsed with NMP (2×2 mL), followed by the addition of DIC (10 eq.). The coupling reaction proceeded for 16 hours. The resin washed with NMP and two additional identical coupling cycles were used to install Fmoc-Gly-OH and Fmoc-Glu(OtBu)-OH.

Fmoc-[(S)-α-Me-Pro]-OH was then coupled as follows: Fmoc-[(S)-α-Me-Pro]-OH (2.0 eq.) was dissolved in 0.546 M HOAt in DMF (2.0 eq.) in a vial. The solution was transferred to the reaction vessel and the vial was rinsed with NMP (0.12 mL), followed by the addition of DIC (2.0 eq.). The reaction was allowed to couple for 16 hours. The resin washed with NMP (4×3 mL) and DCM (4×5 mL). After Fmoc deprotection as described above, the resin washed with DMF (8×3 mL) and Fmoc-(L)-His(Trt)-OH was coupled by adding a solution of the amino acid (5 eq.) in 0.546 M HOAt in DMF (5 eq.) to the resin, followed by the addition of DIC (5 eq.) to the reaction vessel. The coupling reaction proceeded for 16 hours. The resin was rinsed with NMP (4×3 mL) and DCM (4×3 mL). The Fmoc group was removed as described for the previous coupling and the peptidyl-resin was transferred to a manual reactor. DMF (1 mL) was added, followed by the addition of solid CH₃O—CO—OSu (3 eq.). The mixture was vortexed for 16 hours. The resin was rinsed with NMP (4×3 mL) and DCM (4×3 mL) to complete the sequence assembly.

The resin bound peptide was cleaved off the resin by treatment with (94:3:3) TFA/water/TIS (5 mL) for three hours. The resin was filtered and rinsed with 90% TFA/water (2×3 mL). The combined filtrates were evaporated to afford a yellow oil, which yielded a solid upon trituration with ether (10 mL), and cooling to 0° C. for one hour. After drying, the crude solid product was purified by preparative HPLC using a gradient of 0.1% TFA/AcCN in 0.1% TFA/water, 20% to 60% over 20 minutes, 14 mL/min. flow rate with effluent detection at 220 nm on a Phenomenex 100×21.2 mm column. The fractions containing a pure product were pooled and lyophilized, to yield 13.8 mg (32% recovery) of SEQ ID NO: 145 having the following characteristics: ES-MS: (M+H)⁺=1599.8; Analytical HPLC: YMC ODS S3 (4.6×50 mm) column; gradient: 5-80% B in A over 10 min, 2.5 mL/min.; A: 0.1% TFA/water; B: 01% TFA/AcCN; and purity of 95.6%.

### EXAMPLE 14

### Synthesis of a Peptide of SEQ ID NO:324

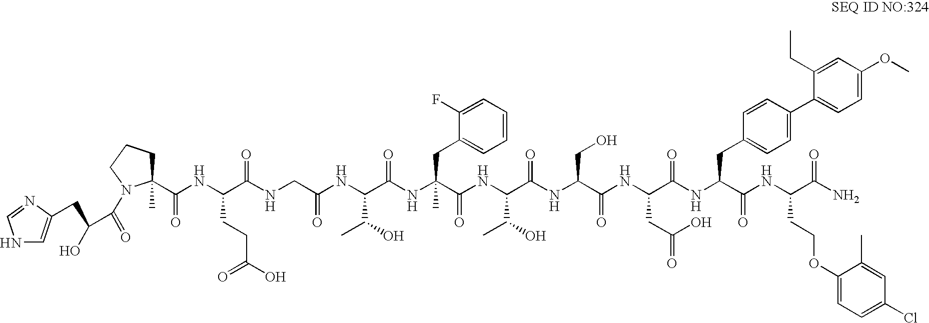

The corresponding Fmoc-protected Xₐₐ₁-Xₐₐ₁₁ peptidyl-resin (0.03 mmole) was prepared as described in Example 13b. After Fmoc removal using two treatments, 5 and 15 minutes each, with 20% piperidine in DMF (3 mL), the resin was washed with DMF (8×3 mL) and α-(L)-Imidazole(2,4-dinitrophenyl)-lactic acid was coupled by adding a solution (5 eq.) in 0.546 M HOAt in DMF (5 eq.), followed by the addition of DIC (5 eq.). The coupling reaction proceeded for 16 hours. The resin was rinsed with NMP (4×3 mL) and DCM (4×3 mL). The 2,4-dinitrophenyl group was removed by treating the resin with 10% Thiophenol/DMF (5 mL) for one hour. The peptidyl-resin was rinsed with DMF (4×5 mL) and DCM (4×5 mL).

The resin-bound peptide was cleaved off the resin with (94:3:3) TFA/water/TIS (5 mL) with vortexing for three hours. The resin was filtered and the resin was rinsed with 90% TFA/water (2×3 mL). The combined filtrates were evaporated to afford a yellow oil. This was purified by preparative HPLC using a gradient of 0.1% TFA/MeCN in 0.1% TFA/water, 25% to 55% over 20 minutes, 14 mL/min. flow rate with effluent UV detection at 220 nm on a Phenomenex 100×21.2 mm column. The fractions containing the product were pooled and lyophilized, to yield 6.8 mg (13% recovery) of SEQ ID NO:324 with the following characteristics: ES-MS: (M+H)+=1542.8; Analytical HPLC: column: YMC ODS S3 (4.6×150 mm); gradient: 35-60% B in A over 30 min, 1.0 mL/min.; A: 0.1% TFA/water; B: 0.1% TFA/AcCN; and a purity of 96.8%.

### EXAMPLE 15

### Synthesis of a Peptide of SEQ ID NO:319

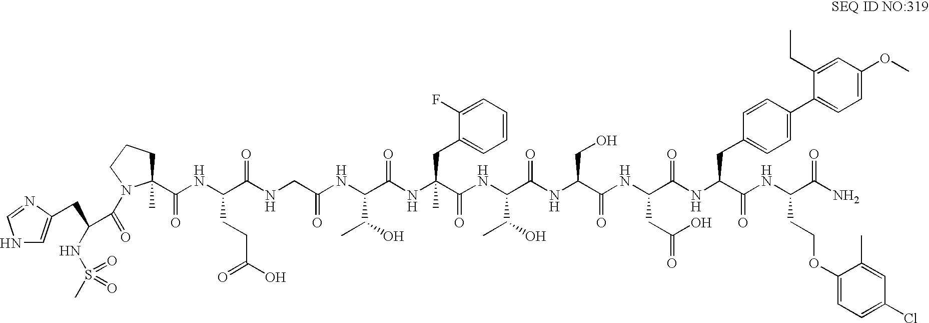

The corresponding Fmoc-protected Xₐₐ₁-Xₐₐ₁₁ peptidyl-resin (0.09 mmole) was prepared as described in Example 13b. After Fmoc removal using two treatments of 5 and 15 minutes each with 20% piperidine in DMF (3 mL), the resin washed with DMF (4×3 mL) and DCM (4×3 mL). A (4:1) DCM/DMF (1 mL) solution was then added, followed by the addition of methanesulfonyl chloride (8 eq.) and diisopropylethylamine (16 eq.). After vortexing for two hours, the resin was rinsed with (4:1) DCM/DMF (4×2 mL) and DCM (4×2 mL). The resin-bound peptide was cleaved off the resin with (94:3:3) TFA/water/TIS (3 mL) with vortexing for three hours. The resin was filtered and the resin was rinsed with 90% TFA/water (2×3 mL). The combined filtrates were evaporated to afford a yellow oil, which yielded a solid upon trituration with ether (10 mL). After drying, the solid product was purified by preparative HPLC using a gradient of 0.1% TFA/MeCN in 0.1% TFA/water, 20% to 60% over 20 minutes, 14 mL/min. flow rate with effluent detection at 220 nm on a Phenomenex 100×21.2 mm column. The fractions containing the product were pooled and lyophilized, to yield 6 mg (15% recovery) of SEQ ID NO:319 and had the following characteristics: ES-MS: (M+H)⁺=1619.8; Analytical HPLC: column, YMC ODS S3 (4.6×50 mm); gradient: 20-55% B in A over 10 min, 2.5 mL/min. A: 0.1% TFA/water; B: 0.1% TFA/AcCN; and purity of 86%.

### EXAMPLE 16

### Synthesis of a Peptide of SEQ ID NO:318

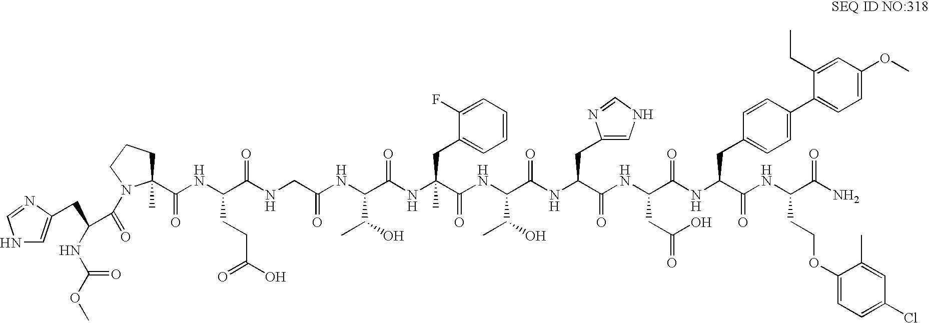

The Fmoc-protected Xₐₐ₂-Xₐₐ₁₁ peptidyl-resin (0.035 mmole) was prepared as described in Example 13b, except that in this case Fmoc-L-His(N-Im-Trt)-OH was used in the fourth coupling (Xₐₐ₈). The Fmoc group was removed as described above and to the peptidyl-resin (0.035 mmole) was coupled CH₃O—CO-(L)-His(N-Im-Trt)-OH by adding a solution of CH₃O-(L)-His(N-Im-Trt)-OH (5 eq.) in 0.546 M HOAt in DMF (5 eq.), followed by the addition of DIC (5 eq.). After 16 hours, the resin was rinsed with NMP (4×3 mL) and DCM (4×3 mL). The resin-bound peptide was cleaved off the resin using (94:3:3) TFA/water/TIS (5 mL) with vortexing for three hours. The resin was filtered off and rinsed with 90% TFA/water (2×3 mL). The combined filtrates were evaporated to afford a yellow oil. This was purified by preparative HPLC using a gradient of 0.1% TFA/MeCN in 0.1% TFA/water, 20% to 60% over 20 minutes, 14 mL/min. flow rate with effluent UV detection at 220 nm on a Phenomenex 100×21.2 mm column. The fractions containing the product were pooled and lyophilized, to yield 20.2 mg (30% recovery) of a peptide of SEQ ID NO:318 and had the following characteristics: ES-MS: (M+H)⁺=1651.0; Analytical HPLC: column, YMC ODS S3 (4.6×150 mm); gradient: 10-55% B in A over 30 min, 1.0 mL/min. A: 0.1% TFA/water; B: 0.1% TFA/AcCN, and purity of 97%.

### EXAMPLE 17

### Synthesis of a Peptide of SEQ ID NO:320

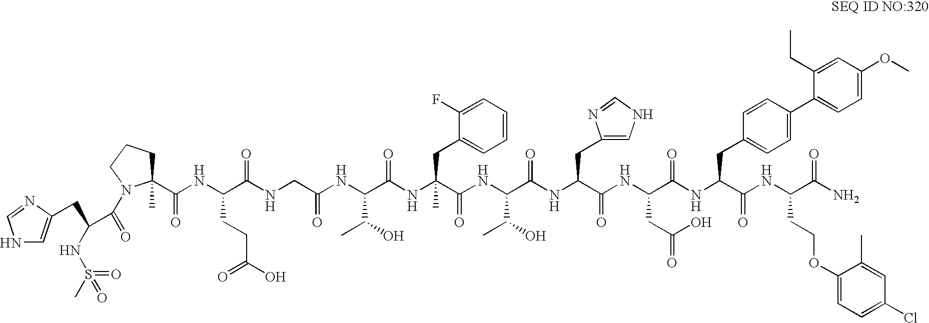

This compound was synthesized as described for the peptide of SEQ ID NO: 319. After release of the peptide from the resin using (94:3:3) TFA/water/TIS, the crude product was purified by preparative HPLC using a gradient of 0.1% TFA/MeCN in 0.1% TFA/water, 20% to 60% over 20 minutes, 14 mL/min. flow rate with effluent UV detection at 220 nm on a Phenomenex 100×21.2 mm column. The fractions containing the product were pooled and lyophilized, to yield 18.1 mg (32% recovery) of a peptide of SEQ ID NO:320 and had the following characteristics: ES-MS: (M+H)⁺=1671.0; Analytical HPLC: column, YMC ODS S3 (4.6×50 mm); gradient: 5-80% B in A over 10 min, 2.5 mL/minutes A: 0.1% TFA/water; B: 0.1% TFA/AcCN and purity of 92%.

### EXAMPLE 18

### Synthesis of a Peptide of SEQ ID NO:321

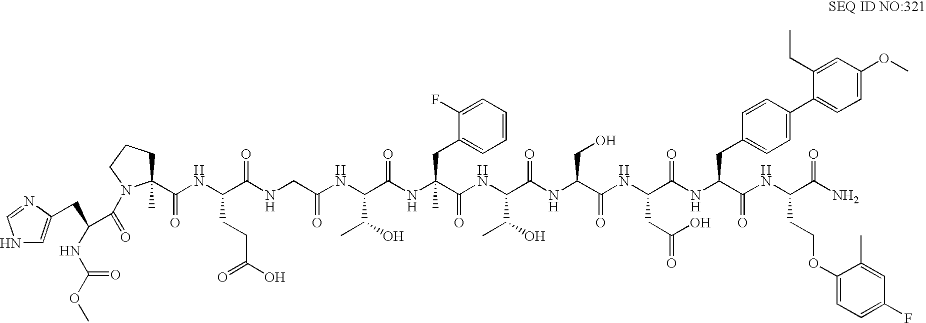

### EXAMPLE 18a

### Procedure for Synthesis of 2-(S)-Fluorenylmethoxycarbonylamino-4-(2-methyl-4-fluoro)phenoxybutanoic acid

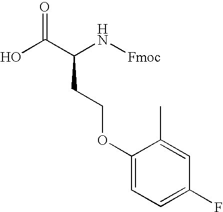

This amino acid can be prepared using procedures similar to those described in Example 13a.

### EXAMPLE 18b

### Procedure for Synthesis of a Peptide of SEQ ID NO:321 via Fragment Condensation

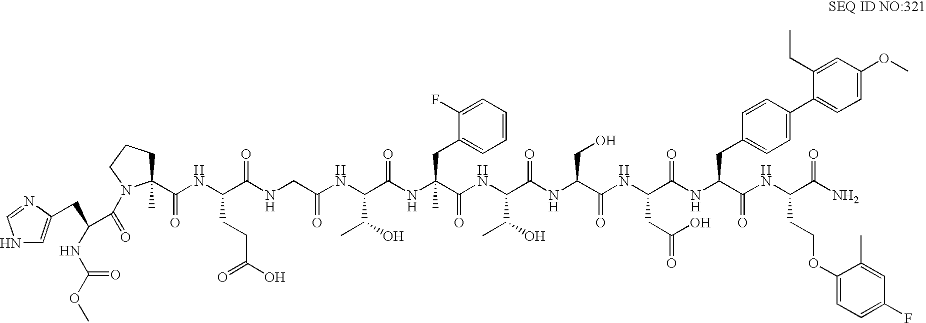

The fully protected peptide (0.033 mmol) was synthesized by fragment condensation as illustrated in Example 6. The protected N-methoxycarbonyl-Xₐₐ₁-Xₐₐ₉ 9-mer used in the fragment condensation step with Xₐₐ₁₀-Xₐₐ₁₁-amide was prepared as described in Example 19. The desired peptide was obtained by deprotection of the protected peptide by treatment with a solution of TFA/TIS (98:2) (1.0 mL) for 1.5 hours. Diisopropyl ether (15 mL) was added to the this solution and the precipitate that formed after one hour was collected by centrifugation. The resulting crude peptide was dissolved in 3% ammonium hydroxide in water (4 mL) and purified by preparative HPLC. The gradient used was from 20% to 60% B in A over 40 minutes. Solvent A: 0.1% TFA in water; Solvent B: 0.1% TFA in acetonitrile. The flow rate was 30 mL/minutes The column was a Phenomenex Luna C18 (2) 5 μm 250×30 mm. The fractions containing the product were pooled and lyophilized, to yield 15.9 mg (28% recovery) and had the following characteristics: HPLC (column: YMC ODS-A S3 (4.6×150 mm); gradient: 10-70% B in A over 30 minutes, 1 mL/min.); ES-MS: (M+H)⁺=1583.7; and purity of 96%.

### EXAMPLE 19

### Procedure for Solid Phase Synthesis of Side Chain Protected N-Methoxycarbonyl-9-Mer Peptide C-Terminal Carboxylic Acid (Scheme 26)

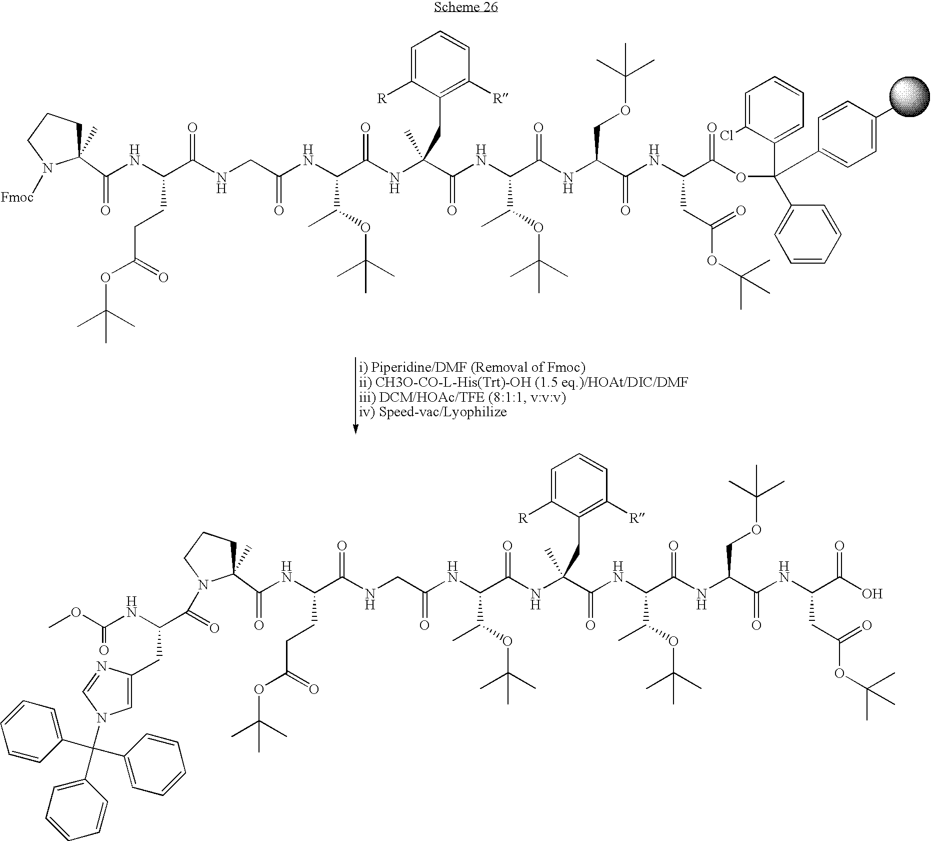

The N-Fmoc side chain protected 8-mer peptidyl-(o-Cl)-Trityl resin (3.5 mmol) was prepared using a procedure similar to that described in Scheme 15. After Fmoc removal and DMF washes, the peptidyl-resin (3.5 mmol) was treated with N-α-Methyloxycarbonyl-N-im-Trityl-L-Histidine (2.4 g, 5.33 mmol) in 0.546 M HOAt in DMF (9.8 mL, 5.33 mmol), followed by addition of DMF (10 mL) and DIC (0.633 mL, 5.33 mmol). After stirring for 72 hours, the N-derivatized 9-mer peptidyl-resin washed with DMF (4×50 mL) and DCM (2×50 mL), and the protected 9-mer peptide C-terminal carboxylic acid was released from the resin by treatment with DCM/AcOH/TFE (8:1:1, v:v:v) for three hours at room temperature. The resin was filtered off and the filtrate was evaporated to dryness, redissolved in AcCN/water (1:1.4) and lyophilized twice, to yield 4.05 g of 71% pure product, which was used in the subsequent fragment coupling steps with no further purification.

### EXAMPLE 20

### Synthesis of a Peptide of SEQ ID NO:322

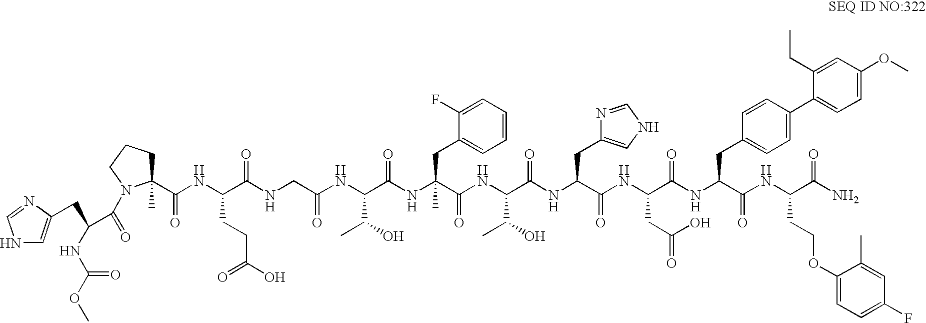

Sieber Amide resin (0.2402 g, 0.135 mmole) was added to a frit-fitted 8 mL SPE cartridge and deprotected using the following manual cycle:

1. DMF wash 1×2 mL×5 minutes

2. 20% piperidine in DMF 1×2 mL×5 minutes

3. 20% piperidine in DMF 1×2 mL×15 minutes

4. DMF washes 4×2 mL×1 minutes

5. NMP washes 4×2 mL×1 minutes

A solution of 2-(S)-Fluorenylmethoxycarbonylamino-4-(2-methyl-4-fluoro)phenoxybutanoic acid (0.083 g, 0.185 mmol), PyBOP (0.185 mmol) and DIEA (0.0704 g, 0.545 mmole) in DMF (1.85 mL) was added to the deprotected resin and the coupling proceeded for 16 hours. The resin washed with DMF and DCM (4×2 mL×1 minute), and was then treated with 10% acetic acid in DCM (1×2 mL×1 hour). The resin washed with DMF (2×2 mL×1 minute) and the Fmoc group was removed using steps 1 to 5 above.

Fmoc-2′-ethyl-4′-methoxy-Biphenylalanine (0.766 g, 1.47 mmole) was dissolved with 0.5 M HOAt in DMF (2.9 mL) and neat DMF (5 mL). DIC (0.189 g, 1.46 mmole) was added to this solution and the resulting solution was adjusted to a final volume of 10 mL with DMF. 1.85 mL of this solution was added to the deprotected resin and the mixture vortexed overnight. The peptide-resin washed with DMF and DCM (4×2 mL×1 min). A Kaiser ninhydrin test was negative. The peptide-resin was dried in vacuo for three hours to give 0.322 g of product.

The dipeptidyl-resin (0.192 g, 0.075 mmol) was deprotected as described above. Fmoc-Asp(OtBu)-OH (0.188 g, 0.457 mmol) was coupled for one hour as a solution in DMF (1 mL) and DCM (0.5 mL). HCTU (0.186 g, 0.451 mmole) and DIEA (0.116 g, 0.898 mmol) was added to the solution. The resin washed with DMF and DCM as described above and was then dried in vacuo overnight to give 0.185 g of peptidyl-resin. Fmoc-His(Trt)-OH (0.140 g, 0.23 mmol) was coupled for two hours as a solution in 0.5 M HOAt in DMF (0.45 mL, 0.23 mmol). DIC (0.029 g, 0.23 mmol) and DCM (0.5 mL) was added to this solution. The resin washed as described. A Kaiser ninhydrin test was negative.

The deprotection cycle was modified as follows:

1. 20% piperidine in DMF 1×1 mL×5 minutes

2. 20% piperidine in DMF 1×1 mL×15 minutes

3. DMF washes 8×1 mL×1 minute

Fmoc-Thr(tBu)-OH (0.150 g, 0.38 mmol) was coupled for 16 hours as a solution in 0.5 M HOAt in DMF (0.75 mL, 0.38 mmol). DIC (0.047 g, 0.37 mmol) was added and adjusted to 2 mL with DMF. The resin washed as described. A Kaiser ninhydrin test was negative. After Fmoc removal, Fmoc-α-Me-Phe(2-F)-OH (0.130 g, 0.31 mmol) was coupled for six hours as a solution in 0.5 M HOAt in DMF (0.60 mL, 0.30 mmol). DIC (0.038 g, 0.31 mmol) was added to this solution with a volume adjustment to 2 mL with DMF. The resin washed and deprotected as described. Fmoc-Thr(tBu)-OH (0.300 g, 0.75 mmol) was coupled for 72 hours as a solution in 0.5 M HOAt in DMF (1.50 mL, 0.75 mmol). DIC (0.101 g, 0.80 mmol) was added to this solution with a volume adjustment to 2 mL with DMF. The resin washed and a sample (˜4 mg) was treated with 2% TIS in TFA for 90 minutes HPLC and MS analysis of the released product showed that the coupling was complete. Fmoc-Gly-OH (0.222 g, 0.75 mmol) was coupled as described for the previous coupling, except that the coupling time was one hour. The peptidyl-resin washed and deprotected as described above. Fmoc-Glu(OtBu)-OH (0.321 g, 0.75 mmol) was coupled for 16 hours as described in the previous coupling.

After washing and Fmoc deprotection as described, Fmoc-α-Me-Pro-OH (0.169 g, 0.46 mmol) coupled for 6.5 hours as solution in 0.5 M HOAt in DMF (0.90 mL, 0.45 mmol). DIC (0.057 g, 0.45 mmol) was added with a final volume adjustment to 2 mL with DMF. The resin washed as described and aliquotted into wells on an Advanced ChemTech 396Q Synthesizer for further elongation. The peptidyl-resin was deprotected on the synthesizer using steps 1 to 3 above. CH₃OCO-His(Trt)-OH (0.0941 g, 0.21 mmol) was coupled for 12 hours as a solution in 0.5 M HOAt/DMF (0.4 mL) and DMF (1 mL) to which DIC (0.0261 g, 0.21 mmol) was added, with a final volume adjustment to 2 mL with DMF.

The resin washed as described, removed from the synthesizer, added to a 4 mL SPE cartridge and treated with 2% TIS in TFA (0.5 mL×5×5 min). The pooled filtrates were kept for another hour at room temperature. The solvent was removed in a speed-vac and the residue was triturated with diisopropylether (15 mL). The resultant solid was collected and dried to yield 25.8 mg of crude peptide. The crude peptide was purified by preparative HPLC after dissolving it in 1.5% ammonium hydroxide (2 mL) with a pH adjustment to 9.5. The gradient used was from 20% to 50% B in A over 60 minutes. Solvent A: 0.1% TFA in water; Solvent B: 0.1% TFA in acetonitrile. The flow rate was 15 mL/min. The column was a Phenomenex Luna C18 (2) 5 μm 250×21.2 mm. The fractions containing the product were pooled and lyophilized, to yield 7.3 mg (24% recovery) of 99% pure product by HPLC (HPLC analysis: column, Phenomenex Luna C18 (2) 5 μm (4.6×150 mm); gradient: 10-60% B in A over 25 minutes, 1 mL/min). ES-MS: (M+H)⁺=1634.1.

### EXAMPLE 21

### Synthesis of a Peptide of SEQ ID NO:323

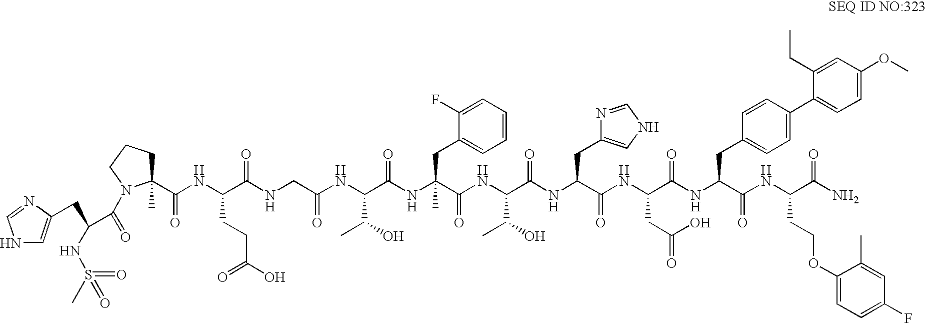

The Fmoc-protected Xₐₐ₂-Xₐₐ₁₁-Sieber resin was prepared and deprotected as described in Example 20. Fmoc-His(Trt)-OH (0.4989 g, 0.81 mmol) was coupled for 12 hours as a solution in 0.5 M HOAt/DMF (0.8 mL) and DMF (2 mL). DIC (0.051 g, 0.40 mmole) was added and adjusted to 4 mL with DMF. After washing, the resin was added to a 4 mL SPE cartridge and was deprotected by performing steps 1 to 3 described in Example 20. The α-amino group of the histidine residue was capped by reaction for two hours with methanesulfonyl chloride (6.8 mg, 0.059 mmol) as a solution in (4:1) DCM/DMF (0.5 mL) to which DIEA (21 μL) was added. After washing as described, the peptidyl-resin was cleaved/deprotected as described in Example 20. The crude peptide was purified by preparative HPLC after dissolving it in 1.5% ammonium hydroxide (2 mL). The gradient used was from 25% to 55% B in A over 60 minutes. Solvent A: 0.1% TFA in water; Solvent B: 0.1% TFA in AcCN. The flow rate was 15 mL/min. The column was a Phenomenex Luna C18 (2) 5 μm 250×21.2 mm. The fractions containing the product were pooled and lyophilized, to yield 11.5 mg (24% recovery) of 96% pure product by HPLC (analytical HPLC: column, Phenomenex Luna C18 (2) 5 μm (4.6×150 mm); gradient: 15-65% B in A over 60 minutes, 1 mL/min.) with an ES-MS: (M+H)⁺=1653.7.

### EXAMPLE 22

### Examples of Xₐₐ₁₁ Side Chains

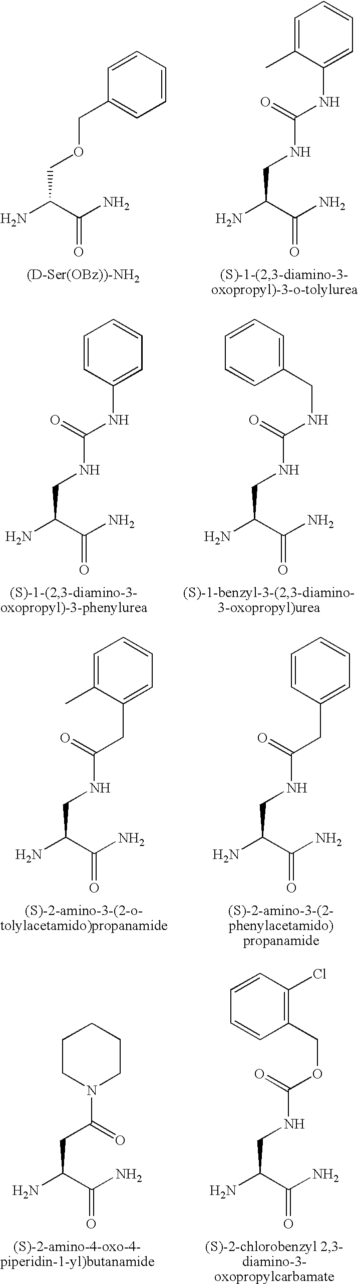

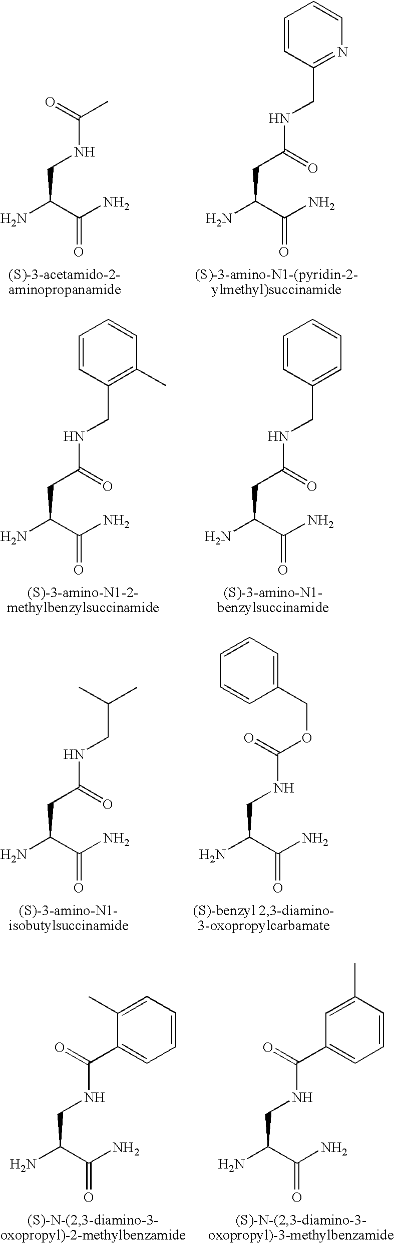

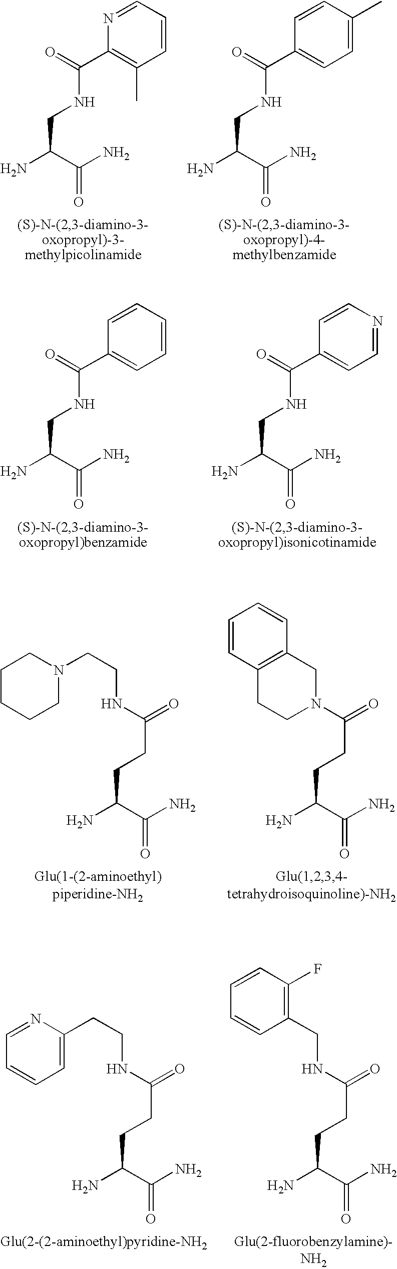

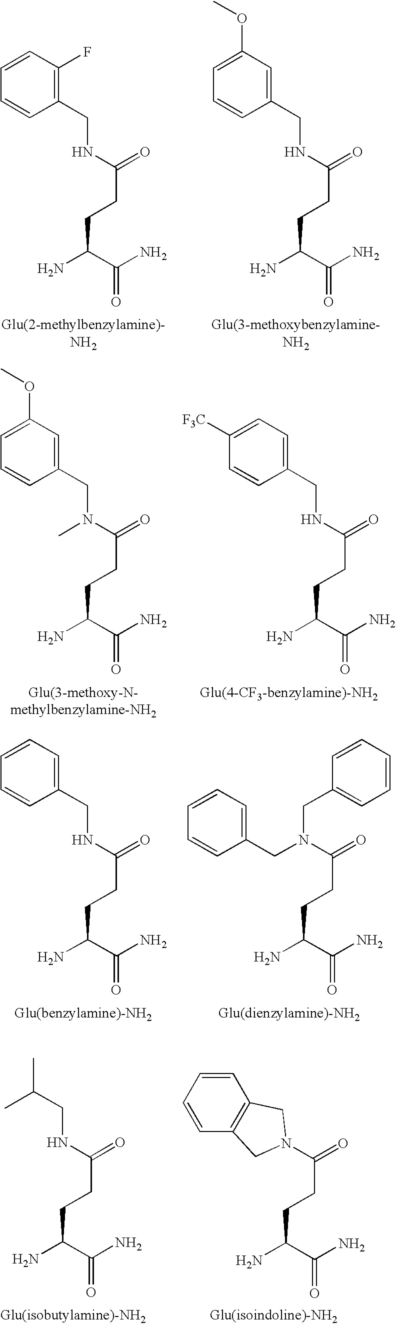

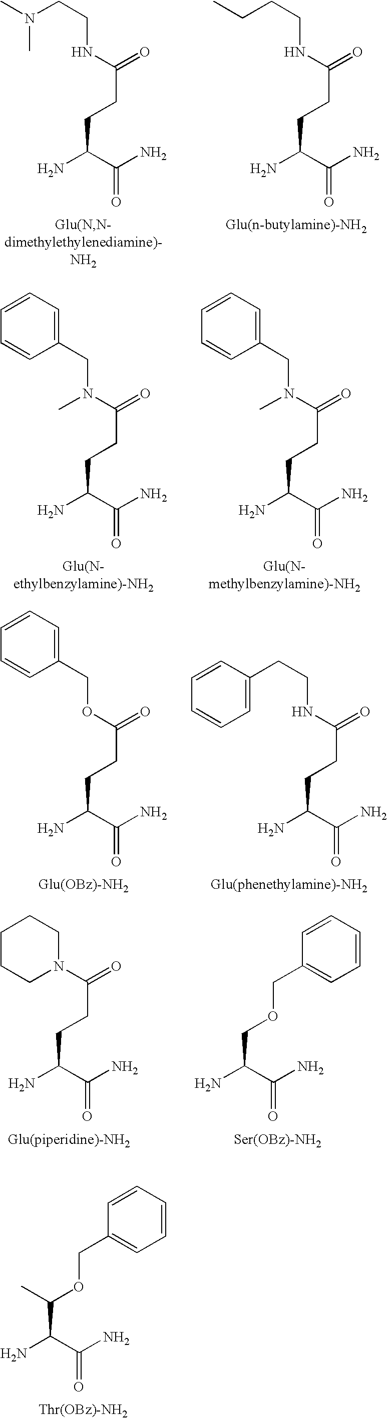

### EXAMPLE 23

### Examples of Additional Xₐₐ₁₁ Side Chains

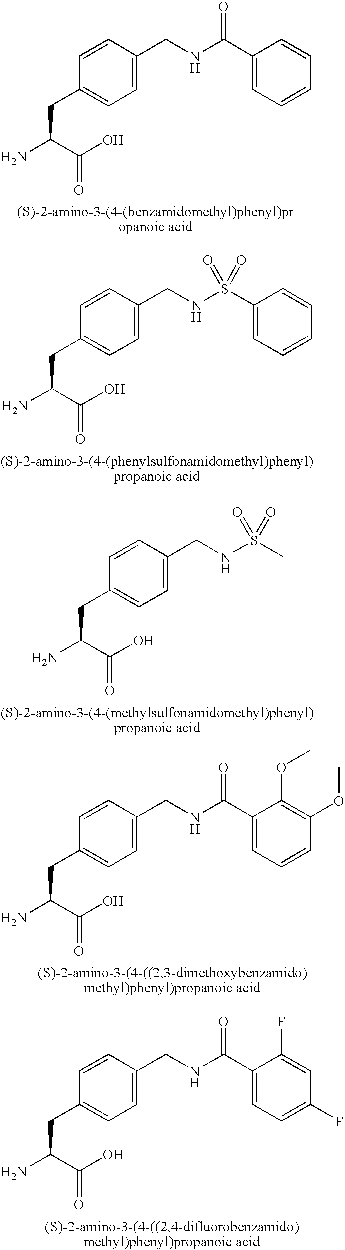

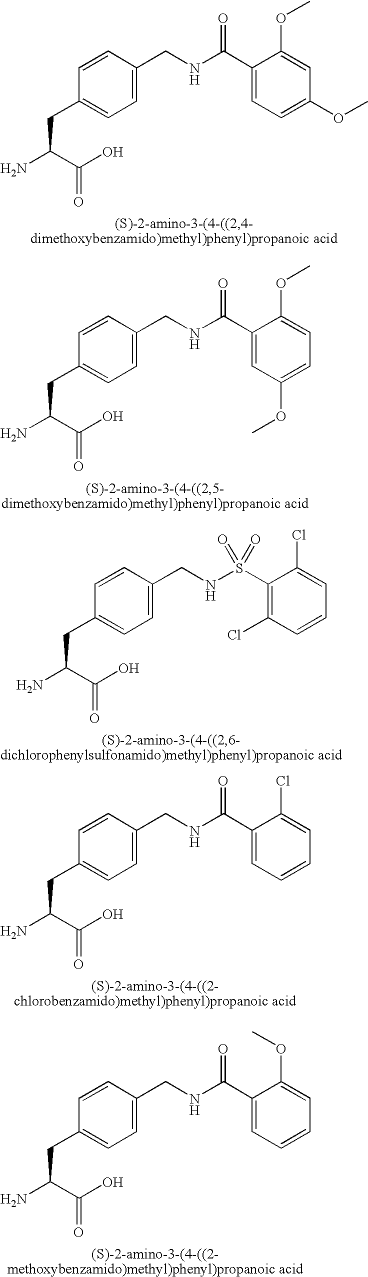

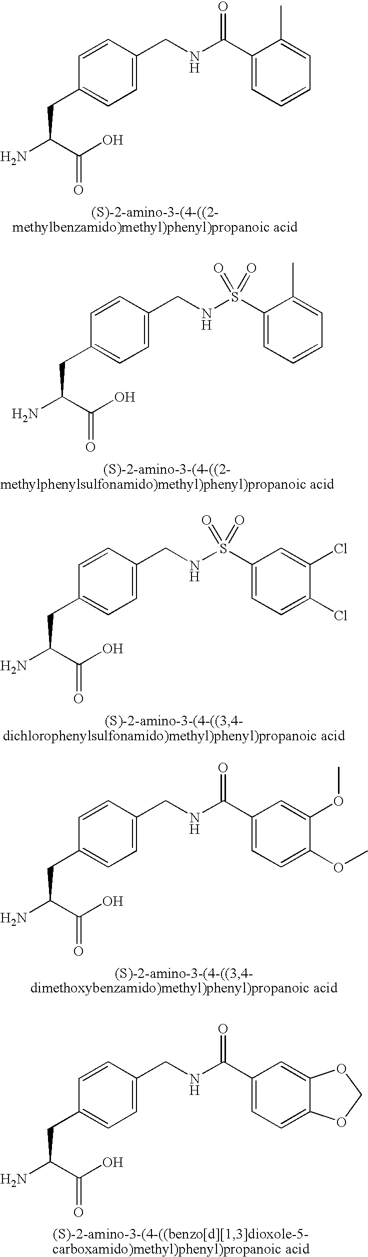

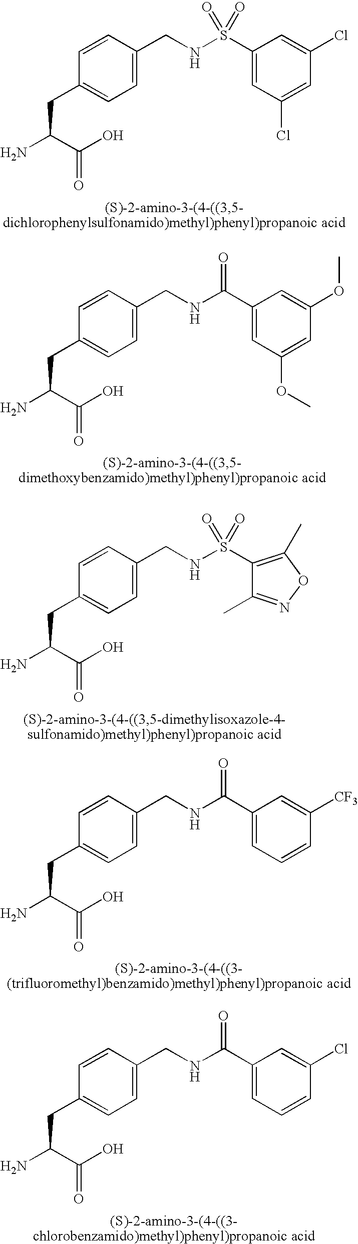

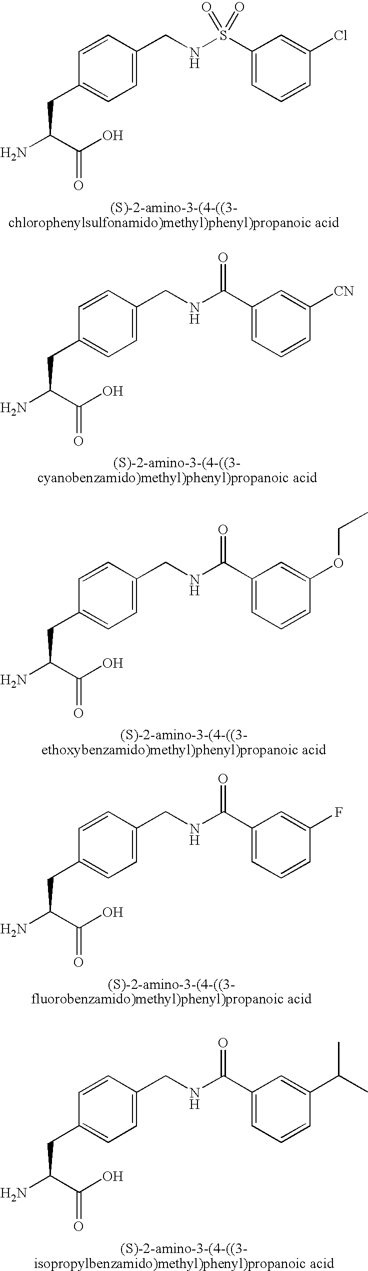

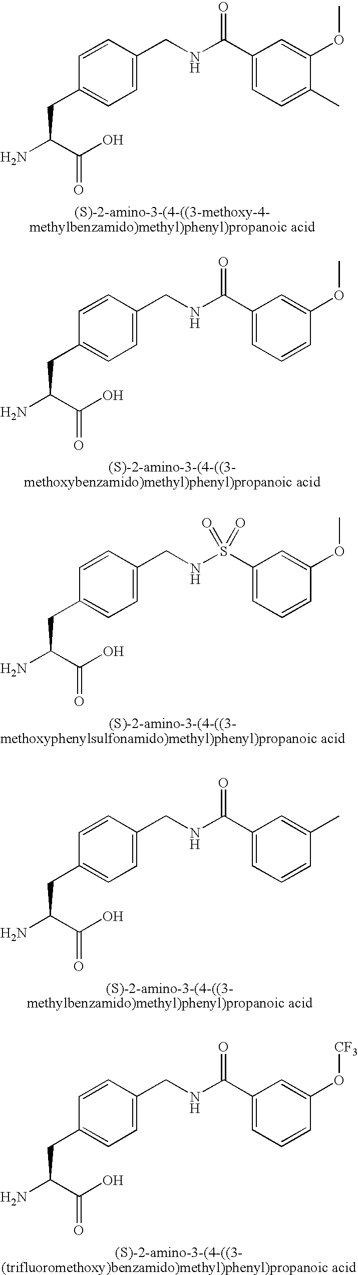

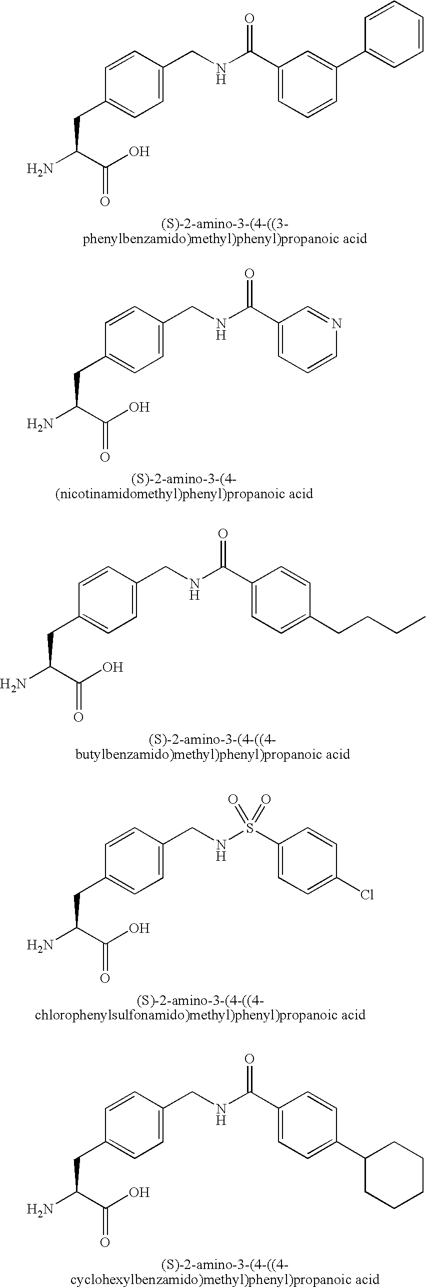

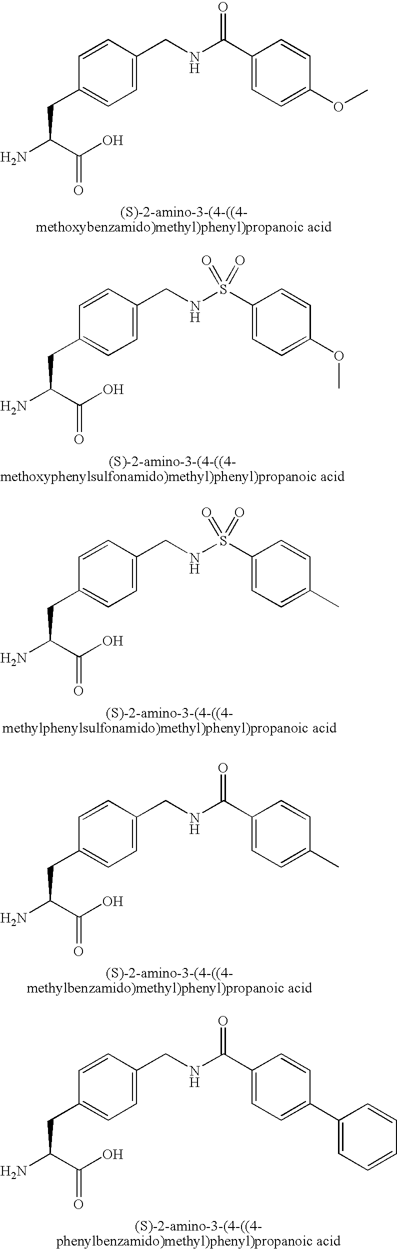

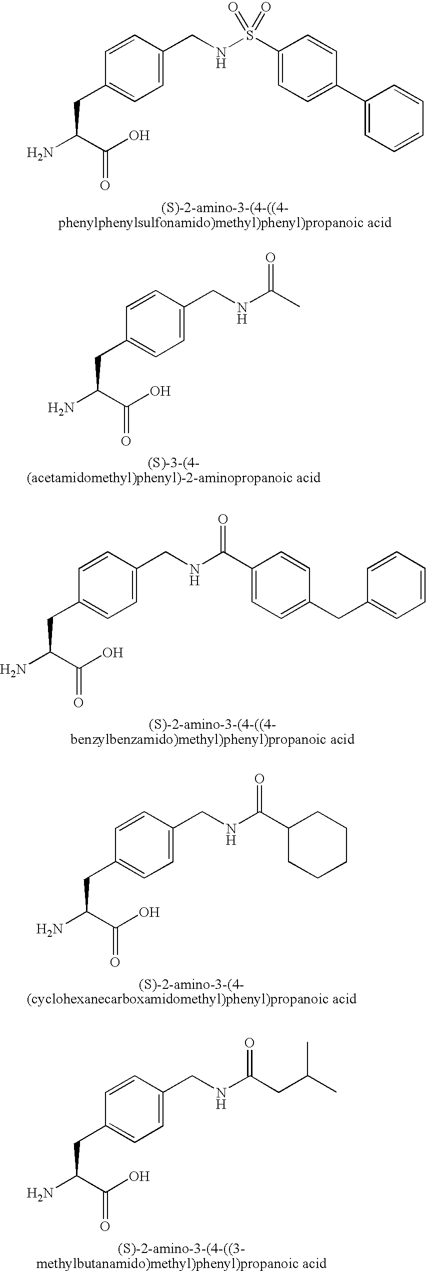

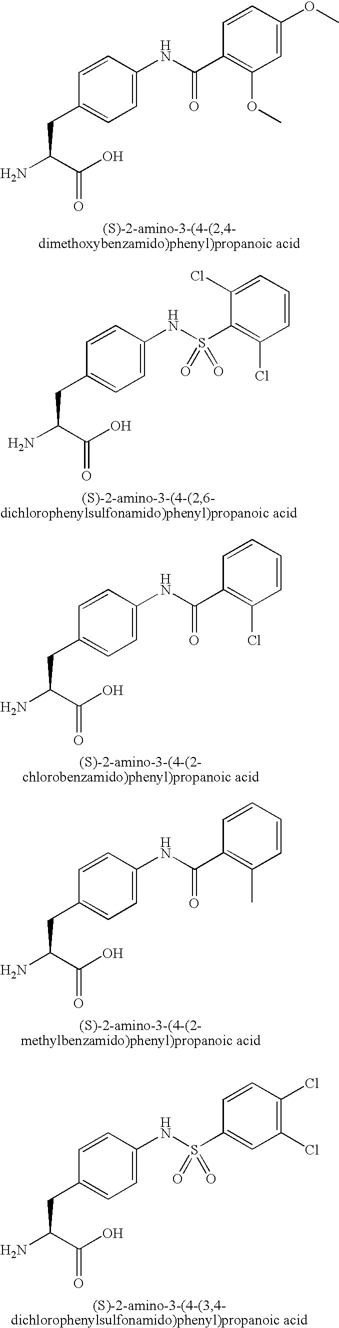

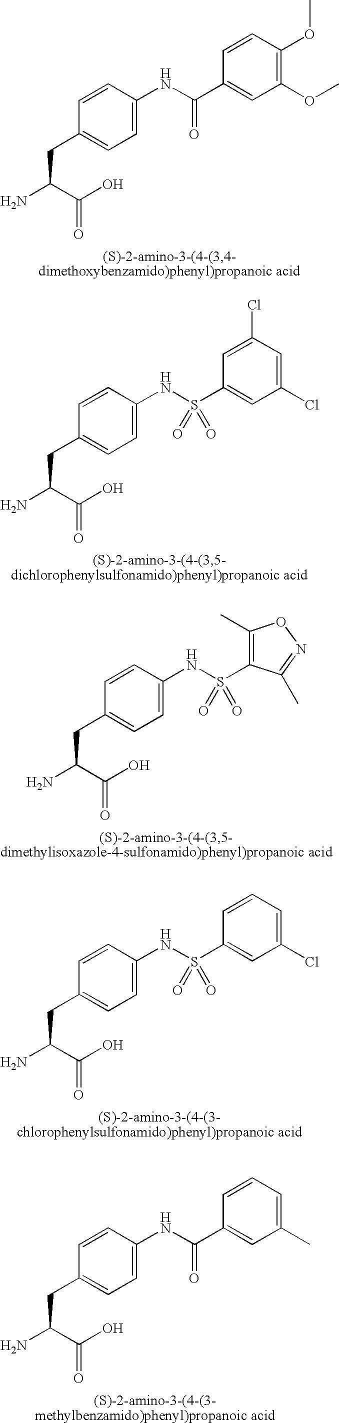

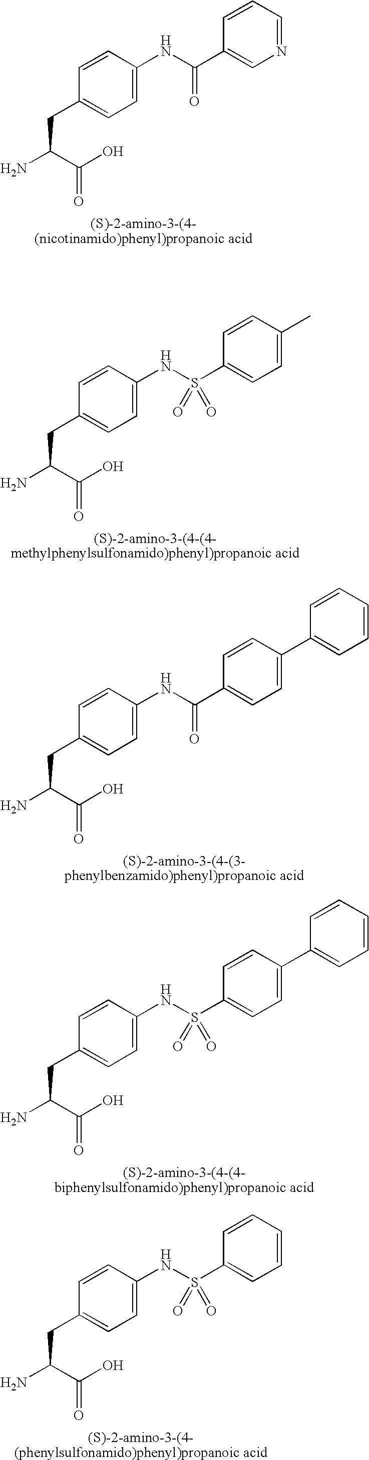

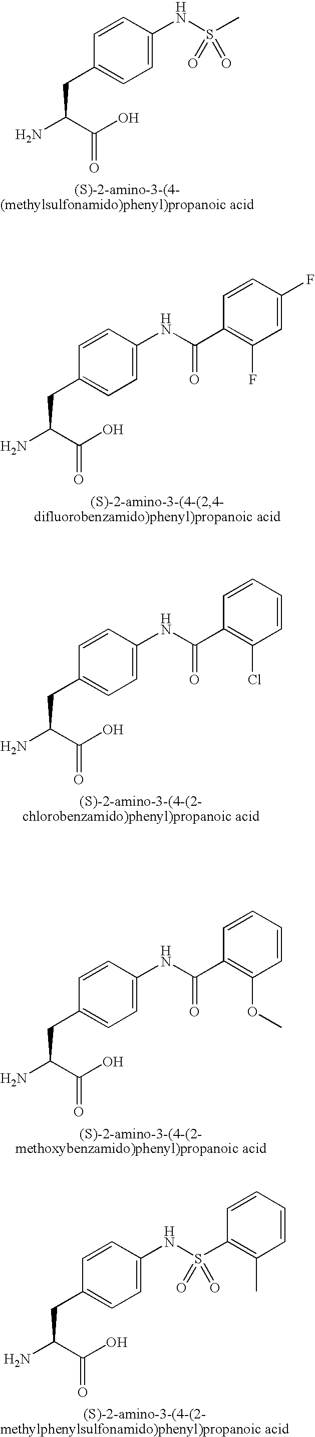

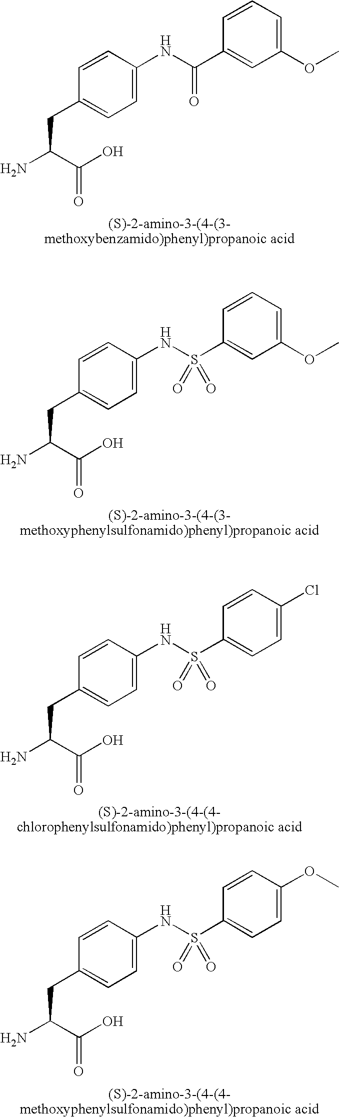

### EXAMPLE 24

### Additional Examples of Xₐₐ₁₁ Side Chains

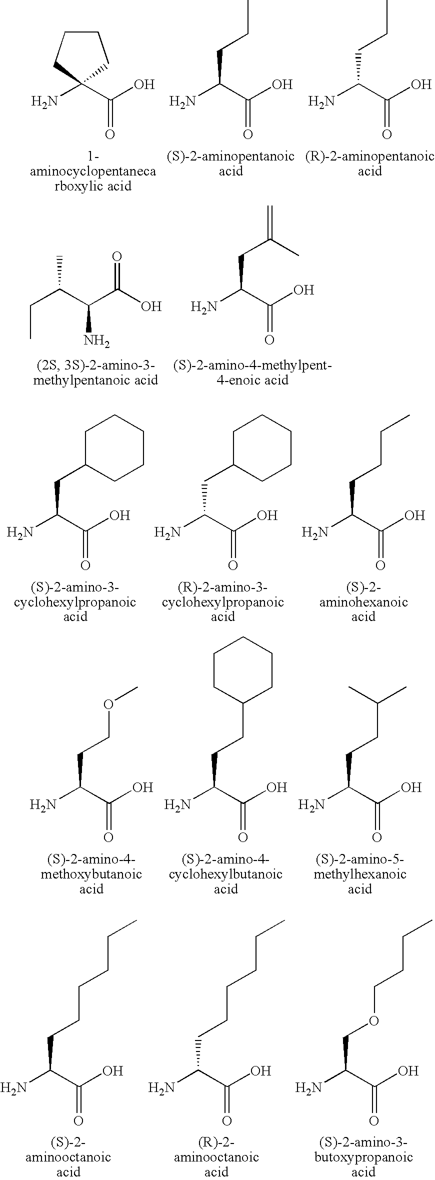

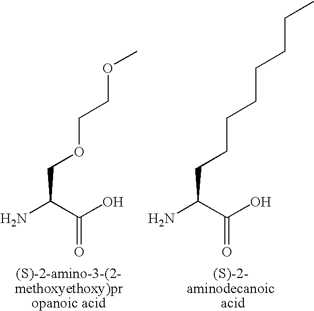

### EXAMPLE 25

### Additional Xₐₐ₁₁ Side Chains

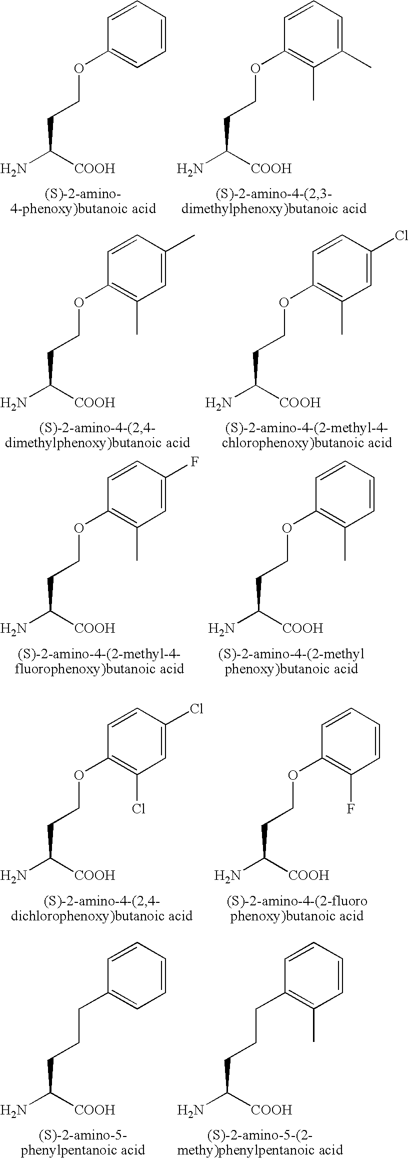

### EXAMPLE 26

### In Vitro Assay Results for Selected Peptides

Description of Cell-Based Human GLP-1R EC₅₀ Assay

CHO cells stably expressing human GLP-1 receptor (HGLP-1R) or mouse GLP1 receptor (MGLP-1R) were plated at 2×10⁴ cells/well in sterile 96-well white clear bottom Costar plates and incubated overnight before assaying. On the assay day, after aspirating the growth media, the cells were treated with 50 μl of compounds at varying concentration or buffer control in phosphate-buffered saline (PBS) without MgCl₂ and CaCl₂, with 0.1 mM IBMX and 0.05% BSA for 20 minutes at room temperature. The solution was then aspirated and 50 μl lysis buffer was added immediately, followed by adding 70 μl of assay buffer containing ¹²⁵I-labeled cAMP tracer, rabbit anti-cAMP antibody and SPA beads that are covalently coated with anti-rabbit antibody (provided by the Amersham cAMP SPA assay kit). The plates were incubated at room temperature for 12 hours before counting on a TriLux Microbeta reader (Perkin Elmer, Boston, Mass.). The cAMP standard curve with 12 concentrations was established independently using a known amount of non-radioactive cAMP. The amount of cAMP from treated cells was converted to picomoles (pmol) of cAMP by interpolating from the cAMP standard curve. The agonist data of compounds are normalized and plotted as the percentage of the response induced by the concentration of 10 nM of GLP-1.

Data and Statistical Analysis

The concentration-response data from cAMP functional experiments is analyzed by fitting the normalized data to the four parameter logistic equation (Equation 205) through the non-linear regression by software XL-fit (built into TA activity base). The EC₅₀ value of compounds is defined as the concentration of peptide which stimulated 50% maximal cAMP synthesis by GLP-1 at the concentration of 10 nM in CHO cells as the positive control by the use of XL-fit.

Results in the form of EC₅₀ values for selected compounds are shown in Table 1. The structures of exemplary compounds are provided in Tables 2-5.

TABLE 1

| SEQ ID NO: | Human GLP-1 cAMP EC₅₀ (nM) |
|---|---|
| 1 | 0.013 |
| 6 | 0.009 |
| 7 | 0.050 |
| 22 | 0.017 |
| 27 | 0.011 |
| 31 | 0.024 |
| 43 | 0.033 |
| 76 | 0.013 |
| 80 | 0.016 |
| 83 | 0.006 |
| 84 | 0.007 |
| 88 | 0.093 |
| 97 | 0.037 |
| 116 | 0.017 |
| 126 | 0.009 |
| 134 | 0.383 |
| 141 | 0.017 |
| 145 | 0.048 |
| 162 | 0.006 |
| 163 | 0.043 |

TABLE 2

| SEQ | | | | | | | | | | | |
|---|---|---|---|---|---|---|---|---|---|---|---|
| ID NO: | Xₐₐ₁ | Xₐₐ₂ | Xₐₐ₃ | Xₐₐ₄ | Xₐₐ₅ | Xₐₐ₆ | Xₐₐ₇ | Xₐₐ₈ | Xₐₐ₉ | Xₐₐ₁₀ | Xₐₐ₁₁ |
| 1 | H | Aib | E | G | T | L-α-Me- | T | S | D | Bip(2′-Et- | (S)-2-amino-5- |
| | | | | | | Phe(2- | | | | 4′-OMe) | phenylpentanamide |
| | | | | | | Fluoro) | | | | | |
| 2 | H | Aib | E | G | T | L-α-Me- | T | S | D | Bip(2′-Et- | (S)-2-amino-5-(pyridin-2-yl) |
| | | | | | | Phe(2- | | | | 4′-OMe) | pentanamide |
| | | | | | | Fluoro) | | | | | |
| 3 | H | Aib | E | G | T | L-α-Me- | T | S | D | Bip(2′-Et- | (S)-2-amino-5-(pyridin-3-yl) |
| | | | | | | Phe(2- | | | | 4′-OMe) | pentanamide |
| | | | | | | Fluoro) | | | | | |
| 4 | H | Aib | E | G | T | L-α-Me- | T | S | D | Bip(2′-Et- | (S)-2-amino-5-(pyrazin-2-yl) |
| | | | | | | Phe(2- | | | | 4′-OMe) | pentanamide |
| | | | | | | Fluoro) | | | | | |
| 5 | H | Aib | E | G | T | L-α-Me- | T | S | D | Bip(2′-Et- | (S)-2-amino-5- |
| | | | | | | Phe(2- | | | | 4′-OMe) | (benzo[d][1,3]dioxol-5-yl) |
| | | | | | | Fluoro) | | | | | pentanamide |
| 6 | H | Aib | E | G | T | L-α-Me- | T | S | D | Bip(2′-Et- | (S)-2-amino-5-(3,5- |
| | | | | | | Phe(2- | | | | 4′-OMe) | dimethylphenyl)pentanamide |
| | | | | | | Fluoro) | | | | | |
| 7 | H | Aib | E | G | T | L-α-Me- | T | S | D | Bip(2′-Et- | (S)-2-amino-5-(2- |
| | | | | | | Phe(2- | | | | 4′-OMe) | methylphenyl)pentanamide |
| | | | | | | Fluoro) | | | | | |
| 8 | H | Aib | E | G | T | L-α-Me- | T | S | D | Bip(2′-Et- | (S)-2-amino-5-(4- |
| | | | | | | Phe(2- | | | | 4′-OMe) | methylphenyl)pentanamide |
| | | | | | | Fluoro) | | | | | |
| 9 | H | (S)-α- | E | G | T | L-α-Me- | T | S | D | Bip(2′-Et- | (S)-2-amino-5-(3- |
| | | Me-Pro | | | | Phe(2- | | | | 4′-OMe) | methylphenyl)pentanamide |
| | | | | | | Fluoro) | | | | | |
| 10 | H | (S)-α- | E | G | T | L-α-Me- | T | S | D | Bip(2′-Et- | (S)-2-amino-5-(4- |
| | | Me-Pro | | | | Phe(2- | | | | 4′-OMe) | methoxyphenyl)pentanamide |
| | | | | | | Fluoro) | | | | | |
| 11 | H | (S)-α- | E | G | T | L-α-Me- | T | S | D | Bip(2′-Et- | (S)-2-amino-5-(5- |
| | | Me-Pro | | | | Phe(2- | | | | 4′-OMe) | methylpyridin-2- |
| | | | | | | Fluoro) | | | | | yl)pentanamide |
| 12 | H | (S)-α- | E | G | T | L-α-Me- | T | S | D | Bip(2′-Et- | (S)-2-amino-5-(4- |
| | | Me-Pro | | | | Phe(2- | | | | 4′-OMe) | propylphenyl)pentanamide |
| | | | | | | Fluoro) | | | | | |
| 13 | H | (S)-α- | E | G | T | L-α-Me- | T | S | D | Bip(2′-Et- | (S)-2-amino-5-(2- |
| | | Me-Pro | | | | Phe(2- | | | | 4′-OMe) | ethylphenyl)pentanamide |
| | | | | | | Fluoro) | | | | | |
| 14 | H | (S)-α- | E | G | T | L-α-Me- | T | S | D | Bip(2′-Et- | (S)-2-amino-5-(4- |
| | | Me-Pro | | | | Phe(2- | | | | 4′-OMe) | methylpyridin-2- |
| | | | | | | Fluoro) | | | | | yl)pentanamide |
| 15 | H | (S)-α- | E | G | T | L-α-Me- | T | S | D | Bip(2′-Et- | (S)-2-amino-5-(2,4- |
| | | Me-Pro | | | | Phe(2- | | | | 4′-OMe) | dimethylphenyl)pentanamide |
| | | | | | | Fluoro) | | | | | |
| 16 | H | (S)-α- | E | G | T | L-α-Me- | T | S | D | Bip(2′-Et- | (S)-2-amino-5-(3- |
| | | Me-Pro | | | | Phe(2- | | | | 4′-OMe) | methoxyphenyl)pentanamide |
| | | | | | | Fluoro) | | | | | |
| 17 | H | (S)-α- | E | G | T | L-α-Me- | T | S | D | Bip(2′-Et- | (S)-2-amino-5- |
| | | Me-Pro | | | | Phe(2- | | | | 4′-OMe) | phenylpentanamide |
| | | | | | | Fluoro) | | | | | |
| 18 | H | (S)-α- | E | G | T | L-α-Me- | T | S | D | Bip(2′-Et- | (S)-2-amino-5-(4- |
| | | Me-Pro | | | | Phe(2- | | | | 4′-OMe) | biphenyl)pentanamide |
| | | | | | | Fluoro) | | | | | |
| 19 | H | (S)-α- | E | G | T | L-α-Me- | T | S | D | Bip(2′-Et- | (S)-2-amino-5-(3- |
| | | Me-Pro | | | | Phe(2- | | | | 4′-OMe) | methylpyridin-2-yl) |
| | | | | | | Fluoro) | | | | | pentanamide |
| 20 | H | (S)-α- | E | G | T | L-α-Me- | T | S | D | Bip(2′-Et- | (S)-2-amino-5-(pyridin-4-yl) |
| | | Me-Pro | | | | Phe(2- | | | | 4′-OMe) | pentanamide |
| | | | | | | Fluoro) | | | | | |
| 21 | H | (S)-α- | E | G | T | L-α-Me- | T | S | D | Bip(2′-Et- | (S)-2-amino-5-(2,3- |
| | | Me-Pro | | | | Phe(2- | | | | 4′-OMe) | dimethylphenyl)pentanamide |
| | | | | | | Fluoro) | | | | | |
| 22 | H | (S)-α- | E | G | T | L-α-Me- | T | S | D | Bip(2′-Et- | (S)-2-amino-5-(quinolin-6- |
| | | Me-Pro | | | | Phe(2- | | | | 4′-OMe) | yl)pentanamide |
| | | | | | | Fluoro) | | | | | |
| 23 | H | (S)-α- | E | G | T | L-α-Me- | T | S | D | Bip(2′-Et- | (S)-2-amino-5-(naphthalen- |
| | | Me-Pro | | | | Phe(2- | | | | 4′-OMe) | 2-yl)pentanamide |
| | | | | | | Fluoro) | | | | | |
| 24 | H | (S)-α- | E | G | T | L-α-Me- | T | S | D | Bip(2′-Et- | (S)-2-amino-5-(6- |
| | | Me-Pro | | | | Phe(2- | | | | 4′-OMe) | methylpyridin-2-yl) |
| | | | | | | Fluoro) | | | | | pentanamide |
| 25 | H | (S)-α- | E | G | T | L-α-Me- | T | S | D | Bip(2′-Et- | (S)-2-amino-5-(2,3- |
| | | Me-Pro | | | | Phe(2- | | | | 4′-OMe) | dihydrobenzo[b][1,4]dioxin- |
| | | | | | | Fluoro) | | | | | 6-yl)pentanamide |
| 26 | H | (S)-α- | E | G | T | L-α-Me- | T | S | D | Bip(2′-Et- | (S)-2-amino-5-(2,6- |
| | | Me-Pro | | | | Phe(2- | | | | 4′-OMe) | dimethylpyridin-3- |
| | | | | | | Fluoro) | | | | | yl)pentanamide |
| 27 | H | (S)-α- | E | G | T | L-α-Me- | T | S | D | Bip(2′-Et- | (S)-2-amino-5-(4- |
| | | Me-Pro | | | | Phe(2- | | | | 4′-OMe) | chlorophenyl)pentanamide |
| | | | | | | Fluoro) | | | | | |
| 28 | H | (S)-α- | E | G | T | L-α-Me- | T | S | D | Bip(2′-Et- | (S)-2-amino-5-(3- |
| | | Me-Pro | | | | Phe(2- | | | | 4′-OMe) | chlorophenyl)pentanamide |
| | | | | | | Fluoro) | | | | | |
| 29 | H | (S)-α- | E | G | T | L-α-Me- | T | S | D | Bip(2′-Et- | (S)-2-amino-5-(4- |
| | | Me-Pro | | | | Phe(2- | | | | 4′-OMe) | (dimethylamino)phenyl) |
| | | | | | | Fluoro) | | | | | pentanamide |
| 30 | H | (S)-α- | E | G | T | L-α-Me- | T | S | D | Bip(2′-Et- | (S)-2-amino-5-(2- |
| | | Me-Pro | | | | Phe(2- | | | | 4′-OMe) | chlorophenyl)pentanamide |
| | | | | | | Fluoro) | | | | | |
| 31 | H | (S)-α- | E | G | T | L-α-Me- | T | S | D | Bip(2′-Et- | (S)-2-amino-5-(4- |
| | | Me-Pro | | | | Phe(2- | | | | 4′-OMe) | (methylsulfonyl)phenyl) |
| | | | | | | Fluoro) | | | | | pentanamide |
| 32 | H | (S)-α- | E | G | T | L-α-Me- | T | S | D | Bip(2′-Et- | (S)-2-amino-5-(quinolin-5- |
| | | Me-Pro | | | | Phe(2- | | | | 4′-OMe) | yl)pentanamide |
| | | | | | | Fluoro) | | | | | |
| 33 | H | (S)-α- | E | G | T | L-α-Me- | T | S | D | Bip(2′-Et- | (S)-2-amino-5-(4- |
| | | Me-Pro | | | | Phe(2- | | | | 4′-OMe) | (trifluoromethyl)phenyl) |
| | | | | | | Fluoro) | | | | | pentanamide |
| 34 | H | (S)-α- | E | G | T | L-α-Me- | T | S | D | Bip(2′-Et- | (S)-2-amino-5-(4- |
| | | Me-Pro | | | | Phe(2- | | | | 4′-OMe) | fluorophenyl)pentanamide |
| | | | | | | Fluoro) | | | | | |
| 35 | H | (S)-α- | E | G | T | L-α-Me- | T | S | D | Bip(2′-Et- | (S)-2-amino-5-(3- |
| | | Me-Pro | | | | Phe(2- | | | | 4′-OMe) | biphenyl)pentanamide |
| | | | | | | Fluoro) | | | | | |
| 36 | H | (S)-α- | E | G | T | L-α-Me- | T | S | D | Bip(2′-Et- | 4-phenylbutan-1-amine |
| | | Me-Pro | | | | Phe(2- | | | | 4′-OMe) | |
| | | | | | | Fluoro) | | | | | |
| 37 | H | (S)-α- | E | G | T | L-α-Me- | T | S | D | Bip(2′-Et- | (S)-2-amino-5-(2,3,5,6- |
| | | Me-Pro | | | | Phe(2- | | | | 4′-OMe) | tetramethylphenyl) |
| | | | | | | Fluoro) | | | | | pentanamide |
| 38 | H | (S)-α- | E | G | T | L-α-Me- | T | S | D | Bip(2′-Et- | (S)-2-amino-5-(4- |
| | | Me-Pro | | | | Phe(2- | | | | 4′-OMe) | isopropylphenyl) |
| | | | | | | Fluoro) | | | | | pentanamide |
| 39 | H | (S)-α- | E | G | T | L-α-Me- | T | S | D | Bip(2′-Et- | (S)-2-amino-5-(3- |
| | | Me-Pro | | | | Phe(2- | | | | 4′-OMe) | biphenyl)pentanamide |
| | | | | | | Fluoro) | | | | | |
| 40 | H | (S)-α- | E | G | T | L-α-Me- | T | S | D | Bip(2′-Et- | (S)-2-amino-5-(4- |
| | | Me-Pro | | | | Phe(2- | | | | 4′-OMe) | (dimethylamino)phenyl) |
| | | | | | | Fluoro) | | | | | pentanamide |
| 41 | H | (S)-α- | E | G | T | L-α-Me- | T | S | D | Bip(2′-Et- | (S)-2-amino-5-(3- |
| | | Me-Pro | | | | Phe(2- | | | | 4′-OMe) | chlorophenyl)pentanamide |
| | | | | | | Fluoro) | | | | | |
| 42 | H | (S)-α- | E | G | T | L-α-Me- | T | S | D | Bip(2′-Et- | (S)-2-amino-5-(4- |
| | | Me-Pro | | | | Phe(2- | | | | 4′-OMe) | fluorophenyl)pentanamide |
| | | | | | | Fluoro) | | | | | |
| 43 | H | (S)-α- | E | G | T | L-α-Me- | T | S | D | Bip(2′-Et- | (S)-2-amino-5-(2- |
| | | Me-Pro | | | | Phe(2- | | | | 4′-OMe) | chlorophenyl)pentanamide |
| | | | | | | Fluoro) | | | | | |
| 44 | H | (S)-α- | E | G | T | L-α-Me- | T | S | D | Bip(2′-Et- | (S)-2-amino-5-(4- |
| | | Me-Pro | | | | Phe(2- | | | | 4′-OMe) | (trifluoromethyl)phenyl) |
| | | | | | | Fluoro) | | | | | pentanamide |
| 45 | H | (S)-α- | E | G | T | L-α-Me- | T | S | D | Bip(2′-Et- | (S)-2-amino-5-(4- |
| | | Me-Pro | | | | Phe(2- | | | | 4′-OMe) | isopropylphenyl) |
| | | | | | | Fluoro) | | | | | pentanamide |
| 46 | H | (S)-α- | E | G | T | L-α-Me- | T | S | D | Bip(2′-Et- | (S)-2-amino-5-(4- |
| | | Me-Pro | | | | Phe(2- | | | | 4′-OMe) | (methylsulfonyl)phenyl) |
| | | | | | | Fluoro) | | | | | pentanamide |
| 47 | H | (S)-α- | E | G | T | L-α-Me- | T | S | D | Bip(2′-Et- | (S)-2-amino-5-(quinolin-5- |
| | | Me-Pro | | | | Phe(2- | | | | 4′-OMe) | yl)pentanamide |
| | | | | | | Fluoro) | | | | | |
| 48 | H | (S)-α- | E | G | T | L-α-Me- | T | S | D | Bip(2′-Et- | (S)-2-amino-5-(2,3,5,6- |
| | | Me-Pro | | | | Phe(2- | | | | 4′-OMe) | tetramethylphenyl) |
| | | | | | | Fluoro) | | | | | pentanamide |
| 49 | H | (S)-α- | E | G | T | L-α-Me- | T | S | D | Bip(2′-Et- | (S)-2-amino-5-(pyridin-2-yl) |
| | | Me-Pro | | | | Phe(2- | | | | 4′-OMe) | pentanamide |
| | | | | | | Fluoro) | | | | | |
| 50 | H | (S)-α- | E | G | T | L-α-Me- | T | S | D | Bip(2′-Et- | (S)-2-amino-5-(pyridin-3-yl) |
| | | Me-Pro | | | | Phe(2- | | | | 4′-OMe) | pentanamide |
| | | | | | | Fluoro) | | | | | |
| 51 | H | (S)-α- | E | G | T | L-α-Me- | T | S | D | Bip(2′-Et- | (S)-2-amino-5-(pyrazin-2-yl) |
| | | Me-Pro | | | | Phe(2- | | | | 4′-OMe) | pentanamide |
| | | | | | | Fluoro) | | | | | |
| 52 | H | (S)-α- | E | G | T | L-α-Me- | T | S | D | Bip(2′-Et- | (S)-2-amino-5- |
| | | Me-Pro | | | | Phe(2- | | | | 4′-OMe) | (benzo[d][1,3]dioxol-5-yl) |
| | | | | | | Fluoro) | | | | | pentanamide |
| 53 | H | (S)-α- | E | G | T | L-α-Me- | T | S | D | Bip(2′-Et- | (S)-2-amino-5-(3,5- |
| | | Me-Pro | | | | Phe(2- | | | | 4′-OMe) | dimethylphenyl)pentanamide |
| | | | | | | Fluoro) | | | | | |
| 54 | H | (S)-α- | E | G | T | L-α-Me- | T | S | D | Bip(2′-Et- | (S)-2-amino-5-o- |
| | | Me-Pro | | | | Phe(2- | | | | 4′-OMe) | tolylpentanamide |
| | | | | | | Fluoro) | | | | | |
| 55 | H | (S)-α- | E | G | T | L-α-Me- | T | S | D | Bip(2′-Et- | (S)-2-amino-5-(6- |
| | | Me-Pro | | | | Phe(2- | | | | 4′-OMe) | methoxypyridin-2-yl) |
| | | | | | | Fluoro) | | | | | pentanamide |
| 56 | H | (S)-α- | E | G | T | L-α-Me- | T | S | D | Bip(2′-Et- | (S)-2-amino-5-(2,3- |
| | | Me-Pro | | | | Phe(2- | | | | 4′-OMe) | dihydrobenzofuran-5-yl) |
| | | | | | | Fluoro) | | | | | pentanamide |
| 57 | H | (S)-α- | E | G | T | L-α-Me- | T | S | D | Bip(2′-Et- | (S)-2-(4,5-diamino-5- |
| | | Me-Pro | | | | Phe(2- | | | | 4′-OMe) | oxopentyl)isonicotinamide |
| | | | | | | Fluoro) | | | | | |

TABLE 3

| SEQ | | | | | | | | | | | |
|---|---|---|---|---|---|---|---|---|---|---|---|
| ID NO: | Xₐₐ₁ | Xₐₐ₂ | Xₐₐ₃ | Xₐₐ₄ | Xₐₐ₅ | Xₐₐ₆ | Xₐₐ₇ | Xₐₐ₈ | Xₐₐ₉ | Xₐₐ₁₀ | Xₐₐ₁₁ |
| 58 | H | Aib | E | G | T | L-α-Me- | T | S | D | Bip(2′- | (R)-2-amino-4-(2- |
| | | | | | | Phe(2- | | | | Et-4′- | chlorophenoxy) |
| | | | | | | Fluoro) | | | | OMe) | butanamide |
| 59 | H | Aib | E | G | T | L-α-Me- | T | S | D | Bip(2′- | (S)-2-amino-4-(2- |
| | | | | | | Phe(2- | | | | Et-4′- | chlorophenoxy) |
| | | | | | | Fluoro) | | | | OMe) | butanamide |
| 60 | H | (S)-α- | E | G | T | L-α-Me- | T | S | D | Bip(2′- | (S)-2-amino-4-(2- |
| | | Me-Pro | | | | Phe(2- | | | | Et-4′- | chlorophenoxy) |
| | | | | | | Fluoro) | | | | OMe) | butanamide |
| 61 | H | Aib | E | G | T | L-α-Me- | T | S | D | Bip(2′- | (S)-2-amino-4- |
| | | | | | | Phe(2- | | | | Et-4′- | phenoxybutanamide |
| | | | | | | Fluoro) | | | | OMe) | |
| 62 | H | (S)-α- | E | G | T | L-α-Me- | T | S | D | Bip(2′- | (S)-2-amino-4- |
| | | Me-Pro | | | | Phe(2- | | | | Et-4′- | phenoxybutanamide |
| | | | | | | Fluoro) | | | | OMe) | |
| 63 | H | Aib | E | G | T | L-α-Me- | T | S | D | Bip(2′- | (S)-2-amino-4-(2,4- |
| | | | | | | Phe(2- | | | | Et-4′- | dimethylphenoxy) |
| | | | | | | Fluoro) | | | | OMe) | butanamide |
| 64 | H | (S)-α- | E | G | T | L-α-Me- | T | S | D | Bip(2′- | (S)-2-amino-4-(2,4- |
| | | Me-Pro | | | | Phe(2- | | | | Et-4′- | dimethylphenoxy) |
| | | | | | | Fluoro) | | | | OMe) | butanamide |
| 65 | H | Aib | E | G | T | L-α-Me- | T | S | D | Bip(2′- | (S)-2-amino-4-(2- |
| | | | | | | Phe(2- | | | | Et-4′- | methylphenoxy) |
| | | | | | | Fluoro) | | | | OMe) | butanamide |
| 66 | H | (S)-α- | E | G | T | L-α-Me- | T | S | D | Bip(2′- | (S)-2-amino-4-(2- |
| | | Me-Pro | | | | Phe(2- | | | | Et-4′- | methylphenoxy) |
| | | | | | | Fluoro) | | | | OMe) | butanamide |
| 67 | H | Aib | E | G | T | L-α-Me- | T | S | D | Bip(2′- | (S)-2-amino-4-(4- |
| | | | | | | Phe(2- | | | | Et-4′- | methoxyphenoxy) |
| | | | | | | Fluoro) | | | | OMe) | butanamide |
| 68 | H | (S)-α- | E | G | T | L-α-Me- | T | S | D | Bip(2′- | (S)-2-amino-4-(4- |
| | | Me-Pro | | | | Phe(2- | | | | Et-4′- | methoxyphenoxy) |
| | | | | | | Fluoro) | | | | OMe) | butanamide |
| 69 | H | Aib | E | G | T | L-α-Me- | T | S | D | Bip(2′- | (S)-2-amino-4-(4- |
| | | | | | | Phe(2- | | | | Et-4′- | methylphenoxy) |
| | | | | | | Fluoro) | | | | OMe) | butanamide |
| 70 | H | (S)-α- | E | G | T | L-α-Me- | T | S | D | Bip(2′- | (S)-2-amino-4-(4- |
| | | Me-Pro | | | | Phe(2- | | | | Et-4′- | methylphenoxy) |
| | | | | | | Fluoro) | | | | OMe) | butanamide |
| 71 | H | Aib | E | G | T | L-α-Me- | T | S | D | Bip(2′- | (S)-2-amino-4-(5- |
| | | | | | | Phe(2- | | | | Et-4′- | methylpyridin-2- |
| | | | | | | Fluoro) | | | | OMe) | yloxy)butanamide |
| 72 | H | (S)-α- | E | G | T | L-α-Me- | T | S | D | Bip(2′- | (S)-2-amino-4-(5- |
| | | Me-Pro | | | | Phe(2- | | | | Et-4′- | methylpyridin-2- |
| | | | | | | Fluoro) | | | | OMe) | yloxy)butanamide |
| 73 | H | Aib | E | G | T | L-α-Me- | T | S | D | Bip(2′- | (S)-2-amino-4-(4- |
| | | | | | | Phe(2- | | | | Et-4′- | fluorophenoxy) |
| | | | | | | Fluoro) | | | | OMe) | butanamide |
| 74 | H | (S)-α- | E | G | T | L-α-Me- | T | S | D | Bip(2′- | (S)-2-amino-4-(4- |
| | | Me-Pro | | | | Phe(2- | | | | Et-4′- | fluorophenoxy) |
| | | | | | | Fluoro) | | | | OMe) | butanamide |
| 75 | H | (S)-α- | E | G | T | L-α-Me- | T | S | D | Bip(2′- | (S)-2-amino-4-(2- |
| | | Me-Pro | | | | Phe(2- | | | | Et-4′- | fluorophenoxy) |
| | | | | | | Fluoro) | | | | OMe) | butanamide |
| 76 | H | (S)-α- | E | G | T | L-α-Me- | T | S | D | Bip(2′- | (S)-2-amino-4-(2,4- |
| | | Me-Pro | | | | Phe(2- | | | | Et-4′- | dichlorophenoxy) |
| | | | | | | Fluoro) | | | | OMe) | butanamide |
| 77 | H | (S)-α- | E | G | T | L-α-Me- | T | S | D | Bip(2′- | (S)-2-amino-4-(2,3- |
| | | Me-Pro | | | | Phe(2- | | | | Et-4′- | dimethylphenoxy) |
| | | | | | | Fluoro) | | | | OMe) | butanamide |
| 78 | H | (S)-α- | E | G | T | L-α-Me- | T | S | D | Bip(2′- | (S)-2-amino-4-(3- |
| | | Me-Pro | | | | Phe(2- | | | | Et-4′- | fluorophenoxy) |
| | | | | | | Fluoro) | | | | OMe) | butanamide |
| 79 | H | (S)-α- | E | G | T | L-α-Me- | T | S | D | Bip(2′- | (S)-2-amino-4-(3- |
| | | Me-Pro | | | | Phe(2- | | | | Et-4′- | chlorophenoxy) |
| | | | | | | Fluoro) | | | | OMe) | butanamide |
| 80 | H | (S)-α- | E | G | T | L-α-Me- | T | S | D | Bip(2′- | (S)-2-amino-4-(3- |
| | | Me-Pro | | | | Phe(2- | | | | Et-4′- | methoxyphenoxy) |
| | | | | | | Fluoro) | | | | OMe) | butanamide |
| 81 | H | (S)-α- | E | G | T | L-α-Me- | T | S | D | Bip(2′- | (S)-2-amino-4-(3- |
| | | Me-Pro | | | | Phe(2- | | | | Et-4′- | methylphenoxy) |
| | | | | | | Fluoro) | | | | OMe) | butanamide |
| 82 | H | (S)-α- | E | G | T | L-α-Me- | T | S | D | Bip(2′- | (S)-2-amino-4-(3,4- |
| | | Me-Pro | | | | Phe(2- | | | | Et-4′- | dimethylphenoxy) |
| | | | | | | Fluoro) | | | | OMe) | butanamide |
| 83 | H | Aib | E | G | T | L-α-Me- | T | S | D | Bip(2′- | (S)-2-amino-4-(4- |
| | | | | | | Phe(2- | | | | Et-4′- | phenylphenoxy) |
| | | | | | | Fluoro) | | | | OMe) | butanamide |
| 84 | H | (S)-α- | E | G | T | L-α-Me- | T | S | D | Bip(2′- | (S)-2-amino-4-(2,3- |
| | | Me-Pro | | | | Phe(2- | | | | Et-4′- | dihydro-1H-inden-5- |
| | | | | | | Fluoro) | | | | OMe) | yloxy)butanamide |
| 85 | H | (S)-α- | E | G | T | L-α-Me- | T | S | D | Bip(2′- | (S)-2-amino-4-(4-chloro- |
| | | Me-Pro | | | | Phe(2- | | | | Et-4′- | 2-methylphenoxy)-N- |
| | | | | | | Fluoro) | | | | OMe) | methylbutanamide |
| 86 | H | (S)-α- | E | G | T | L-α-Me- | T | S | D | Bip(2′- | (R)-2-amino-4-(4-(1H- |
| | | Me-Pro | | | | Phe(2- | | | | Et-4′- | 1,2,4-triazol-1-yl- |
| | | | | | | Fluoro) | | | | OMe) | phenoxy)butanamide |
| 87 | H | (S)-α- | E | G | T | L-α-Me- | T | S | D | Bip(2′- | (S)-2-amino-4-(2-CN- |
| | | Me-Pro | | | | Phe(2- | | | | Et-4′- | phenoxy)butanamide |
| | | | | | | Fluoro) | | | | OMe) | |
| 88 | H | (S)-α- | E | G | T | L-α-Me- | T | S | D | Bip(2′- | (S)-2-amino-4-(2-MeO- |
| | | Me-Pro | | | | Phe(2- | | | | Et-4′- | phenoxy)butanamide |
| | | | | | | Fluoro) | | | | OMe) | |
| 89 | H | (S)-α- | E | G | T | L-α-Me- | T | S | D | Bip(2′- | (S)-2-amino-4-(2- |
| | | Me-Pro | | | | Phe(2- | | | | Et-4′- | phenylphenoxy) |
| | | | | | | Fluoro) | | | | OMe) | butanamide |
| 90 | H | (S)-α- | E | G | T | L-α-Me- | T | S | D | Bip(2′- | (S)-2-amino-4-(2-CF₃- |
| | | Me-Pro | | | | Phe(2- | | | | Et-4′- | phenoxy)butanamide |
| | | | | | | Fluoro) | | | | OMe) | |
| 91 | H | (S)-α- | E | G | T | L-α-Me- | T | S | D | Bip(2′- | (S)-2-amino-4-(3- |
| | | Me-Pro | | | | Phe(2- | | | | Et-4′- | acetamido--phenoxy) |
| | | | | | | Fluoro) | | | | OMe) | butanamide |
| 92 | H | (S)-α- | E | G | T | L-α-Me- | T | S | D | Bip(2′- | (S)-2-amino-4-(3- |
| | | Me-Pro | | | | Phe(2- | | | | Et-4′- | dimethylamino- |
| | | | | | | Fluoro) | | | | OMe) | phenoxy)butanamide |
| 93 | H | (S)-α- | E | G | T | L-α-Me- | T | S | D | Bip(2′- | (S)-2-amino-4-(3- |
| | | Me-Pro | | | | Phe(2- | | | | Et-4′- | phenylphenoxy) |
| | | | | | | Fluoro) | | | | OMe) | butanamide |
| 94 | H | (S)-α- | E | G | T | L-α-Me- | T | S | D | Bip(2′- | (S)-2-amino-4-(4-Cl-2- |
| | | Me-Pro | | | | Phe(2- | | | | Et-4′- | Me-phenoxy)butanamide |
| | | | | | | Fluoro) | | | | OMe) | |
| 95 | H | (S)-α- | E | G | T | L-α-Me- | T | S | D | Bip(2′- | (S)-2-amino-4-(4- |
| | | Me-Pro | | | | Phe(2- | | | | Et-4′- | phenoxy--phenoxy) |
| | | | | | | Fluoro) | | | | OMe) | butanamide |
| 96 | H | (S)-α- | E | G | T | L-α-Me- | T | S | D | Bip(2′- | (S)-2-amino-4-(4-CF₃- |
| | | Me-Pro | | | | Phe(2- | | | | Et-4′- | phenoxy)butanamide |
| | | | | | | Fluoro) | | | | OMe) | |
| 97 | H | (S)-α- | E | G | T | L-α-Me- | T | S | D | Bip(2′- | (S)-2-amino-4-(4- |
| | | Me-Pro | | | | Phe(2- | | | | Et-4′- | isopropyl-phenoxy) |
| | | | | | | Fluoro) | | | | OMe) | butanamide |
| 98 | H | (S)-α- | E | G | T | L-α-Me- | T | S | D | Bip(2′- | (S)-2-amino-4-(1- |
| | | Me-Pro | | | | Phe(2- | | | | Et-4′- | naphthoxy)butanamide |
| | | | | | | Fluoro) | | | | OMe) | |
| 99 | H | (S)-α- | E | G | T | L-α-Me- | T | S | D | Bip(2′- | (S)-2-amino-4-(2- |
| | | Me-Pro | | | | Phe(2- | | | | Et-4′- | naphthoxy)butanamide |
| | | | | | | Fluoro) | | | | OMe) | |
| 100 | H | (S)-α- | E | G | T | L-α-Me- | T | S | D | Bip(2′- | 2-amino-4-(4-(imidazol-1- |
| | | Me-Pro | | | | Phe(2- | | | | Et-4′- | yl)-phenoxy)butanamide |
| | | | | | | Fluoro) | | | | OMe) | |
| 101 | H | (S)-α- | E | G | T | L-α-Me- | T | S | D | Bip(2′- | (S)-2-amino-5- |
| | | Me-Pro | | | | Phe(2- | | | | Et-4′- | (benzo[d][1,3]dioxol-5- |
| | | | | | | Fluoro) | | | | OMe) | yl)pentanamide |
| 102 | H | (S)-α- | E | G | T | L-α-Me- | T | S | D | Bip(2′- | (S)-2-amino-4-(3- |
| | | Me-Pro | | | | Phe(2- | | | | Et-4′- | dimethylamino- |
| | | | | | | Fluoro) | | | | OMe) | phenoxy)butanamide |
| 103 | H | (S)-α- | E | G | T | L-α-Me- | T | S | D | Bip(2′- | 2-amino-4-(quinolin-6- |
| | | Me-Pro | | | | Phe(2- | | | | Et-4′- | yloxy)butanamide |
| | | | | | | Fluoro) | | | | OMe) | |
| 104 | H | (S)-α- | E | G | T | L-α-Me- | T | S | D | Bip(2′- | (S)-2-amino-4-(3- |
| | | Me-Pro | | | | Phe(2- | | | | Et-4′- | trifluoromethoxy- |
| | | | | | | Fluoro) | | | | OMe) | phenoxy)butanamide |
| 105 | H | (S)-α- | E | G | T | L-α-Me- | T | S | D | Bip(2′- | 2-amino-4-(4- |
| | | Me-Pro | | | | Phe(2- | | | | Et-4′- | methylpyridin-2- |
| | | | | | | Fluoro) | | | | OMe) | yloxy)butanamide |
| 106 | H | (S)-α- | E | G | T | L-α-Me- | T | S | D | Bip(2′- | 2(S)-amino-4-(2-(1H- |
| | | Me-Pro | | | | Phe(2- | | | | Et-4′- | prazol-3-yl- |
| | | | | | | Fluoro) | | | | OMe) | phenoxy)butanamide |
| 107 | H | (S)-α- | E | G | T | L-α-Me- | T | S | D | Bip(2′- | 2(S)-amino-4-(4-(1H- |
| | | Me-Pro | | | | Phe(2- | | | | Et-4′- | 1,2,4-triazol-1-yl- |
| | | | | | | Fluoro) | | | | OMe) | phenoxy)butanamide |

TABLE 4

| SEQ | | | | | | | | | | | |
|---|---|---|---|---|---|---|---|---|---|---|---|
| ID NO: | Xₐₐ₁ | Xₐₐ₂ | Xₐₐ₃ | Xₐₐ₄ | Xₐₐ₅ | Xₐₐ₆ | Xₐₐ₇ | Xₐₐ₈ | Xₐₐ₉ | Xₐₐ₁₀ | Xₐₐ₁₁ |
| 108 | des- | (S)-α- | E | G | T | L-α-Me- | T | S | D | Bip(2′- | (S)-4-(3,4-diamino-4- |
| | amino | Me-Pro | | | | Phe(2- | | | | Et-4′- | oxobutoxy)benzamide |
| | His | | | | | Fluoro) | | | | OMe) | |
| 109 | des- | (S)-α- | E | G | T | L-α-Me- | T | S | D | Bip(2′- | (S)-2-amino-4- |
| | amino | Me-Pro | | | | Phe(2- | | | | Et-4′- | phenoxybutanamide |
| | His | | | | | Fluoro) | | | | OMe) | |
| 110 | des- | (S)-α- | E | G | T | L-α-Me- | T | S | D | Bip(2′- | (S)-2-amino-4-(2- |
| | amino | Me-Pro | | | | Phe(2- | | | | Et-4′- | fluorophenoxy) |
| | His | | | | | Fluoro) | | | | OMe) | butanamide |
| 111 | des- | (S)-α- | E | G | T | L-α-Me- | T | S | D | Bip(2′- | (S)-2-amino-4-(2- |
| | amino | Me-Pro | | | | Phe(2- | | | | Et-4′- | phenylphenoxy) |
| | His | | | | | Fluoro) | | | | OMe) | butanamide |
| 112 | des- | (S)-α- | E | G | T | L-α-Me- | T | S | D | Bip(2′- | (S)-2-amino-4-(2- |
| | amino | Me-Pro | | | | Phe(2- | | | | Et-4′- | trifluoromethylphenoxy) |
| | His | | | | | Fluoro) | | | | OMe) | butanamide |
| 113 | des- | (S)-α- | E | G | T | L-α-Me- | T | S | D | Bip(2′- | (S)-2-amino-4-(2- |
| | amino | Me-Pro | | | | Phe(2- | | | | Et-4′- | methylphenoxy) |
| | His | | | | | Fluoro) | | | | OMe) | butanamide |
| 114 | des- | (S)-α- | E | G | T | L-α-Me- | T | S | D | Bip(2′- | (S)-2-amino-4-(2,4- |
| | amino | Me-Pro | | | | Phe(2- | | | | Et-4′- | dimethylphenoxy) |
| | His | | | | | Fluoro) | | | | OMe) | butanamide |
| 115 | des- | (S)-α- | E | G | T | L-α-Me- | T | S | D | Bip(2′- | (S)-2-amino-4-(3- |
| | amino | Me-Pro | | | | Phe(2- | | | | Et-4′- | acetamidophenoxy) |
| | His | | | | | Fluoro) | | | | OMe) | butanamide |
| 116 | des- | (S)-α- | E | G | T | L-α-Me- | T | S | D | Bip(2′- | (S)-2-amino-4-(2- |
| | amino | Me-Pro | | | | Phe(2- | | | | Et-4′- | methyl-4- |
| | His | | | | | Fluoro) | | | | OMe) | chlorophenoxy) |
| | | | | | | | | | | | butanamide |
| 117 | des- | (S)-α- | E | G | T | L-α-Me- | T | S | D | Bip(2′- | (S)-2-amino-4-(4- |
| | amino | Me-Pro | | | | Phe(2- | | | | Et-4′- | phenoxyphenoxy) |
| | His | | | | | Fluoro) | | | | OMe) | butanamide |
| 118 | des- | (S)-α- | E | G | T | L-α-Me- | T | S | D | Bip(2′- | (S)-2-amino-4-(2- |
| | amino | Me-Pro | | | | Phe(2- | | | | Et-4′- | methoxyphenoxy) |
| | His | | | | | Fluoro) | | | | OMe) | butanamide |
| 119 | des- | (S)-α- | E | G | T | L-α-Me- | T | S | D | Bip(2′- | (S)-2-amino-4-(4- |
| | amino | Me-Pro | | | | Phe(2- | | | | Et-4′- | trifluoromethylphenoxy) |
| | His | | | | | Fluoro) | | | | OMe) | butanamide |
| 120 | des- | (S)-α- | E | G | T | L-α-Me- | T | S | D | Bip(2′- | (S)-2-amino-4-(2,3- |
| | amino | Me-Pro | | | | Phe(2- | | | | Et-4′- | dihydro-1H-inden-5- |
| | His | | | | | Fluoro) | | | | OMe) | yloxy)butanamide |
| 121 | des- | (S)-α- | E | G | T | L-α-Me- | T | S | D | Bip(2′- | (S)-2-amino-4-(1- |
| | amino | Me-Pro | | | | Phe(2- | | | | Et-4′- | naphthoxy)butanamide |
| | His | | | | | Fluoro) | | | | OMe) | |
| 122 | des- | (S)-α- | E | G | T | L-α-Me- | T | S | D | Bip(2′- | (S)-2-amino-4-(2- |
| | amino | Me-Pro | | | | Phe(2- | | | | Et-4′- | naphthoxy)butanamide |
| | His | | | | | Fluoro) | | | | OMe) | |
| 123 | des- | (S)-α- | E | G | T | L-α-Me- | T | S | D | Bip(2′- | (S)-2-amino-4- |
| | amino | Me-Pro | | | | Phe(2- | | | | Et-4′- | (benzo[d][1,3]dioxol-5- |
| | His | | | | | Fluoro) | | | | OMe) | yloxy)butanamide |
| 124 | des- | (S)-α- | E | G | T | L-α-Me- | T | S | D | Bip(2′- | (S)-2-amino-4-(quinolin- |
| | amino | Me-Pro | | | | Phe(2- | | | | Et-4′- | 6-yloxy)butanamide |
| | His | | | | | Fluoro) | | | | OMe) | |
| 125 | des- | (S)-α- | E | G | T | L-α-Me- | T | S | D | Bip(2′- | (S)-2-amino-5- |
| | amino | Me-Pro | | | | Phe(2- | | | | Et-4′- | phenylpentanamide |
| | His | | | | | Fluoro) | | | | OMe) | |
| 126 | des- | (S)-α- | E | G | T | L-α-Me- | T | S | D | Bip(2′- | (S)-2-amino-5-(3- |
| | amino | Me-Pro | | | | Phe(2- | | | | Et-4′- | chlorophenyl) |
| | His | | | | | Fluoro) | | | | OMe) | pentanamide |
| 127 | des- | (S)-α- | E | G | T | L-α-Me- | T | S | D | Bip(2′- | (S)-2-amino-5- |
| | amino | Me-Pro | | | | Phe(2- | | | | Et-4′- | (benzo[d][1,3]dioxol-5- |
| | His | | | | | Fluoro) | | | | OMe) | yl)pentanamide |
| 128 | des- | (S)-α- | E | G | T | L-α-Me- | T | S | D | Bip(2′- | (S)-2-amino-5-(3,5- |
| | amino | Me-Pro | | | | Phe(2- | | | | Et-4′- | dimethylphenyl) |
| | His | | | | | Fluoro) | | | | OMe) | pentanamide |
| 129 | des- | (S)-α- | E | G | T | L-α-Me- | T | S | D | Bip(2′- | (S)-2-amino-5-(3- |
| | amino | Me-Pro | | | | Phe(2- | | | | Et-4′- | methoxyphenyl) |
| | His | | | | | Fluoro) | | | | OMe) | pentanamide |
| 130 | des- | (S)-α- | E | G | T | L-α-Me- | T | S | D | Bip(2′- | (S)-2-amino-5- |
| | amino | Me-Pro | | | | Phe(2- | | | | Et-4′- | (naphthalen-2- |
| | His | | | | | Fluoro) | | | | OMe) | yl)pentanamide |
| 131 | des- | (S)-α- | E | G | T | L-α-Me- | T | S | D | Bip(2′- | (S)-2-amino-5-(6- |
| | amino | Me-Pro | | | | Phe(2- | | | | Et-4′- | methylpyridin-2- |
| | His | | | | | Fluoro) | | | | OMe) | yl)pentanamide |
| 132 | des- | (S)-α- | E | G | T | L-α-Me- | T | S | D | Bip(2′- | (R)-2-amino-5-(4- |
| | amino | Me-Pro | | | | Phe(2- | | | | Et-4′- | chloro-2-methylphenyl) |
| | His | | | | | Fluoro) | | | | OMe) | pentanamide |
| 133 | des- | (S)-α- | E | G | T | L-α-Me- | T | S | D | Bip(2′- | (S)-2-amino-4-(4-chloro- |
| | amino | Me-Pro | | | | Phe(2- | | | | Et-4′- | 2-methylphenoxy)-N- |
| | His | | | | | Fluoro) | | | | OMe) | methylbutanamide |
| 134 | des- | (S)-α- | E | G | T | L-α-Me- | T | S | D | Bip(2′- | (R)-2-amino-4-(4- |
| | amino | Me-Pro | | | | Phe(2- | | | | Et-4′- | chloro-2- |
| | His | | | | | Fluoro) | | | | OMe) | methylphenoxy)-N- |
| | | | | | | | | | | | methylbutanamide |

TABLE 5

| SEQ | | | | | | | | | | | |
|---|---|---|---|---|---|---|---|---|---|---|---|
| ID NO: | Xₐₐ₁ | Xₐₐ₂ | Xₐₐ₃ | Xₐₐ₄ | Xₐₐ₅ | Xₐₐ₆ | Xₐₐ₇ | Xₐₐ₈ | Xₐₐ₉ | Xₐₐ₁₀ | Xₐₐ₁₁ |
| 135 | H₃C—O—CO- | (S)-α- | E | G | T | L-α-Me- | T | S | D | Bip(2′- | (S)-2-amino-5- |
| | His | Me-Pro | | | | Phe(2- | | | | Et-4′- | phenylpentanoyl-NH—Me |
| | | | | | | Fluoro) | | | | OMe) | |
| 136 | H₃C—O—CO- | (S)-α- | E | G | T | L-α-Me- | T | S | D | Bip(2′- | (S)-2-amino-4- |
| | His | Me-Pro | | | | Phe(2- | | | | Et-4′- | phenoxybutanamide |
| | | | | | | Fluoro) | | | | OMe) | |
| 137 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | fluorophenoxy) |
| | | | | | | Fluoro) | | | | OMe) | butanamide |
| 138 | H₃C—O—CO- | (S)-α- | E | G | T | L-α-Me- | T | S | D | Bip(2′- | (S)-2-amino-4-(2,4- |
| | His | Me-Pro | | | | Phe(2- | | | | Et-4′- | dichlorophenoxy) |
| | | | | | | Fluoro) | | | | OMe) | butanamide |
| 139 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | phenylphenoxy) |
| | | | | | | Fluoro) | | | | OMe) | butanamide |
| 140 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | trifluoromethylphenoxy) |
| | | | | | | Fluoro) | | | | OMe) | butanamide |
| 141 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | methylphenoxy) |
| | | | | | | Fluoro) | | | | OMe) | butanamide |
| 142 | H₃C—O—CO- | (S)-α- | E | G | T | L-α-Me- | T | S | D | Bip(2′- | (S)-2-amino-4-(2,3- |
| | His | Me-Pro | | | | Phe(2- | | | | Et-4′- | dimethylphenoxy) |
| | | | | | | Fluoro) | | | | OMe) | butanamide |
| 143 | H₃C—O—CO- | (S)-α- | E | G | T | L-α-Me- | T | S | D | Bip(2′- | (S)-2-amino-4-(3- |
| | His | Me-Pro | | | | Phe(2- | | | | Et-4′- | acetamidophenoxy) |
| | | | | | | Fluoro) | | | | OMe) | butanamide |
| 144 | H₃C—O—CO- | (S)-α- | E | G | T | L-α-Me- | T | S | D | Bip(2′- | (S)-2-amino-4-(3- |
| | His | Me-Pro | | | | Phe(2- | | | | Et-4′- | phenylphenoxy) |
| | | | | | | Fluoro) | | | | OMe) | butanamide |
| 145 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | methyl-4- |
| | | | | | | Fluoro) | | | | OMe) | chlorophenoxy) |
| | | | | | | | | | | | butanamide |
| 146 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | methyl-4- |
| | | | | | | Fluoro) | | | | OMe) | phenoxyphenoxy) |
| | | | | | | | | | | | butanamide |
| 147 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | methyl-4- |
| | | | | | | Fluoro) | | | | OMe) | methoxyphenoxy) |
| | | | | | | | | | | | butanamide |
| 148 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | methyl-4- |
| | | | | | | Fluoro) | | | | OMe) | trifluoromethylphenoxy) |
| | | | | | | | | | | | butanamide |
| 149 | H₃C—O—CO- | (S)-α- | E | G | T | L-α-Me- | T | S | D | Bip(2′- | (S)-2-amino-4-(2,3- |
| | His | Me-Pro | | | | Phe(2- | | | | Et-4′- | dihydro-1H-inden-5- |
| | | | | | | Fluoro) | | | | OMe) | yloxy)butanamide |
| 150 | H₃C—O—CO- | (S)-α- | E | G | T | L-α-Me- | T | S | D | Bip(2′- | (S)-2-amino-4-(1- |
| | His | Me-Pro | | | | Phe(2- | | | | Et-4′- | naphthoxy)butanamide |
| | | | | | | Fluoro) | | | | OMe) | |
| 151 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | naphthoxy)butanamide |
| | | | | | | Fluoro) | | | | OMe) | |
| 152 | H₃C—O—CO- | (S)-α- | E | G | T | L-α-Me- | T | S | D | Bip(2′- | (S)-2-amino-4- |
| | His | Me-Pro | | | | Phe(2- | | | | Et-4′- | (benzo[d][1,3]dioxol-5- |
| | | | | | | Fluoro) | | | | OMe) | yloxy)butanamide |
| 153 | H₃C—O—CO- | (S)-α- | E | G | T | L-α-Me- | T | S | D | Bip(2′- | (S)-2-amino-4-(quinolin- |
| | His | Me-Pro | | | | Phe(2- | | | | Et-4′- | 4-yloxy)butanamide |
| | | | | | | Fluoro) | | | | OMe) | |
| 154 | H₃C—O—CO- | (S)-α- | E | G | T | L-α-Me- | T | S | D | Bip(2′- | (S)-2-amino-4-(quinolin- |
| | His | Me-Pro | | | | Phe(2- | | | | Et-4′- | 6-yloxy)butanamide |
| | | | | | | Fluoro) | | | | OMe) | |
| 155 | H₃C—O—CO- | (S)-α- | E | G | T | L-α-Me- | T | S | D | Bip(2′- | (S)-2-amino-5- |
| | His | Me-Pro | | | | Phe(2- | | | | Et-4′- | phenylpentanamide |
| | | | | | | Fluoro) | | | | OMe) | |
| 156 | H₃C—O—CO- | (S)-α- | E | G | T | L-α-Me- | T | S | D | Bip(2′- | (S)-2-amino-5-(4- |
| | His | Me-Pro | | | | Phe(2- | | | | Et-4′- | chlorophenyl) |
| | | | | | | Fluoro) | | | | OMe) | pentanamide |
| 157 | H₃C—O—CO- | (S)-α- | E | G | T | L-α-Me- | T | S | D | Bip(2′- | (S)-2-amino-5-(3- |
| | His | Me-Pro | | | | Phe(2- | | | | Et-4′- | chlorophenyl) |
| | | | | | | Fluoro) | | | | OMe) | pentanamide |
| 158 | H₃C—O—CO- | (S)-α- | E | G | T | L-α-Me- | T | S | D | Bip(2′- | (S)-2-amino-5- |
| | His | Me-Pro | | | | Phe(2- | | | | Et-4′- | (benzo[d][1,3]dioxol-5- |
| | | | | | | Fluoro) | | | | OMe) | yl)pentanamide |
| 159 | H₃C—O—CO- | (S)-α- | E | G | T | L-α-Me- | T | S | D | Bip(2′- | (S)-2-amino-5-(3,5- |
| | His | Me-Pro | | | | Phe(2- | | | | Et-4′- | dimethylphenyl) |
| | | | | | | Fluoro) | | | | OMe) | pentanamide |
| 160 | H₃C—O—CO- | (S)-α- | E | G | T | L-α-Me- | T | S | D | Bip(2′- | (S)-2-amino-5-(o- |
| | His | Me-Pro | | | | Phe(2- | | | | Et-4′- | tolyl)pentanamide |
| | | | | | | Fluoro) | | | | OMe) | |
| 161 | H₃C—O—CO- | (S)-α- | E | G | T | L-α-Me- | T | S | D | Bip(2′- | (S)-2-amino-5-(3- |
| | His | Me-Pro | | | | Phe(2- | | | | Et-4′- | methoxyphenyl) |
| | | | | | | Fluoro) | | | | OMe) | pentanamide |
| 162 | H₃C—O—CO- | (S)-α- | E | G | T | L-α-Me- | T | S | D | Bip(2′- | (S)-2-amino-5- |
| | His | Me-Pro | | | | Phe(2- | | | | Et-4′- | (naphthalen-2- |
| | | | | | | Fluoro) | | | | OMe) | yl)pentanamide |
| 163 | H₃C—O—CO- | (S)-α- | E | G | T | L-α-Me- | T | S | D | Bip(2′- | (S)-2-amino-5-(6- |
| | His | Me-Pro | | | | Phe(2- | | | | Et-4′- | methylpyridin-2- |
| | | | | | | Fluoro) | | | | OMe) | yl)pentanamide |
| 164 | H₃C—O—CO- | (S)-α- | E | G | T | L-α-Me- | T | S | D | Bip(2′- | (S)-2-amino-5-(quinolin- |
| | His | Me-Pro | | | | Phe(2- | | | | Et-4′- | 6-yl)pentanamide |
| | | | | | | Fluoro) | | | | OMe) | |
| 165 | H₃C—O—CO- | (S)-α- | E | G | T | L-α-Me- | T | S | D | Bip(2′- | (S)-2-amino-5-(2,3- |
| | His | Me-Pro | | | | Phe(2- | | | | Et-4′- | dimethylphenyl) |
| | | | | | | Fluoro) | | | | OMe) | pentanamide |
| 166 | H₃C—O—CO- | (S)-α- | E | G | T | L-α-Me- | T | S | D | Bip(2′- | 2(R)-2-amino-4-(2-Me-4- |
| | His | Me-Pro | | | | Phe(2- | | | | Et-4′- | Cl-phenoxy)butanamide |
| | | | | | | Fluoro) | | | | OMe) | |
| 167 | H₃C—O—CO- | (S)-α- | E | G | T | L-α-Me- | T | S | D | Bip(2′- | (S)-2-amino-4-(4-chloro- |
| | His | Me-Pro | | | | Phe(2- | | | | Et-4′- | 2-methylphenoxy)-N- |
| | | | | | | Fluoro) | | | | OMe) | methylbutanamide |
| 168 | H₃C—O—CO- | (S)-α- | E | G | T | L-α-Me- | T | S | D | Bip(2′- | (R)-2-amino-4-(4- |
| | His | Me-Pro | | | | Phe(2- | | | | Et-4′- | chloro-2- |
| | | | | | | Fluoro) | | | | OMe) | methylphenoxy)-N- |
| | | | | | | | | | | | methylbutanamide |

Results in the form of EC₅₀ values for selected compounds are shown in Table 6. The structures of exemplary compounds are provided in Table 7.

TABLE 6

| SEQ ID NO: | Human GLP-1 cAMP EC₅₀ (nM) |
|---|---|
| 169 | 0.149 |
| 177 | 0.034 |
| 181 | 0.092 |
| 183 | 0.079 |
| 185 | 0.128 |
| 191 | 0.044 |
| 196 | 0.087 |
| 201 | 0.071 |
| 204 | 0.040 |
| 205 | 0.033 |
| 206 | 0.029 |
| 207 | 0.083 |
| 210 | 0.076 |
| 211 | 0.093 |
| 212 | 0.020 |

TABLE 7

| SEQ ID | | | | | | | | | | | |
|---|---|---|---|---|---|---|---|---|---|---|---|
| NO: | Xₐₐ₁ | Xₐₐ₂ | Xₐₐ₃ | Xₐₐ₄ | Xₐₐ₅ | Xₐₐ₆ | Xₐₐ₇ | Xₐₐ₈ | Xₐₐ₉ | Xₐₐ₁₀ | Xₐₐ₁₁-NH₂ |
| 169 | H | Aib | E | G | T | L-α-Me- | T | S | D | Bip(2′- | (D-Ser(OBz))-NH₂ |
| | | | | | | Phe(2- | | | | Et-4′- | |
| | | | | | | Fluoro) | | | | OMe) | |
| 170 | H | Aib | E | G | T | L-α-Me- | T | S | D | Bip(2′- | (S)-1-(2,3-diamino-3- |
| | | | | | | Phe(2- | | | | Et-4′- | oxopropyl)-3-o-tolylurea |
| | | | | | | Fluoro) | | | | OMe) | |
| 171 | H | Aib | E | G | T | L-α-Me- | T | S | D | Bip(2′- | (S)-1-(2,3-diamino-3- |
| | | | | | | Phe(2- | | | | Et-4′- | oxopropyl)-3-phenylurea |
| | | | | | | Fluoro) | | | | OMe) | |
| 172 | H | (S)-α- | E | G | T | L-α-Me- | T | S | D | Bip(2′- | (S)-1-benzyl-3-(2,3- |
| | | Me-Pro | | | | Phe(2- | | | | Et-4′- | diamino-3-oxopropyl)urea |
| | | | | | | Fluoro) | | | | OMe) | |
| 173 | H | (S)-α- | E | G | T | L-α-Me- | T | S | D | Bip(2′- | (S)-1-phenyl-3-(2,3- |
| | | Me-Pro | | | | Phe(2- | | | | Et-4′- | diamino-3-oxopropyl)urea |
| | | | | | | Fluoro) | | | | OMe) | |
| 174 | H | Aib | E | G | T | L-α-Me- | T | S | D | Bip(2′- | (S)-2-amino-3-(2-o- |
| | | | | | | Phe(2- | | | | Et-4′- | tolylacetamido) |
| | | | | | | Fluoro) | | | | OMe) | propanamide |
| 175 | H | Aib | E | G | T | L-α-Me- | T | S | D | Bip(2′- | (S)-2-amino-3-(2- |
| | | | | | | Phe(2- | | | | Et-4′- | phenylacetamido) |
| | | | | | | Fluoro) | | | | OMe) | propanamide |
| 176 | H | Aib | E | G | T | L-α-Me- | T | S | D | Bip(2′- | (S)-2-amino-4-oxo-4- |
| | | | | | | Phe(2- | | | | Et-4′- | (piperidin-1-yl)butanamide |
| | | | | | | Fluoro) | | | | OMe) | |
| 177 | H | (S)-α- | E | G | T | L-α-Me- | T | S | D | Bip(2′- | (S)-2-chlorobenzyl 2,3- |
| | | Me-Pro | | | | Phe(2- | | | | Et-4-′- | diamino-3- |
| | | | | | | Fluoro) | | | | OMe) | oxopropylcarbamate |
| 178 | H | Aib | E | G | T | L-α-Me- | T | S | D | Bip(2′- | (S)-3-acetamido-2- |
| | | | | | | Phe(2- | | | | Et-4′- | aminopropanamide |
| | | | | | | Fluoro) | | | | OMe) | |
| 179 | H | Aib | E | G | T | L-α-Me- | T | S | D | Bip(2′- | (s)-3-amino-N1-(pyridin- |
| | | | | | | Phe(2- | | | | Et-4′- | 2-ylmethyl)succinamide |
| | | | | | | Fluoro) | | | | OMe) | |
| 180 | H | Aib | E | G | T | L-α-Me- | T | S | D | Bip(2′- | (S)-3-amino-N1-2- |
| | | | | | | Phe(2- | | | | Et-4′- | methylbenzylsuccinamide |
| | | | | | | Fluoro) | | | | OMe) | |
| 181 | H | Aib | E | G | T | L-α-Me- | T | S | D | Bip(2′- | (S)-3-amino-N1- |
| | | | | | | Phe(2- | | | | Et-4′- | benzylsuccinamide |
| | | | | | | Fluoro) | | | | OMe) | |
| 182 | H | Aib | E | G | T | L-α-Me- | T | S | D | Bip(2′- | (S)-3-amino-N1- |
| | | | | | | Phe(2- | | | | Et-4′- | isobutylsuccinamide |
| | | | | | | Fluoro) | | | | OMe) | |
| 183 | H | (S)-α- | E | G | T | L-α-Me- | T | S | D | Bip(2′- | (S)-benzyl 2,3-diamino-3- |
| | | Me-Pro | | | | Phe(2- | | | | Et-4′- | oxopropylcarbamate |
| | | | | | | Fluoro) | | | | OMe) | |
| 184 | H | Aib | E | G | T | L-α-Me- | T | S | D | Bip(2′- | (S)—N-(2,3-diamino-3- |
| | | | | | | Phe(2- | | | | Et-4′- | oxopropyl)-2- |
| | | | | | | Fluoro) | | | | OMe) | methylbenzamide |
| 185 | H | Aib | E | G | T | L-α-Me- | T | S | D | Bip(2′- | (S)—N-(2,3-diamino-3- |
| | | | | | | Phe(2- | | | | Et-4′- | oxopropyl)-3- |
| | | | | | | Fluoro) | | | | OMe) | methylbenzamide |
| 186 | H | Aib | E | G | T | L-α-Me- | T | S | D | Bip(2′- | (S)—N-(2,3-diamino-3- |
| | | | | | | Phe(2- | | | | Et-4′- | oxopropyl)-3- |
| | | | | | | Fluoro) | | | | OMe) | methylpicolinamide |
| 187 | H | Aib | E | G | T | L-α-Me- | T | S | D | Bip(2′- | (S)—N-(2,3-diamino-3- |
| | | | | | | Phe(2- | | | | Et-4′- | oxopropyl)-4- |
| | | | | | | Fluoro) | | | | OMe) | methylbenzamide |
| 188 | H | Aib | E | G | T | L-α-Me- | T | S | D | Bip(2′- | (S)—N-(2,3-diamino-3- |
| | | | | | | Phe(2- | | | | Et-4′- | oxopropyl)benzamide |
| | | | | | | Fluoro) | | | | OMe) | |
| 189 | H | Aib | E | G | T | L-α-Me- | T | S | D | Bip(2′- | (S)—N-(2,3-diamino-3- |
| | | | | | | Phe(2- | | | | Et-4′- | oxopropyl)isonicotinamide |
| | | | | | | Fluoro) | | | | OMe) | |
| 190 | H | Aib | E | G | T | L-α-Me- | T | S | D | Bip(2′- | Glu(1-(2-aminoethyl) |
| | | | | | | Phe(2- | | | | Et-4′- | piperidine)-NH₂ |
| | | | | | | Fluoro) | | | | OMe) | |
| 191 | H | (S)-α- | E | G | T | L-α-Me- | T | S | D | Bip(2′- | Glu(1,2,3,4- |
| | | Me-Pro | | | | Phe(2- | | | | Et-4′- | tetrahydroisoquinoline)- |
| | | | | | | Fluoro) | | | | OMe) | NH₂ |
| 192 | H | Aib | E | G | T | L-α-Me- | T | S | D | Bip(2′- | Glu(2-(2- |
| | | | | | | Phe(2- | | | | Et-4′- | aminoethyl)pyridine)-NH₂ |
| | | | | | | Fluoro) | | | | OMe) | |
| 193 | H | Aib | E | G | T | L-α-Me- | T | S | D | Bip(2′- | Glu(2-fluorobenzylamine)- |
| | | | | | | Phe(2- | | | | Et-4′- | NH₂ |
| | | | | | | Fluoro) | | | | OMe) | |
| 194 | H | Aib | E | G | T | L-α-Me- | T | S | D | Bip(2′- | Glu(2- |
| | | | | | | Phe(2- | | | | Et-4′- | methylbenzylamine)-NH₂ |
| | | | | | | Fluoro) | | | | OMe) | |
| 195 | H | Aib | E | G | T | L-α-Me- | T | S | D | Bip(2′- | Glu(3- |
| | | | | | | Phe(2- | | | | Et-4′- | methoxybenzylamine)- |
| | | | | | | Fluoro) | | | | OMe) | NH₂ |
| 196 | H | (S)-α- | E | G | T | L-α-Me- | T | S | D | Bip(2′- | Glu(3-methoxy-N- |
| | | Me-Pro | | | | Phe(2- | | | | Et-4′- | methylbenzylamine)-NH₂ |
| | | | | | | Fluoro) | | | | OMe) | |
| 197 | H | Aib | E | G | T | L-α-Me- | T | S | D | Bip(2′- | Glu(4-CF₃-benzylamine)- |
| | | | | | | Phe(2- | | | | Et-4′- | NH₂ |
| | | | | | | Fluoro) | | | | OMe) | |
| 198 | H | Aib | E | G | T | L-α-Me- | T | S | D | Bip(2′- | Glu(benzylamine)-NH₂ |
| | | | | | | Phe(2- | | | | Et-4′- | |
| | | | | | | Fluoro) | | | | OMe) | |
| 199 | H | (S)-α- | E | G | T | L-α-Me- | T | S | D | Bip(2′- | Glu(dibenzylamine)-NH₂ |
| | | Me-Pro | | | | Phe(2- | | | | Et-4′- | |
| | | | | | | Fluoro | | | | OMe) | |
| 200 | H | Aib | E | G | T | L-α-Me- | T | S | D | Bip(2′- | Glu(isobutylamine)-NH₂ |
| | | | | | | Phe(2- | | | | Et-4′- | |
| | | | | | | Fluoro) | | | | OMe) | |
| 201 | H | (S)-α- | E | G | T | L-α-Me- | T | S | D | Bip(2′- | Glu(isoindoline)-NH₂ |
| | | Me-Pro | | | | Phe(2- | | | | Et-4′- | |
| | | | | | | Fluoro) | | | | OMe) | |
| 202 | H | Aib | E | G | T | L-α-Me- | T | S | D | Bip(2′- | Glu(N,N- |
| | | | | | | Phe(2- | | | | Et-4′- | dimethylethylenediamine)- |
| | | | | | | Fluoro) | | | | OMe) | NH₂ |
| 203 | H | Aib | E | G | T | L-α-Me- | T | S | D | Bip(2′- | Glu(n-butylamine)-NH₂ |
| | | | | | | Phe(2- | | | | Et-4′- | |
| | | | | | | Fluoro) | | | | OMe) | |
| 204 | H | (S)-α- | E | G | T | L-α-Me- | T | S | D | Bip(2′- | Glu(N-ethylbenzylamine)- |
| | | Me-Pro | | | | Phe(2- | | | | Et-4′- | NH₂ |
| | | | | | | Fluoro) | | | | OMe) | |
| 205 | H | (S)-α- | E | G | T | L-α-Me- | T | S | D | Bip(2′- | Glu(N- |
| | | Me-Pro | | | | Phe(2- | | | | Et-4′- | methylbenzylamine)-NH₂ |
| | | | | | | Fluoro) | | | | OMe) | |
| 206 | H | Aib | E | G | T | L-α-Me- | T | S | D | Bip(2′- | Glu(OBz)-NH₂ |
| | | | | | | Phe(2- | | | | Et-4′- | |
| | | | | | | Fluoro) | | | | OMe) | |
| 207 | H | (S)-α- | E | G | T | L-α-Me- | T | S | D | Bip(2′- | Glu(OBz)-NH₂ |
| | | Me-Pro | | | | Phe(2- | | | | Et-4′- | |
| | | | | | | Fluoro) | | | | OMe) | |
| 208 | H | Aib | E | G | T | L-α-Me- | T | S | D | Bip(2′- | Glu(phenethylamine)-NH₂ |
| | | | | | | Phe(2- | | | | Et-4′- | |
| | | | | | | Fluoro) | | | | OMe) | |
| 209 | H | Aib | E | G | T | L-α-Me- | T | S | D | Bip(2′- | Glu(piperidine)-NH₂ |
| | | | | | | Phe(2- | | | | Et-4′- | |
| | | | | | | Fluoro) | | | | OMe) | |
| 210 | H | Aib | E | G | T | L-α-Me- | T | S | D | Bip(2′- | Ser(Bzl)-NH₂ |
| | | | | | | Phe(2- | | | | Et-4′- | |
| | | | | | | Fluoro) | | | | OMe) | |
| 211 | H | Aib | E | G | T | L-α-Me- | T | S | D | Bip(2′- | Thr(OBz)-NH₂ |
| | | | | | | Phe(2- | | | | Et-4′- | |
| | | | | | | Fluoro) | | | | OMe) | |
| 212 | H | Aib | E | G | T | L-α-Me- | T | S | D | Bip(2′- | L-Asp(OBz)-NH₂ |
| | | | | | | Phe(2- | | | | Et-4′- | |
| | | | | | | Fluoro) | | | | OMe) | |

Results in the form of EC₅₀ values for selected compounds are shown in Table 8. The structures of exemplary compounds are provided in Table 9.

TABLE 8

| SEQ ID NO: | Human GLP-1 cAMP EC₅₀ (nM) |
|---|---|
| 224 | 0.060 |
| 227 | 0.254 |
| 235 | 0.065 |
| 236 | 0.066 |
| 239 | 0.043 |
| 254 | 0.385 |
| 255 | 0.884 |
| 267 | 0.322 |
| 273 | 0.032 |
| 274 | 0.023 |
| 275 | 0.029 |
| 276 | 0.084 |
| 287 | 0.068 |
| 288 | 0.032 |
| 292 | 0.094 |

TABLE 9

| SEQ | | | | | | | | | | | |
|---|---|---|---|---|---|---|---|---|---|---|---|
| ID NO: | Xₐₐ₁ | Xₐₐ₂ | Xₐₐ₃ | Xₐₐ₄ | Xₐₐ₅ | Xₐₐ₆ | Xₐₐ₇ | Xₐₐ₈ | Xₐₐ₉ | Xₐₐ₁₀ | Xₐₐ₁₁-NH₂ |
| 213 | H | Aib | E | G | T | L-α-Me- | T | S | D | Bip(2′- | (S)-2-amino-3-(4- |
| | | | | | | Phe(2- | | | | Et-4′- | (benzamidomethyl)phenyl) |
| | | | | | | Fluoro) | | | | OMe) | propanoic acid |
| 214 | H | Aib | E | G | T | L-α-Me- | T | S | D | Bip(2′- | (S)-2-amino-3-(4- |
| | | | | | | Phe(2- | | | | Et-4′- | (phenylsulfonamidomethyl) |
| | | | | | | Fluoro) | | | | OMe) | phenyl)propanoic acid |
| 215 | H | Aib | E | G | T | L-α-Me- | T | S | D | Bip(2′- | (S)-2-amino-3-(4- |
| | | | | | | Phe(2- | | | | Et-4′- | (methylsulfonamidomethyl) |
| | | | | | | Fluoro) | | | | OMe) | phenyl)propanoic acid |
| 216 | H | Aib | E | G | T | L-α-Me- | T | S | D | Bip(2′- | (S)-2-amino-3-(4-((2,3- |
| | | | | | | Phe(2- | | | | Et-4′- | dimethoxybenzamido) |
| | | | | | | Fluoro) | | | | OMe) | methyl)phenyl)propanoic) |
| | | | | | | | | | | | acid |
| 217 | H | Aib | E | G | T | L-α-Me- | T | S | D | Bip(2′- | (S)-2-amino-3-(4-((2,4- |
| | | | | | | Phe(2- | | | | Et-4′- | difluorobenzamido)methyl) |
| | | | | | | Fluoro) | | | | OMe) | phenyl)propanoic acid |
| 218 | H | Aib | E | G | T | L-α-Me- | T | S | D | Bip(2′- | (S)-2-amino-3-(4-((2,4- |
| | | | | | | Phe(2- | | | | Et-4′- | dimethoxybenzamido) |
| | | | | | | Fluoro) | | | | OMe) | methyl)phenyl)propanoic |
| | | | | | | | | | | | acid |
| 219 | H | Aib | E | G | T | L-α-Me- | T | S | D | Bip(2′- | (S)-2-amino-3-(4-((2,5- |
| | | | | | | Phe(2- | | | | Et-4′- | dimethoxybenzamido) |
| | | | | | | Fluoro) | | | | OMe) | methyl)phenyl)propanoic |
| | | | | | | | | | | | acid |
| 220 | H | Aib | E | G | T | L-α-Me- | T | S | D | Bip(2′- | (S)-2-amino-3-(4-((2,6- |
| | | | | | | Phe(2- | | | | Et-4′- | dichlorophenylsulfonamido) |
| | | | | | | Fluoro) | | | | OMe) | methyl)phenyl)propanoic |
| | | | | | | | | | | | acid |
| 221 | H | Aib | E | G | T | L-α-Me- | T | S | D | Bip(2′- | (S)-2-amino-3-(4-((2- |
| | | | | | | Phe(2- | | | | Et-4′- | chlorobenzamido)methyl) |
| | | | | | | Fluoro) | | | | OMe) | phenyl)propanoic acid |
| 222 | H | Aib | E | G | T | L-α-Me- | T | S | D | Bip(2′- | (S)-2-amino-3-(4-((2- |
| | | | | | | Phe(2- | | | | Et-4′- | methoxybenzamido)methyl) |
| | | | | | | Fluoro) | | | | OMe) | phenyl)propanoic acid |
| 223 | H | Aib | E | G | T | L-α-Me- | T | S | D | Bip(2′- | (S)-2-amino-3-(4-((2- |
| | | | | | | Phe(2- | | | | Et-4′- | methylbenzamido)methyl) |
| | | | | | | Fluoro) | | | | OMe) | phenyl)propanoic acid |
| 224 | H | Aib | E | G | T | L-α-Me- | T | S | D | Bip(2′- | (S)-2-amino-3-(4-((2- |
| | | | | | | Phe(2- | | | | Et-4′- | methylphenylsulfonamido) |
| | | | | | | Fluoro) | | | | OMe) | methyl)phenyl)propanoic |
| | | | | | | | | | | | acid |
| 225 | H | Aib | E | G | T | L-α-Me- | T | S | D | Bip(2′- | (S)-2-amino-3-(4-((3,4- |
| | | | | | | Phe(2- | | | | Et-4′- | dichlorophenylsulfonamido) |
| | | | | | | Fluoro | | | | OMe) | methyl)phenyl)propanoic |
| | | | | | | | | | | | acid |
| 226 | H | Aib | E | G | T | L-α-Me- | T | S | D | Bip(2′- | (S)-2-amino-3-(4-((3,4- |
| | | | | | | Phe(2- | | | | Et-4′- | dimethoxybenzamido) |
| | | | | | | Fluoro) | | | | OMe) | methyl)phenyl)propanoic |
| | | | | | | | | | | | acid |
| 227 | H | Aib | E | G | T | L-α-Me- | T | S | D | Bip(2′- | (S)-2-amino-3-(4- |
| | | | | | | Phe(2- | | | | Et-4′- | ((benzo[d][1,3]dioxole-5- |
| | | | | | | Fluoro) | | | | OMe) | carboxamido)methyl)phenyl) |
| | | | | | | | | | | | propanoic acid |
| 228 | H | Aib | E | G | T | L-α-Me- | T | S | D | Bip(2′- | (S)-2-amino-3-(4-((3,5- |
| | | | | | | Phe(2- | | | | Et-4′- | dichlorophenylsulfonamido) |
| | | | | | | Fluoro) | | | | OMe) | methyl)phenyl)propanoic |
| | | | | | | | | | | | acid |
| 229 | H | Aib | E | G | T | L-α-Me- | T | S | D | Bip(2′- | (S)-2-amino-3-(4-((3,5- |
| | | | | | | Phe(2- | | | | Et-4′- | dimethoxybenzamido) |
| | | | | | | Fluoro) | | | | OMe) | methyl)phenyl)propanoic |
| | | | | | | | | | | | acid |
| 230 | H | Aib | E | G | T | L-α-Me- | T | S | D | Bip(2′- | (S)-2-amino-3-(4-((3,5- |
| | | | | | | Phe(2- | | | | Et-4′- | dimethylisoxazole-4- |
| | | | | | | Fluoro) | | | | OMe) | sulfonamido)methyl)phenyl) |
| | | | | | | | | | | | propanoic acid |
| 231 | H | Aib | E | G | T | L-α-Me- | T | S | D | Bip(2′- | (S)-2-amino-3-(4-((3- |
| | | | | | | Phe(2- | | | | Et-4′- | (trifluoromethyl)benzamido) |
| | | | | | | Fluoro) | | | | OMe) | methyl)phenyl)propanoic |
| | | | | | | | | | | | acid |
| 232 | H | Aib | E | G | T | L-α-Me- | T | S | D | Bip(2′- | (S)-2-amino-3-(4-((3- |
| | | | | | | Phe(2- | | | | Et-4′- | chlorobenzamido)methyl) |
| | | | | | | Fluoro) | | | | OMe) | phenyl)propanoic acid |
| 233 | H | Aib | E | G | T | L-α-Me- | T | S | D | Bip(2′- | (S)-2-amino-3-(4-((3- |
| | | | | | | Phe(2- | | | | Et-4′- | chlorophenylsulfonamido) |
| | | | | | | Fluoro) | | | | OMe) | methyl)phenyl)propanoic |
| | | | | | | | | | | | acid |
| 234 | H | Aib | E | G | T | L-α-Me- | T | S | D | Bip(2′- | (S)-2-amino-3-(4-((3- |
| | | | | | | Phe(2- | | | | Et-4′- | cyanobenzamido)methyl) |
| | | | | | | Fluoro) | | | | OMe) | phenyl)propanoic acid |
| 235 | H | Aib | E | G | T | L-α-Me- | T | S | D | Bip(2′- | (S)-2-amino-3-(4-((3- |
| | | | | | | Phe(2- | | | | Et-4′- | ethoxybenzamido)methyl) |
| | | | | | | Fluoro) | | | | OMe) | phenyl)propanoic acid |
| 236 | H | Aib | E | G | T | L-α-Me- | T | S | D | Bip(2′- | (S)-2-amino-3-(4-((3- |
| | | | | | | Phe(2- | | | | Et-4′- | fluorobenzamido)methyl) |
| | | | | | | Fluoro) | | | | OMe) | phenyl)propanoic acid |
| 237 | H | Aib | E | G | T | L-α-Me- | T | S | D | Bip(2′- | (S)-2-amino-3-(4-((3- |
| | | | | | | Phe(2- | | | | Et-4′- | isopropylbenzamido)methyl) |
| | | | | | | Fluoro) | | | | OMe) | phenyl)propanoic acid |
| 238 | H | Aib | E | G | T | L-α-Me- | T | S | D | Bip(2′- | (S)-2-amino-3-(4-((3- |
| | | | | | | Phe(2- | | | | Et-4′- | methoxy-4- |
| | | | | | | Fluoro) | | | | OMe) | methylbenzamido)methyl) |
| | | | | | | | | | | | phenyl)propanoic acid |
| 239 | H | Aib | E | G | T | L-α-Me- | T | S | D | Bip(2′- | (S)-2-amino-3-(4-((3- |
| | | | | | | Phe(2- | | | | Et-4′- | methoxybenzamido)methyl) |
| | | | | | | Fluoro) | | | | OMe) | phenyl)propanoic acid |
| 240 | H | Aib | E | G | T | L-α-Me- | T | S | D | Bip(2′- | (S)-2-amino-3-(4-((3- |
| | | | | | | Phe(2- | | | | Et-4′- | methoxyphenylsulfonamido) |
| | | | | | | Fluoro) | | | | OMe) | methyl)phenyl)propanoic |
| | | | | | | | | | | | acid |
| 241 | H | Aib | E | G | T | L-α-Me- | T | S | D | Bip(2′- | (S)-2-amino-3-(4-((3- |
| | | | | | | Phe(2- | | | | Et-4′- | methylbenzamido)methyl) |
| | | | | | | Fluoro) | | | | OMe) | phenyl)propanoic acid |
| 242 | H | Aib | E | G | T | L-α-Me- | T | S | D | Bip(2′- | (S)-2-amino-3-(4-((3- |
| | | | | | | Phe(2- | | | | Et-4′- | (trifluoromethoxy) |
| | | | | | | Fluoro) | | | | OMe) | benzamido)methyl)phenyl) |
| | | | | | | | | | | | propanoic acid |
| 243 | H | Aib | E | G | T | L-α-Me- | T | S | D | Bip(2′- | (S)-2-amino-3-(4-((3- |
| | | | | | | Phe(2- | | | | Et-4′- | phenylbenzamido)methyl) |
| | | | | | | Fluoro) | | | | OMe) | phenyl)propanoic acid |
| 244 | H | Aib | E | G | T | L-α-Me- | T | S | D | Bip(2′- | (S)-2-amino-3-(4- |
| | | | | | | Phe(2- | | | | Et-4′- | (nicotinamidomethyl)phenyl) |
| | | | | | | Fluoro) | | | | OMe) | propanoic acid |
| 245 | H | Aib | E | G | T | L-α-Me- | T | S | D | Bip(2′- | (S)-2-amino-3-(4-((4- |
| | | | | | | Phe(2- | | | | Et-4′- | butylbenzamido)methyl) |
| | | | | | | Fluoro) | | | | OMe) | phenyl)propanoic acid |
| 246 | H | Aib | E | G | T | L-α-Me- | T | S | D | Bip(2′- | (S)-2-amino-3-(4-((4- |
| | | | | | | Phe(2- | | | | Et-4′- | chlorophenylsulfonamido) |
| | | | | | | Fluoro) | | | | OMe) | methyl)phenyl)propanoic |
| | | | | | | | | | | | acid |
| 247 | H | Aib | E | G | T | L-α-Me- | T | S | D | Bip(2′- | (S)-2-amino-3-(4-((4- |
| | | | | | | Phe(2- | | | | Et-4′- | cyclohexylbenzamido) |
| | | | | | | Fluoro) | | | | OMe) | methyl)phenyl)propanoic |
| | | | | | | | | | | | acid |
| 248 | H | Aib | E | G | T | L-α-Me- | T | S | D | Bip(2′- | (S)-2-amino-3-(4-((4- |
| | | | | | | Phe(2- | | | | Et-4′- | methoxybenzamido)methyl) |
| | | | | | | Fluoro) | | | | OMe) | phenyl)propanoic acid |
| 249 | H | Aib | E | G | T | L-α-Me- | T | S | D | Bip(2′- | (S)-2-amino-3-(4-((4- |
| | | | | | | Phe(2- | | | | Et-4′- | methylphenylsulfonamido) |
| | | | | | | Fluoro) | | | | OMe) | methyl)phenyl)propanoic |
| | | | | | | | | | | | acid |
| 250 | H | Aib | E | G | T | L-α-Me- | T | S | D | Bip(2′- | (S)-2-amino-3-(4-((4- |
| | | | | | | Phe(2- | | | | Et-4′- | methylbenzamido)methyl) |
| | | | | | | Fluoro) | | | | OMe) | phenyl)propanoic acid |
| 251 | H | Aib | E | G | T | L-α-Me- | T | S | D | Bip(2′- | (S)-2-amino-3-(4-((4- |
| | | | | | | Phe(2- | | | | Et-4′- | benzylbenzamido)methyl) |
| | | | | | | Fluoro) | | | | OMe) | phenyl)propanoic acid |
| 252 | H | Aib | E | G | T | L-α-Me- | T | S | D | Bip(2′- | (S)-2-amino-3-(4-((4- |
| | | | | | | Phe(2- | | | | Et-4′- | phenylphenylsulfonamido) |
| | | | | | | Fluoro) | | | | OMe) | methyl)phenyl)propanoic |
| | | | | | | | | | | | acid |
| 253 | H | Aib | E | G | T | L-α-Me- | T | S | D | Bip(2′- | (S)-2-amino-3-(4-((4- |
| | | | | | | Phe(2- | | | | Et-4′- | phenylphenylsulfonamido) |
| | | | | | | Fluoro) | | | | OMe) | methyl)phenyl)propanoic |
| | | | | | | | | | | | acid |
| 254 | H | Aib | E | G | T | L-α-Me- | T | S | D | Bip(2′- | (S)-3-(4-(acetamidomethyl) |
| | | | | | | Phe(2- | | | | Et-4′- | phenyl)-2-aminopropanoic |
| | | | | | | Fluoro) | | | | OMe) | acid |
| 255 | H | Aib | E | G | T | L-α-Me- | T | S | D | Bip(2′- | (S)-2-amino-3-(4- |
| | | | | | | Phe(2- | | | | Et-4′- | (cyclohexanecarboxamidome |
| | | | | | | Fluoro) | | | | OMe) | thyl)phenyl)propanoic acid |
| 256 | H | Aib | E | G | T | L-α-Me- | T | S | D | Bip(2′- | (S)-2-amino-3-(4-((3- |
| | | | | | | Phe(2- | | | | Et-4′- | methylbutanamido)methyl) |
| | | | | | | Fluoro) | | | | OMe) | phenyl)propanoic acid |
| 257 | H | Aib | E | G | T | L-α-Me- | T | S | D | Bip(2′- | (S)-2-amino-3-(4-(2,4- |
| | | | | | | Phe(2- | | | | Et-4′- | dimethoxybenzamido) |
| | | | | | | Fluoro) | | | | OMe) | phenyl)propanoic acid |
| 258 | H | Aib | E | G | T | L-α-Me- | T | S | D | Bip(2′- | (S)-2-amino-3-(4-(2,6- |
| | | | | | | Phe(2- | | | | Et-4′- | dichlorophenylsulfonamido) |
| | | | | | | Fluoro) | | | | OMe) | phenyl)propanoic acid |
| 259 | H | Aib | E | G | T | L-α-Me- | T | S | D | Bip(2′- | (S)-2-amino-3-(4-(2- |
| | | | | | | Phe(2- | | | | Et-4′- | chlorobenzamido)phenyl) |
| | | | | | | Fluoro) | | | | OMe) | propanoic acid |
| 260 | H | Aib | E | G | T | L-α-Me- | T | S | D | Bip(2′- | (S)-2-amino-3-(4-(2- |
| | | | | | | Phe(2- | | | | Et-4′- | methylbenzamido)phenyl) |
| | | | | | | Fluoro) | | | | OMe) | propanoic acid |
| 261 | H | Aib | E | G | T | L-α-Me- | T | S | D | Bip(2′- | (S)-2-amino-3-(4-(3,4- |
| | | | | | | Phe(2- | | | | Et-4′- | dichlorophenylsulfonamido) |
| | | | | | | Fluoro) | | | | OMe) | phenyl)propanoic acid |
| 262 | H | Aib | E | G | T | L-α-Me- | T | S | D | Bip(2′- | (S)-2-amino-3-(4-(3,4- |
| | | | | | | Phe(2- | | | | Et-4′- | dimethoxybenzamido) |
| | | | | | | Fluoro) | | | | OMe) | phenyl)propanoic acid |
| 263 | H | Aib | E | G | T | L-α-Me- | T | S | D | Bip(2′- | (S)-2-amino-3-(4-(3,5- |
| | | | | | | Phe(2- | | | | Et-4′- | dichlorophenylsulfonamido) |
| | | | | | | Fluoro) | | | | OMe) | phenyl)propanoic acid |
| 264 | H | Aib | E | G | T | L-α-Me- | T | S | D | Bip(2′- | (S)-2-amino-3-(4-(3,5- |
| | | | | | | Phe(2- | | | | Et-4′- | dimethylisoxazole-4- |
| | | | | | | Fluoro) | | | | OMe) | sulfonamido)phenyl) |
| | | | | | | | | | | | propanoic acid |
| 265 | H | Aib | E | G | T | L-α-Me- | T | S | D | Bip(2′- | (S)-2-amino-3-(4-(3- |
| | | | | | | Phe(2- | | | | Et-4′- | chlorophenylsulfonamido) |
| | | | | | | Fluoro) | | | | OMe) | phenyl)propanoic acid |
| 266 | H | Aib | E | G | T | L-α-Me- | T | S | D | Bip(2′- | (S)-2-amino-3-(4-(3- |
| | | | | | | Phe(2- | | | | Et-4′- | methylbenzamido)phenyl) |
| | | | | | | Fluoro) | | | | OMe) | propanoic acid |
| 267 | H | Aib | E | G | T | L-α-Me- | T | S | D | Bip(2′- | (S)-2-amino-3-(4- |
| | | | | | | Phe(2- | | | | Et-4′- | (nicotinamido)phenyl) |
| | | | | | | Fluoro) | | | | OMe) | propanoic acid |
| 268 | H | Aib | E | G | T | L-α-Me- | T | S | D | Bip(2′- | (S)-2-amino-3-(4-(4- |
| | | | | | | Phe(2- | | | | Et-4′- | methylphenylsulfonamido) |
| | | | | | | Fluoro) | | | | OMe) | phenyl)propanoic acid |
| 269 | H | Aib | E | G | T | L-α-Me- | T | S | D | Bip(2′- | (S)-2-amino-3-(4-(3- |
| | | | | | | Phe(2- | | | | Et-4′- | phenylbenzamido)phenyl) |
| | | | | | | Fluoro) | | | | OMe) | propanoic acid |
| 270 | H | Aib | E | G | T | L-α-Me- | T | S | D | Bip(2′- | (S)-2-amino-3-(4-(4- |
| | | | | | | Phe(2- | | | | Et-4′- | biphenylsulfonamido) |
| | | | | | | Fluoro) | | | | OMe) | phenyl)propanoic acid |
| 271 | H | Aib | E | G | T | L-α-Me- | T | S | D | Bip(2′- | (S)-2-amino-3-(4- |
| | | | | | | Phe(2- | | | | Et-4′- | (phenylsulfonamido)phenyl) |
| | | | | | | Fluoro) | | | | OMe) | propanoic acid |
| 272 | H | Aib | E | G | T | L-α-Me- | T | S | D | Bip(2′- | (S)-2-amino-3-(4- |
| | | | | | | Phe(2- | | | | Et-4′- | (methylsulfonamido)phenyl) |
| | | | | | | Fluoro) | | | | OMe) | propanoic acid |
| 273 | H | Aib | E | G | T | L-α-Me- | T | S | D | Bip(2′- | (S)-2-amino-3-(4-(2,4- |
| | | | | | | Phe(2- | | | | Et-4′- | difluorobenzamido)phenyl) |
| | | | | | | Fluoro) | | | | OMe) | propanoic acid |
| 274 | H | Aib | E | G | T | L-α-Me- | T | S | D | Bip(2′- | (S)-2-amino-3-(4-(2- |
| | | | | | | Phe(2- | | | | Et-4′- | chlorobenzamido)phenyl) |
| | | | | | | Fluoro) | | | | OMe) | propanoic acid |
| 275 | H | Aib | E | G | T | L-α-Me- | T | S | D | Bip(2′- | (S)-2-amino-3-(4-(2- |
| | | | | | | Phe(2- | | | | Et-4′- | methoxybenzamido)phenyl) |
| | | | | | | Fluoro) | | | | OMe) | propanoic acid |
| 276 | H | Aib | E | G | T | L-α-Me- | T | S | D | Bip(2′- | (S)-2-amino-3-(4-(2- |
| | | | | | | Phe(2- | | | | Et-4′- | methylphenylsulfonamido) |
| | | | | | | Fluoro) | | | | OMe) | phenyl)propanoic acid |
| 277 | H | Aib | E | G | T | L-α-Me- | T | S | D | Bip(2′- | (S)-2-amino-3-(4-(3- |
| | | | | | | Phe(2- | | | | Et-4′- | methoxybenzamido)phenyl) |
| | | | | | | Fluoro) | | | | OMe) | propanoic acid |
| 278 | H | Aib | E | G | T | L-α-Me- | T | S | D | Bip(2′- | (S)-2-amino-3-(4-(3- |
| | | | | | | Phe(2- | | | | Et-4′- | methoxyphenylsulfonamido) |
| | | | | | | Fluoro) | | | | OMe) | phenyl)propanoic acid |
| 279 | H | Aib | E | G | T | L-α-Me- | T | S | D | Bip(2′- | (S)-2-amino-3-(4-(4- |
| | | | | | | Phe(2- | | | | Et-4′- | chlorophenylsulfonamido) |
| | | | | | | Fluoro) | | | | OMe) | phenyl)propanoic acid |
| 280 | H | Aib | E | G | T | L-α-Me- | T | S | D | Bip(2′- | (S)-2-amino-3-(4-(4- |
| | | | | | | Phe(2- | | | | Et-4′- | methoxyphenylsulfonamido) |
| | | | | | | Fluoro) | | | | OMe) | phenyl)propanoic acid |
| 281 | H | (S)-α- | E | G | T | L-α-Me- | T | S | D | Bip(2′- | (S)-2-amino-3-(4-((2,3- |
| | | Me-Pro | | | | Phe(2- | | | | Et-4′- | dimethoxybenzamido) |
| | | | | | | Fluoro) | | | | OMe) | methyl)phenyl)propanoic |
| | | | | | | | | | | | acid |
| 282 | H | (S)-α- | E | G | T | L-α-Me- | T | S | D | Bip(2′- | (S)-2-amino-3-(4-((2,5- |
| | | Me-Pro | | | | Phe(2- | | | | Et-4′- | dimethoxybenzamido) |
| | | | | | | Fluoro) | | | | OMe) | methyl)phenyl)propanoic |
| | | | | | | | | | | | acid |
| 283 | H | (S)-α- | E | G | T | L-α-Me- | T | S | D | Bip(2′- | (S)-2-amino-3-(4- |
| | | Me-Pro | | | | Phe(2- | | | | Et-4′- | ((benzo[d][1,3]dioxole-5- |
| | | | | | | Fluoro) | | | | OMe) | carboxamido)methyl)phenyl) |
| | | | | | | | | | | | propanoic acid |
| 284 | H | (S)-α- | E | G | T | L-α-Me- | T | S | D | Bip(2′- | (S)-2-amino-3-(4-((3,5- |
| | | Me-Pro | | | | Phe(2- | | | | Et-4′- | dimethoxybenzamido) |
| | | | | | | Fluoro) | | | | OMe) | methyl)phenyl)propanoic |
| | | | | | | | | | | | acid |
| 285 | H | (S)-α- | E | G | T | L-α-Me- | T | S | D | Bip(2′- | (S)-2-amino-3-(4-((3- |
| | | Me-Pro | | | | Phe(2- | | | | Et-4′- | (trifluoromethyl)benzamido) |
| | | | | | | Fluoro) | | | | OMe) | methyl)phenyl)propanoic |
| | | | | | | | | | | | acid |
| 286 | H | (S)-α- | E | G | T | L-α-Me- | T | S | D | Bip(2′- | (S)-2-amino-3-(4-((3- |
| | | Me-Pro | | | | Phe(2- | | | | Et-4′- | cyanobenzamido)methyl) |
| | | | | | | Fluoro) | | | | OMe) | phenyl)propanoic acid |
| 287 | H | (S)-α- | E | G | T | L-α-Me- | T | S | D | Bip(2′- | (S)-2-amino-3-(4-((3- |
| | | Me-Pro | | | | Phe(2- | | | | Et-4′- | ethoxybenzamido)methyl) |
| | | | | | | Fluoro) | | | | OMe) | phenyl)propanoic acid |
| 288 | H | (S)-α- | E | G | T | L-α-Me- | T | S | D | Bip(2′- | (S)-2-amino-3-(4-((3- |
| | | Me-Pro | | | | Phe(2- | | | | Et-4′- | fluorobenzamido)methyl) |
| | | | | | | Fluoro) | | | | OMe) | phenyl)propanoic acid |
| 289 | H | (S)-α- | E | G | T | L-α-Me- | T | S | D | Bip(2′- | (S)-2-amino-3-(4-((3- |
| | | Me-Pro | | | | Phe(2- | | | | Et-4′- | methoxy-4- |
| | | | | | | Fluoro) | | | | OMe) | methylbenzamido)methyl) |
| | | | | | | | | | | | phenyl)propanoic acid |
| 290 | H | (S)-α- | E | G | T | L-α-Me- | T | S | D | Bip(2′- | (S)-2-amino-3-(4-((3- |
| | | Me-Pro | | | | Phe(2- | | | | Et-4′- | methylbenzamido)methyl) |
| | | | | | | Fluoro) | | | | OMe) | phenyl)propanoic acid |
| 291 | H | (S)-α- | E | G | T | L-α-Me- | T | S | D | Bip(2′- | (S)-2-amino-3-(4-((3- |
| | | Me-Pro | | | | Phe(2- | | | | Et-4′- | (trifluoromethoxy) |
| | | | | | | Fluoro) | | | | OMe) | benzamido)methyl)phenyl) |
| | | | | | | | | | | | propanoic acid |
| 292 | H | (S)-α- | E | G | T | L-α-Me- | T | S | D | Bip(2′- | (S)-2-amino-3-(4-((3- |
| | | Me-Pro | | | | Phe(2- | | | | Et-4′- | phenylbenzamido)methyl) |
| | | | | | | Fluoro) | | | | OMe) | phenyl)propanoic acid |
| 293 | H | (S)-α- | E | G | T | L-α-Me- | T | S | D | Bip(2′- | (S)-2-amino-3-(4-((4- |
| | | Me-Pro | | | | Phe(2- | | | | Et-4′- | butylbenzamido)methyl) |
| | | | | | | Fluoro) | | | | OMe) | phenyl)propanoic acid |
| 294 | H | (S)-α- | E | G | T | L-α-Me- | T | S | D | Bip(2′- | (S)-2-amino-3-(4-((4- |
| | | Me-Pro | | | | Phe(2- | | | | Et-4′- | cyclohexylbenzamido) |
| | | | | | | Fluoro) | | | | OMe) | methyl)phenyl)propanoic |
| | | | | | | | | | | | acid |
| 295 | H | (S)-α- | E | G | T | L-α-Me- | T | S | D | Bip(2′- | (S)-2-amino-3-(4-((4- |
| | | Me-Pro | | | | Phe(2- | | | | Et-4′- | methoxybenzamido)methyl) |
| | | | | | | Fluoro) | | | | OMe) | phenyl)propanoic acid |
| 296 | H | (S)-α- | E | G | T | L-α-Me- | T | S | D | Bip(2′- | (S)-2-amino-3-(4-((4- |
| | | Me-Pro | | | | Phe(2- | | | | Et-4′- | benzylbenzamido)methyl) |
| | | | | | | Fluoro) | | | | OMe) | phenyl)propanoic acid |
| 297 | H | (S)-α- | E | G | T | L-α-Me- | T | S | D | Bip(2′- | (S)-3-(4- |
| | | Me-Pro | | | | Phe(2- | | | | Et-4′- | (acetamidomethyl)phenyl)-2- |
| | | | | | | Fluoro) | | | | OMe) | aminopropanoic acid |
| 298 | H | (S)-α- | E | G | T | L-α-Me- | T | S | D | Bip(2′- | (S)-2-amino-3-(4-((3- |
| | | Me-Pro | | | | Phe(2- | | | | Et-4′- | methylbutanamido)methyl) |
| | | | | | | Fluoro) | | | | OMe) | phenyl)propanoic acid |

Results in the form of EC₅₀ values for selected compounds are shown in Table 10. The structures of exemplary compounds are provided in Table 11.

TABLE 10

| SEQ ID NO: | Human GLP-1 cAMP EC₅₀ (nM) |
|---|---|
| 300 | 0.085 |
| 303 | 0.059 |
| 305 | 0.061 |
| 308 | 0.023 |
| 310 | 0.045 |
| 311 | 0.032 |
| 315 | 0.080 |
| 317 | 0.062 |

TABLE 11

| SEQ | | | | | | | | | | | |
|---|---|---|---|---|---|---|---|---|---|---|---|
| ID NO: | Xₐₐ₁ | Xₐₐ₂ | Xₐₐ₃ | Xₐₐ₄ | Xₐₐ₅ | Xₐₐ₆ | Xₐₐ₇ | Xₐₐ₈ | Xₐₐ₉ | Xₐₐ₁₀ | Xₐₐ₁₁-NH₂ |
| 299 | H | Aib | E | G | T | L-α-Me- | T | S | D | Bip(2′- | (S)-2-amino-5- |
| | | | | | | Phe(2- | | | | Et-4′- | methylhexanoic acid |
| | | | | | | Fluoro) | | | | OMe) | |
| 300 | H | Aib | E | G | T | L-α-Me- | T | S | D | Bip(2′- | 1- |
| | | | | | | Phe(2- | | | | Et-4′- | aminocyclopentanecarboxylic |
| | | | | | | Fluoro) | | | | OMe) | acid |
| 301 | H | Aib | E | G | T | L-α-Me- | T | S | D | Bip(2′- | (S)-2-aminooctanoic acid |
| | | | | | | Phe(2- | | | | Et-4′- | |
| | | | | | | Fluoro) | | | | OMe) | |
| 302 | H | Aib | E | G | T | L-α-Me- | T | S | D | Bip(2′- | (S)-2-aminodecanoic acid |
| | | | | | | Phe(2- | | | | Et-4′- | |
| | | | | | | Fluoro) | | | | OMe) | |
| 303 | H | Aib | E | G | T | L-α-Me- | T | S | D | Bip(2′- | (S)-2-aminohexanoic acid |
| | | | | | | Phe(2- | | | | Et-4′- | |
| | | | | | | Fluoro) | | | | OMe) | |
| 304 | H | Aib | E | G | T | L-α-Me- | T | S | D | Bip(2′- | (S)-2-amino-4- |
| | | | | | | Phe(2- | | | | Et-4′- | methoxybutanoic acid |
| | | | | | | Fluoro) | | | | OMe) | |
| 305 | H | (S)-α- | E | G | T | L-α-Me- | T | S | D | Bip(2′- | (S)-2-aminooctanoic acid |
| | | Me-Pro | | | | Phe(2- | | | | Et-4′- | |
| | | | | | | Fluoro) | | | | OMe) | |
| 306 | H | Aib | E | G | T | L-α-Me- | T | S | D | Bip(2′- | (S)-2-amino-3- |
| | | | | | | Phe(2- | | | | Et-4′- | butoxypropanoic acid |
| | | | | | | Fluoro) | | | | OMe) | |
| 307 | H | Aib | E | G | T | L-α-Me- | T | S | D | Bip(2′- | (S)-2-amino-3-(2- |
| | | | | | | Phe(2- | | | | Et-4′- | methoxyethoxy)propanoic |
| | | | | | | Fluoro) | | | | OMe) | acid |
| 308 | H | (S)-α- | E | G | T | L-α-Me- | T | S | D | Bip(2′- | (S)-2-amino-3- |
| | | Me-Pro | | | | Phe(2- | | | | Et-4′- | butoxypropanoic acid |
| | | | | | | Fluoro) | | | | OMe) | |
| 309 | H | (S)-α- | E | G | T | L-α-Me- | T | S | D | Bip(2′- | (S)-2-amino-3-(2- |
| | | Me-Pro | | | | Phe(2- | | | | Et-4′- | methoxyethoxy)propanoic |
| | | | | | | Fluoro) | | | | OMe) | acid |
| 310 | H | (S)-α- | E | G | T | L-α-Me- | T | S | D | Bip(2′- | (S)-2-amino-3- |
| | | Me-Pro | | | | Phe(2- | | | | Et-4′- | cyclohexylpropanoic acid |
| | | | | | | Fluoro) | | | | OMe) | |
| 311 | H | (S)-α- | E | G | T | L-α-Me- | T | S | D | Bip(2′- | (S)-2-amino-4- |
| | | Me-Pro | | | | Phe(2- | | | | Et-4′- | cyclohexylbutanoic acid |
| | | | | | | Fluoro) | | | | OMe) | |
| 312 | H | (S)-α- | E | G | T | L-α-Me- | T | S | D | Bip(2′- | (R)-2-amino-3- |
| | | Me-Pro | | | | Phe(2- | | | | Et-4′- | cyclohexylpropanoic acid |
| | | | | | | Fluoro) | | | | OMe) | |
| 313 | H | (S)-α- | E | G | T | L-α-Me- | T | S | D | Bip(2′- | (2S,3S)-2-amino-3- |
| | | Me-Pro | | | | Phe(2- | | | | Et-4′- | methylpentanoic acid |
| | | | | | | Fluoro) | | | | OMe) | |
| 314 | H | (S)-α- | E | G | T | L-α-Me- | T | S | D | Bip(2′- | (S)-2-amino-4-methylpent-4- |
| | | Me-Pro | | | | Phe(2- | | | | Et-4′- | enoic acid |
| | | | | | | Fluoro) | | | | OMe) | |
| 315 | H | (S)-α- | E | G | T | L-α-Me- | T | S | D | Bip(2′- | (S)-2-aminopentanoic acid |
| | | Me-Pro | | | | Phe(2- | | | | Et-4′- | |
| | | | | | | Fluoro) | | | | OMe) | |
| 316 | H | (S)-α- | E | G | T | L-α-Me- | T | S | D | Bip(2′- | (R)-2-aminopentanoic acid |
| | | Me-Pro | | | | Phe(2- | | | | Et-4′- | |
| | | | | | | Fluoro) | | | | OMe) | |
| 317 | H | (S)-α- | E | G | T | L-α-Me- | T | S | D | Bip(2′- | (R)-2-aminooctanoic acid |
| | | Me-Pro | | | | Phe(2- | | | | Et-4′- | |
| | | | | | | Fluoro) | | | | OMe) | |

TABLE 12

| SEQ ID NO: | Human GLP-1 cAMP EC₅₀ (nM) |
|---|---|
| 318 | 0.051 |
| 319 | 0.038 |
| 320 | 0.073 |
| 321 | 0.035 |
| 322 | 0.047 |
| 323 | 0.056 |
| 324 | 0.043 |

TABLE 13

| SEQ | | | | | | | | | | | |
|---|---|---|---|---|---|---|---|---|---|---|---|
| ID NO: | Xₐₐ₁ | Xₐₐ₂ | Xₐₐ₃ | Xₐₐ₄ | Xₐₐ₅ | Xₐₐ₆ | Xₐₐ₇ | Xₐₐ₈ | Xₐₐ₉ | Xₐₐ₁₀ | Xₐₐ₁₁-NH₂ |
| 318 | H₃C—O—CO- | (S)-α- | E | G | T | L-α-Me- | T | H | D | Bip(2′- | (S)-2-amino-4-(2- |
| | His | Me-Pro | | | | Phe(2- | | | | Et-4′- | methyl-4- |
| | | | | | | Fluoro) | | | | OMe) | chlorophenoxy) |
| | | | | | | | | | | | butanamide |
| 319 | H₃C—SO₂- | (S)-α- | E | G | T | L-α-Me- | T | S | D | Bip(2′- | (S)-2-amino-4-(2- |
| | His | Me-Pro | | | | Phe(2- | | | | Et-4′- | methyl-4- |
| | | | | | | Fluoro) | | | | OMe) | chlorophenoxy) |
| | | | | | | | | | | | butanamide |
| 320 | H₃C—SO₂- | (S)-α- | E | G | T | L-α-Me- | T | H | D | Bip(2′- | (S)-2-amino-4-(2- |
| | His | Me-Pro | | | | Phe(2- | | | | Et-4′- | methyl-4- |
| | | | | | | Fluoro) | | | | OMe) | chlorophenoxy) |
| | | | | | | | | | | | butanamide |
| 321 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | methyl-4- |
| | | | | | | Fluoro) | | | | OMe) | fluorophenoxy) |
| | | | | | | | | | | | butanamide |
| 322 | H₃C—O—CO- | (S)-α- | E | G | T | L-α-Me- | T | H | D | Bip(2′- | (S)-2-amino-4-(2- |
| | His | Me-Pro | | | | Phe(2- | | | | Et-4′- | methyl-4- |
| | | | | | | Fluoro) | | | | OMe) | fluorophenoxy) |
| | | | | | | | | | | | butanamide |
| 323 | H₃C—SO₂- | (S)-α- | E | G | T | L-α-Me- | T | H | D | Bip(2′- | (S)-2-amino-4-(2- |
| | His | Me-Pro | | | | Phe(2- | | | | Et-4′- | methyl-4- |
| | | | | | | Fluoro) | | | | OMe) | fluorophenoxy) |
| | | | | | | | | | | | butanamide |
| 324 | (L)-β- | (S)-α- | E | G | T | L-α-Me- | T | S | D | Bip(2′- | (S)-2-amino-4-(2- |
| | Imidazo | Me-Pro | | | | Phe(2- | | | | Et-4′- | methyl-4- |
| | lelactyl | | | | | Fluoro) | | | | OMe) | chlorophenoxy) |
| | | | | | | | | | | | butanamide |

### EXAMPLE 27

### In Vivo Studies

Compounds were dissolved in an appropriate vehicle at a concentration in nmol/ml equivalent to the dose that was to be administered in nmol/kg so that each mouse would receive the same volume/weight of dosing solution. Male C57BL/6J-ob/ob mice (10 weeks old) were randomized into groups of six mice per group based on fed plasma glucose and body weight. After an overnight fast, mice were weighed and placed in the experimental lab. After 30 minutes in the environment, the mice were bled via tail tip at −30 min and immediately injected subcutaneously (sc) with vehicle or peptide dissolved in vehicle (0.1 ml solution/100 g body weight). At time 0 the mice were bled and then injected intraperitoneally with 50% glucose (2 g/kg) to initiate the intraperitoneal glucose tolerance test (ipGTT). The mice were bled 30, 60, 120 and 180 min after the glucose injection. Blood samples were drawn into potassium EDTA, placed on ice during the study and subsequently centrifuged for 10 min at 3000 rpm at 4° C. Plasma samples were diluted 11-fold for glucose analysis in the Cobas System. Another 5 μl plasma sample was diluted 5-fold with 20 μl of Sample Diluent (Insulin ELISA assay kit, Crystal Chem Inc.) and stored at −20° C. for subsequent analysis using the Ultra Sensitive Mouse Insulin ELISA kit (Crystal Chem Inc.).

The in vivo glucose lowering properties for the compounds of SEQ ID NOs: 141, 145, 167, 318, 319, 320, 321, 322, 323 and 324 in ob/ob mice (a mouse model of insulin resistance) are summarized in Table 14 (below).

### EXAMPLE 31

### Dog Pharmacokinetic Studies

The pharmacokinetic parameters of the Compounds of the peptides in Table 14 were determined in male beagle dogs (n=4, 14±1 kg). Following an overnight fast, each animal received the compound by subcutaneous injection given at near the shoulder blades (67 μg/kg). Each animal received subcutaneous doses with a one-week washout between doses following a crossover design. The dosing vehicle for both routes of administration was 0.2 M Tris (pH 8.0). Serial blood samples were collected in EDTA-containing microcentrifuge tubes at predose, 0.083, 0.25, 0.5, 0.75, 1, 2, 4, 6, 8, 24, and 30 hours post-dose after intravenous administration; at predose, 0.25, 0.5, 0.75, 1, 2, 4, 6, 8, 24, and 30 hours post-dose after subcutaneous administration. Approximately 0.3 mL of blood was collected at each time point. Blood samples were immediately centrifuged at 4° C. The obtained plasma was frozen with dry ice and stored at −20° C. Plasma drug levels were determined using the LC-MS/MS assay described below.

### Compound Quantitation by LC-MS/MS

Plasma samples from an in vivo dog study were prepared for analysis by precipitating plasma proteins with two volumes of acetonitrile containing an internal standard. The samples were vortex mixed and removed the precipitated proteins by centrifugation. The resulting supernatants were transferred to a 96-well plate and 10 μL were injected for analysis. Samples were prepared with the Packard Multiprobe II and Quadra 96 Liquid Handling System.

The HPLC system used two Shimadzu LC10AD pumps (Columbia, Md.), a CTC PAL autosampler (Leap Technologies, Switzerland). The column used was a YMC Hydrosphere C18 (2.0×50 mm, 3 μm) (YMC, Inc., Milford, Mass.). The column temperature was maintained at 50° C. and the flow rate was 0.3 mL/minute. The mobile phase A consisted of 10 mM ammonium formate and 0.1% formic acid in water and mobile phase B consisted of 0.1% formic acid in acetonitrile. The initial mobile phase composition was 5% B, and remained at 5% B for one minute to equilibrate the column. The composition was ramped to 95% B over two minutes and held there for one additional minute. The mobile phase was then returned to initial conditions in one minute. Total analysis time was five minutes. A switching valve was used. The eluents between 0-1 minute were diverted to the waste.

The HPLC was interfaced to a Sciex API 4000 mass spectrometer, (Applied Biosystems, Foster City, Calif.) and was equipped with a TurboIonspray ionization source. Ultra high purity nitrogen was used as the nebulizing and turbo gas. The temperature of turbo gas was set at 300° C. and the interface heater was set at 60° C. Data acquisition utilized selected reaction monitoring (SRM).

The compounds disclosed and claimed herein show superior potency, with comparable exposures, in an efficacy model of glucose lowering (ob/ob mouse model) and superior pharmacokinetics (as measured by subcutaneous injection in dogs), as illustrated in Table 14.

TABLE 14

| | Potency in ob/ob mice: | |
|---|---|---|
| | % AUC Reduction in | |
| | Plasma Glucose in an | |
| Peptide | IP Glucose Tolerance | |
| SEQ ID | Test after SC | Exposure in |
| NO | Injection of Compound* | dogs**(sc@67 μg/kg) |
| 141 | −66% (p < 0.001) | 517 nM * h |
| | (10 nmol/kg) | |
| 145 | −43% (p < 0.05) | 964 nM * h |
| | (1 nmol/kg) | |
| 167 | 67% (p < 0.01) | 1030 nM * h |
| | (10 nmol/kg) | |
| 318 | −62% (p < 0.01) | 1242 nM * h |
| | (3 nmol/kg) | |
| 319 | −38% (p < 0.05) | 1366 nM * h |
| | (3 nmol/kg) | |
| 320 | −46% (p < 0.05) | 815 nM * h |
| | (1 nmol/kg) | |
| 321 | −59% (p < 0.01) | 705 nM * h |
| | (3 nmol/kg) | |
| 322 | −43% (p < 0.01) | 601 nM * h |
| | (1 nmol/kg) | |
| 323 | −50% (p < 0.01) | Not tested |
| | (3 nmol/kg) | |
| 324 | −43% (p < 0.01) | 210 nM * h |
| | (1 nmol/kg) | |

*AUC = area under the curve. AUC values are calculated using the fasting plasma glucose value as the baseline in each individual animal. The percentage change in the AUC is calculated relative to the AUC for the vehicle-treated group in the same study. The p values given are determined by comparison to the vehicle-treated group using analysis of variance (ANOVA) followed by Fisher's post-hoc test.
**Dosing vehicle: 0.2 M Tris buffer (pH 8.0).

### Utilities and Combinations

A. Utilities

The subject matter described herein provides novel compounds which have superior properties and act as GLP-1 receptor modulators, for example such that the compounds have agonist activity for the GLP-1 receptor. Further, compounds described herein exhibit increased stability to proteolytic cleavage as compared to GLP-1 native sequences.

Accordingly, compounds described herein can be administered to mammals, preferably humans, for the treatment of a variety of conditions and disorders, including, but not limited to, treating or delaying the progression or onset of diabetes (preferably Type II, impaired glucose tolerance, insulin resistance, and diabetic complications, such as nephropathy, retinopathy, neuropathy and cataracts), hyperglycemia, hyperinsulinemia, hypercholesterolemia, elevated blood levels of free fatty acids or glycerol, hyperlipidemia, hypertriglyceridemia, obesity, wound healing, tissue ischemia, atherosclerosis, hypertension, AIDS, intestinal diseases (such as necrotizing enteritis, microvillus inclusion disease or celiac disease), inflammatory bowel syndrome, chemotherapy-induced intestinal mucosal atrophy or injury, anorexia nervosa, osteoporosis, dysmetabolic syndrome, as well as inflammatory bowel disease (such as Crohn's disease and ulcerative colitis). The compounds described herein may also be utilized to increase the blood levels of high density lipoprotein (HDL).

In addition, the conditions, diseases, and maladies collectively referenced to as “Syndrome X” or Metabolic Syndrome as detailed in Johansson J. Clin. Endocrinol. Metab., 82, 727-34 (1997), may be treated employing the compounds described herein.

B. Combinations

The subject matter described and claimed herein includes pharmaceutical compositions comprising, as an active ingredient, a therapeutically effective amount of at least one of the compounds of Formula I, alone or in combination with a pharmaceutical carrier or diluent. Optionally, the compounds described herein can be used alone, in combination with other compounds described herein, or in combination with one or more other therapeutic agent(s), e.g. an antidiabetic agent or other pharmaceutically active material.

The compounds described herein may be employed in combination with other GLP-1 receptor modulators (e.g., agonists or partial agonists, such as a peptide agonist) or other suitable therapeutic agents useful in the treatment of the aforementioned disorders including: anti-diabetic agents; anti-hyperglycemic agents; hypolipidemic/lipid lowering agents; anti-obesity agents (including appetite suppressants/modulators) and anti-hypertensive agents. In addition, the compounds described herein may be combined with one or more of the following therapeutic agents; infertility agents, agents for treating polycystic ovary syndrome, agents for treating growth disorders, agents for treating frailty, agents for treating arthritis, agents for preventing allograft rejection in transplantation, agents for treating autoimmune diseases, anti-AIDS agents, anti-osteoporosis agents, agents for treating immunomodulatory diseases, antithrombotic agents, agents for the treatment of cardiovascular disease, antibiotic agents, anti-psychotic agents, agents for treating chronic inflammatory bowel disease or syndrome and/or agents for treating anorexia nervosa.

Examples of suitable anti-diabetic agents for use in combination with the compounds described herein include biguanides (e.g., metformin or phenformin), glucosidase inhibitors (e.g., acarbose or miglitol), insulins (including insulin secretagogues or insulin sensitizers), meglitinides (e.g., repaglinide), sulfonylureas (e.g., glimepiride, glyburide, gliclazide, chlorpropamide and glipizide), biguanide/glyburide combinations (e.g., Glucovance®), thiazolidinediones (e.g., troglitazone, rosiglitazone and pioglitazone), PPAR-alpha agonists, PPAR-gamma agonists, PPAR alpha/gamma dual agonists, glycogen phosphorylase inhibitors, inhibitors of fatty acid binding protein (aP2), DPP-IV inhibitors, and SGLT2 inhibitors.

Other suitable thiazolidinediones include Mitsubishi's MCC-555 (disclosed in U.S. Pat. No. 5,594,016), Glaxo-Wellcome's GL-262570, englitazone (CP-68722, Pfizer) or darglitazone (CP-86325, Pfizer, isaglitazone (MIT/J&J), JTT-501 (JPNT/P&U), L-895645 (Merck), R-119702 (Sankyo/WL), N,N-2344 (Dr. Reddy/NN), or YM-440 (Yamanouchi).

Suitable PPAR alpha/gamma dual agonists include muraglitazar (Bristol-Myers Squibb), AR-HO39242 (Astra/Zeneca), GW-409544 (Glaxo-Wellcome), KRP297 (Kyorin Merck) as well as those disclosed by Murakami et al, “A Novel Insulin Sensitizer Acts As a Coligand for Peroxisome Proliferation—Activated Receptor Alpha (PPAR alpha) and PPAR gamma. Effect on PPAR alpha Activation on Abnormal Lipid Metabolism in Liver of Zucker Fatty Rats”, Diabetes 47, 1841-1847 (1998), and in U.S. application Ser. No. 09/644,598, filed Sep. 18, 2000, the disclosure of which is incorporated herein by reference, employing dosages as set out therein, which compounds designated as preferred are preferred for use herein.

Suitable aP2 inhibitors include those disclosed in U.S. application Ser. No. 09/391,053, filed Sep. 7, 1999, and in U.S. application Ser. No. 09/519,079, filed Mar. 6, 2000, employing dosages as set out herein.

Suitable DPP4 inhibitors that may be used in combination with the compounds described herein include those disclosed in WO99/38501, WO99/46272, WO99/67279 (PROBIODRUG), WO99/67278 (PROBIODRUG), WO99/61431 (PROBIODRUG), NVP-DPP728A (1-[[[2-[(5-cyanopyridin-2-yl)amino]ethyl]amino]acetyl]-2-cyano-(S)-pyrrolidine) (Novartis) as disclosed by Hughes et al, Biochemistry, 38(36), 11597-11603, 1999, LAF237, saxagliptin, MK0431, TSL-225 (tryptophyl-1,2,3,4-tetrahydroisoquinoline-3-carboxylic acid (disclosed by Yamada et al, Bioorg. & Med. Chem. Lett. 8 (1998) 1537-1540, 2-cyanopyrrolidides and 4-cyanopyrrolidides, as disclosed by Ashworth et al, Bioorg. & Med. Chem. Lett., Vol. 6, No. 22, pp 1163-1166 and 2745-2748 (1996) employing dosages as set out in the above references.

Suitable meglitinides include nateglinide (Novartis) or KAD1229 (PF/Kissei).

Examples of other suitable glucagon-like peptide-1 (GLP-1,) compounds that may be used in combination with the GLP-1 receptor modulators (e.g. agonists or partial agonists) described herein include GLP-1 (1-36) amide, GLP-1 (7-36) amide, GLP-1 (7-37) (as disclosed in U.S. Pat. No. 5,614,492 to Habener), as well as AC2993 (Amylin), LY-315902 (Lilly) and NN2211 (Novo Nordisk).

Examples of suitable hypolipidemic/lipid lowering agents for use in combination with the compounds described herein include one or more MTP inhibitors, HMG CoA reductase inhibitors, squalene synthetase inhibitors, fibric acid derivatives, ACAT inhibitors, lipoxygenase inhibitors, cholesterol absorption inhibitors, ileal Na+/bile acid cotransporter inhibitors, upregulators of LDL receptor activity, bile acid sequestrants, cholesterol ester transfer protein inhibitors (e.g., CP-529414 (Pfizer)) and/or nicotinic acid and derivatives thereof.

MTP inhibitors which may be employed as described above include those disclosed in U.S. Pat. No. 5,595,872, U.S. Pat. No. 5,739,135, U.S. Pat. No. 5,712,279, U.S. Pat. No. 5,760,246, U.S. Pat. No. 5,827,875, U.S. Pat. No. 5,885,983 and U.S. Pat. No. 5,962,440, all of which are incorporated by reference herein.

The HMG CoA reductase inhibitors which may be employed in combination with one or more compounds of Formula I include mevastatin and related compounds, as disclosed in U.S. Pat. No. 3,983,140, lovastatin (mevinolin) and related compounds, as disclosed in U.S. Pat. No. 4,231,938, pravastatin and related compounds, such as disclosed in U.S. Pat. No. 4,346,227, simvastatin and related compounds, as disclosed in U.S. Pat. Nos. 4,448,784 and 4,450,171. Other HMG CoA reductase inhibitors which may be employed herein include, but are not limited to, fluvastatin, disclosed in U.S. Pat. No. 5,354,772, cerivastatin, as disclosed in U.S. Pat. Nos. 5,006,530 and 5,177,080, atorvastatin, as disclosed in U.S. Pat. Nos. 4,681,893, 5,273,995, 5,385,929 and 5,686,104, atavastatin (Nissan/Sankyo's nisvastatin (NK-104)), as disclosed in U.S. Pat. No. 5,011,930, visastatin (Shionogi-Astra/Zeneca (ZD-4522)), as disclosed in U.S. Pat. No. 5,260,440, and related statin compounds disclosed in U.S. Pat. No. 5,753,675, pyrazole analogs of mevalonolactone derivatives, as disclosed in U.S. Pat. No. 4,613,610, indene analogs of mevalonolactone derivatives, as disclosed in PCT application WO 86/03488, 6-[2-(substituted-pyrrol-1-yl)-alkyl)pyran-2-ones and derivatives thereof, as disclosed in U.S. Pat. No. 4,647,576, Searle's SC-45355 (a 3-substituted pentanedioic acid derivative) dichloroacetate, imidazole analogs of mevalonolactone, as disclosed in PCT application WO 86/07054, 3-carboxy-2-hydroxy-propane-phosphonic acid derivatives, as disclosed in French Patent No. 2,596,393, 2,3-disubstituted pyrrole, furan and thiophene derivatives, as disclosed in European Patent Application No. 0221025, naphthyl analogs of mevalonolactone, as disclosed in U.S. Pat. No. 4,686,237, octahydronaphthalenes, such as disclosed in U.S. Pat. No. 4,499,289, keto analogs of mevinolin (lovastatin), as disclosed in European Patent Application No. 0142146 A2, and quinoline and pyridine derivatives, as disclosed in U.S. Pat. Nos. 5,506,219 and 5,691,322.

Desired hypolipidemic agents are pravastatin, lovastatin, simvastatin, atorvastatin, fluvastatin, cerivastatin, atavastatin and ZD-4522.

In addition, phosphinic acid compounds useful in inhibiting HMG CoA reductase, such as those disclosed in GB 2205837, are suitable for use in combination with the compounds described herein.

The squalene synthetase inhibitors suitable for use herein include, but are not limited to, α-phosphono-sulfonates disclosed in U.S. Pat. No. 5,712,396, those disclosed by Biller et al, J. Med. Chem., 1988, Vol. 31, No. 10, pp 1869-1871, including isoprenoid (phosphinyl-methyl)phosphonates, as well as other known squalene synthetase inhibitors, for example, as disclosed in U.S. Pat. Nos. 4,871,721 and 4,924,024 and in Biller, S. A., Neuenschwander, K., Ponpipom, M. M., and Poulter, C. D., Current Pharmaceutical Design, 2, 1-40 (1996).

In addition, other squalene synthetase inhibitors suitable for use herein include the terpenoid pyrophosphates disclosed by P. Ortiz de Montellano et al, J. Med. Chem., 1977, 20, 243-249, the farnesyl diphosphate analog A and presqualene pyrophosphate (PSQ-PP) analogs as disclosed by Corey and Volante, J. Am. Chem. Soc., 1976, 98, 1291-1293, phosphinylphosphonates reported by McClard, R. W. et al, J.A.C.S., 1987, 109, 5544 and cyclopropanes reported by Capson, T. L., PhD dissertation, June, 1987, Dept. Med. Chem. U of Utah, Abstract, Table of Contents, pp 16, 17, 40-43, 48-51, Summary.

The fibric acid derivatives which may be employed in combination with one or more compounds of Formula I include fenofibrate, gemfibrozil, clofibrate, bezafibrate, ciprofibrate, clinofibrate and the like, probucol, and related compounds, as disclosed in U.S. Pat. No. 3,674,836, probucol and gemfibrozil being preferred, bile acid sequestrants, such as cholestyramine, colestipol and DEAE-Sephadex (Secholex®, Policexide®), as well as lipostabil (Rhone-Poulenc), Eisai E-5050 (an N-substituted ethanolamine derivative), imanixil (HOE-402), tetrahydrolipstatin (THL), istigmastanylphos-phorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid, acipimox, acifran, neomycin, p-aminosalicylic acid, aspirin, poly(diallylmethylamine) derivatives, such as disclosed in U.S. Pat. No. 4,759,923, quaternary amine poly(diallyldimethylammonium chloride) and ionenes, such as disclosed in U.S. Pat. No. 4,027,009, and other known serum cholesterol lowering agents.

The ACAT inhibitor which may be employed in combination with one or more compounds of Formula I include those disclosed in Drugs of the Future 24, 9-15 (1999), (Avasimibe); “The ACAT inhibitor, Cl-1011 is effective in the prevention and regression of aortic fatty streak area in hamsters”, Nicolosi et al, Atherosclerosis (Shannon, Irel). (1998), 137(1), 77-85; “The pharmacological profile of FCE 27677: a novel ACAT inhibitor with potent hypolipidemic activity mediated by selective suppression of the hepatic secretion of ApoB100-containing lipoprotein”, Ghiselli, Giancarlo, Cardiovasc. Drug Rev. (1998), 16(1), 16-30; “RP 73163: a bioavailable alkylsulfinyl-diphenylimidazole ACAT inhibitor”, Smith, C., et al, Bioorg. Med. Chem. Lett. (1996), 6(1), 47-50; “ACAT inhibitors: physiologic mechanisms for hypolipidemic and anti-atherosclerotic activities in experimental animals”, Krause et al, Editor(s): Ruffolo, Robert R., Jr.; Hollinger, Mannfred A., Inflammation: Mediators Pathways (1995), 173-98, Publisher: CRC, Boca Raton, Fla.; “ACAT inhibitors: potential anti-atherosclerotic agents”, Sliskovic et al, Curr. Med. Chem. (1994), 1(3), 204-25; “Inhibitors of acyl-CoA:cholesterol O-acyl transferase (ACAT) as hypocholesterolemic agents. 6. The first water-soluble ACAT inhibitor with lipid-regulating activity. Inhibitors of acyl-CoA:cholesterol acyltransferase (ACAT). 7. Development of a series of substituted N-phenyl-N′-[(1-phenylcyclopentyl)methyl]ureas with enhanced hypocholesterolemic activity”, Stout et al, Chemtracts: Org. Chem. (1995), 8(6), 359-62, or TS-962 (Taisho Pharmaceutical Co. Ltd).

The hypolipidemic agent may be an upregulator of LD2 receptor activity, such as MD-700 (Taisho Pharmaceutical Co. Ltd) and LY295427 (Eli Lilly).

Examples of suitable cholesterol absorption inhibitor for use in combination with the compounds described herein include SCH48461 (Schering-Plough), as well as those disclosed in Atherosclerosis 115, 45-63 (1995) and J. Med. Chem. 41, 973 (1998).

Examples of suitable ileal Na+/bile acid cotransporter inhibitors for use in combination with the compounds described herein include compounds as disclosed in Drugs of the Future, 24, 425-430 (1999).

The lipoxygenase inhibitors which may be employed in combination with one or more compounds of Formula I include 15-lipoxygenase (15-LO) inhibitors, such as benzimidazole derivatives, as disclosed in WO 97/12615, 15-LO inhibitors, as disclosed in WO 97/12613, isothiazolones, as disclosed in WO 96/38144, and 15-LO inhibitors, as disclosed by Sendobry et al “Attenuation of diet-induced atherosclerosis in rabbits with a highly selective 15-lipoxygenase inhibitor lacking significant antioxidant properties”, Brit. J. Pharmacology (1997) 120, 1199-1206, and Cornicelli et al, “15-Lipoxygenase and its Inhibition: A Novel Therapeutic Target for Vascular Disease”, Current Pharmaceutical Design, 1999, 5, 11-20.

Examples of suitable anti-hypertensive agents for use in combination with the compounds described herein include beta adrenergic blockers, calcium channel blockers (L-type and T-type; e.g. diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetamide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), and nitrates.

Examples of suitable anti-obesity agents for use in combination with the compounds described herein include a NPY receptor antagonist, a NPY-Y2 or NPY-Y4 receptor agonist, Oxyntomodulin, a MCH antagonist, a GHSR antagonist, a CRH antagonist, a beta 3 adrenergic agonist, a lipase inhibitor, a serotonin (and dopamine) reuptake inhibitor, a thyroid receptor beta drug, a CB-1 antagonist and/or an anorectic agent.

The beta 3 adrenergic agonists which may be optionally employed in combination with compounds described herein include AJ9677 (Takeda/Dainippon), L750355 (Merck), or CP331648 (Pfizer,) or other known beta 3 agonists, as disclosed in U.S. Pat. Nos. 5,541,204, 5,770,615, 5,491,134, 5,776,983 and 5,488,064, with AJ9677, L750,355 and CP331648 being preferred.

Examples of lipase inhibitors which may be optionally employed in combination with compounds described herein include orlistat or ATL-962 (Alizyme), with orlistat being preferred.

The serotonin (and dopamine) reuptake inhibitor which may be optionally employed in combination with a compound of Formula I may be sibutramine, topiramate (Johnson & Johnson) or axokine (Regeneron), with sibutramine and topiramate being preferred.

Examples of thyroid receptor beta compounds which may be optionally employed in combination with compounds described herein include thyroid receptor ligands, such as those disclosed in WO97/21993 (U. Cal SF), WO99/00353 (KaroBio) and WO 00/039077 (KaroBio), with compounds of the KaroBio applications being preferred.

Examples of CB-1 antagonists which may be optionally employed in combination with compounds described herein include CB-1 antagonists and rimonabant (SR141716A).

Examples of NPY-Y2 and NPY-Y4 receptor agonists include PYY (3-36) and Pancreatic Polypeptide (PP), respectively.

The anorectic agent which may be optionally employed in combination with compounds described herein include dexamphetamine, phentermine, phenylpropanolamine or mazindol, with dexamphetamine being preferred.

Examples of suitable anti-psychotic agents include clozapine, haloperidol, olanzapine (Zyprexa®), Prozac® and aripiprazole (Abilify®).

The aforementioned patents and patent applications are incorporated herein by reference.

The above other therapeutic agents, when employed in combination with the compounds described herein may be used, for example, in those amounts indicated in the Physician's Desk Reference, as in the patents set out above or as otherwise determined by one of ordinary skill in the art.

### Dosage and Formulation

A suitable peptide of Formula I can be administered to patients to treat diabetes and other related diseases as the compound alone and or mixed with an acceptable carrier in the form of pharmaceutical formulations. Those skilled in the art of treating diabetes can easily determine the dosage and route of administration of the compound to mammals, including humans, in need of such treatment. The route of administration may include but is not limited to oral, intraoral, rectal, transdermal, buccal, intranasal, pulmonary, subcutaneous, intramuscular, intradermal, sublingual, intracolonic, intraoccular, intravenous, or intestinal administration. The compound is formulated according to the route of administration based on acceptable pharmacy practice (Fingl et al., in “The Pharmacological Basis of Therapeutics”, Ch. 1, p. 1, 1975; “Remington's Pharmaceutical Sciences”, 18th ed., Mack Publishing Co, Easton, Pa., 1990).

The pharmaceutically acceptable peptide compositions described herein can be administered in multiple dosage forms such as tablets, capsules (each of which includes sustained release or timed release formulations), pills, powders, granules, elixirs, in situ gels, microspheres, crystalline complexes, liposomes, micro-emulsions, tinctures, suspensions, syrups, aerosol sprays and emulsions. The compositions described herein can also be administered in oral, intravenous (bolus or infusion), intraperitoneal, subcutaneous, transdermally or intramuscular form, all using dosage forms well known to those of ordinary skill in the pharmaceutical arts. The compositions may be administered alone, but generally will be administered with a pharmaceutical carrier selected on the basis of the chosen route of administration and standard pharmaceutical practice.

The dosage regimen for the compositions described herein will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the disease state.

By way of general guidance, the daily oral dosage of the active ingredient, when used for the indicated effects, will range between about 0.001 to 1000 mg/kg of body weight, preferably between about 0.01 to 100 mg/kg of body weight per day, and most preferably between about 0.6 to 20 mg/kg/day. Intravenously, the daily dosage of the active ingredient when used for the indicated effects will range between 0.001 ng to 100.0 ng per min/per Kg of body weight during a constant rate infusion. Such constant intravenous infusion can be preferably administered at a rate of 0.01 ng to 50 ng per min per Kg body weight and most preferably at 0.01 ng to 10.0 mg per min per Kg body weight. The compositions described herein may be administered in a single daily dose, or the total daily dosage may be administered in divided doses of two, three, or four times daily. The compositions described herein may also be administered by a depot formulation that will allow sustained release of the drug over a period of days/weeks/months as desired.

The compositions described herein can be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compositions are typically administered in a mixture with suitable pharmaceutical diluents, excipients, or carriers (collectively referred to herein as pharmaceutical carriers) suitably selected with respect to the intended form of administration, that is, oral tablets, capsules, elixirs, aerosol sprays generated with or without propellant and syrups, and consistent with conventional pharmaceutical practices.

For instance, for oral administration in the form of a tablet or capsule, the active drug component can be combined with an oral, non-toxic, pharmaceutically acceptable, inert carrier such as but not limited to, lactose, starch, sucrose, glucose, methyl cellulose, magnesium stearate, dicalcium phosphate, calcium sulfate, mannitol, and sorbitol; for oral administration in liquid form, the oral drug components can be combined with any oral, non-toxic, pharmaceutically acceptable inert carrier such as, but not limited to, ethanol, glycerol, and water. Moreover, when desired or necessary, suitable binders, lubricants, disintegrating agents, and coloring agents can also be incorporated into the mixture. Suitable binders include, but not limited to, starch, gelatin, natural sugars such as, but not limited to, glucose or beta-lactose, corn sweeteners, natural and synthetic gums such as acacia, tragacanth, or sodium alginate, carboxymethylcellulose, polyethylene glycol, and waxes. Lubricants used in these dosage forms include sodium oleate, sodium stearate, magnesium stearate, sodium benzoate, sodium acetate, and sodium chloride. Disintegrants include, but are not limited to, starch, methyl cellulose, agar, bentonite, and xanthan gum.

The compositions described herein may also be administered in the form of mixed micellar or liposome delivery systems, such as small unilamellar vesicles, large unilamellar vesicles, and multilamellar vesicles. Liposomes can be formed from a variety of phospholipids, such as cholesterol, stearylamine, or phosphatidylcholines. Permeation enhancers may be added to enhance drug absorption.

Since prodrugs are known to enhance numerous desirable qualities of pharmaceuticals (i.e., solubility, bioavailability, manufacturing, etc.) the compounds described herein may be delivered in prodrug form. Thus, the subject matter described herein is intended to cover prodrugs of the presently claimed compounds, methods of delivering the same, and compositions containing the same.

The compositions described herein may also be coupled with soluble polymers as targetable drug carriers. Such polymers can include polyvinyl-pyrrolidone, pyran copolymer, polyhydroxypropyl-methacrylamide-phenol, polyhydroxyethylaspartamidephenol, or polyethyleneoxide-polylysine substituted with palmitoyl residues. Furthermore, the compositions described herein may be combined with a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Dosage forms (pharmaceutical compositions) suitable for administration may contain from about 0.01 milligram to about 500 milligrams of active ingredient per dosage unit. In these pharmaceutical compositions the active ingredient will ordinarily be present in an amount of about 0.5-95% by weight based on the total weight of the composition.

Gelatin capsules may contain the active ingredient and powdered carriers, such as lactose, starch, cellulose derivative, magnesium stearate, and stearic acid. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solution for parenteral administration preferably contains a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington: “The Science and Practice of Pharmacy”, Nineteenth Edition, Mack Publishing Company, 1995, a standard reference text in this field

Representative useful pharmaceutical dosage forms for administration of the compounds described herein can be illustrated as follows:

Capsules

A large number of unit capsules can be prepared by filling standard two-piece hard gelatin capsules with 100 milligrams of powdered active ingredient, 150 milligrams of lactose, 50 milligrams of cellulose, and six milligrams magnesium stearate.

Soft Gelatin Capsules

A mixture of active ingredient in a digestible oil such as soybean oil, cottonseed oil or olive oil may be prepared and injected by means of a positive displacement pump into gelatin to form soft gelatin capsules containing 100 milligrams of the active ingredient. The capsules should be washed and dried.

Tablets

Tablets may be prepared by conventional procedures so that the dosage unit, for example is 100 milligrams of active ingredient, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

Injectable

An injectable formulation of a peptide composition described herein may or may not require the use of excipients such as those that have been approved by regulatory bodies. These excipients include, but are not limited to, solvents and co-solvents, solubilizing, emulsifying or thickening agents, chelating agents, anti-oxidants and reducing agents, antimicrobial preservatives, buffers and pH adjusting agents, bulking agents, protectants and tonicity adjustors and special additives. An injectable formulation has to be sterile, pyrogen free and, in the case of solutions, free of particulate matter.

A parenteral composition suitable for administration by injection may be prepared by stirring for example, 1.5% by weight of active ingredient in a pharmaceutically acceptable buffer that may or may not contain a co-solvent or other excipient. The solution should be made isotonic with sodium chloride and sterilized.

Suspension

An aqueous suspension can be prepared for oral and/or parenteral administration so that, for example, each 5 mL contains 100 mg of finely divided active ingredient, 20 mg of sodium carboxymethyl cellulose, 5 mg of sodium benzoate, 1.0 g of sorbitol solution, U.S.P., and 0.025 mL of vanillin or other palatable flavoring.

Biodegradable Microparticles

A sustained-release parenteral composition suitable for administration by injection may be prepared, for example, by dissolving a suitable biodegradable polymer in a solvent, adding to the polymer solution the active agent to be incorporated, and removing the solvent from the matrix thereby forming the matrix of the polymer with the active agent distributed throughout the matrix.

Numerous modifications and variations of the subject matter described and claimed herein are possible in light of the above teachings. It is therefore understood that within the scope of the appended claims, the subject matter recited in the claims may be practiced otherwise than as specifically described herein.

The subject matter recited in the claims is not to be limited in scope by the specific embodiments described that are intended as single embodiments of the claimed subject matter. Functionally equivalent methods and components in addition to those shown and described herein will become apparent to those skilled in the art from the foregoing description and accompanying drawings. Such modifications are intended to fall within the scope of the appended claims. All references cited herein are hereby incorporated by reference in their entirety.

What is claimed is:

1. An isolated polypeptide comprising a sequence of Formula I
Xₐₐ₁-Xₐₐ₂-Xₐₐ₃-Xₐₐ₄-Xₐₐ₅-Xₐₐ₆-Xₐₐ₇-Xₐₐ₈-Xₐₐ₉-Xₐₐ₁₀-Xₐₐ₁₁ I
wherein,
Xₐₐ₁ is a naturally or non-naturally occurring amino acid comprising an imidazole, such as histidine; wherein any of the carbon atoms of said amino acid are optionally substituted with hydrogen, or with one or more alkyl groups, wherein the free amino group of said amino acid is optionally substituted with hydrogen, alkyl, acyl, benzoyl, L-lactyl, alkyloxycarbonyl, aryloxycarbonyl, arylalkyloxycarbonyl, heterocyclyloxycarbonyl, heteroarylalkyloxycarbonyl, alkylcarbamoyl, arylcarbamoyl, arylalkylcarbamoyl, heterocyclylsulfonyl, alkylsulfonyl, arylsulfonyl, arylalkylsulfonyl, heteroarylalkylsulfonyl or heteroarylsulfonyl; and wherein the amino group of Xₐₐ₁ is optionally absent, such that Xₐₐ₁ is the des-amino acid of histidine in which any of the carbon atoms are optionally substituted with hydrogen or one or more alkyl groups;
Xₐₐ₂ is a naturally or non-naturally occurring amino acid selected from the group consisting of D-alanine, α-amino-isobutyric acid (Aib), N-methyl-D-alanine, N-ethyl-D-alanine, 2-methyl-azetidine-2-carboxylic acid, alpha-methyl-(L)-proline, 2-methylpiperidine-2-carboxylic acid and isovaline;
Xₐₐ₃ is a naturally or non-naturally occurring amino acid comprising an amino acid side chain which contains a carboxylic acid, for example aspartic acid or glutamic acid; or wherein Xₐₐ₃ is a naturally or non-naturally occurring amino acid containing an imidazole side chain, for example histidine, and wherein any of the carbon atoms of said amino acid are optionally substituted with one or more alkyl groups;
Xₐₐ₄ is glycine;
Xₐₐ₅ is a naturally or non-naturally occurring amino acid selected from the group consisting of (L)-threonine, and (L)-norvaline; and wherein any of the carbon atoms of said amino acid are optionally substituted with one or more alkyl groups;
Xₐₐ₆ is a naturally or non-naturally occurring amino acid comprising an alpha carbon which is di-substituted; wherein one of the side chains of said amino acid contains an aromatic ring, for example alpha-methyl-phenylalanine, alpha-methyl-2-fluorophenylalanine, and alpha-methyl-2,6-difluorophenylalanine; wherein any of the carbon atoms of said amino acid are optionally substituted with one or more alkyl groups or one or more halo groups;
Xₐₐ₇ is a naturally or non-naturally occurring amino acid comprising an amino acid side chain which is substituted with a hydroxyl group, for example L-threonine;
wherein any of the carbon atoms of said amino acid are optionally substituted with one or more alkyl groups;
Xₐₐ₈ is a naturally or non-naturally occurring amino acid selected from the group consisting of L-serine, and L-histidine; wherein one or more of the carbon atoms of said amino acid is optionally substituted with one or more alkyl groups;
Xₐₐ₉ is a naturally or non-naturally occurring amino acid comprising an amino acid side chain which contains a carboxylic acid, for example L-aspartic acid or L-glutamic acid; wherein one or more of the carbon atoms of said amino acid is optionally substituted with one or more alkyl groups;
Xₐₐ₁₀ is a naturally or non-naturally occurring amino acid of Formula II: 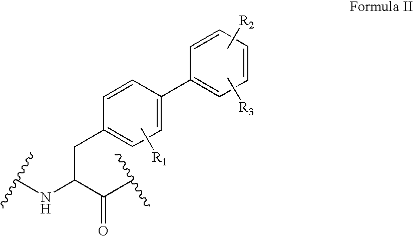
wherein
R₁ is selected from the group consisting of hydrogen, alkyl, and halo; and
R₂ and R₃ are each independently selected from the group consisting of hydrogen, halo, methyl, ethyl, alkyl, hydroxyl, methoxy, and alkoxy;
Xₐₐ₁₁ is a naturally or non-naturally occurring amino acid of any of Formulae III, IV, V, or VI, wherein:
Formula III is: 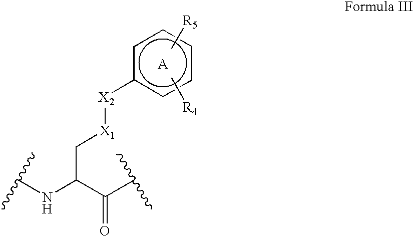
wherein the C-terminal carbonyl carbon of said amino acid is attached to a nitrogen to form a carboxamide (NH₂);
wherein ring A is selected from the group consisting of aryl and heteroaryl;
wherein R₄ and R₅ are each independently selected from the group consisting of hydrogen, halo, methyl, ethyl, alkyl, hydroxyl, methoxy, alkoxy, aryl, heteroaryl; and
wherein X₁ and X₂ are each CH-alkyl, CH₂, NH, S or O; and
Formula III may further comprise at least one R₄ or R₅ groups, and, if more than one are present, may or may not be equivalent;
Formula IV is: 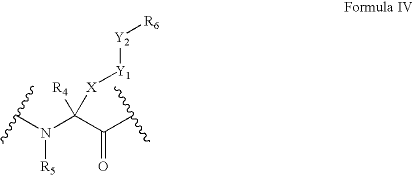
wherein the C-terminal carbonyl carbon of said amino acid is attached to a nitrogen to form a carboxamide (NH₂);
wherein R₄ is selected from the group consisting of hydrogen, hydroxyl, methyl, ethyl, alkyl, methoxy, alkoxy, aryl, heteroaryl;
wherein R₅ is selected from the group consisting of hydrogen, methyl, ethyl, alkyl, cycloalkyl, aryl, heteroaryl, alkylaryl, or heteroalkylaryl;
wherein X is selected from the group consisting of CH₂, CH₂CH₂, or CHCH₃;
wherein Y₁ is selected from the group consisting of —NH—, —O—, and —C═O—;
wherein Y₂ is selected from the group consisting of —C═O—, —O═C—O— and —SO₂— when Y₁ is NH or O;
wherein Y₂ is selected from the group consisting of —NH—, —N—, or —O— when Y₁ is C═O; and
wherein R₆ is selected from the group consisting of hydrogen, methyl, ethyl, alkyl, cycloalkyl, aryl, heteroaryl, alkylaryl, or alkylheteroaryl; and
Formula IV further comprises at least one R₆ group and, if more than one are present, may or may not, be equivalent;
Formula V is: 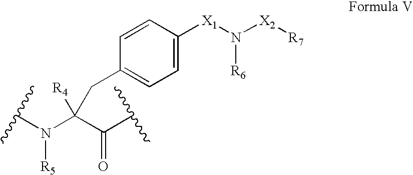
wherein the C-terminal carbonyl carbon of said amino acid is attached to a nitrogen to form a carboxamide (NH₂);
wherein R₄ is selected from the group consisting of hydrogen, hydroxyl, methyl, ethyl, alkyl, methoxy, alkoxy, aryl, heteroaryl;
wherein R₅ is selected from the group consisting of hydrogen, methyl, ethyl, alkyl, cycloalkyl, aryl, heteroaryl, alkylaryl, or heteroalkylaryl;
wherein X₁ is either absent or consists of CH₂;
wherein X₂ is selected from the group consisting of —CO—, CO—N(−)₂, —CO—O—, —SO—, and —SO₂—;
wherein R₆ and R₇ are independently selected from the group consisting of hydrogen, alkyl, alkenyl, alkynyl, cycloalkyl, aryl, heteroaryl, alkylaryl, or alkylheteroaryl; and
Formula V comprises at least one R₇ group, and, if more than one are present, may or may not be equivalent.
Formula VI is: 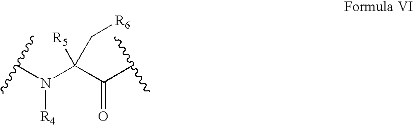
wherein the C-terminal carbonyl carbon of said amino acid is attached to a nitrogen to form a carboxamide (NH₂);
wherein R₄ is selected from the group consisting of hydrogen, methyl, ethyl, alkyl, cycloalkyl, aryl, heteroaryl, alkylaryl, or heteroalkylaryl;
wherein R₅ is selected from the group consisting of hydrogen, hydroxyl, methyl, ethyl, alkyl, methoxy, and alkoxy;
wherein R₆ is selected from the group consisting of hydrogen, methyl, ethyl, alkyl, cycloalkyl, heterocycloalkyl, hydroxyl, methoxy, and alkoxy; and
Formula VI may further comprise at least one R₆ group, and, if more than one are present, may or may not be equivalent and may further comprise R₅ and R₆ groups which together form a cycloalkyl, heterocycloalkyl, cycloalkylaryl, or cycloalkylheteroaryl group.

2. The isolated polypeptide of claim 1, wherein said Xₐₐ₁ is L-Histidine and wherein the terminal amino group is optionally substituted with hydrogen, alkyl, dialkyl, acyl, benzoyl, L-lactyl, alkyloxycarbonyl, aryloxycarbonyl, arylalkyloxycarbonyl, heterocyclyloxycarbonyl, heteroarylalkyloxycarbonyl, alkylcarbamoyl, arylcarbamoyl, arylalkylcarbamoyl, heterocyclylsulfonyl, alkylsulfonyl, arylsulfonyl, arylalkylsulfonyl, heteroarylalkylsulfonyl or heteroarylsulfonyl.

3. The isolated polypeptide of claim 1, wherein said Xₐₐ₁ is selected from the group consisting of L-His, L-N-methyl-His, L-α-methyl-His, des-amino-His, 3-(1H-imidazol-4-yl)-2-methylpropanoyl, and (S)-3-(1H-imidazol-4-yl)-2-hydroxypropanoyl(L-β-imidazolelactyl).

4. The isolated polypeptide of claim 1, wherein said Xₐₐ₂ is selected from the group consisting of α-amino-isobutyric acid (Aib), D-alanine, N-methyl-D-alanine, alpha-methyl-(L)-proline, 2-methyl-azetidine-2-carboxylic acid and 2-methylpiperidine-2-carboxylic acid.

5. The isolated polypeptide of claim 1, wherein said Xₐₐ₃ is selected from the group consisting of L-glutamic acid and L-aspartic acid.

6. The isolated polypeptide of claim 1, wherein said Xₐₐ₄ is Gly.

7. The isolated polypeptide of claim 1, wherein said Xₐₐ₅ is selected from the group consisting of L-Thr, and L-Nva.

8. The isolated polypeptide of claim 1, wherein said Xₐₐ₆ is selected from the group consisting of L-α-Me-Phe, L-α-Me-2-fluoro-Phe, and L-α-Me-2,6-difluoro-Phe.

9. The isolated polypeptide of claim 1, wherein said Xₐₐ₇ is L-Thr.

10. The isolated polypeptide of claim 1, wherein said Xₐₐ₈ is selected from the group consisting of L-Ser, and L-His.

11. The isolated polypeptide of claim 1, wherein said Xₐₐ₉ is L-Asp.

12. The isolated polypeptide of claim 1, wherein said Xₐₐ₁₀ of Formula II is selected from the group consisting of 4-phenyl-phenylalanine, 4-[(4′-methoxy-2′-ethyl)phenyl]phenylalanine, 4-[(4′-ethoxy-2′-ethyl)phenyl]phenylalanine, 4-[(4′-methoxy-2′-methyl)phenyl]phenylalanine, 4-[(4′-ethoxy-2′-methyl)phenyl]phenylalanine, 4-(2′-ethylphenyl)phenylalanine, 4-(2′-methylphenyl)phenylalanine, 4-[(3′,5′-dimethyl)phenyl]phenylalanine and 4-[(3′,4′-dimethoxy)phenyl]phenylalanine.

13. The isolated polypeptide of claim 1, wherein said Xₐₐ₁₁ is an amino acid of Formula III and selected from the group consisting of (S)-2-amino-5-phenylpentanoic acid, (S)-2-amino-4-phenoxybutanoic acid, (S)-2-amino-5-(4-chlorophenyl)pentanoic acid, (S)-2-amino-5-(quinolin-5-yl)pentanoic acid, and (S)-2-amino-4-(2-chlorophenoxy)butanoic acid; (S)-2-amino-4-(2-methylphenoxy)butanoic acid; and (S)-2-amino-4-(2-methyl-4-chlorophenoxy)butanoic acid, wherein the C-terminal carbonyl carbon of said amino acid is attached to a nitrogen to form a carboxamide (NH₂).

14. The isolated polypeptide of claim 1, wherein said Xₐₐ₁₁ is an amino acid of Formula IV and selected from the group consisting of L-Asp(OBz)-OH, L-Glu(OBz)-OH, L-Ser(OBz)-OH, D-Ser(OBz)-OH, L-Thr(OBz)-OH, (S)-2-amino-5-(benzyl(methyl)amino)-5-oxopentanoic acid, (S)-3-((2-chlorobenzyloxy)carbonyl)-2-aminopropanoic acid, (S)-2-amino-5-benzyl(ethyl)amino-5-oxopentanoic acid, (S)-5-((3-methoxybenzyl)(methyl)amino)-2-amino-5-oxopentanoic acid, (S)-2-amino-4-(benzylamino)-4-oxobutanoic acid, (S)-2-amino-3-(3-methylbenzamido)propanoic acid, (S)-2-amino-3-(2-methylbenzamido)propanoic acid, (S)-2-amino-5-(isoindolin-2-yl)-5-oxopentanoic acid, (S)-2-amino-3-(benzyloxycarbonyl)propanoic acid, (S)-5-(2-methylbenzylamino)-2-amino-5-oxopentanoic acid, (S)-5-(2-fluorobenzylamino)-2-amino-5-oxopentanoic acid, (S)-2-amino-4-oxo-4-(piperidin-1-yl)butanoic acid, (S)-5-(4-(trifluoromethyl)benzylamino)-2-amino-5-oxopentanoic acid, (S)-2-amino-5-(dibenzylamino)-5-oxopentanoic acid, (S)-2-amino-3-(3-phenylureido)propanoic acid, (S)-2-amino-3-(3-benzylureido)propanoic acid, (S)-2-amino-3-(3-o-tolylureido)propanoic acid, (S)-2-amino-3-(2-o-tolylacetamido)propanoic acid, (S)-2-amino-5-(benzylamino)-5-oxopentanoic acid, (S)-5-(3-methoxybenzylamino)-2-amino-5-oxopentanoic acid, (S)-2-amino-3-(3-methylpicolinamido)propanoic acid, (S)-2-amino-3-(isonicotinamido)propanoic acid, (S)-2-amino-3-(2-phenylacetamido)propanoic acid, (S)-2-amino-5-(3,4-dihydroisoquinolin-2(1H)-yl)-5-oxopentanoic acid, (S)-2-amino-5-(butylamino)-5-oxopentanoic acid, (S)-2-amino-3-benzamidopropanoic acid, (S)-2-amino-5-oxo-5-(piperidin-1-yl)pentanoic acid, (S)-2-amino-5-(isobutylamino)-5-oxopentanoic acid, (S)-2-amino-4-oxo-4-(pyridin-2-ylmethylamino)butanoic acid, (S)-4-(2-methylbenzylamino)-2-amino-4-oxobutanoic acid, (S)-2-amino-4-(isobutylamino)-4-oxobutanoic acid, (S)-2-amino-3-(4-methylbenzamido)propanoic acid, (S)-2-amino-5-oxo-5-(2-(piperidin-1-yl)ethylamino)pentanoic acid, (S)-2-amino-5-oxo-5-(2-(pyridin-2-yl)ethylamino)pentanoic acid, (S)-2-amino-5-(2-(dimethylamino)ethylamino)-5-oxopentanoic acid, (S)-2-amino-5-oxo-5-(phenethylamino)pentanoic acid, and (S)-3-acetamido-2-aminopropanoic acid; wherein the C-terminal carbonyl carbon of said amino acid is attached to a nitrogen to form a carboxamide (NH₂); and wherein R₆ is selected from the group consisting of hydrogen and methyl.

15. The isolated polypeptide of claim 1, wherein said Xₐₐ₁₁ is an amino acid of Formula V selected from the group consisting of (S)-2-amino-3-(4-(benzamidomethyl)phenyl)propanoic acid, (S)-2-amino-3-(4-(phenylsulfonamidomethyl)phenyl)propanoic acid, (S)-2-amino-3-(4-(methylsulfonamidomethyl)phenyl)propanoic acid, (S)-2-amino-3-(4-((2,3-dimethoxybenzamido)methyl)phenyl)propanoic acid, (S)-2-amino-3-(4-((2,4-difluorobenzamido)methyl)phenyl)propanoic acid, (S)-2-amino-3-(4-((2,4-dimethoxybenzamido)methyl)phenyl)propanoic acid, (S)-2-amino-3-(4-((2,5-dimethoxybenzamido)methyl)phenyl)propanoic acid, (S)-2-amino-3-(4-((2,6-dichlorophenylsulfonamido)methyl)phenyl)propanoic acid, (S)-2-amino-3-(4-((2-chlorobenzamido)methyl)phenyl)propanoic acid, (S)-2-amino-3-(4-((2-methoxybenzamido)methyl)phenyl)propanoic acid, (S)-2-amino-3-(4-((2-methylbenzamido)methyl)phenyl)propanoic acid, (S)-2-amino-3-(4-((2-methylphenylsulfonamido)methyl)phenyl)propanoic acid, (S)-2-amino-3-(4-((3,4-dichlorophenylsulfonamido)methyl)phenyl)propanoic acid, (S)-2-amino-3-(4-((3,4-dimethoxybenzamido)methyl)phenyl)propanoic acid, (S)-2-amino-3-(4-((benzo[d][1,3]dioxole-5-carboxamido)methyl)phenyl)propanoic acid, (S)-2-amino-3-(4-((3,5-dichlorophenylsulfonamido)methyl)phenyl)propanoic acid, (S)-2-amino-3-(4-((3,5-dimethoxybenzamido)methyl)phenyl)propanoic acid, (S)-2-amino-3-(4-((3,5-dimethylisoxazole-4-sulfonamido)methyl)phenyl)propanoic acid, (S)-2-amino-3-(4-((3-(trifluoromethyl)benzamido)methyl)phenyl)propanoic acid, (S)-2-amino-3-(4-((3-chlorobenzamido)methyl)phenyl)propanoic acid, (S)-2-amino-3-(4-((3-chlorophenylsulfonamido)methyl)phenyl)propanoic acid, (S)-2-amino-3-(4-((3-cyanobenzamido)methyl)phenyl)propanoic acid, (S)-2-amino-3-(4-((3-ethoxybenzamido)methyl)phenyl)propanoic acid, (S)-2-amino-3-(4-((3-fluorobenzamido)methyl)phenyl)propanoic acid, (S)-2-amino-3-(4-((3-isopropylbenzamido)methyl)phenyl)propanoic acid, (S)-2-amino-3-(4-((3-methoxy-4-methylbenzamido)methyl)phenyl)propanoic acid, (S)-2-amino-3-(4-((3-methoxybenzamido)methyl)phenyl)propanoic acid, (S)-2-amino-3-(4-((3-methoxyphenylsulfonamido)methyl)phenyl)propanoic acid, (S)-2-amino-3-(4-((3-methylbenzamido)methyl)phenyl)propanoic acid, (S)-2-amino-3-(4-((3-(trifluoromethoxy)benzamido)methyl)phenyl)propanoic acid, (S)-2-amino-3-(4-((3-phenylbenzamido)methyl)phenyl)propanoic acid, (S)-2-amino-3-(4-(nicotinamidomethyl)phenyl)propanoic acid, (S)-2-amino-3-(4-((4-butylbenzamido)methyl)phenyl)propanoic acid, (S)-2-amino-3-(4-((4-chlorophenylsulfonamido)methyl)phenyl)propanoic acid, (S)-2-amino-3-(4-((4-cyclohexylbenzamido)methyl)phenyl)propanoic acid, (S)-2-amino-3-(4-((4-methoxybenzamido)methyl)phenyl)propanoic acid, (S)-2-amino-3-(4-((4-methylphenylsulfonamido)methyl)phenyl)propanoic acid, (S)-2-amino-3-(4-((4-methylbenzamido)methyl)phenyl)propanoic acid, (S)-2-amino-3-(4-((4-benzylbenzamido)methyl)phenyl)propanoic acid, (S)-2-amino-3-(4-((4-phenylphenylsulfonamido)methyl)phenyl)propanoic acid, (S)-2-amino-3-(4-((4-phenylphenylsulfonamido)methyl)phenyl)propanoic acid, (S)-3-(4-(acetamidomethyl)phenyl)-2-aminopropanoic acid, (S)-2-amino-3-(4-(cyclohexanecarboxamidomethyl)phenyl)propanoic acid, (S)-2-amino-3-(4-((3-methylbutanamido)methyl)phenyl)propanoic acid, (S)-2-amino-3-(4-(2,4-dimethoxybenzamido)phenyl)propanoic acid, (S)-2-amino-3-(4-(2,6-dichlorophenylsulfonamido)phenyl)propanoic acid, (S)-2-amino-3-(4-(2-chlorobenzamido)phenyl)propanoic acid, (S)-2-amino-3-(4-(2-methylbenzamido)phenyl)propanoic acid, (S)-2-amino-3-(4-(3,4-dichlorophenylsulfonamido)phenyl)propanoic acid, (S)-2-amino-3-(4-(3,4-dimethoxybenzamido)phenyl)propanoic acid, (S)-2-amino-3-(4-(3,5-dichlorophenylsulfonamido)phenyl)propanoic acid, (S)-2-amino-3-(4-(3,5-dimethylisoxazole-4-sulfonamido)phenyl)propanoic acid, (S)-2-amino-3-(4-(3-chlorophenylsulfonamido)phenyl)propanoic acid, (S)-2-amino-3-(4-(3-methylbenzamido)phenyl)propanoic acid, (S)-2-amino-3-(4-(nicotinamido)phenyl)propanoic acid, (S)-2-amino-3-(4-(4-methylphenylsulfonamido)phenyl)propanoic acid, (S)-2-amino-3-(4-(3-phenylbenzamido)phenyl)propanoic acid, (S)-2-amino-3-(4-(4-biphenylsulfonamido)phenyl)propanoic acid, (S)-2-amino-3-(4-(phenylsulfonamido)phenyl)propanoic acid, (S)-2-amino-3-(4-(methylsulfonamido)phenyl)propanoic acid, (S)-2-amino-3-(4-(2,4-difluorobenzamido)phenyl)propanoic acid, (S)-2-amino-3-(4-(2-chlorobenzamido)phenyl)propanoic acid, (S)-2-amino-3-(4-(2-methoxybenzamido)phenyl)propanoic acid, (S)-2-amino-3-(4-(2-methylphenylsulfonamido)phenyl)propanoic acid, (S)-2-amino-3-(4-(3-methoxybenzamido)phenyl)propanoic acid, (S)-2-amino-3-(4-(3-methoxyphenylsulfonamido)phenyl)propanoic acid, (S)-2-amino-3-(4-(4-chlorophenylsulfonamido)phenyl)propanoic acid, and (S)-2-amino-3-(4-(4-methoxyphenylsulfonamido)phenyl)propanoic acid; wherein the C-terminal carbonyl carbon of said amino acid is attached to a nitrogen to form a carboxamide (NH₂); and wherein R₆ is selected from the group consisting of hydrogen and methyl.

16. The isolated polypeptide of claim 1, wherein said Xₐₐ₁₁ is an amino acid of Formula VI selected from the group consisting of (2S,3S)-2-amino-3-methylpentanoic acid, (R)-2-amino-3-cyclohexylpropanoic acid, (R)-2-aminooctanoic acid, (R)-2-aminopentanoic acid, (S)-2-amino-3-(2-methoxyethoxy)propanoic acid, (S)-2-amino-3-butoxypropanoic acid, (S)-2-amino-3-cyclohexylpropanoic acid, (S)-2-amino-4-cyclohexylbutanoic acid, (S)-2-amino-4-methoxybutanoic acid, (S)-2-amino-4-methylpent-4-enoic acid, (S)-2-amino-5-methylhexanoic acid, (S)-2-aminodecanoic acid, (S)-2-aminohexanoic acid, (S)-2-aminooctanoic acid, (S)-2-aminopentanoic acid, and 1-aminocyclopentanecarboxylic acid; wherein the C-terminal carbonyl carbon of said amino acid is attached to a nitrogen to form a carboxamide (NH₂); and
wherein R₅ is chosen from the group consisting of hydrogen and methyl.

17. An isolated polypeptide of claim 1 comprising the following structure: 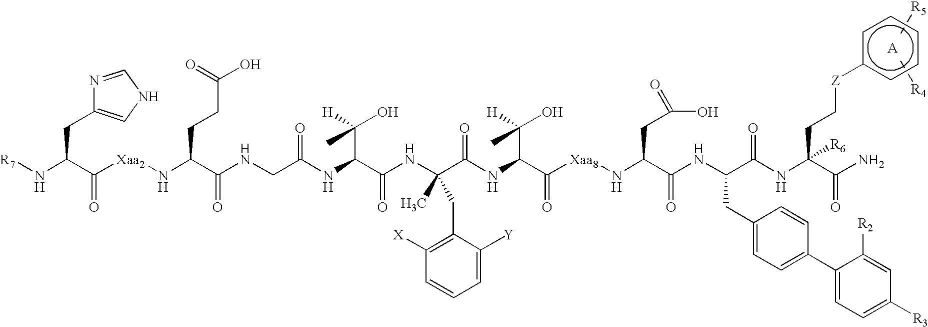
wherein:
Xₐₐ₂ is an amino acid selected from the group consisting of D-Ala, N-methyl-D-Ala, α-methyl-L-Pro, 2-methyl-azetidine-2-carboxylic acid, 2-methylpiperidine-2-carboxylic acid and α-aminoisobutyric (Aib);
X and Y are each independently selected from the group consisting of hydrogen and fluoro;
Xₐₐ₈ is an amino acid selected from the group consisting of L-Ser and L-His;
R₂ is selected from the group consisting of hydrogen, methyl and ethyl;
R₃ is selected from the group consisting of hydrogen, hydroxy, methoxy and ethoxy;
Z is selected from the group consisting of CH₂ and O;
Ring A is selected from the group consisting of aryl and heteroaryl;
R₄ is selected from the group consisting of hydrogen, fluoro, methyl and ethyl;
R₅ is selected from the group consisting of hydrogen, methyl and methoxy; and
R₆ is selected from the group consisting of hydrogen and methyl; and
R₇ is selected from the group consisting of hydrogen and methyl.

18. The isolated polypeptide of claim 14 wherein
Xₐₐ₂ is an amino acid selected from the group consisting of N-methyl-D-Ala, α-methyl-L-Pro and α-aminoisobutyric (Aib); wherein
X is fluoro;
Y is hydrogen;
Z is selected from the group consisting of CH₂ and O;
Xₐₐ₈ is an amino acid selected from the group consisting of L-Ser and L-His;
R₂ is ethyl;
R₃ is methoxy;
R₄ is selected from the group consisting of hydrogen, methyl and ethyl;
R₅ is selected from the group consisting of hydrogen, methyl and ethyl; and
R₇ is hydrogen.

19. An isolated polypeptide comprising the following structure: 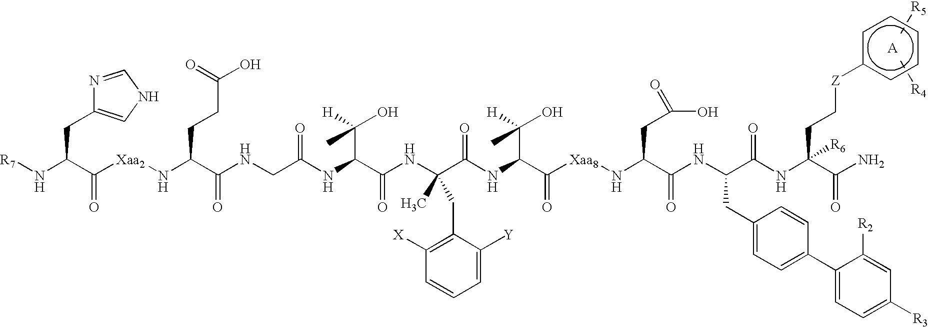
wherein R₇ is selected from the group consisting of methyl, ethyl, 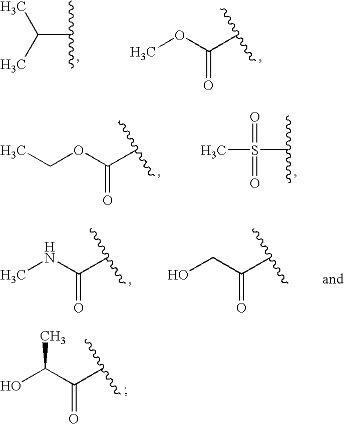
Xₐₐ₂ is an amino acid selected from the group consisting of D-Ala, N-methyl-D-Ala, α-methyl-L-Pro, α-aminoisobutyric (Aib), 2-methyl-azetidine-2-carboxylic acid, and 2-methylpiperidine-2-carboxylic acid;
X and Y are each independently selected from the group consisting of hydrogen and fluoro;
Ring A is selected from the group consisting of aryl and heteroaryl;
Z is selected from the group consisting of CH₂ and O;
Xₐₐ₈ is an amino acid selected from the group consisting of L-Ser and L-His;
R₂ is methyl or ethyl;
R₃ is selected from the group consisting of hydrogen, methyl, ethyl, and methoxy;
R₄ and R₅ are selected from the group consisting of hydrogen, methyl, ethyl, aryl, halo, or alkoxy; and
R₆ is selected from the group consisting of hydrogen, and methyl.

20. An isolated polypeptide of claim 16 wherein R₇ is selected from the group consisting of methyl, and 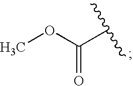
Xₐₐ₂ is an amino acid selected from the group consisting of N-methyl-D-Ala, α-methyl-L-Pro and aminoisobutyric acid (Aib);
X is fluoro;
Y is hydrogen;
Z is selected from the group consisting of CH₂ and O;
Xₐₐ₈ is an amino acid selected from the group consisting of L-Ser and L-His;
R₂ is ethyl;
R₃ is methoxy;
R₄ is selected from the group consisting of methyl and ethyl;
R₅ is selected from the group consisting of halo and hydrogen; and
R₆ is selected from the group consisting of hydrogen and methyl.

21. An isolated polypeptide of claim 1 comprising the following structure: 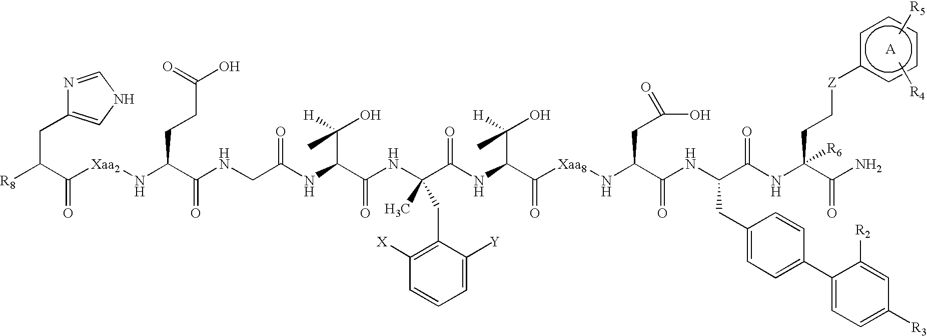
wherein R₈ is selected from the group consisting of hydrogen, methyl and alkyl;
Xₐₐ₂ is an amino acid selected from the group consisting of D-Ala, N-methyl-D-Ala, α-methyl-L-Pro, α-aminoisobutyric acid (Aib), 2-methyl-azetidine-2-carboxylic acid and 2-methylpiperidine-2-carboxylic acid;
X and Y are each independently selected from the group consisting of hydrogen and fluoro;
Xₐₐ₈ is an amino acid selected from the group consisting of L-Ser and L-His;
Z is chosen from the group consisting of CH₂ and O;
Ring A is selected from the group consisting of aryl and heteroaryl;
R₂ is selected from the group consisting of methyl and ethyl;
R₃ is selected from the group consisting of hydrogen, methyl, methoxy and ethyl;
R₄ and R₅ are selected from the group consisting of hydrogen, methyl, ethyl, aryl, halo, or alkoxy; and
R₆ is selected from the group consisting of hydrogen and methyl.

22. The isolated polypeptide of claim 21 wherein R₈ is selected from the group consisting of hydrogen and methyl;
Xₐₐ₂ is an amino acid selected from the group consisting of N-methyl-D-Ala, α-methyl-L-Pro, and α-aminoisobutyric acid (Aib);
X is fluoro;
Y is hydrogen;
Xₐₐ₈ is an amino acid selected from the group consisting of L-Ser and L-His;
R₂ is ethyl;
R₃ is methoxy;
R₄ is selected from the group consisting of methyl and ethyl;
R₅ is selected from the group consisting of halo and hydrogen; and
R₆ is selected from the group consisting of hydrogen and methyl.

23. An isolated polypeptide of claim 1 comprising the following structure: 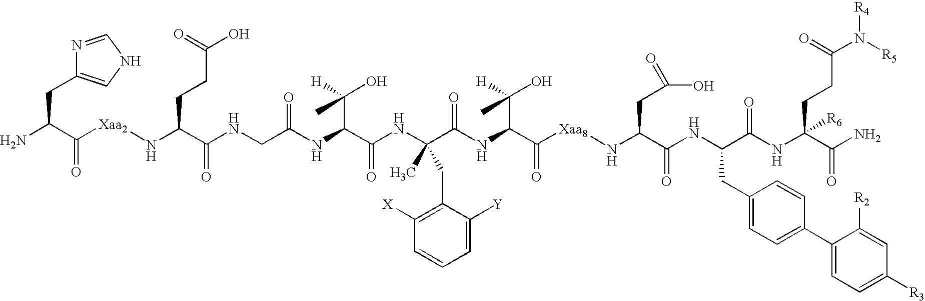
wherein:
Xₐₐ₂ is an amino acid selected from the group consisting of D-Ala, N-methyl-D-Ala, α-methyl-L-Pro, 2-methyl-azetidine-2-carboxylic acid, 2-methylpiperidine-2-carboxylic acid and α-aminoisobutyric (Aib);
X and Y are each independently selected from the group consisting of hydrogen and fluoro;
Xₐₐ₈ is an amino acid selected from the group consisting of L-Ser and L-His;
R₂ is selected from the group consisting of hydrogen, methyl and ethyl;
R₃ is selected from the group consisting of hydrogen, hydroxy, methoxy and ethoxy;
R₄ and R₅ are individually selected from the group consisting of hydrogen, alkyl, alkylaryl, alkylheteroaryl, or together selected from the group consisting of cycloalkyl, cycloalkylaryl, and cycloalkylheteroaryl; and
R₆ is selected from the group consisting of hydrogen and methyl.

24. An isolated polypeptide of claim 23 wherein:
Xₐₐ₂ is an amino acid selected from the group consisting of N-methyl-D-Ala, α-methyl-L-Pro and α-aminoisobutyric (Aib);
X is fluoro;
Y is hydrogen;
Xₐₐ₈ is an amino acid selected from the group consisting of L-Ser and L-His;
R₂ is ethyl;
R₃ is methoxy;
R₄ is selected from the group consisting of methyl and ethyl, alkylaryl and alkylheteroaryl;
R₅ is selected from the group consisting of hydrogen, methyl, ethyl, alkylaryl, and alkylheteroaryl; R₄ and R₅ together comprise a cyclic moiety; and
R₆ is hydrogen.

25. An isolated polypeptide of claim 1 comprising the following structure, 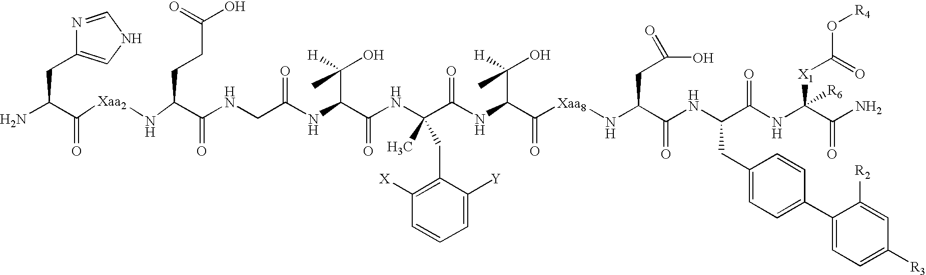
wherein:
Xₐₐ₂ is an amino acid selected from the group consisting of D-Ala, N-methyl-D-Ala, α-methyl-L-Pro, α-aminoisobutyric (Aib), 2-methyl-azetidine-2-carboxylic acid, and 2-methylpiperidine-2-carboxylic acid;
X and Y are each independently selected from the group consisting of hydrogen and fluoro;
X₁ is selected from the group consisting of CH₂ and CH₂CH₂;
Xₐₐ₈ is an amino acid selected from the group consisting of L-Ser and L-His;
R₂ is methyl or ethyl;
R₃ is selected from the group consisting of hydrogen, methyl, ethyl, and methoxy;
R₄ is selected from the group consisting of hydrogen, methyl, ethyl, alkyl, hydroxyl, methoxy, alkoxy, aryl, heteroaryl, alkylaryl, alkylheteroaryl; and
R₆ is hydrogen.

26. An isolated polypeptide of claim 1 comprising the following structure: 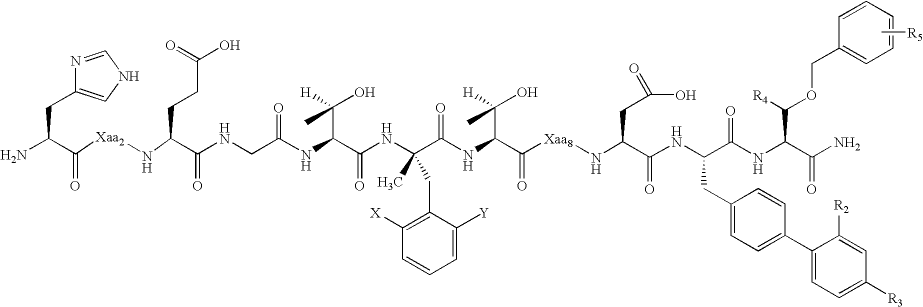
wherein:
Xₐₐ₂ is an amino acid selected from the group consisting of D-Ala, N-methyl-D-Ala, α-methyl-L-Pro, α-aminoisobutyric acid (Aib), 2-methyl-azetidine-2-carboxylic acid and 2-methylpiperidine-2-carboxylic acid;
X and Y are each independently selected from the group consisting of hydrogen and fluoro;
Xₐₐ₈ is an amino acid selected from the group consisting of L-Ser and L-His;
R₂ is methyl or ethyl;
R₃ is selected from the group consisting of hydrogen, methyl, methoxy and ethyl;
R₄ is hydrogen or methyl;
the polypeptide further comprises at least one R₅ group, and
R₅ is selected from the group consisting of hydrogen, halo, methyl, ethyl, alkyl, hydroxyl, methoxy, alkoxy, aryl, heteroaryl, alkylaryl, alkylheteroaryl.

27. An isolated polypeptide of claim 26 wherein:
Xₐₐ₂ is an amino acid selected from the group consisting of N-methyl-D-Ala, α-methyl-L-Pro, and α-aminoisobutyric acid (Aib);
X is fluoro;
Y is hydrogen;
Xₐₐ₈ is an amino acid selected from the group consisting of L-Ser and L-His;
R₂ is ethyl;
R₃ is methoxy;
R₄ is hydrogen or methyl; and
R₅ is hydrogen.

28. An isolated polypeptide of claim 1 comprising the following structure: 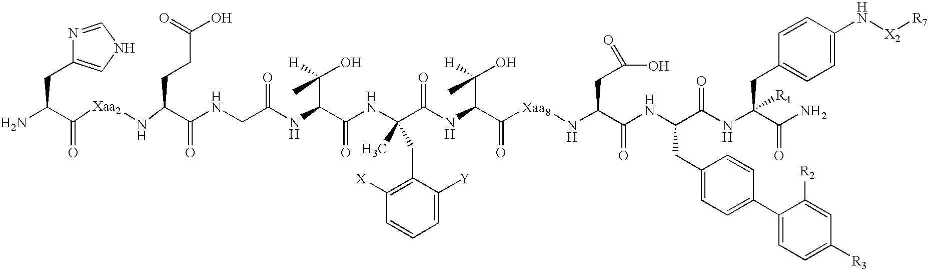
wherein:
Xₐₐ₂ is an amino acid selected from the group consisting of D-Ala, N-methyl-D-Ala, α-methyl-L-Pro, 2-methyl-azetidine-2-carboxylic acid, 2-methylpiperidine-2-carboxylic acid and α-aminoisobutyric (Aib);
X and Y are each independently selected from the group consisting of hydrogen and fluoro;
Xₐₐ₈ is an amino acid selected from the group consisting of L-Ser and L-His;
R₂ is selected from the group of hydrogen, methyl and ethyl;
R₃ is selected from the group of hydrogen, hydroxy, methoxy and ethoxy;
R₄ is selected from the group consisting of hydrogen and methyl;
X₂ is selected from the group consisting of —CO— and —SO₂—;
said polypeptide comprises at least one R₇ group and R₇ is selected from the group consisting of alkyl, cycloalkyl, aryl, heteroaryl, alkylaryl, or alkylheteroaryl.

29. An isolated polypeptide of claim 1 comprising the following structure: 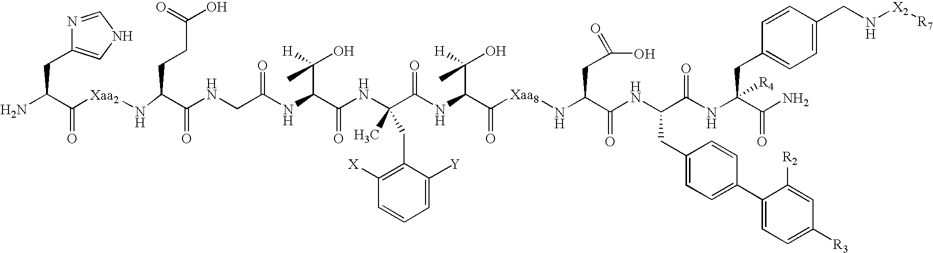
wherein:
Xₐₐ₂ is an amino acid selected from the group consisting of D-Ala, N-methyl-D-Ala, α-methyl-L-Pro, α-aminoisobutyric (Aib), 2-methyl-azetidine-2-carboxylic acid, and 2-methylpiperidine-2-carboxylic acid;
X and Y are each independently selected from the group consisting of hydrogen and fluoro;
Xₐₐ₈ is an amino acid selected from the group consisting of L-Ser and L-His;
R₂ is methyl or ethyl;
R₃ is selected from the group of hydrogen, methyl, ethyl, and methoxy;
R₄ is selected from the group consisting of hydrogen and methyl;
wherein X₂ is selected from the group consisting of —CO— and —SO₂—;
said polypeptide comprise at least one R₇ group; and
wherein R₇ is selected from the group consisting of alkyl, cycloalkyl, aryl, heteroaryl, alkylaryl, or alkylheteroaryl.

30. An isolated polypeptide of claim 1 comprising the following structure: 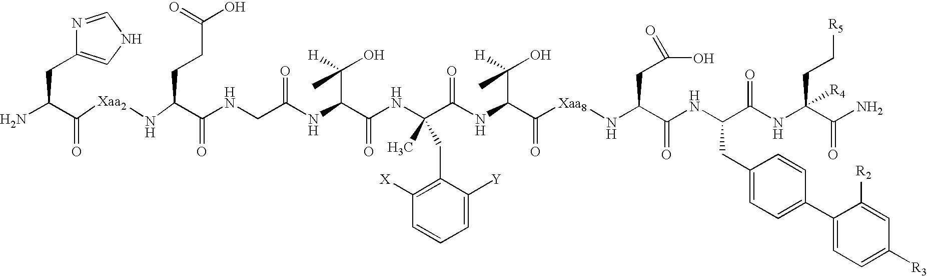
wherein:
Xₐₐ₂ is an amino acid selected from the group consisting of D-Ala, N-methyl-D-Ala, α-methyl-L-Pro, 2-methyl-azetidine-2-carboxylic acid, 2-methylpiperidine-2-carboxylic acid and α-aminoisobutyric (Aib);
X and Y are each independently selected from the group consisting of hydrogen and fluoro;
Xₐₐ₈ is an amino acid selected from the group consisting of L-Ser and L-His;
R₂ is selected from the group of hydrogen, methyl and ethyl;
R₃ is selected from the group of hydrogen, hydroxy, methoxy and ethoxy;
R₄ is selected from the group of hydrogen and methyl; and
R₅ is selected from the group consisting of alkyl, heteroalkyl, cycloalkyl, or heterocycloalkyl.

31. An isolated polypeptide of claim 30 wherein:
Xₐₐ₂ is an amino acid selected from the group consisting of N-methyl-D-Ala, α-methyl-L-Pro and α-aminoisobutyric (Aib);
X is fluoro;
Y is hydrogen;
Xₐₐ₈ is an amino acid selected from the group consisting of L-Ser and L-His;
R₂ is ethyl;
R₃ is methoxy;
R₄ is selected from the group of hydrogen and methyl; and
R₅ is selected from the group of methyl, ethyl, propyl, butyl, hexyl, cyclohexyl, and methylcyclohexyl.

32. An isolated polypeptide of claim 1 comprising the following structure: 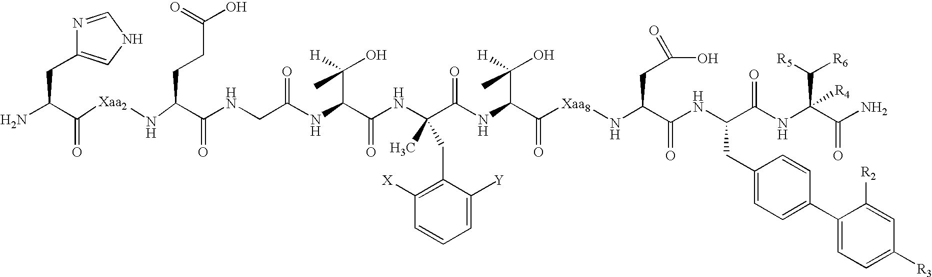
wherein:
Xₐₐ₂ is an amino acid selected from the group consisting of D-Ala, N-methyl-D-Ala, α-methyl-L-Pro, α-aminoisobutyric (Aib), 2-methyl-azetidine-2-carboxylic acid, and 2-methylpiperidine-2-carboxylic acid;
X and Y are each independently selected from the group consisting of hydrogen and fluoro;
Xₐₐ₈ is an amino acid selected from the group consisting of L-Ser and L-His;
R₂ is ethyl;
R₃ is methoxy;
R₄ is selected from the group of hydrogen and methyl;
R₅ is methyl;
R₆ is selected from the group of alkyl, heteroalkyl, cycloalkyl, and heterocycloalkyl.

33. An isolated polypeptide of claim 1 comprising the following structure, 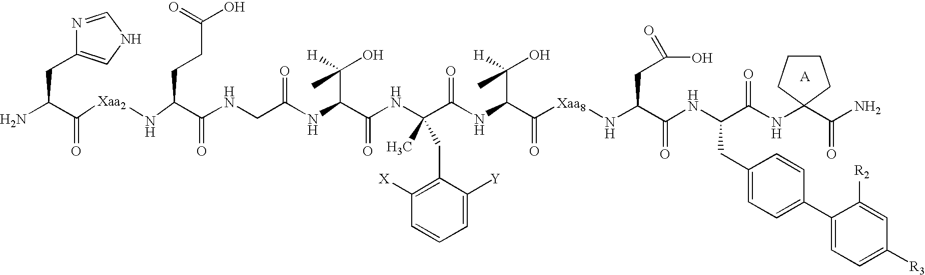
wherein:
Xₐₐ₂ is an amino acid selected from the group consisting of D-Ala, N-methyl-D-Ala, α-methyl-L-Pro, α-aminoisobutyric acid (Aib), 2-methyl-azetidine-2-carboxylic acid and 2-methylpiperidine-2-carboxylic acid;
X and Y are each independently selected from the group consisting of hydrogen and fluoro;
Xₐₐ₈ is an amino acid selected from the group consisting of L-Ser and L-His;
R₂ is methyl or ethyl;
R₃ is selected from the group of hydrogen, methyl, methoxy and ethyl; and
Ring A is selected from the group of a cycloalkyl, cycloalkylaryl, heterocycloalkyl or cycloalkylheteroaryl.

34. An isolated polypeptide of claim 33 wherein:
Xₐₐ₂ is an amino acid selected from the group consisting of N-methyl-D-Ala, α-methyl-L-Pro, and α-aminoisobutyric acid (Aib);
X is fluoro;
Y is hydrogen;
Xₐₐ₈ is an amino acid selected from the group consisting of L-Ser and L-His;
R₂ is ethyl;
R₃ is methoxy; and
Ring A is cyclopentyl.

35. An isolated polypeptide of claim 1, wherein said isolated polypeptide selected from the group consisting of:

| SEQ | | | | | | | | | | | |
|---|---|---|---|---|---|---|---|---|---|---|---|
| ID | | | | | | | | | | | |
| NO: | Xₐₐ₁ | Xₐₐ₂ | Xₐₐ₃ | Xₐₐ₄ | Xₐₐ₅ | Xₐₐ₆ | Xₐₐ₇ | Xₐₐ₈ | Xₐₐ₉ | Xₐₐ₁₀ | Xₐₐ₁₁ |
| 1 | H | Aib | E | G | T | L-α-Me- | T | S | D | Bip(2′-Et- | (S)-2-amino-5- |
| | | | | | | Phe(2- | | | | 4′-OMe) | phenylpentanamide |
| | | | | | | Fluoro) | | | | | |
| 2 | H | Aib | E | G | T | L-α-Me- | T | S | D | Bip(2′-Et- | (S)-2-amino-5-(pyridin-2-yl) |
| | | | | | | Phe(2- | | | | 4′-OMe) | pentanamide |
| | | | | | | Fluoro) | | | | | |
| 3 | H | Aib | E | G | T | L-α-Me- | T | S | D | Bip(2′-Et- | (S)-2-amino-5-(pyridin-3-yl) |
| | | | | | | Phe(2- | | | | 4′-OMe) | pentanamide |
| | | | | | | Fluoro) | | | | | |
| 4 | H | Aib | E | G | T | L-α-Me- | T | S | D | Bip(2′-Et- | (S)-2-amino-5-(pyrazin-2-yl) |
| | | | | | | Phe(2- | | | | 4′-OMe) | pentanamide |
| | | | | | | Fluoro) | | | | | |
| 5 | H | Aib | E | G | T | L-α-Me- | T | S | D | Bip(2′-Et- | (S)-2-amino-5- |
| | | | | | | Phe(2- | | | | 4′-OMe) | (benzo[d][1,3]dioxol-5-yl) |
| | | | | | | Fluoro) | | | | | pentanamide |
| 6 | H | Aib | E | G | T | L-α-Me- | T | S | D | Bip(2′-Et- | (S)-2-amino-5-(3,5- |
| | | | | | | Phe(2- | | | | 4′-OMe) | dimethylphenyl)pentanamide |
| | | | | | | Fluoro) | | | | | |
| 7 | H | Aib | E | G | T | L-α-Me- | T | S | D | Bip(2′-Et- | (S)-2-amino-5-(2- |
| | | | | | | Phe(2- | | | | 4′-OMe) | methylphenyl)pentanamide |
| | | | | | | Fluoro) | | | | | |
| 8 | H | Aib | E | G | T | L-α-Me- | T | S | D | Bip(2′-Et- | (S)-2-amino-5-(4- |
| | | | | | | Phe(2- | | | | 4′-OMe) | methylphenyl)pentanamide |
| | | | | | | Fluoro) | | | | | |
| 9 | H | (S)-α- | E | G | T | L-α-Me- | T | S | D | Bip(2′-Et- | (S)-2-amino-5-(3- |
| | | Me- | | | | Phe(2- | | | | 4′-OMe) | methylphenyl)pentanamide |
| | | Pro | | | | Fluoro) | | | | | |
| 10 | H | (S)-α- | E | G | T | L-α-Me- | T | S | D | Bip(2′-Et- | (S)-2-amino-5-(4- |
| | | Me- | | | | Phe(2- | | | | 4′-OMe) | methoxyphenyl)pentanamide |
| | | Pro | | | | Fluoro) | | | | | |
| 11 | H | (S)-α- | E | G | T | L-α-Me- | T | S | D | Bip(2′-Et- | (S)-2-amino-5-(5- |
| | | Me- | | | | Phe(2- | | | | 4′-OMe) | methylpyridin-2-yl)pentanamide |
| | | Pro | | | | Fluoro) | | | | | |
| 12 | H | (S)-α- | E | G | T | L-α-Me- | T | S | D | Bip(2′-Et- | (S)-2-amino-5-(4- |
| | | Me- | | | | Phe(2- | | | | 4′-OMe) | propylphenyl)pentanamide |
| | | Pro | | | | Fluoro) | | | | | |
| 13 | H | (S)-α- | E | G | T | L-α-Me- | T | S | D | Bip(2′-Et- | (S)-2-amino-5-(2- |
| | | Me- | | | | Phe(2- | | | | 4′-OMe) | ethylphenyl)pentanamide |
| | | Pro | | | | Fluoro) | | | | | |
| 14 | H | (S)-α- | E | G | T | L-α-Me- | T | S | D | Bip(2′-Et- | (S)-2-amino-5-(4- |
| | | Me- | | | | Phe(2- | | | | 4′-OMe) | methylpyridin-2-yl)pentanamide |
| | | Pro | | | | Fluoro) | | | | | |
| 15 | H | (S)-α- | E | G | T | L-α-Me- | T | S | D | Bip(2′-Et- | (S)-2-amino-5-(2,4- |
| | | Me- | | | | Phe(2- | | | | 4′-OMe) | dimethylphenyl)pentanamide |
| | | Pro | | | | Fluoro) | | | | | |
| 16 | H | (S)-α- | E | G | T | L-α-Me- | T | S | D | Bip(2′-Et- | (S)-2-amino-5-(3- |
| | | Me- | | | | Phe(2- | | | | 4′-OMe) | methoxyphenyl)pentanamide |
| | | Pro | | | | Fluoro) | | | | | |
| 17 | H | (S)-α- | E | G | T | L-α-Me- | T | S | D | Bip(2′-Et- | (S)-2-amino-5- |
| | | Me- | | | | Phe(2- | | | | 4′-OMe) | phenylpentanamide |
| | | Pro | | | | Fluoro) | | | | | |
| 18 | H | (S)-α- | E | G | T | L-α-Me- | T | S | D | Bip(2′-Et- | (S)-2-amino-5-(4- |
| | | Me- | | | | Phe(2- | | | | 4′-OMe) | biphenyl)pentanamide |
| | | Pro | | | | Fluoro) | | | | | |
| 19 | H | (S)-α- | E | G | T | L-α-Me- | T | S | D | Bip(2′-Et- | (S)-2-amino-5-(3- |
| | | Me- | | | | Phe(2- | | | | 4′-OMe) | methylpyridin-2-yl) |
| | | Pro | | | | Fluoro) | | | | | pentanamide |
| 20 | H | (S)-α- | E | G | T | L-α-Me- | T | S | D | Bip(2′-Et- | (S)-2-amino-5-(pyridin-4-yl) |
| | | Me- | | | | Phe(2- | | | | 4′-OMe) | pentanamide |
| | | Pro | | | | Fluoro) | | | | | |
| 21 | H | (S)-α- | E | G | T | L-α-Me- | T | S | D | Bip(2′-Et- | (S)-2-amino-5-(2,3- |
| | | Me- | | | | Phe(2- | | | | 4′-OMe) | dimethylphenyl)pentanamide |
| | | Pro | | | | Fluoro) | | | | | |
| 22 | H | (S)-α- | E | G | T | L-α-Me- | T | S | D | Bip(2′-Et- | (S)-2-amino-5-(quinolin-6- |
| | | Me- | | | | Phe(2- | | | | 4′-OMe) | yl)pentanamide |
| | | Pro | | | | Fluoro) | | | | | |
| 23 | H | (S)-α- | E | G | T | L-α-Me- | T | S | D | Bip(2′-Et- | (S)-2-amino-5-(naphthalen-2- |
| | | Me- | | | | Phe(2- | | | | 4′-OMe) | yl)pentanamide |
| | | Pro | | | | Fluoro) | | | | | |
| 24 | H | (S)-α- | E | G | T | L-α-Me- | T | S | D | Bip(2′-Et- | (S)-2-amino-5-(6- |
| | | Me- | | | | Phe(2- | | | | 4′-OMe) | methylpyridin-2-yl) |
| | | Pro | | | | Fluoro) | | | | | pentanamide |
| 25 | H | (S)-α- | E | G | T | L-α-Me- | T | S | D | Bip(2′-Et- | (S)-2-amino-5-(2,3- |
| | | Me- | | | | Phe(2- | | | | 4′-OMe) | dihydrobenzo[b][1,4]dioxin-6- |
| | | Pro | | | | Fluoro) | | | | | yl)pentanamide |
| 26 | H | (S)-α- | E | G | T | L-α-Me- | T | S | D | Bip(2′-Et- | (S)-2-amino-5-(2,6- |
| | | Me- | | | | Phe(2- | | | | 4′-OMe) | dimethylpyridin-3- |
| | | Pro | | | | Fluoro) | | | | | yl)pentanamide |
| 27 | H | (S)-α- | E | G | T | L-α-Me- | T | S | D | Bip(2′-Et- | (S)-2-amino-5-(4- |
| | | Me- | | | | Phe(2- | | | | 4′-OMe) | chlorophenyl)pentanamide |
| | | Pro | | | | Fluoro) | | | | | |
| 28 | H | (S)-α- | E | G | T | L-α-Me- | T | S | D | Bip(2′-Et- | (S)-2-amino-5-(3- |
| | | Me- | | | | Phe(2- | | | | 4′-OMe) | chlorophenyl)pentanamide |
| | | Pro | | | | Fluoro) | | | | | |
| 29 | H | (S)-α- | E | G | T | L-α-Me- | T | S | D | Bip(2′-Et- | (S)-2-amino-5-(4- |
| | | Me- | | | | Phe(2- | | | | 4′-OMe) | (dimethylamino)phenyl) |
| | | Pro | | | | Fluoro) | | | | | pentanamide |
| 30 | H | (S)-α- | E | G | T | L-α-Me- | T | S | D | Bip(2′-Et- | (S)-2-amino-5-(2- |
| | | Me- | | | | Phe(2- | | | | 4′-OMe) | chlorophenyl)pentanamide |
| | | Pro | | | | Fluoro) | | | | | |
| 31 | H | (S)-α- | E | G | T | L-α-Me- | T | S | D | Bip(2′-Et- | (S)-2-amino-5-(4- |
| | | Me- | | | | Phe(2- | | | | 4′-OMe) | methylsulfonyl)phenyl) |
| | | Pro | | | | Fluoro) | | | | | pentanamide |
| 32 | H | (S)-α- | E | G | T | L-α-Me- | T | S | D | Bip(2′-Et- | (S)-2-amino-5-(quinolin-5- |
| | | Me- | | | | Phe(2- | | | | 4′-OMe) | yl)pentanamide |
| | | Pro | | | | Fluoro) | | | | | |
| 33 | H | (S)-α- | E | G | T | L-α-Me- | T | S | D | Bip(2′-Et- | (S)-2-amino-5-(4- |
| | | Me- | | | | Phe(2- | | | | 4′-OMe) | (trifluoromethyl)phenyl) |
| | | Pro | | | | Fluoro) | | | | | pentanamide |
| 34 | H | (S)-α- | E | G | T | L-α-Me- | T | S | D | Bip(2′-Et- | (S)-2-amino-5-(4- |
| | | Me- | | | | Phe(2- | | | | 4′-OMe) | fluorophenyl)pentanamide |
| | | Pro | | | | Fluoro) | | | | | |
| 35 | H | (S)-α- | E | G | T | L-α-Me- | T | S | D | Bip(2′-Et- | (S)-2-amino-5-(3- |
| | | Me- | | | | Phe(2- | | | | 4′-OMe) | biphenyl)pentanamide |
| | | Pro | | | | Fluoro) | | | | | |
| 36 | H | (S)-α- | E | G | T | L-α-Me- | T | S | D | Bip(2′-Et- | 4-phenylbutan-1-amine |
| | | Me- | | | | Phe(2- | | | | 4′-OMe) | |
| | | Pro | | | | Fluoro) | | | | | |
| 37 | H | (S)-α- | E | G | T | L-α-Me- | T | S | D | Bip(2′-Et- | (S)-2-amino-5-(2,3,5,6- |
| | | Me- | | | | Phe(2- | | | | 4′-OMe) | tetramethylphenyl)pentanamide |
| | | Pro | | | | Fluoro) | | | | | |
| 38 | H | (S)-α- | E | G | T | L-α-Me- | T | S | D | Bip(2′-Et- | (S)-2-amino-5-(4- |
| | | Me- | | | | Phe(2- | | | | 4′-OMe) | isopropylphenyl)pentanamide |
| | | Pro | | | | Fluoro) | | | | | |
| 39 | H | (S)-α- | E | G | T | L-α-Me- | T | S | D | Bip(2′-Et- | (S)-2-amino-5-(3- |
| | | Me- | | | | Phe(2- | | | | 4′-OMe) | biphenyl)pentanamide |
| | | Pro | | | | Fluoro) | | | | | |
| 40 | H | (S)-α- | E | G | T | L-α-Me- | T | S | D | Bip(2′-Et- | (S)-2-amino-5-(4- |
| | | Me- | | | | Phe(2- | | | | 4′-OMe) | (dimethylamino)phenyl) |
| | | Pro | | | | Fluoro) | | | | | pentanamide |
| 41 | H | (S)-α- | E | G | T | L-α-Me- | T | S | D | Bip(2′-Et- | (S)-2-amino-5-(3- |
| | | Me- | | | | Phe(2- | | | | 4′-OMe) | chlorophenyl)pentanamide |
| | | Pro | | | | Fluoro) | | | | | |
| 42 | H | (S)-α- | E | G | T | L-α-Me- | T | S | D | Bip(2′-Et- | (S)-2-amino-5-(4- |
| | | Me- | | | | Phe(2- | | | | 4′-OMe) | fluorophenyl)pentanamide |
| | | Pro | | | | Fluoro) | | | | | |
| 43 | H | (S)-α- | E | G | T | L-α-Me- | T | S | D | Bip(2′-Et- | (S)-2-amino-5-(2- |
| | | Me- | | | | Phe(2- | | | | 4′-OMe) | chlorophenyl)pentanamide |
| | | Pro | | | | Fluoro) | | | | | |
| 44 | H | (S)-α- | E | G | T | L-α-Me- | T | S | D | Bip(2′-Et- | (S)-2-amino-5-(4- |
| | | Me- | | | | Phe(2- | | | | 4′-OMe) | (trifluoromethyl)phenyl) |
| | | Pro | | | | Fluoro) | | | | | pentanamide |
| 45 | H | (S)-α- | E | G | T | L-α-Me- | T | S | D | Bip(2′-Et- | (S)-2-amino-5-(4- |
| | | Me- | | | | Phe(2- | | | | 4′-OMe) | isopropylphenyl)pentanamide |
| | | Pro | | | | Fluoro) | | | | | |
| 46 | H | (S)-α- | E | G | T | L-α-Me- | T | S | D | Bip(2′-Et- | (S)-2-amino-5-(4- |
| | | Me- | | | | Phe(2- | | | | 4′-OMe) | (methylsulfonyl)phenyl) |
| | | Pro | | | | Fluoro) | | | | | pentanamide |
| 47 | H | (S)-α- | E | G | T | L-α-Me- | T | S | D | Bip(2′-Et- | (S)-2-amino-5-(quinolin-5- |
| | | Me- | | | | Phe(2- | | | | 4′-OMe) | yl)pentanamide |
| | | Pro | | | | Fluoro) | | | | | |
| 48 | H | (S)-α- | E | G | T | L-α-Me- | T | S | D | Bip(2′-Et- | (S)-2-amino-5-(2,3,5,6- |
| | | Me- | | | | Phe(2- | | | | 4′-OMe) | tetramethylphenyl)pentanamide |
| | | Pro | | | | Fluoro) | | | | | |
| 49 | H | (S)-α- | E | G | T | L-α-Me- | T | S | D | Bip(2′-Et- | (S)-2-amino-5-(pyridin-2-yl) |
| | | Me- | | | | Phe(2- | | | | 4′-OMe) | pentanamide |
| | | Pro | | | | Fluoro) | | | | | |
| 50 | H | (S)-α- | E | G | T | L-α-Me- | T | S | D | Bip(2′-Et- | (S)-2-amino-5-(pyridin-3-yl) |
| | | Me- | | | | Phe(2- | | | | 4′-OMe) | pentanamide |
| | | Pro | | | | Fluoro) | | | | | |
| 51 | H | (S)-α- | E | G | T | L-α-Me- | T | S | D | Bip(2′-Et- | (S)-2-amino-5-(pyrazin-2-yl) |
| | | Me- | | | | Phe(2- | | | | 4′-OMe) | pentanamide |
| | | Pro | | | | Fluoro) | | | | | |
| 52 | H | (S)-α- | E | G | T | L-α-Me- | T | S | D | Bip(2′-Et- | (S)-2-amino-5- |
| | | Me- | | | | Phe(2- | | | | 4′-OMe) | (benzo[d][1,3]dioxol-5-yl) |
| | | Pro | | | | Fluoro) | | | | | pentanamide |
| 53 | H | (S)-α- | E | G | T | L-α-Me- | T | S | D | Bip(2′-Et- | (S)-2-amino-5-(3,5- |
| | | Me- | | | | Phe(2- | | | | 4′-OMe) | dimethylphenyl)pentanamide |
| | | Pro | | | | Fluoro) | | | | | |
| 54 | H | (S)-α- | E | G | T | L-α-Me- | T | S | D | Bip(2′-Et- | (S)-2-amino-5-o- |
| | | Me- | | | | Phe(2- | | | | 4′-OMe) | tolylpentanamide |
| | | Pro | | | | Fluoro) | | | | | |
| 55 | H | (S)-α- | E | G | T | L-α-Me- | T | S | D | Bip(2′-Et- | (S)-2-amino-5-(6- |
| | | Me- | | | | Phe(2- | | | | 4′-OMe) | methoxypyridin-2-yl) |
| | | Pro | | | | Fluoro) | | | | | pentanamide |
| 56 | H | (S)-α- | E | G | T | L-α-Me- | T | S | D | Bip(2′-Et- | (S)-2-amino-5-(2,3- |
| | | Me- | | | | Phe(2- | | | | 4′-OMe) | dihydrobenzofuran-5-yl) |
| | | Pro | | | | Fluoro) | | | | | pentanamide |
| 57 | H | (S)-α- | E | G | T | L-α-Me- | T | S | D | Bip(2′-Et- | (S)-2-(4,5-diamino-5- |
| | | Me- | | | | Phe(2- | | | | 4′-OMe) | oxopentyl)isonicotinamide |
| | | Pro | | | | Fluoro) | | | | | |
| 58 | H | Aib | E | G | T | L-α-Me- | T | S | D | Bip(2′-Et- | (R)-2-amino-4-(2- |
| | | | | | | Phe(2- | | | | 4′-OMe) | chlorophenoxy)butanamide |
| | | | | | | Fluoro) | | | | | |
| 59 | H | Aib | E | G | T | L-α-Me- | T | S | D | Bip(2′-Et- | (S)-2-amino-4-(2- |
| | | | | | | Phe(2- | | | | 4′-OMe) | chlorophenoxy)butanamide |
| | | | | | | Fluoro) | | | | | |
| 60 | H | (S)-α- | E | G | T | L-α-Me- | T | S | D | Bip(2′-Et- | (S)-2-amino-4-(2- |
| | | Me- | | | | Phe(2- | | | | 4′-OMe) | chlorophenoxy)butanamide |
| | | Pro | | | | Fluoro) | | | | | |
| 61 | H | Aib | E | G | T | L-α-Me- | T | S | D | Bip(2′-Et- | (S)-2-amino-4- |
| | | | | | | Phe(2- | | | | 4′-OMe) | phenoxybutanamide |
| | | | | | | Fluoro) | | | | | |
| 62 | H | (S)-α- | E | G | T | L-α-Me- | T | S | D | Bip(2′-Et- | (S)-2-amino-4- |
| | | Me- | | | | Phe(2- | | | | 4′-OMe) | phenoxybutanamide |
| | | Pro | | | | Fluoro) | | | | | |
| 63 | H | Aib | E | G | T | L-α-Me- | T | S | D | Bip(2′-Et- | (S)-2-amino-4-(2,4- |
| | | | | | | Phe(2- | | | | 4′-OMe) | dimethylphenoxy)butanamide |
| | | | | | | Fluoro) | | | | | |
| 64 | H | (S)-α- | E | G | T | L-α-Me- | T | S | D | Bip(2′-Et- | (S)-2-amino-4-(2,4- |
| | | Me- | | | | Phe(2- | | | | 4′-OMe) | dimethylphenoxy)butanamide |
| | | Pro | | | | Fluoro) | | | | | |
| 65 | H | Aib | E | G | T | L-α-Me- | T | S | D | Bip(2′-Et- | (S)-2-amino-4-(2- |
| | | | | | | Phe(2- | | | | 4′-OMe) | methylphenoxy)butanamide |
| | | | | | | Fluoro) | | | | | |
| 66 | H | (S)-α- | E | G | T | L-α-Me- | T | S | D | Bip(2′-Et- | (S)-2-amino-4-(2- |
| | | Me- | | | | Phe(2- | | | | 4′-OMe) | methylphenoxy)butanamide |
| | | Pro | | | | Fluoro) | | | | | |
| 67 | H | Aib | E | G | T | L-α-Me- | T | S | D | Bip(2′-Et- | (S)-2-amino-4-(4- |
| | | | | | | Phe(2- | | | | 4′-OMe) | methoxyphenoxy)butanamide |
| | | | | | | Fluoro) | | | | | |
| 68 | H | (S)-α- | E | G | T | L-α-Me- | T | S | D | Bip(2′-Et- | (S)-2-amino-4-(4- |
| | | Me- | | | | Phe(2- | | | | 4′-OMe) | methoxyphenoxy)butanamide |
| | | Pro | | | | Fluoro) | | | | | |
| 69 | H | Aib | E | G | T | L-α-Me- | T | S | D | Bip(2′-Et- | (S)-2-amino-4-(4- |
| | | | | | | Phe(2- | | | | 4′-OMe) | methylphenoxy)butanamide |
| | | | | | | Fluoro) | | | | | |
| 70 | H | (S)-α- | E | G | T | L-α-Me- | T | S | D | Bip(2′-Et- | (S)-2-amino-4-(4- |
| | | Me- | | | | Phe(2- | | | | 4′-OMe) | methylphenoxy)butanamide |
| | | Pro | | | | Fluoro) | | | | | |
| 71 | H | Aib | E | G | T | L-α-Me- | T | S | D | Bip(2′-Et- | (S)-2-amino-4-(5- |
| | | | | | | Phe(2- | | | | 4′-OMe) | methylpyridin-2- |
| | | | | | | Fluoro) | | | | | yloxy)butanamide |
| 72 | H | (S)-α- | E | G | T | L-α-Me- | T | S | D | Bip(2′-Et- | (S)-2-amino-4-(5- |
| | | Me- | | | | Phe(2- | | | | 4′-OMe) | methylpyridin-2- |
| | | Pro | | | | Fluoro) | | | | | yloxy)butanamide |
| 73 | H | Aib | E | G | T | L-α-Me- | T | S | D | Bip(2′-Et- | (S)-2-amino-4-(4- |
| | | | | | | Phe(2- | | | | 4′-OMe) | fluorophenoxy)butanamide |
| | | | | | | Fluoro) | | | | | |
| 74 | H | (S)-α- | E | G | T | L-α-Me- | T | S | D | Bip(2′-Et- | (S)-2-amino-4-(4- |
| | | Me- | | | | Phe(2- | | | | 4′-OMe) | fluorophenoxy)butanamide |
| | | Pro | | | | Fluoro) | | | | | |
| 75 | H | (S)-α- | E | G | T | L-α-Me- | T | S | D | Bip(2′-Et- | (S)-2-amino-4-(2- |
| | | Me- | | | | Phe(2- | | | | 4′-OMe) | fluorophenoxy)butanamide |
| | | Pro | | | | Fluoro) | | | | | |
| 76 | H | (S)-α- | E | G | T | L-α-Me- | T | S | D | Bip(2′-Et- | (S)-2-amino-4-(2,4- |
| | | Me- | | | | Phe(2- | | | | 4′-OMe) | dichlorophenoxy)butanamide |
| | | Pro | | | | Fluoro) | | | | | |
| 77 | H | (S)-α- | E | G | T | L-α-Me- | T | S | D | Bip(2′-Et- | (S)-2-amino-4-(2,3- |
| | | Me- | | | | Phe(2- | | | | 4′-OMe) | dimethylphenoxy)butanamide |
| | | Pro | | | | Fluoro) | | | | | |
| 78 | H | (S)-α- | E | G | T | L-α-Me- | T | S | D | Bip(2′-Et- | (S)-2-amino-4-(3- |
| | | Me- | | | | Phe(2- | | | | 4′-OMe) | fluorophenoxy)butanamide |
| | | Pro | | | | Fluoro) | | | | | |
| 79 | H | (S)-α- | E | G | T | L-α-Me- | T | S | D | Bip(2′-Et- | (S)-2-amino-4-(3- |
| | | Me- | | | | Phe(2- | | | | 4′-OMe) | chlorophenoxy)butanamide |
| | | Pro | | | | Fluoro) | | | | | |
| 80 | H | (S)-α- | E | G | T | L-α-Me- | T | S | D | Bip(2′-Et- | (S)-2-amino-4-(3- |
| | | Me- | | | | Phe(2- | | | | 4′-OMe) | methoxyphenoxy)butanamide |
| | | Pro | | | | Fluoro) | | | | | |
| 81 | H | (S)-α- | E | G | T | L-α-Me- | T | S | D | Bip(2′-Et- | (S)-2-amino-4-(3- |
| | | Me- | | | | Phe(2- | | | | 4′-OMe) | methylphenoxy)butanamide |
| | | Pro | | | | Fluoro) | | | | | |
| 82 | H | (S)-α- | E | G | T | L-α-Me- | T | S | D | Bip(2′-Et- | (S)-2-amino-4-(3,4- |
| | | Me- | | | | Phe(2- | | | | 4′-OMe) | dimethylphenoxy)butanamide |
| | | Pro | | | | Fluoro) | | | | | |
| 83 | H | Aib | E | G | T | L-α-Me- | T | S | D | Bip(2′-Et- | (S)-2-amino-4-(4- |
| | | | | | | Phe(2- | | | | 4′-OMe) | phenylphenoxy)butanamide |
| | | | | | | Fluoro) | | | | | |
| 84 | H | (S)-α- | E | G | T | L-α-Me- | T | S | D | Bip(2′-Et- | (S)-2-amino-4-(2,3-dihydro-1H- |
| | | Me- | | | | Phe(2- | | | | 4′-OMe) | inden-5-yloxy)butanamide |
| | | Pro | | | | Fluoro) | | | | | |
| 85 | H | (S)-α- | E | G | T | L-α-Me- | T | S | D | Bip(2′-Et- | (S)-2-amino-4-(4-chloro-2- |
| | | Me- | | | | Phe(2- | | | | 4′-OMe) | methylphenoxy)-N- |
| | | Pro | | | | Fluoro) | | | | | methylbutanamide |
| 86 | H | (S)-α- | E | G | T | L-α-Me- | T | S | D | Bip(2′-Et- | (R)-2-amino-4-(4-(1H-1,2,4- |
| | | Me- | | | | Phe(2- | | | | 4′-OMe) | triazol-1-yl- |
| | | Pro | | | | Fluoro) | | | | | phenoxy)butanamide |
| 87 | H | (S)-α- | E | G | T | L-α-Me- | T | S | D | Bip(2′-Et- | (S)-2-amino-4-(2-CN- |
| | | Me- | | | | Phe(2- | | | | 4′-OMe) | phenoxy)butanamide |
| | | Pro | | | | Fluoro) | | | | | |
| 88 | H | (S)-α- | E | G | T | L-α-Me- | T | S | D | Bip(2′-Et- | (S)-2-amino-4-(2-MeO- |
| | | Me- | | | | Phe(2- | | | | 4′-OMe) | phenoxy)butanamide |
| | | Pro | | | | Fluoro) | | | | | |
| 89 | H | (S)-α- | E | G | T | L-α-Me- | T | S | D | Bip(2′-Et- | (S)-2-amino-4-(2- |
| | | Me- | | | | Phe(2- | | | | 4′-OMe) | phenylphenoxy)butanamide |
| | | Pro | | | | Fluoro) | | | | | |
| 90 | H | (S)-α- | E | G | T | L-α-Me- | T | S | D | Bip(2′-Et- | (S)-2-amino-4-(2-CF₃- |
| | | Me- | | | | Phe(2- | | | | 4′-OMe) | phenoxy)butanamide |
| | | Pro | | | | Fluoro) | | | | | |
| 91 | H | (S)-α- | E | G | T | L-α-Me- | T | S | D | Bip(2′-Et- | (S)-2-amino-4-(3-acetamido-- |
| | | Me- | | | | Phe(2- | | | | 4′-OMe) | phenoxy)butanamide |
| | | Pro | | | | Fluoro) | | | | | |
| 92 | H | (S)-α- | E | G | T | L-α-Me- | T | S | D | Bip(2′-Et- | (S)-2-amino-4-(3- |
| | | Me- | | | | Phe(2- | | | | 4′-OMe) | dimethylamino- |
| | | Pro | | | | Fluoro) | | | | | phenoxy)butanamide |
| 93 | H | (S)-α- | E | G | T | L-α-Me- | T | S | D | Bip(2′-Et- | (S)-2-amino-4-(3- |
| | | Me- | | | | Phe(2- | | | | 4′-OMe) | phenylphenoxy)butanamide |
| | | Pro | | | | Fluoro) | | | | | |
| 94 | H | (S)-α- | E | G | T | L-α-Me- | T | S | D | Bip(2′-Et- | (S)-2-amino-4-(4-Cl-2-Me- |
| | | Me- | | | | Phe(2- | | | | 4′-OMe) | phenoxy)butanamide |
| | | Pro | | | | Fluoro) | | | | | |
| 95 | H | (S)-α- | E | G | T | L-α-Me- | T | S | D | Bip(2′-Et- | (S)-2-amino-4-(4-phenoxy-- |
| | | Me- | | | | Phe(2- | | | | 4′-OMe) | phenoxy)butanamide |
| | | Pro | | | | Fluoro) | | | | | |
| 96 | H | (S)-α- | E | G | T | L-α-Me- | T | S | D | Bip(2′-Et- | (S)-2-amino-4-(4-CF₃- |
| | | Me- | | | | Phe(2- | | | | 4′-OMe) | phenoxy)butanamide |
| | | Pro | | | | Fluoro) | | | | | |
| 97 | H | (S)-α- | E | G | T | L-α-Me- | T | S | D | Bip(2′-Et- | (S)-2-amino-4-(4-isopropyl- |
| | | Me- | | | | Phe(2- | | | | 4′-OMe) | phenoxy)butanamide |
| | | Pro | | | | Fluoro) | | | | | |
| 98 | H | (S)-α- | E | G | T | L-α-Me- | T | S | D | Bip(2′-Et- | (S)-2-amino-4-(1- |
| | | Me- | | | | Phe(2- | | | | 4′-OMe) | naphthoxy)butanamide |
| | | Pro | | | | Fluoro) | | | | | |
| 99 | H | (S)-α- | E | G | T | L-α-Me- | T | S | D | Bip(2′-Et- | (S)-2-amino-4-(2- |
| | | Me- | | | | Phe(2- | | | | 4′-OMe) | naphthoxy)butanamide |
| | | Pro | | | | Fluoro) | | | | | |
| 100 | H | (S)-α- | E | G | T | L-α-Me- | T | S | D | Bip(2′-Et- | 2-amino-4-(4-(imidazol-1-yl)- |
| | | Me- | | | | Phe(2- | | | | 4′-OMe) | phenoxy)butanamide |
| | | Pro | | | | Fluoro) | | | | | |
| 101 | H | (S)-α- | E | G | T | L-α-Me- | T | S | D | Bip(2′-Et- | (S)-2-amino-5- |
| | | Me- | | | | Phe(2- | | | | 4′-OMe) | (benzo[d][1,3]dioxol-5- |
| | | Pro | | | | Fluoro) | | | | | yl)pentanamide |
| 102 | H | (S)-α- | E | G | T | L-α-Me- | T | S | D | Bip(2′-Et- | (S)-2-amino-4-(3- |
| | | Me- | | | | Phe(2- | | | | 4′-OMe) | dimethylamino- |
| | | Pro | | | | Fluoro) | | | | | phenoxy)butanamide |
| 103 | H | (S)-α- | E | G | T | L-α-Me- | T | S | D | Bip(2′-Et- | 2-amino-4-(quinolin-6- |
| | | Me- | | | | Phe(2- | | | | 4′-OMe) | yloxy)butanamide |
| | | Pro | | | | Fluoro) | | | | | |
| 104 | H | (S)-α- | E | G | T | L-α-Me- | T | S | D | Bip(2′-Et- | (S)-2-amino-4-(3- |
| | | Me- | | | | Phe(2- | | | | 4′-OMe) | trifluoromethoxy- |
| | | Pro | | | | Fluoro) | | | | | phenoxy)butanamide |
| 105 | H | (S)-α- | E | G | T | L-α-Me- | T | S | D | Bip(2′-Et- | 2-amino-4-(4-methylpyridin-2- |
| | | Me- | | | | Phe(2- | | | | 4′-OMe) | yloxy)butanamide |
| | | Pro | | | | Fluoro) | | | | | |
| 106 | H | (S)-α- | E | G | T | L-α-Me- | T | S | D | Bip(2′-Et- | 2(S)-amino-4-(2-(1H-prazol-3- |
| | | Me- | | | | Phe(2- | | | | 4′-OMe) | yl-phenoxy)butanamide |
| | | Pro | | | | Fluoro) | | | | | |
| 107 | H | (S)-α- | E | G | T | L-α-Me- | T | S | D | Bip(2′-Et- | 2(S)-amino-4-(4-(1H-1,2,4- |
| | | Me- | | | | Phe(2- | | | | 4′-OMe) | triazol-1-yl- |
| | | Pro | | | | Fluoro) | | | | | phenoxy)butanamide |
| 108 | des-amino | (S)-α- | E | G | T | L-α-Me- | T | S | D | Bip(2′-Et- | (S)-4-(3,4-diamino-4- |
| | His | Me- | | | | Phe(2- | | | | 4′-OMe) | oxobutoxy)benzamide |
| | | Pro | | | | Fluoro) | | | | | |
| 109 | des-amino | (S)-α- | E | G | T | L-α-Me- | T | S | D | Bip(2′-Et- | (S)-2-amino-4- |
| | His | Me- | | | | Phe(2- | | | | 4′-OMe) | phenoxybutanamide |
| | | Pro | | | | Fluoro) | | | | | |
| 110 | des-amino | (S)-α- | E | G | T | L-α-Me- | T | S | D | Bip(2′-Et- | (S)-2-amino-4-(2- |
| | His | Me- | | | | Phe(2- | | | | 4′-OMe) | fluorophenoxy)butanamide |
| | | Pro | | | | Fluoro) | | | | | |
| 111 | des-amino | (S)-α- | E | G | T | L-α-Me- | T | S | D | Bip(2′-Et- | (S)-2-amino-4-(2- |
| | His | Me- | | | | Phe(2- | | | | 4′-OMe) | phenylphenoxy)butanamide |
| | | Pro | | | | Fluoro) | | | | | |
| 112 | des-amino | (S)-α- | E | G | T | L-α-Me- | T | S | D | Bip(2′-Et- | (S)-2-amino-4-(2- |
| | His | Me- | | | | Phe(2- | | | | 4′-OMe) | trifluoromethylphenoxy)butanamide |
| | | Pro | | | | Fluoro) | | | | | |
| 113 | des-amino | (S)-α- | E | G | T | L-α-Me- | T | S | D | Bip(2′-Et- | (S)-2-amino-4-(2- |
| | His | Me- | | | | Phe(2- | | | | 4′-OMe) | methylphenoxy)butanamide |
| | | Pro | | | | Fluoro) | | | | | |
| 114 | des-amino | (S)-α- | E | G | T | L-α-Me- | T | S | D | Bip(2′-Et- | (S)-2-amino-4-(2,4- |
| | His | Me- | | | | Phe(2- | | | | 4′-OMe) | dimethylphenoxy)butanamide |
| | | Pro | | | | Fluoro) | | | | | |
| 115 | des-amino | (S)-α- | E | G | T | L-α-Me- | T | S | D | Bip(2′-Et- | (S)-2-amino-4-(3- |
| | His | Me- | | | | Phe(2- | | | | 4′-OMe) | acetamidophenoxy)butanamide |
| | | Pro | | | | Fluoro) | | | | | |
| 116 | des-amino | (S)-α- | E | G | T | L-α-Me- | T | S | D | Bip(2′-Et- | (S)-2-amino-4-(2-methyl-4- |
| | His | Me- | | | | Phe(2- | | | | 4′-OMe) | chlorophenoxy)butanamide |
| | | Pro | | | | Fluoro) | | | | | |
| 117 | des-amino | (S)-α- | E | G | T | L-α-Me- | T | S | D | Bip(2′-Et- | (S)-2-amino-4-(4- |
| | His | Me- | | | | Phe(2- | | | | 4′-OMe) | phenoxyphenoxy)butanamide |
| | | Pro | | | | Fluoro) | | | | | |
| 118 | des-amino | (S)-α- | E | G | T | L-α-Me- | T | S | D | Bip(2′-Et- | (S)-2-amino-4-(2- |
| | His | Me- | | | | Phe(2- | | | | 4′-OMe) | methoxyphenoxy)butanamide |
| | | Pro | | | | Fluoro) | | | | | |
| 119 | des-amino | (S)-α- | E | G | T | L-α-Me- | T | S | D | Bip(2′-Et- | (S)-2-amino-4-(4- |
| | His | Me- | | | | Phe(2- | | | | 4′-OMe) | trifluoromethylphenoxy)butanamide |
| | | Pro | | | | Fluoro) | | | | | |
| 120 | des-amino | (S)-α- | E | G | T | L-α-Me- | T | S | D | Bip(2′-Et- | (S)-2-amino-4-(2,3-dihydro-1H- |
| | His | Me- | | | | Phe(2- | | | | 4′-OMe) | inden-5-yloxy)butanamide |
| | | Pro | | | | Fluoro) | | | | | |
| 121 | des-amino | (S)-α- | E | G | T | L-α-Me- | T | S | D | Bip(2′-Et- | (S)-2-amino-4-(1- |
| | His | Me- | | | | Phe(2- | | | | 4′-OMe) | naphthoxy)butanamide |
| | | Pro | | | | Fluoro) | | | | | |
| 122 | des-amino | (S)-α- | E | G | T | L-α-Me- | T | S | D | Bip(2′-Et- | (S)-2-amino-4-(2- |
| | His | Me- | | | | Phe(2- | | | | 4′-OMe) | naphthoxy)butanamide |
| | | Pro | | | | Fluoro) | | | | | |
| 123 | des-amino | (S)-α- | E | G | T | L-α-Me- | T | S | D | Bip(2′-Et- | (S)-2-amino-4- |
| | His | Me- | | | | Phe(2- | | | | 4′-OMe) | (benzo[d][1,3]dioxol-5- |
| | | Pro | | | | Fluoro) | | | | | yloxy)butanamide |
| 124 | des-amino | (S)-α- | E | G | T | L-α-Me- | T | S | D | Bip(2′-Et- | (S)-2-amino-4-(quinolin-6- |
| | His | Me- | | | | Phe(2- | | | | 4′-OMe) | yloxy)butanamide |
| | | Pro | | | | Fluoro) | | | | | |
| 125 | des-amino | (S)-α- | E | G | T | L-α-Me- | T | S | D | Bip(2′-Et- | (S)-2-amino-5- |
| | His | Me- | | | | Phe(2- | | | | 4′-OMe) | phenylpentanamide |
| | | Pro | | | | Fluoro) | | | | | |
| 126 | des-amino | (S)-α- | E | G | T | L-α-Me- | T | S | D | Bip(2′-Et- | (S)-2-amino-5-(3-chlorophenyl) |
| | His | Me- | | | | Phe(2- | | | | 4′-OMe) | pentanamide |
| | | Pro | | | | Fluoro) | | | | | |
| 127 | des-amino | (S)-α- | E | G | T | L-α-Me- | T | S | D | Bip(2′-Et- | (S)-2-amino-5- |
| | His | Me- | | | | Phe(2- | | | | 4′-OMe) | (benzo[d][1,3]dioxol-5- |
| | | Pro | | | | Fluoro) | | | | | yl)pentanamide |
| 128 | des-amino | (S)-α- | E | G | T | L-α-Me- | T | S | D | Bip(2′-Et- | (S)-2-amino-5-(3,5- |
| | His | Me- | | | | Phe(2- | | | | 4′-OMe) | dimethylphenyl)pentanamide |
| | | Pro | | | | Fluoro) | | | | | |
| 129 | des-amino | (S)-α- | E | G | T | L-α-Me- | T | S | D | Bip(2′-Et- | (S)-2-amino-5-(3- |
| | His | Me- | | | | Phe(2- | | | | 4′-OMe) | methoxyphenyl)pentanamide |
| | | Pro | | | | Fluoro) | | | | | |
| 130 | des-amino | (S)-α- | E | G | T | L-α-Me- | T | S | D | Bip(2′-Et- | (S)-2-amino-5-(naphthalen-2- |
| | His | Me- | | | | Phe(2- | | | | 4′-OMe) | yl)pentanamide |
| | | Pro | | | | Fluoro) | | | | | |
| 131 | des-amino | (S)-α- | E | G | T | L-α-Me- | T | S | D | Bip(2′-Et- | (S)-2-amino-5-(6- |
| | His | Me- | | | | Phe(2- | | | | 4′-OMe) | methylpyridin-2-yl)pentanamide |
| | | Pro | | | | Fluoro) | | | | | |
| 132 | des-amino | (S)-α- | E | G | T | L-α-Me- | T | S | D | Bip(2′-Et- | (R)-2-amino-5-(4-chloro-2- |
| | His | Me- | | | | Phe(2- | | | | 4′-OMe) | methylphenyl)pentanamide |
| | | Pro | | | | Fluoro) | | | | | |
| 133 | des-amino | (S)-α- | E | G | T | L-α-Me- | T | S | D | Bip(2′-Et- | (S)-2-amino-4-(4-chloro-2- |
| | His | Me- | | | | Phe(2- | | | | 4′-OMe) | methylphenoxy-N- |
| | | Pro | | | | Fluoro) | | | | | methylbutanamide |
| 134 | des-amino | (S)-α- | E | G | T | L-α-Me- | T | S | D | Bip(2′-Et- | (R)-2-amino-4-(4-chloro-2- |
| | His | Me- | | | | Phe(2- | | | | 4′-OMe) | methylphenoxy)-N- |
| | | Pro | | | | Fluoro) | | | | | methylbutanamide. |
| 135 | H₃C—O—CO- | (S)-α- | E | G | T | L-α-Me- | T | S | D | Bip(2′- | (S)-2-amino-5- |
| | His | Me-Pro | | | | Phe(2- | | | | Et-4′- | phenylpentanoyl-NH—Me |
| | | | | | | Fluoro) | | | | OMe) | |
| 136 | H₃C—O—CO- | (S)-α- | E | G | T | L-α-Me- | T | S | D | Bip(2′- | (S)-2-amino-4- |
| | His | Me-Pro | | | | Phe(2- | | | | Et-4′- | phenoxybutanamide |
| | | | | | | Fluoro) | | | | OMe) | |
| 137 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | fluorophenoxy) |
| | | | | | | Fluoro) | | | | OMe) | butanamide |
| 138 | H₃C—O—CO- | (S)-α- | E | G | T | L-α-Me- | T | S | D | Bip(2′- | (S)-2-amino-4-(2,4- |
| | His | Me-Pro | | | | Phe(2- | | | | Et-4′- | dichlorophenoxy) |
| | | | | | | Fluoro) | | | | OMe) | butanamide |
| 139 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | phenylphenoxy) |
| | | | | | | Fluoro) | | | | OMe) | butanamide |
| 140 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | trifluoromethylphenoxy) |
| | | | | | | Fluoro) | | | | OMe) | butanamide |
| 141 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | methylphenoxy) |
| | | | | | | Fluoro) | | | | OMe) | butanamide |
| 142 | H₃C—O—CO- | (S)-α- | E | G | T | L-α-Me- | T | S | D | Bip(2′- | (S)-2-amino-4-(2,3- |
| | His | Me-Pro | | | | Phe(2- | | | | Et-4′- | dimethylphenoxy) |
| | | | | | | Fluoro) | | | | OMe) | butanamide |
| 143 | H₃C—O—CO- | (S)-α- | E | G | T | L-α-Me- | T | S | D | Bip(2′- | (S)-2-amino-4-(3- |
| | His | Me-Pro | | | | Phe(2- | | | | Et-4′- | acetamidophenoxy) |
| | | | | | | Fluoro) | | | | OMe) | butanamide |
| 144 | H₃C—O—CO- | (S)-α- | E | G | T | L-α-Me- | T | S | D | Bip(2′- | (S)-2-amino-4-(3- |
| | His | Me-Pro | | | | Phe(2- | | | | Et-4′- | phenylphenoxy) |
| | | | | | | Fluoro) | | | | OMe) | butanamide |
| 145 | H₃C—O—CO- | (S)-α- | E | G | T | L-α-Me- | T | S | D | Bip(2′- | (S)-2-amino-4-(2-methyl- |
| | His | Me-Pro | | | | Phe(2- | | | | Et-4′- | 4-chlorophenoxy) |
| | | | | | | Fluoro) | | | | OMe) | butanamide |
| 146 | H₃C—O—CO- | (S)-α- | E | G | T | L-α-Me- | T | S | D | Bip(2′- | (S)-2-amino-4-(2-methyl- |
| | His | Me-Pro | | | | Phe(2- | | | | Et-4′- | 4-phenoxyphenoxy) |
| | | | | | | Fluoro) | | | | OMe) | butanamide |
| 147 | H₃C—O—CO- | (S)-α- | E | G | T | L-α-Me- | T | S | D | Bip(2′- | (S)-2-amino-4-(2-methyl- |
| | His | Me-Pro | | | | Phe(2- | | | | Et-4′- | 4-methoxyphenoxy) |
| | | | | | | Fluoro) | | | | OMe) | butanamide |
| 148 | H₃C—O—CO- | (S)-α- | E | G | T | L-α-Me- | T | S | D | Bip(2′- | (S)-2-amino-4-(2-methyl- |
| | His | Me-Pro | | | | Phe(2- | | | | Et-4′- | 4- |
| | | | | | | Fluoro) | | | | OMe) | trifluoromethylphenoxy) |
| | | | | | | | | | | | butanamide |
| 149 | H₃C—O—CO- | (S)-α- | E | G | T | L-α-Me- | T | S | D | Bip(2′- | (S)-2-amino-4-(2,3- |
| | His | Me-Pro | | | | Phe(2- | | | | Et-4′- | dihydro-1H-inden-5- |
| | | | | | | Fluoro) | | | | OMe) | yloxy)butanamide |
| 150 | H₃C—O—CO- | (S)-α- | E | G | T | L-α-Me- | T | S | D | Bip(2′- | (S)-2-amino-4-(1- |
| | His | Me-Pro | | | | Phe(2- | | | | Et-4′- | naphthoxy)butanamide |
| | | | | | | Fluoro) | | | | OMe) | |
| 151 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | naphthoxy)butanamide |
| | | | | | | Fluoro) | | | | OMe) | |
| 152 | H₃C—O—CO- | (S)-α- | E | G | T | L-α-Me- | T | S | D | Bip(2′- | (S)-2-amino-4- |
| | His | Me-Pro | | | | Phe(2- | | | | Et-4′- | (benzo[d][1,3]dioxol-5- |
| | | | | | | Fluoro) | | | | OMe) | yloxy)butanamide |
| 153 | H₃C—O—CO- | (S)-α- | E | G | T | L-α-Me- | T | S | D | Bip(2′- | (S)-2-amino-4-(quinolin- |
| | His | Me-Pro | | | | Phe(2- | | | | Et-4′- | 4-yloxy)butanamide |
| | | | | | | Fluoro) | | | | OMe) | |
| 154 | H₃C—O—CO- | (S)-α- | E | G | T | L-α-Me- | T | S | D | Bip(2′- | (S)-2-amino-4-(quinolin- |
| | His | Me-Pro | | | | Phe(2- | | | | Et-4′- | 6-yloxy)butanamide |
| | | | | | | Fluoro) | | | | OMe) | |
| 155 | H₃C—O—CO- | (S)-α- | E | G | T | L-α-Me- | T | S | D | Bip(2′- | (S)-2-amino-5- |
| | His | Me-Pro | | | | Phe(2- | | | | Et-4′- | phenylpentanamide |
| | | | | | | Fluoro) | | | | OMe) | |
| 156 | H₃C—O—CO- | (S)-α- | E | G | T | L-α-Me- | T | S | D | Bip(2′- | (S)-2-amino-5-(4- |
| | His | Me-Pro | | | | Phe(2- | | | | Et-4′- | chlorophenyl) |
| | | | | | | Fluoro) | | | | OMe) | pentanamide |
| 157 | H₃C—O—CO- | (S)-α- | E | G | T | L-α-Me- | T | S | D | Bip(2′- | (S)-2-amino-5-(3- |
| | His | Me-Pro | | | | Phe(2- | | | | Et-4′- | chlorophenyl) |
| | | | | | | Fluoro) | | | | OMe) | pentanamide |
| 158 | H₃C—O—CO- | (S)-α- | E | G | T | L-α-Me- | T | S | D | Bip(2′- | (S)-2-amino-5- |
| | His | Me-Pro | | | | Phe(2- | | | | Et-4′- | (benzo[d][1,3]dioxol-5- |
| | | | | | | Fluoro) | | | | OMe) | yl)pentanamide |
| 159 | H₃C—O—CO- | (S)-α- | E | G | T | L-α-Me- | T | S | D | Bip(2′- | (S)-2-amino-5-(3,5- |
| | His | Me-Pro | | | | Phe(2- | | | | Et-4′- | dimethylphenyl) |
| | | | | | | Fluoro) | | | | OMe) | pentanamide |
| 160 | H₃C—O—CO- | (S)-α- | E | G | T | L-α-Me- | T | S | D | Bip(2′- | (S)-2-amino-5-(o- |
| | His | Me-Pro | | | | Phe(2- | | | | Et-4′- | tolyl)pentanamide |
| | | | | | | Fluoro) | | | | OMe) | |
| 161 | H₃C—O—CO- | (S)-α- | E | G | T | L-α-Me- | T | S | D | Bip(2′- | (S)-2-amino-5-(3- |
| | His | Me-Pro | | | | Phe(2- | | | | Et-4′- | methoxyphenyl) |
| | | | | | | Fluoro) | | | | OMe) | pentanamide |
| 162 | H₃C—O—CO- | (S)-α- | E | G | T | L-α-Me- | T | S | D | Bip(2′- | (S)-2-amino-5- |
| | His | Me-Pro | | | | Phe(2- | | | | Et-4′- | (naphthalen-2- |
| | | | | | | Fluoro) | | | | OMe) | yl)pentanamide |
| 163 | H₃C—O—CO- | (S)-α- | E | G | T | L-α-Me- | T | S | D | Bip(2′- | (S)-2-amino-5-(6- |
| | His | Me-Pro | | | | Phe(2- | | | | Et-4′- | methylpyridin-2- |
| | | | | | | Fluoro) | | | | OMe) | yl)pentanamide |
| 164 | H₃C—O—CO- | (S)-α- | E | G | T | L-α-Me- | T | S | D | Bip(2′- | (S)-2-amino-5-(quinolin- |
| | His | Me-Pro | | | | Phe(2- | | | | Et-4′- | 6-yl)pentanamide |
| | | | | | | Fluoro) | | | | OMe) | |
| 165 | H₃C—O—CO- | (S)-α- | E | G | T | L-α-Me- | T | S | D | Bip(2′- | (S)-2-amino-5-(2,3- |
| | His | Me-Pro | | | | Phe(2- | | | | Et-4′- | dimethylphenyl) |
| | | | | | | Fluoro) | | | | OMe) | pentanamide |
| 166 | H₃C—O—CO- | (S)-α- | E | G | T | L-α-Me- | T | S | D | Bip(2′- | 2(R)-amino-4-(2-Me-4-Cl- |
| | His | Me-Pro | | | | Phe(2- | | | | Et-4′- | phenoxy)butanamide |
| | | | | | | Fluoro) | | | | OMe) | |
| 167 | H₃C—O—CO- | (S)-α- | E | G | T | L-α-Me- | T | S | D | Bip(2′- | (S)-2-amino-4-(4-chloro- |
| | His | Me-Pro | | | | Phe(2- | | | | Et-4′- | 2-methylphenoxy)-N- |
| | | | | | | Fluoro) | | | | OMe) | methylbutanamide; and |
| 168 | H₃C—O—CO- | (S)-α- | E | G | T | L-α-Me- | T | S | D | Bip(2′- | (R)-2-amino-4-(4-chloro- |
| | His | Me-Pro | | | | Phe(2- | | | | Et-4′- | 2-methylphenoxy)-N- |
| | | | | | | Fluoro) | | | | OMe) | methylbutanamide. |

36. An isolated polypeptide of claim 1, wherein said isolated polypeptide is selected from the group consisting of:

| SEQ ID | | | | | | | | | | | |
|---|---|---|---|---|---|---|---|---|---|---|---|
| NO: | Xₐₐ₁ | Xₐₐ₂ | Xₐₐ₃ | Xₐₐ₄ | Xₐₐ₅ | Xₐₐ₆ | Xₐₐ₇ | Xₐₐ₈ | Xₐₐ₉ | Xₐₐ₁₀ | Xₐₐ₁₁—NH₂ |
| 169 | H | Aib | E | G | T | L-α-Me- | T | S | D | Bip(2′- | (D-Ser(OBz))-NH₂ |
| | | | | | | Phe(2- | | | | Et-4′- | |
| | | | | | | Fluoro) | | | | OMe) | |
| 170 | H | Aib | E | G | T | L-α-Me- | T | S | D | Bip(2′- | (S)-1-(2,3-diamino-3- |
| | | | | | | Phe(2- | | | | Et-4′- | oxopropyl)-3-o-tolylurea |
| | | | | | | Fluoro) | | | | OMe) | |
| 171 | H | Aib | E | G | T | L-α-Me- | T | S | D | Bip(2′- | (S)-1-(2,3-diamino-3- |
| | | | | | | Phe(2- | | | | Et-4′- | oxopropyl)-3-phenylurea |
| | | | | | | Fluoro) | | | | OMe) | |
| 172 | H | (S)-α- | E | G | T | L-α-Me- | T | S | D | Bip(2′- | (S)-1-benzyl-3-(2,3- |
| | | Me-Pro | | | | Phe(2- | | | | Et-4′- | diamino-3-oxopropyl)urea |
| | | | | | | Fluoro) | | | | OMe) | |
| 173 | H | (S)-α- | E | G | T | L-α-Me- | T | S | D | Bip(2′- | (S)-1-phenyl-3-(2,3- |
| | | Me-Pro | | | | Phe(2- | | | | Et-4′- | diamino-3-oxopropyl)urea |
| | | | | | | Fluoro) | | | | OMe) | |
| 174 | H | Aib | E | G | T | L-α-Me- | T | S | D | Bip(2′- | (S)-2-amino-3-(2-o- |
| | | | | | | Phe(2- | | | | Et-4′- | tolylacetamido) |
| | | | | | | Fluoro) | | | | OMe) | propanamide |
| 175 | H | Aib | E | G | T | L-α-Me- | T | S | D | Bip(2′- | (S)-2-amino-3-(2- |
| | | | | | | Phe(2- | | | | Et-4′- | phenylacetamido) |
| | | | | | | Fluoro) | | | | OMe) | propanamide |
| 176 | H | Aib | E | G | T | L-α-Me- | T | S | D | Bip(2′- | (S)-2-amino-4-oxo-4- |
| | | | | | | Phe(2- | | | | Et-4′- | (piperidin-1-yl)butanamide |
| | | | | | | Fluoro) | | | | OMe) | |
| 177 | H | (S)-α- | E | G | T | L-α-Me- | T | S | D | Bip(2′- | (S)-2-chlorobenzyl 2,3- |
| | | Me-Pro | | | | Phe(2- | | | | Et-4′- | diamino-3- |
| | | | | | | Fluoro) | | | | OMe) | oxopropylcarbamate |
| 178 | H | Aib | E | G | T | L-α-Me- | T | S | D | Bip(2′- | (S)-3-acetamido-2- |
| | | | | | | Phe(2- | | | | Et-4′- | aminopropanamide |
| | | | | | | Fluoro) | | | | OMe) | |
| 179 | H | Aib | E | G | T | L-α-Me- | T | S | D | Bip(2′- | (S)-3-amino-N1-(pyridin- |
| | | | | | | Phe(2- | | | | Et-4′- | 2-ylmethyl)succinamide |
| | | | | | | Fluoro) | | | | OMe) | |
| 180 | H | Aib | E | G | T | L-α-Me- | T | S | D | Bip(2′- | (S)-3-amino-N1-2- |
| | | | | | | Phe(2- | | | | Et-4′- | methylbenzylsuccinamide |
| | | | | | | Fluoro) | | | | OMe) | |
| 181 | H | Aib | E | G | T | L-α-Me- | T | S | D | Bip(2′- | (S)-3-amino-N1- |
| | | | | | | Phe(2- | | | | Et-4′- | benzylsuccinamide |
| | | | | | | Fluoro) | | | | OMe) | |
| 182 | H | Aib | E | G | T | L-α-Me- | T | S | D | Bip(2′- | (S)-3-amino-N1- |
| | | | | | | Phe(2- | | | | Et-4′- | isobutylsuccinamide |
| | | | | | | Fluoro) | | | | OMe) | |
| 183 | H | (S)-α- | E | G | T | L-α-Me- | T | S | D | Bip(2′- | (S)-benzyl 2,3-diamino-3- |
| | | Me-Pro | | | | Phe(2- | | | | Et-4′- | oxopropylcarbamate |
| | | | | | | Fluoro) | | | | OMe) | |
| 184 | H | Aib | E | G | T | L-α-Me- | T | S | D | Bip(2′- | (S)—N-(2,3-diamino-3- |
| | | | | | | Phe(2- | | | | Et-4′- | oxopropyl)-2- |
| | | | | | | Fluoro) | | | | OMe) | methylbenzamide |
| 185 | H | Aib | E | G | T | L-α-Me- | T | S | D | Bip(2′- | (S)—N-(2,3-diamino-3- |
| | | | | | | Phe(2- | | | | Et-4′- | oxopropyl)-3- |
| | | | | | | Fluoro) | | | | OMe) | methylbenzamide |
| 186 | H | Aib | E | G | T | L-α-Me- | T | S | D | Bip(2′- | (S)—N-(2,3-diamino-3- |
| | | | | | | Phe(2- | | | | Et-4′- | oxopropyl)-3- |
| | | | | | | Fluoro) | | | | OMe) | methylpicolinamide |
| 187 | H | Aib | E | G | T | L-α-Me- | T | S | D | Bip(2′- | (S)—N-(2,3-diamino-3- |
| | | | | | | Phe(2- | | | | Et-4′- | oxopropyl)-4- |
| | | | | | | Fluoro) | | | | OMe) | methylbenzamide |
| 188 | H | Aib | E | G | T | L-α-Me- | T | S | D | Bip(2′- | (S)—N-(2,3-diamino-3- |
| | | | | | | Phe(2- | | | | Et-4′- | oxopropyl)benzamide |
| | | | | | | Fluoro) | | | | OMe) | |
| 189 | H | Aib | E | G | T | L-α-Me- | T | S | D | Bip-(2′- | (S)—N-(2,3-diamino-3- |
| | | | | | | Phe(2- | | | | Et-4′- | oxopropyl)isonicotinamide |
| | | | | | | Fluoro) | | | | OMe) | |
| 190 | H | Aib | E | G | T | L-α-Me- | T | S | D | Bip(2′- | Glu(1-(2-aminoethyl) |
| | | | | | | Phe(2- | | | | Et-4′- | piperidine)-NH₂ |
| | | | | | | Fluoro) | | | | OMe) | |
| 191 | H | (S)-α- | E | G | T | L-α-Me- | T | S | D | Bip(2′- | Glu(1,2,3,4- |
| | | Me-Pro | | | | Phe(2- | | | | Et-4′- | tetrahydroisoquinoline)- |
| | | | | | | Fluoro) | | | | OMe) | NH₂ |
| 192 | H | Aib | E | G | T | L-α-Me- | T | S | D | Bip(2′- | Glu(2-(2- |
| | | | | | | Phe(2- | | | | Et-4′- | aminoethyl)pyridine)-NH₂ |
| | | | | | | Fluoro) | | | | OMe) | |
| 193 | H | Aib | E | G | T | L-α-Me- | T | S | D | Bip(2′- | Glu(2-fluorobenzylamine)- |
| | | | | | | Phe(2- | | | | Et-4′- | NH₂ |
| | | | | | | Fluoro) | | | | OMe) | |
| 194 | H | Aib | E | G | T | L-α-Me- | T | S | D | Bip(2′- | Glu(2- |
| | | | | | | Phe(2- | | | | Et-4′- | methylbenzylamine)-NH₂ |
| | | | | | | Fluoro) | | | | OMe) | |
| 195 | H | Aib | E | G | T | L-α-Me- | T | S | D | Bip(2′- | Glu(3- |
| | | | | | | Phe(2- | | | | Et-4′- | methoxybenzylamine)- |
| | | | | | | Fluoro) | | | | OMe) | NH₂ |
| 196 | H | (S)-α- | E | G | T | L-α-Me- | T | S | D | Bip(2′- | Glu(3-methoxy-N- |
| | | Me-Pro | | | | Phe(2- | | | | Et-4′- | methylbenzylamine)-NH₂ |
| | | | | | | Fluoro) | | | | OMe) | |
| 197 | H | Aib | E | G | T | L-α-Me- | T | S | D | Bip(2′- | Glu(4-CF₃-benzylamine)- |
| | | | | | | Phe(2- | | | | Et-4′- | NH₂ |
| | | | | | | Fluoro) | | | | OMe) | |
| 198 | H | Aib | E | G | T | L-α-Me- | T | S | D | Bip(2′- | Glu(benzylamine)-NH₂ |
| | | | | | | Phe(2- | | | | Et-4′- | |
| | | | | | | Fluoro) | | | | OMe) | |
| 199 | H | (S)-α- | E | G | T | L-α-Me- | T | S | D | Bip(2′- | Glu(dibenzylamine)-NH₂ |
| | | Me-Pro | | | | Phe(2- | | | | Et-4′- | |
| | | | | | | Fluoro) | | | | OMe) | |
| 200 | H | Aib | E | G | T | L-α-Me- | T | S | D | Bip(2′- | Glu(isobutylamine)-NH₂ |
| | | | | | | Phe(2- | | | | Et-4′- | |
| | | | | | | Fluoro) | | | | OMe) | |
| 201 | H | (S)-α- | E | G | T | L-α-Me- | T | S | D | Bip(2′- | Glu(isoindoline)-NH₂ |
| | | Me-Pro | | | | Phe(2- | | | | Et-4′- | |
| | | | | | | Fluoro) | | | | OMe) | |
| 202 | H | Aib | E | G | T | L-α-Me- | T | S | D | Bip(2′- | Glu(N,N- |
| | | | | | | Phe(2- | | | | Et-4′- | dimethylethylenediamine)- |
| | | | | | | Fluoro) | | | | OMe) | NH₂ |
| 203 | H | Aib | E | G | T | L-α-Me- | T | S | D | Bip(2′- | Glu(n-butylamine)-NH₂ |
| | | | | | | (Phe(2- | | | | Et-4′- | |
| | | | | | | Fluoro) | | | | OMe) | |
| 204 | H | (S)-α- | E | G | T | L-α-Me- | T | S | D | Bip(2′- | Glu(N-ethylbenzylamine)- |
| | | Me-Pro | | | | Phe(2- | | | | Et-4′- | NH₂ |
| | | | | | | Fluoro) | | | | OMe) | |
| 205 | H | (S)-α- | E | G | T | L-α-Me- | T | S | D | Bip(2′- | Glu(N- |
| | | Me-Pro | | | | Phe(2- | | | | Et-4′- | methylbenzylamine)-NH₂ |
| | | | | | | Fluoro) | | | | OMe) | |
| 206 | H | Aib | E | G | T | L-α-Me- | T | S | D | Bip(2′- | Glu(OBz)-NH₂ |
| | | | | | | Phe(2- | | | | Et-4′- | |
| | | | | | | Fluoro) | | | | OMe) | |
| 207 | H | (S)-α- | E | G | T | L-α-Me- | T | S | D | Bip(2′- | Glu(OBz)-NH₂ |
| | | Me-Pro | | | | Phe(2- | | | | Et-4′- | |
| | | | | | | Fluoro) | | | | OMe) | |
| 208 | H | Aib | E | G | T | L-α-Me- | T | S | D | Bip(2′- | Glu(phenethylamine)-NH₂ |
| | | | | | | Phe(2- | | | | Et-4′- | |
| | | | | | | Fluoro) | | | | OMe) | |
| 209 | H | Aib | E | G | T | L-α-Me- | T | S | D | Bip(2′- | Glu(piperidine)-NH₂ |
| | | | | | | Phe(2- | | | | Et-4′- | |
| | | | | | | Fluoro) | | | | OMe) | |
| 210 | H | Aib | E | G | T | L-α-Me- | T | S | D | Bip(2′- | Ser(Bzl)-NH₂ |
| | | | | | | Phe(2- | | | | Et-4′- | |
| | | | | | | Fluoro) | | | | OMe) | |
| 211 | H | Aib | E | G | T | L-α-Me- | T | S | D | Bip(2′- | Thr(OBz)-NH₂; and |
| | | | | | | Phe(2- | | | | Et-4′- | |
| | | | | | | Fluoro) | | | | OMe) | |
| 212 | H | Aib | E | G | T | L-α-Me- | T | S | D | Bip(2′- | L-Asp(OBz)-NH_{2.} |
| | | | | | | Phe(2- | | | | Et-4′- | |
| | | | | | | Fluoro) | | | | OMe) | |

37. An isolated polypeptide of claim 1, wherein said isolated polypeptide is selected from the group consisting of:

| SEQ | | | | | | | | | | | |
|---|---|---|---|---|---|---|---|---|---|---|---|
| ID NO: | Xₐₐ₁ | Xₐₐ₂ | Xₐₐ₃ | Xₐₐ₄ | Xₐₐ₅ | Xₐₐ₆ | Xₐₐ₇ | Xₐₐ₈ | Xₐₐ₉ | Xₐₐ₁₀ | Xₐₐ₁₁—NH₂ |
| 213 | H | Aib | E | G | T | L-α-Me- | T | S | D | Bip(2′- | (S)-2-amino-3-(4- |
| | | | | | | Phe(2- | | | | Et-4′- | (benzamidomethyl)phenyl) |
| | | | | | | Fluoro) | | | | OMe) | propanoic acid |
| 214 | H | Aib | E | G | T | L-α-Me- | T | S | D | Bip(2′- | (S)-2-amino-3-(4- |
| | | | | | | Phe(2- | | | | Et-4′- | (phenylsulfonamidomethyl) |
| | | | | | | Fluoro) | | | | OMe) | phenyl)propanoic acid |
| 215 | H | Aib | E | G | T | L-α-Me- | T | S | D | Bip(2′- | (S)-2-amino-3-(4- |
| | | | | | | Phe(2- | | | | Et-4′- | (methylsulfonamidomethyl) |
| | | | | | | Fluoro) | | | | OMe) | phenyl)propanoic acid |
| 216 | H | Aib | E | G | T | L-α-Me- | T | S | D | Bip(2′- | (S)-2-amino-3-(4-((2,3- |
| | | | | | | Phe(2- | | | | Et-4′- | dimethoxybenzamido) |
| | | | | | | Fluoro) | | | | OMe) | methyl)phenyl)propanoic |
| | | | | | | | | | | | acid |
| 217 | H | Aib | E | G | T | L-α-Me- | T | S | D | Bip(2′- | (S)-2-amino-3-(4-((2,4- |
| | | | | | | Phe(2- | | | | Et-4′- | difluorobenzamido)methyl) |
| | | | | | | Fluoro) | | | | OMe) | phenyl)propanoic acid |
| 218 | H | Aib | E | G | T | L-α-Me- | T | S | D | Bip(2′- | (S)-2-amino-3-(4-((2,4- |
| | | | | | | Phe(2- | | | | Et-4′- | dimethoxybenzamido) |
| | | | | | | Fluoro) | | | | OMe) | methyl)phenyl)propanoic |
| | | | | | | | | | | | acid |
| 219 | H | Aib | E | G | T | L-α-Me- | T | S | D | Bip(2′- | (S)-2-amino-3-(4-((2,5- |
| | | | | | | Phe(2- | | | | Et-4′- | dimethoxybenzamido) |
| | | | | | | Fluoro) | | | | OMe) | methyl)phenyl)propanoic |
| | | | | | | | | | | | acid |
| 220 | H | Aib | E | G | T | L-α-Me- | T | S | D | Bip(2′- | (S)-2-amino-3-(4-((2,6- |
| | | | | | | Phe(2- | | | | Et-4′- | dichlorophenylsulfonamido) |
| | | | | | | Fluoro) | | | | OMe) | methyl)phenyl)propanoic |
| | | | | | | | | | | | acid |
| 221 | H | Aib | E | G | T | L-α-Me- | T | S | D | Bip(2′- | (S)-2-amino-3-(4-((2- |
| | | | | | | Phe(2- | | | | Et-4′- | chlorobenzamido)methyl) |
| | | | | | | Fluoro) | | | | OMe) | phenyl)propanoic acid |
| 222 | H | Aib | E | G | T | L-α-Me- | T | S | D | Bip(2′- | (S)-2-amino-3-(4-((2- |
| | | | | | | Phe(2- | | | | Et-4′- | methoxybenzamido)methyl) |
| | | | | | | Fluoro) | | | | OMe) | phenyl)propanoic acid |
| 223 | H | Aib | E | G | T | L-α-Me- | T | S | D | Bip(2′- | (S)-2-amino-3-(4-((2- |
| | | | | | | Phe(2- | | | | Et-′- | methylbenzamido)methyl) |
| | | | | | | Fluoro) | | | | OMe) | phenyl)propanoic acid |
| 224 | H | Aib | E | G | T | L-α-Me- | T | S | D | Bip(2′- | (S)-2-amino-3-(4-((2- |
| | | | | | | Phe(2- | | | | Et-4′- | methylphenylsulfonamido) |
| | | | | | | Fluoro) | | | | OMe) | methyl)phenyl)propanoic |
| | | | | | | | | | | | acid |
| 225 | H | Aib | E | G | T | L-α-Me- | T | S | D | Bip(2′- | (S)-2-amino-3-(4-((3,4- |
| | | | | | | Phe(2- | | | | Et-4′- | dichlorophenylsulfonamido) |
| | | | | | | Fluoro) | | | | OMe) | methyl)phenyl)propanoic |
| | | | | | | | | | | | acid |
| 226 | H | Aib | E | G | T | L-α-Me- | T | S | D | Bip(2′- | (S)-2-amino-3-(4-((3,4- |
| | | | | | | Phe(2- | | | | Et-4′- | dimethoxybenzamido) |
| | | | | | | Fluoro) | | | | OMe) | methyl)phenyl)propanoic |
| | | | | | | | | | | | acid |
| 227 | H | Aib | E | G | T | L-α-Me- | T | S | D | Bip(2′- | (S)-2-amino-3-(4- |
| | | | | | | Phe(2- | | | | Et-4′- | ((benzo[d][1,3]dioxole-5- |
| | | | | | | Fluoro) | | | | OMe) | carboxamido)methyl)phenyl) |
| | | | | | | | | | | | propanoic acid |
| 228 | H | Aib | E | G | T | L-α-Me- | T | S | D | Bip(2′- | (S)-2-amino-3-(4-((3,5- |
| | | | | | | Phe(2- | | | | Et-4′- | dichlorophenylsulfonamido) |
| | | | | | | Fluoro) | | | | OMe) | methyl)phenyl)propanoic |
| | | | | | | | | | | | acid |
| 229 | H | Aib | E | G | T | L-α-Me- | T | S | D | Bip(2′- | (S)-2-amino-3-(4-((3,5- |
| | | | | | | Phe(2- | | | | Et-4′- | dimethoxybenzamido) |
| | | | | | | Fluoro) | | | | OMe) | methyl)phenyl)propanoic |
| | | | | | | | | | | | acid |
| 230 | H | Aib | E | G | T | L-α-Me- | T | S | D | Bip(2′- | (S)-2-amino-3-(4-((3,5- |
| | | | | | | Phe(2- | | | | Et-4′- | dimethylisoxazole-4- |
| | | | | | | Fluoro) | | | | OMe) | sulfonamido)methyl)phenyl) |
| | | | | | | | | | | | propanoic acid |
| 231 | H | Aib | E | G | T | L-α-Me- | T | S | D | Bip(2′- | (S)-2-amino-3-(4-((3- |
| | | | | | | Phe(2- | | | | Et-4′- | (trifluoromethyl)benzamido) |
| | | | | | | Fluoro) | | | | OMe) | methyl)phenyl)propanoic |
| | | | | | | | | | | | acid |
| 232 | H | Aib | E | G | T | L-α-Me- | T | S | D | Bip(2′- | (S)-2-amino-3-(4-((3- |
| | | | | | | Phe(2- | | | | Et-4′- | chlorobenzamido)methyl) |
| | | | | | | Fluoro) | | | | OMe) | phenyl)propanoic acid |
| 233 | H | Aib | E | G | T | L-α-Me- | T | S | D | Bip(2′- | (S)-2-amino-3-(4-((3- |
| | | | | | | Phe(2- | | | | Et-4′- | chlorophenylsulfonamido) |
| | | | | | | Fluoro) | | | | OMe) | methyl)phenyl)propanoic |
| | | | | | | | | | | | acid |
| 234 | H | Aib | E | G | T | L-α-Me- | T | S | D | Bip(2′- | (S)-2-amino-3-(4-((3- |
| | | | | | | Phe(2- | | | | Et-4′- | cyanobenzamido)methyl) |
| | | | | | | Fluoro) | | | | OMe) | phenyl)propanoic acid |
| 235 | H | Aib | E | G | T | L-α-Me- | T | S | D | Bip(2′- | (S)-2-amino-3-(4-((3- |
| | | | | | | Phe(2- | | | | Et-4′- | ethoxybenzamido)methyl) |
| | | | | | | Fluoro) | | | | OMe) | phenyl)propanoic acid |
| 236 | H | Aib | E | G | T | L-α-Me- | T | S | D | Bip(2′- | (S)-2-amino-3-(4-((3- |
| | | | | | | Phe(2- | | | | Et-4′- | fluorobenzamido)methyl) |
| | | | | | | Fluoro) | | | | OMe) | phenyl)propanoic acid |
| 237 | H | Aib | E | G | T | L-α-Me- | T | S | D | Bip(2′- | (S)-2-amino-3-(4-((3- |
| | | | | | | Phe(2- | | | | Et-4′- | isopropylbenzamido)methyl) |
| | | | | | | Fluoro) | | | | OMe) | phenyl)propanoic acid |
| 238 | H | Aib | E | G | T | L-α-Me- | T | S | D | Bip(2′- | (S)-2-amino-3-(4-((3- |
| | | | | | | Phe(2- | | | | Et-4′- | methoxy-4- |
| | | | | | | Fluoro) | | | | OMe) | methylbenzamido)methyl) |
| | | | | | | | | | | | phenyl)propanoic acid |
| 239 | H | Aib | E | G | T | L-α-Me- | T | S | D | Bip(2′- | (S)-2-amino-3-(4-((3- |
| | | | | | | Phe(2- | | | | Et-4′- | methoxybenzamido)methyl) |
| | | | | | | Fluoro) | | | | OMe) | phenyl)propanoic acid |
| 240 | H | Aib | E | G | T | L-α-Me- | T | S | D | Bip(2′- | (S)-2-amino-3-(4-((3- |
| | | | | | | Phe(2- | | | | Et-4′- | methoxyphenylsulfonamido) |
| | | | | | | Fluoro) | | | | OMe) | methyl)phenyl)propanoic |
| | | | | | | | | | | | acid |
| 241 | H | Aib | E | G | T | L-α-Me- | T | S | D | Bip(2′- | (S)-2-amino-3-(4-((3- |
| | | | | | | Phe(2- | | | | Et-4′- | methylbenzamido)methyl) |
| | | | | | | Fluoro) | | | | OMe) | phenyl)propanoic acid |
| 242 | H | Aib | E | G | T | L-α-Me- | T | S | D | Bip(2′- | (S)-2-amino-3-(4-((3- |
| | | | | | | Phe(2- | | | | Et-4′- | (trifluoromethoxy) |
| | | | | | | Fluoro) | | | | OMe) | benzamido)methyl)phenyl) |
| | | | | | | | | | | | propanoic acid |
| 243 | H | Aib | E | G | T | L-α-Me- | T | S | D | Bip(2′- | (S)-2-amino-3-(4-((3- |
| | | | | | | Phe(2- | | | | Et-4′- | phenylbenzamido)methyl) |
| | | | | | | Fluoro) | | | | OMe) | phenyl)propanoic acid |
| 244 | H | Aib | E | G | T | L-α-Me- | T | S | D | Bip(2′- | (S)-2-amino-3-(4- |
| | | | | | | Phe(2- | | | | Et-4′- | (nicotinamidomethyl)phenyl) |
| | | | | | | Fluoro) | | | | OMe) | propanoic acid |
| 245 | H | Aib | E | G | T | L-α-Me- | T | S | D | Bip(2′- | (S)-2-amino-3-(4-((4- |
| | | | | | | Phe(2- | | | | Et-4′- | butylbenzamido)methyl) |
| | | | | | | Fluoro) | | | | OMe) | phenyl)propanoic acid |
| 246 | H | Aib | E | G | T | L-α-Me- | T | S | D | Bip(2′- | (S)-2-amino-3-(4-((4- |
| | | | | | | Phe(2- | | | | Et-4′- | chlorophenylsulfonamido) |
| | | | | | | Fluoro) | | | | OMe) | methyl)phenyl)propanoic |
| | | | | | | | | | | | acid |
| 247 | H | Aib | E | G | T | L-α-Me- | T | S | D | Bip(2′- | (S)-2-amino-3-(4-((4- |
| | | | | | | Phe(2- | | | | Et-4′- | cyclohexylbenzamido) |
| | | | | | | Fluoro) | | | | OMe) | methyl)phenyl)propanoic |
| | | | | | | | | | | | acid |
| 248 | H | Aib | E | G | T | L-α-Me- | T | S | D | Bip(2′- | (S)-2-amino-3-(4-((4- |
| | | | | | | Phe(2- | | | | Et-4′- | methoxybenzamido)methyl) |
| | | | | | | Fluoro) | | | | OMe) | phenyl)propanoic acid |
| 249 | H | Aib | E | G | T | L-α-Me- | T | S | D | Bip(2′- | (S)-2-amino-3-(4-((4- |
| | | | | | | Phe(2- | | | | Et-4′- | methylphenylsulfonamido) |
| | | | | | | Fluoro) | | | | OMe) | methyl)phenyl)propanoic |
| | | | | | | | | | | | acid |
| 250 | H | Aib | E | G | T | L-α-Me- | T | S | D | Bip(2′- | (S)-2-amino-3-(4-((4- |
| | | | | | | Phe(2- | | | | Et-4′- | methylbenzamido)methyl) |
| | | | | | | Fluoro) | | | | OMe) | phenyl)propanoic acid |
| 251 | H | Aib | E | G | T | L-α-Me- | T | S | D | Bip(2′- | (S)-2-amino-3-(4-((4- |
| | | | | | | Phe(2- | | | | Et-4′- | benzylbenzamido)methyl) |
| | | | | | | Fluoro) | | | | OMe) | phenyl)propanoic acid |
| 252 | H | Aib | E | G | T | L-α-Me- | T | S | D | Bip(2′- | (S)-2-amino-3-(4-((4- |
| | | | | | | Phe(2- | | | | Et-4′- | phenylphenylsulfonamido) |
| | | | | | | Fluoro) | | | | OMe) | methyl)phenyl)propanoic |
| | | | | | | | | | | | acid |
| 253 | H | Aib | E | G | T | L-α-Me- | T | S | D | Bip(2′- | (S)-2-amino-3-(4-((4- |
| | | | | | | Phe(2- | | | | Et-4′- | phenylphenylsulfonamido) |
| | | | | | | Fluoro) | | | | OMe) | methyl)phenyl)propanoic |
| | | | | | | | | | | | acid |
| 254 | H | Aib | E | G | T | L-α-Me- | T | S | D | Bip(2′- | (S)-3-(4-(acetamidomethyl) |
| | | | | | | Phe(2- | | | | Et-4′- | phenyl)-2-aminopropanoic |
| | | | | | | Fluoro) | | | | OMe) | acid |
| 255 | H | Aib | E | G | T | L-α-Me- | T | S | D | Bip(2′- | (S)-2-amino-3-(4- |
| | | | | | | Phe(2- | | | | Et-4′- | (cyclohexanecarboxamidome |
| | | | | | | Fluoro) | | | | OMe) | thyl)phenyl)propanoic acid |
| 256 | H | Aib | E | G | T | L-α-Me- | T | S | D | Bip(2′- | (S)-2-amino-3-(4-((3- |
| | | | | | | Phe(2- | | | | Et-4′- | methylbutanamido)methyl) |
| | | | | | | Fluoro) | | | | OMe) | phenyl)propanoic acid |
| 257 | H | Aib | E | G | T | L-α-Me- | T | S | D | Bip(2′- | (S)-2-amino-3-(4-(2,4- |
| | | | | | | Phe(2- | | | | Et-4′- | dimethoxybenzamido) |
| | | | | | | Fluoro) | | | | OMe) | phenyl)propanoic acid |
| 258 | H | Aib | E | G | T | L-α-Me- | T | S | D | Bip(2′- | (S)-2-amino-3-(4-(2,6- |
| | | | | | | Phe(2- | | | | Et-4′- | dichlorophenylsulfonamido) |
| | | | | | | Fluoro) | | | | OMe) | phenyl)propanoic acid |
| 259 | H | Aib | E | G | T | L-α-Me- | T | S | D | Bip(2′- | (S)-2-amino-3-(4-(2- |
| | | | | | | Phe(2- | | | | Et-4′- | chlorobenzamido)phenyl) |
| | | | | | | Fluoro) | | | | OMe) | propanoic acid |
| 260 | H | Aib | E | G | T | L-α-Me- | T | S | D | Bip(2′- | (S)-2-amino-3-(4-(2- |
| | | | | | | Phe(2- | | | | Et-4′- | methylbenzamido)phenyl) |
| | | | | | | Fluoro) | | | | OMe) | propanoic acid |
| 261 | H | Aib | E | G | T | L-α-Me- | T | S | D | Bip(2′- | (S)-2-amino-3-(4-(3,4- |
| | | | | | | Phe(2- | | | | Et-4′- | dichlorophenylsulfonamido) |
| | | | | | | Fluoro) | | | | OMe) | phenyl)propanoic acid |
| 262 | H | Aib | E | G | T | L-α-Me- | T | S | D | Bip(2′- | (S)-2-amino-3-(4-(3,4- |
| | | | | | | Phe(2- | | | | Et-4′- | dimethoxybenzamido) |
| | | | | | | Fluoro) | | | | OMe) | phenyl)propanoic acid |
| 263 | H | Aib | E | G | T | L-α-Me- | T | S | D | Bip(2′- | (S)-2-amino-3-(4-(3,5- |
| | | | | | | Phe(2- | | | | Et-4′- | dichlorophenylsulfonamido) |
| | | | | | | Fluoro) | | | | OMe) | phenyl)propanoic acid |
| 264 | H | Aib | E | G | T | L-α-Me- | T | S | D | Bip(2′- | (S)-2-amino-3-(4-(3,5- |
| | | | | | | Phe(2- | | | | Et-4′- | dimethylisoxazole-4- |
| | | | | | | Fluoro) | | | | OMe) | sulfonamido)phenyl) |
| | | | | | | | | | | | propanoic acid |
| 265 | H | Aib | E | G | T | L-α-Me- | T | S | D | Bip(2′- | (S)-2-amino-3-(4-(3- |
| | | | | | | Phe(2- | | | | Et-4′- | chlorophenylsulfonamido) |
| | | | | | | Fluoro) | | | | OMe) | phenyl)propanoic acid |
| 266 | H | Aib | E | G | T | L-α-Me- | T | S | D | Bip(2′- | (S)-2-amino-3-(4-(3- |
| | | | | | | Phe(2- | | | | Et-4′- | methylbenzamido)phenyl) |
| | | | | | | Fluoro) | | | | OMe) | propanoic acid |
| 267 | H | Aib | E | G | T | L-α-Me- | T | S | D | Bip(2′- | (S)-2-amino-3-(4- |
| | | | | | | Phe(2- | | | | Et-4′- | (nicotinamido)phenyl) |
| | | | | | | Fluoro) | | | | OMe) | propanoic acid |
| 268 | H | Aib | E | G | T | L-α-Me- | T | S | D | Bip(2′- | (S)-2-amino-3-(4-(4- |
| | | | | | | Phe(2- | | | | Et-4′- | methylphenylsulfonamido) |
| | | | | | | Fluoro) | | | | OMe) | phenyl)propanoic acid |
| 269 | H | Aib | E | G | T | L-α-Me- | T | S | D | Bip(2′- | (S)-2-amino-3-(4-(3- |
| | | | | | | Phe(2- | | | | Et-4′- | phenylbenzamido)phenyl) |
| | | | | | | Fluoro) | | | | OMe) | propanoic acid |
| 270 | H | Aib | E | G | T | L-α-Me- | T | S | D | Bip(2′- | (S)-2-amino-3-(4-(4- |
| | | | | | | Phe(2- | | | | Et-4′- | biphenylsulfonamido) |
| | | | | | | Fluoro) | | | | OMe) | phenyl)propanoic acid |
| 271 | H | Aib | E | G | T | L-α-Me- | T | S | D | Bip(2′- | (S)-2-amino-3-(4- |
| | | | | | | Phe(2- | | | | Et-4′- | (phenylsulfonamido)phenyl) |
| | | | | | | Fluoro) | | | | OMe) | propanoic acid |
| 272 | H | Aib | E | G | T | L-α-Me- | T | S | D | Bip(2′- | (S)-2-amino-3-(4- |
| | | | | | | Phe(2- | | | | Et-4′- | (methylsulfonamido)phenyl) |
| | | | | | | Fluoro) | | | | OMe) | propanoic acid |
| 273 | H | Aib | E | G | T | L-α-Me- | T | S | D | Bip(2′- | (S)-2-amino-3-(4-(2,4- |
| | | | | | | Phe(2- | | | | Et-4′- | difluorobenzamido)phenyl) |
| | | | | | | Fluoro) | | | | OMe) | propanoic acid |
| 274 | H | Aib | E | G | T | L-α-Me- | T | S | D | Bip(2′- | (S)-2-amino-3-(4-(2- |
| | | | | | | Phe(2- | | | | Et-4′- | chlorobenzamido)phenyl) |
| | | | | | | Fluoro) | | | | OMe) | propanoic acid |
| 275 | H | Aib | E | G | T | L-α-Me- | T | S | D | Bip(2′- | (S)-2-amino-3-(4-(2- |
| | | | | | | Phe(2- | | | | Et-4′- | methoxybenzamido)phenyl) |
| | | | | | | Fluoro) | | | | OMe) | propanoic acid |
| 276 | H | Aib | E | G | T | L-α-Me- | T | S | D | Bip(2′- | (S)-2-amino-3-(4-(2- |
| | | | | | | Phe(2- | | | | Et-4′- | methylphenylsulfonamido) |
| | | | | | | Fluoro) | | | | OMe) | phenyl)propanoic acid |
| 277 | H | Aib | E | G | T | L-α-Me- | T | S | D | Bip(2′- | (S)-2-amino-3-(4-(3- |
| | | | | | | Phe(2- | | | | Et-4′- | methoxybenzamido)phenyl) |
| | | | | | | Fluoro) | | | | OMe) | propanoic acid |
| 278 | H | Aib | E | G | T | L-α-Me- | T | S | D | Bip(2′- | (S)-2-amino-3-(4-(3- |
| | | | | | | Phe(2- | | | | Et-4′- | methoxyphenylsulfonamido) |
| | | | | | | Fluoro) | | | | OMe) | phenyl)propanoic acid |
| 279 | H | Aib | E | G | T | L-α-Me- | T | S | D | Bip(2′- | (S)-2-amino-3-(4-(4- |
| | | | | | | Phe(2- | | | | Et-4′- | chlorophenylsulfonamido) |
| | | | | | | Fluoro) | | | | OMe) | phenyl)propanoic acid |
| 280 | H | Aib | E | G | T | L-α-Me- | T | S | D | Bip(2′- | (S)-2-amino-3-(4-(4- |
| | | | | | | Phe(2- | | | | Et-4′- | methoxyphenylsulfonamido) |
| | | | | | | Fluoro) | | | | OMe) | phenyl)propanoic acid |
| 281 | H | (S)-α- | E | G | T | L-α-Me- | T | S | D | Bip(2′- | (S)-2-amino-3-(4-((2,3- |
| | | Me-Pro | | | | Phe(2- | | | | Et-4′- | dimethoxybenzamido) |
| | | | | | | Fluoro) | | | | OMe) | methyl)phenyl)propanoic |
| | | | | | | | | | | | acid |
| 282 | H | (S)-α- | E | G | T | L-α-Me- | T | S | D | Bip(2′- | (S)-2-amino-3-(4-((2,5- |
| | | Me-Pro | | | | Phe(2- | | | | Et-4′- | dimethoxybenzamido) |
| | | | | | | Fluoro) | | | | OMe) | methyl)phenyl)propanoic |
| | | | | | | | | | | | acid |
| 283 | H | (S)-α- | E | G | T | L-α-Me- | T | S | D | Bip(2′- | (S)-2-amino-3-(4- |
| | | Me-Pro | | | | Phe(2- | | | | Et-4′- | ((benzo[d][1,3]dioxole-5- |
| | | | | | | Fluoro) | | | | OMe) | carboxamido)methyl)phenyl) |
| | | | | | | | | | | | propanoic acid |
| 284 | H | (S)-α- | E | G | T | L-α-Me- | T | S | D | Bip(2′- | (S)-2-amino-3-(4-((3,5- |
| | | Me-Pro | | | | Phe(2- | | | | Et-4′- | dimethoxybenzamido) |
| | | | | | | Fluoro) | | | | OMe) | methyl)phenyl)propanoic |
| | | | | | | | | | | | acid |
| 285 | H | (S)-αa- | E | G | T | L-α-Me- | T | S | D | Bip(2′- | (S)-2-amino-3-(4-((3- |
| | | Me-Pro | | | | Phe(2- | | | | Et-4′- | (trifluoromethyl)benzamido) |
| | | | | | | Fluoro) | | | | OMe) | methyl)phenyl)propanoic |
| | | | | | | | | | | | acid |
| 286 | H | (S)-α- | E | G | T | L-α-Me- | T | S | D | Bip(2′- | (S)-2-amino-3-(4-((3- |
| | | Me-Pro | | | | Phe(2- | | | | Et-4′- | cyanobenzamido)methyl) |
| | | | | | | Fluoro) | | | | OMe) | phenyl)propanoic acid |
| 287 | H | (S)-α- | E | G | T | L-α-Me- | T | S | D | Bip(2′- | (S)-2-amino-3-(4-((3- |
| | | Me-Pro | | | | Phe(2- | | | | Et-4′- | ethoxybenzamido)methyl) |
| | | | | | | Fluoro) | | | | OMe) | phenyl)propanoic acid |
| 288 | H | (S)-α- | E | G | T | L-α-Me- | T | S | D | Bip(2′- | (S)-2-amino-3-(4-((3- |
| | | Me-Pro | | | | Phe(2- | | | | Et-4′- | fluorobenzamido)methyl) |
| | | | | | | Fluoro) | | | | OMe) | phenyl)propanoic acid |
| 289 | H | (S)-α- | E | G | T | L-α-Me- | T | S | D | Bip(2′- | (S)-2-amino-3-(4-((3- |
| | | Me-Pro | | | | Phe(2- | | | | Et-4′- | methoxy-4- |
| | | | | | | Fluoro) | | | | OMe) | methylbenzamido)methyl) |
| | | | | | | | | | | | phenyl)propanoic acid |
| 290 | H | (S)-α- | E | G | T | L-α-Me- | T | S | D | Bip(2′- | (S)-2-amino-3-(4-((3- |
| | | Me-Pro | | | | Phe(2- | | | | Et-4′- | methylbenzamido)methyl) |
| | | | | | | Fluoro) | | | | OMe) | phenyl)propanoic acid |
| 291 | H | (S)-α- | E | G | T | L-α-Me- | T | S | D | Bip(2′- | (S)-2-amino-3-(4-((3- |
| | | Me-Pro | | | | Phe(2- | | | | Et-4′- | (trifluoromethoxy) |
| | | | | | | Fluoro) | | | | OMe) | benzamido)methyl)phenyl) |
| | | | | | | | | | | | propanoic acid |
| 292 | H | (S)-α- | E | G | T | L-α-Me- | T | S | D | Bip(2′- | (S)-2-amino-3-(4-((3- |
| | | Me-Pro | | | | Phe(2- | | | | Et-4′- | phenylbenzamido)methyl) |
| | | | | | | Fluoro) | | | | OMe) | phenyl)propanoic acid |
| 293 | H | (S)-α- | E | G | T | L-α-Me- | T | S | D | Bip(2′- | (S)-2-amino-3-(4-((4- |
| | | Me-Pro | | | | Phe(2- | | | | Et-4′- | butylbenzamido)methyl) |
| | | | | | | Fluoro) | | | | OMe) | phenyl)propanoic acid |
| 294 | H | (S)-α- | E | G | T | L-α-Me- | T | S | D | Bip(2′- | (S)-2-amino-3-(4-((4- |
| | | Me-Pro | | | | Phe(2- | | | | Et-4′- | cyclohexylbenzamido) |
| | | | | | | Fluoro) | | | | OMe) | methyl)phenyl)propanoic |
| | | | | | | | | | | | acid |
| 295 | H | (S)-α- | E | G | T | L-α-Me- | T | S | D | Bip(2′- | (S)-2-amino-3-(4-((4- |
| | | Me-Pro | | | | Phe(2- | | | | Et-4′- | methoxybenzamido)methyl) |
| | | | | | | Fluoro) | | | | OMe) | phenyl)propanoic acid |
| 296 | H | (S)-α- | E | G | T | L-α-Me- | T | S | D | Bip(2′- | (S)-2-amino-3-(4-((4- |
| | | Me-Pro | | | | Phe(2- | | | | Et-4′- | benzylbenzamido)methyl) |
| | | | | | | Fluoro) | | | | OMe) | phenyl)propanoic acid |
| 297 | H | (S)-α- | E | G | T | L-α-Me- | T | S | D | Bip(2′- | (S)-3-(4- |
| | | Me-Pro | | | | Phe(2- | | | | Et-4′- | (acetamidomethyl)phenyl)-2- |
| | | | | | | Fluoro) | | | | OMe) | aminopropanoic acid; and |
| 298 | H | (S)-α- | E | G | T | L-α-Me- | T | S | D | Bip(2′- | (S)-2-amino-3-(4-((3- |
| | | Me-Pro | | | | Phe(2- | | | | Et-4′- | methylbutanamido)methyl) |
| | | | | | | Fluoro) | | | | OMe) | phenyl)propanoic acid. |

38. An isolated polypeptide of claim 1, wherein said isolated polypeptide is selected from the group consisting of:

| SEQ | | | | | | | | | | | |
|---|---|---|---|---|---|---|---|---|---|---|---|
| ID NO: | Xₐₐ₁ | Xₐₐ₂ | Xₐₐ₃ | Xₐₐ₄ | Xₐₐ₅ | Xₐₐ₆ | Xₐₐ₇ | Xₐₐ₈ | Xₐₐ₉ | Xₐₐ₁₀ | Xₐₐ₁₁—NH₂ |
| 299 | H | Aib | E | G | T | L-α-Me- | T | S | D | Bip(2′- | (S)-2-amino-5- |
| | | | | | | Phe(2- | | | | Et-4′- | methylhexanoic acid |
| | | | | | | Fluoro) | | | | OMe) | |
| 300 | H | Aib | E | G | T | L-α-Me- | T | S | D | Bip(2′- | 1- |
| | | | | | | Phe(2- | | | | Et-4′- | aminocyclopentanecarboxylic |
| | | | | | | Fluoro) | | | | OMe) | acid |
| 301 | H | Aib | E | G | T | L-α-Me- | T | S | D | Bip(2′- | (S)-2-aminooctanoic acid |
| | | | | | | Phe(2- | | | | Et-4′- | |
| | | | | | | Fluoro) | | | | OMe) | |
| 302 | H | Aib | E | G | T | L-α-Me- | T | S | D | Bip(2′- | (S)-2-aminodecanoic acid |
| | | | | | | Phe(2- | | | | Et-4′- | |
| | | | | | | Fluoro) | | | | OMe) | |
| 303 | H | Aib | E | G | T | L-α-Me- | T | S | D | Bip(2′- | (S)-2-aminohexanoic acid |
| | | | | | | Phe(2- | | | | Et-4′- | |
| | | | | | | Fluoro) | | | | OMe) | |
| 304 | H | Aib | E | G | T | L-α-Me- | T | S | D | Bip(2′- | (S)-2-amino-4- |
| | | | | | | Phe(2- | | | | Et-4′- | methoxybutanoic acid |
| | | | | | | Fluoro) | | | | OMe) | |
| 305 | H | (S)-α- | E | G | T | L-α-Me- | T | S | D | Bip(2′- | (S)-2-aminooctanoic acid |
| | | Me-Pro | | | | Phe(2- | | | | Et-4′- | |
| | | | | | | Fluoro) | | | | OMe) | |
| 306 | H | Aib | E | G | T | L-α-Me- | T | S | D | Bip(2′- | (S)-2-amino-3- |
| | | | | | | Phe(2- | | | | Et-4′- | butoxypropanoic acid |
| | | | | | | Fluoro) | | | | OMe) | |
| 307 | H | Aib | E | G | T | L-α-Me- | T | S | D | Bip(2′- | (S)-2-amino-3-(2- |
| | | | | | | Phe(2- | | | | Et-4′- | methoxyethoxy)propanoic |
| | | | | | | Fluoro) | | | | OMe) | acid |
| 308 | H | (S)-α- | E | G | T | L-α-Me- | T | S | D | Bip(2′- | (S)-2-amino-3- |
| | | Me-Pro | | | | Phe(2- | | | | Et-4′- | butoxypropanoic acid |
| | | | | | | Fluoro) | | | | OMe) | |
| 309 | H | (S)-α- | E | G | T | L-α-Me- | T | S | D | Bip(2′- | (S)-2-amino-3-(2- |
| | | Me-Pro | | | | Phe(2- | | | | Et-4′- | methoxyethoxy)propanoic |
| | | | | | | Fluoro) | | | | OMe) | acid |
| 310 | H | (S)-α- | E | G | T | L-α-Me- | T | S | D | Bip(2′- | (S)-2-amino-3- |
| | | Me-Pro | | | | Phe(2- | | | | Et-4′- | cyclohexylpropanoic acid |
| | | | | | | Fluoro) | | | | OMe) | |
| 311 | H | (S)-α- | E | G | T | L-α-Me- | T | S | D | Bip(2′- | (S)-2-amino-4- |
| | | Me-Pro | | | | Phe(2- | | | | Et-4′- | cyclohexylbutanoic acid |
| | | | | | | Fluoro) | | | | OMe) | |
| 312 | H | (S)-α- | E | G | T | L-α-Me- | T | S | D | Bip(2′- | (R)-2-amino-3- |
| | | Me-Pro | | | | Phe(2- | | | | Et-4′- | cyclohexylpropanoic acid |
| | | | | | | Fluoro) | | | | OMe) | |
| 313 | H | (S)-α- | E | G | T | L-α-Me- | T | S | D | Bip(2′- | (2S,3S)-2-amino-3- |
| | | Me-Pro | | | | Phe(2- | | | | Et-4′- | methylpentanoic acid |
| | | | | | | Fluoro) | | | | OMe) | |
| 314 | H | (S)-α- | E | G | T | L-α-Me- | T | S | D | Bip(2′- | (S)-2-amino-4-methylpent-4- |
| | | Me-Pro | | | | Phe(2- | | | | Et-4′- | enoic acid |
| | | | | | | Fluoro) | | | | OMe) | |
| 315 | H | (S)-α- | E | G | T | L-α-Me- | T | S | D | Bip(2′- | (S)-2-aminopentanoic acid |
| | | Me-Pro | | | | Phe(2- | | | | Et-4′- | |
| | | | | | | Fluoro) | | | | OMe) | |
| 316 | H | (S)-α- | E | G | T | L-α-Me- | T | S | D | Bip(2′- | (R)-2-aminopentanoic acid, |
| | | Me-Pro | | | | Phe(2- | | | | Et-4′- | and |
| | | | | | | Fluoro) | | | | OMe) | |
| 317 | H | (S)-α- | E | G | T | L-α-Me- | T | S | D | Bip(2′- | (R)-2-aminooctanoic acid. |
| | | Me-Pro | | | | Phe(2- | | | | Et-4′- | |
| | | | | | | Fluoro) | | | | OMe) | |

39. An isolated polypeptide of claim 1, wherein said isolated polypeptide is selected from the group consisting of:

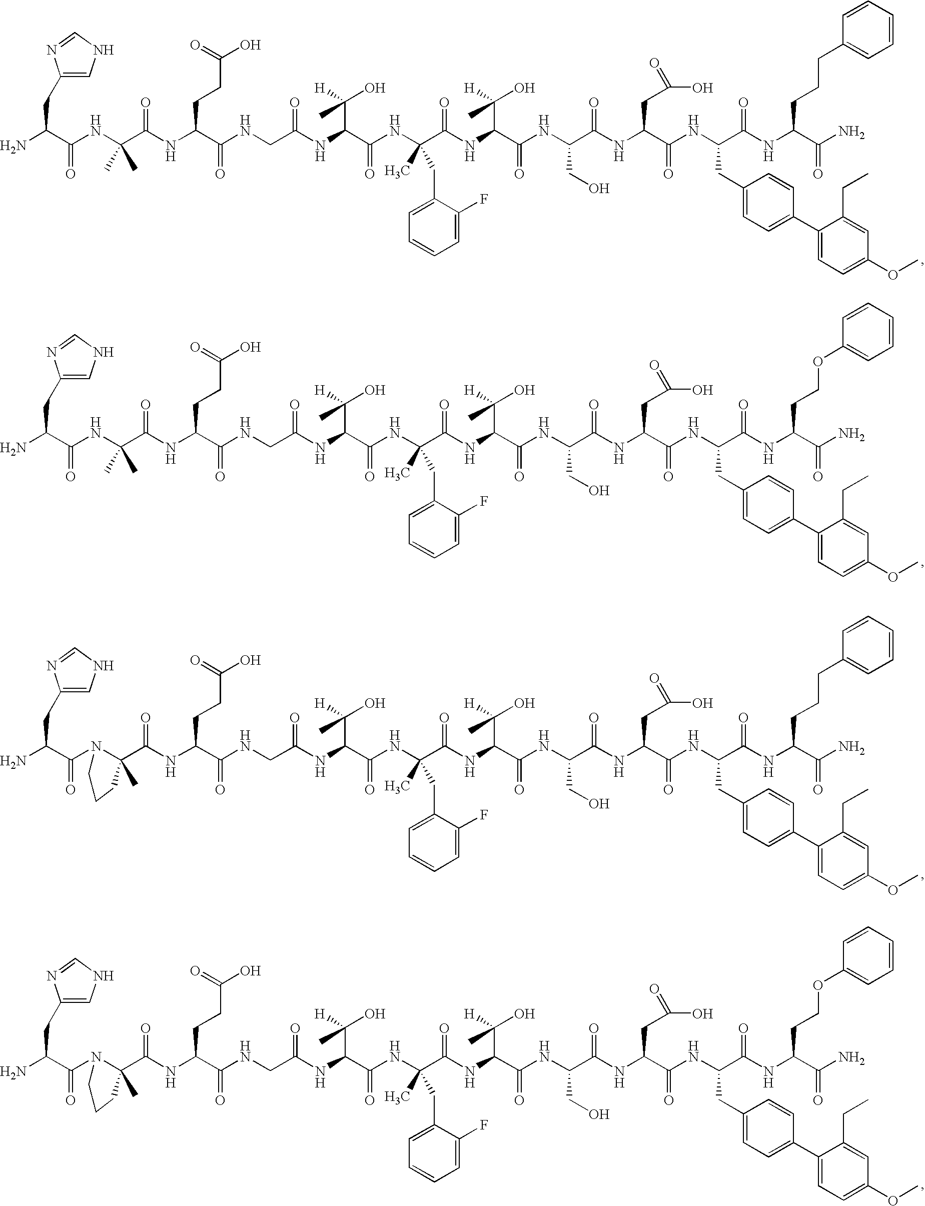

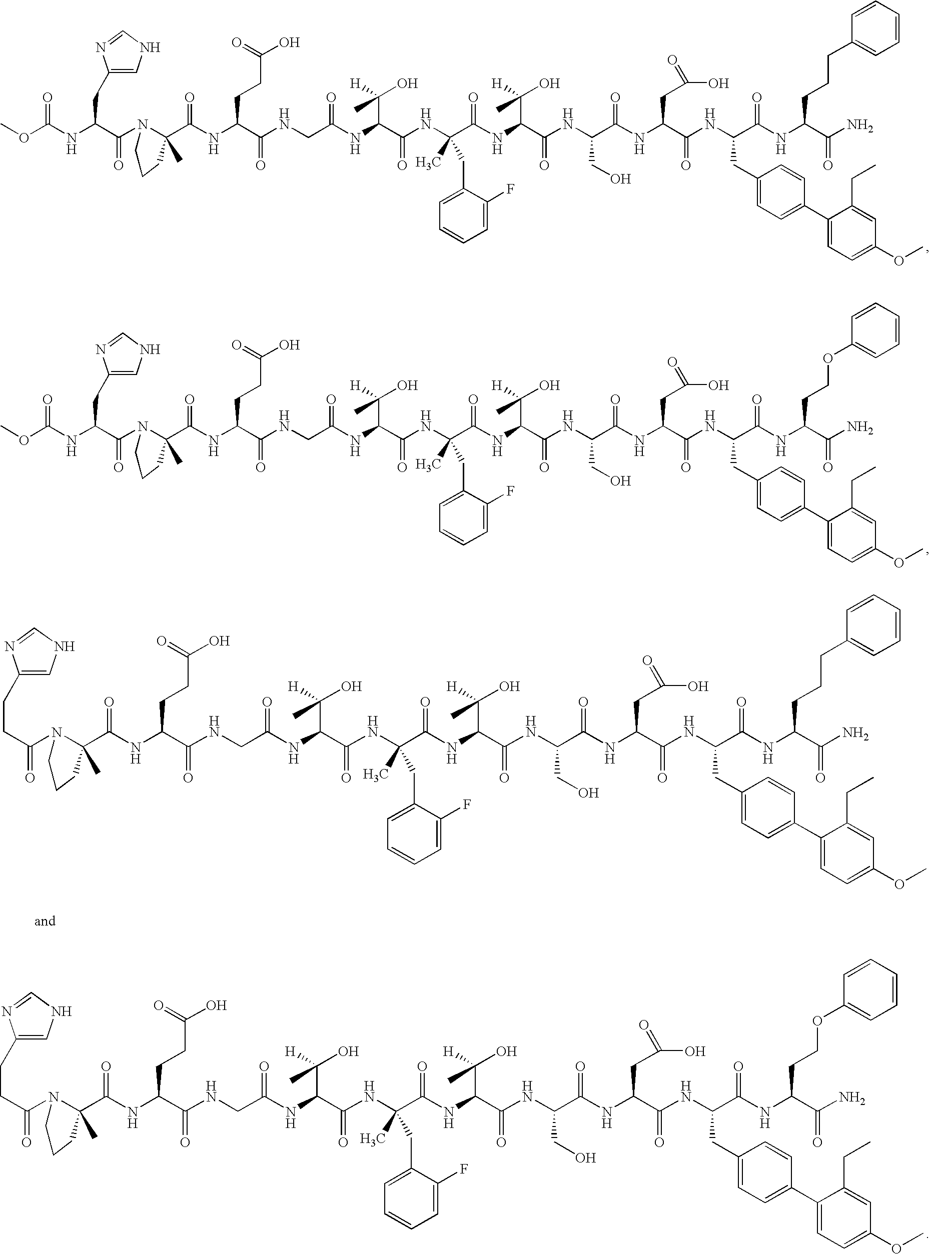

40. An isolated polypeptide of claim 1, wherein said isolated polypeptide is selected from the group consisting of:

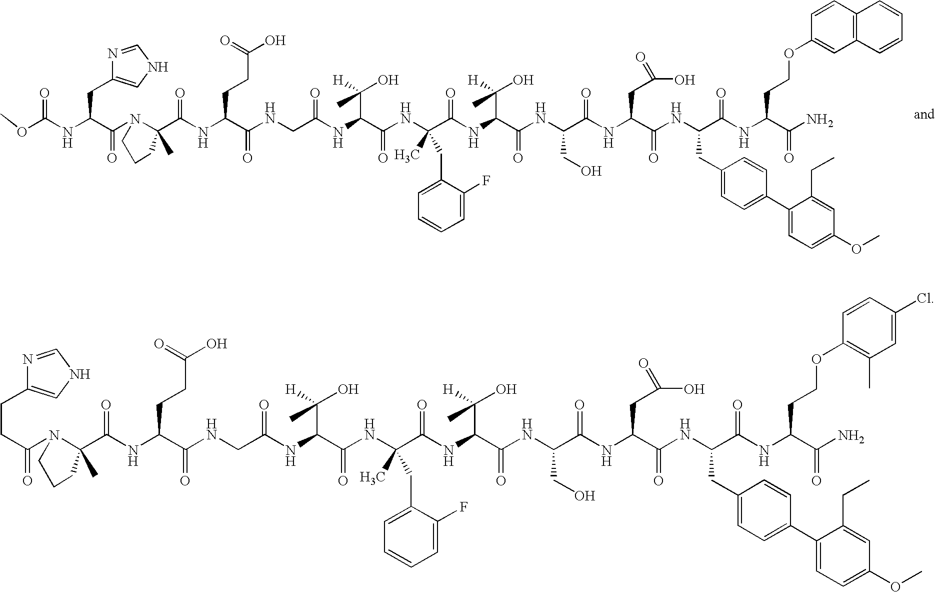

41. An isolated polypeptide of claim 1, wherein said isolated polypeptide is selected from the group consisting of:

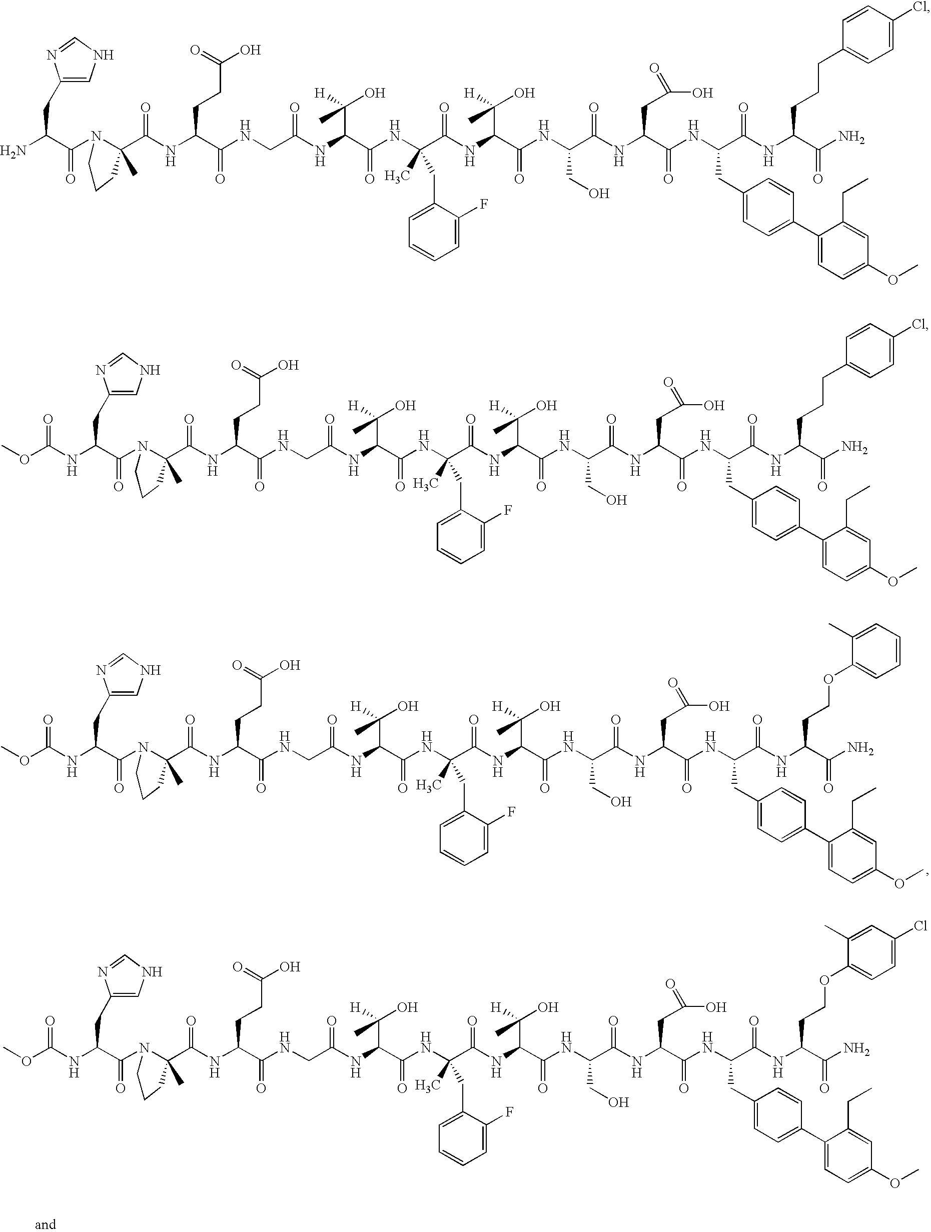

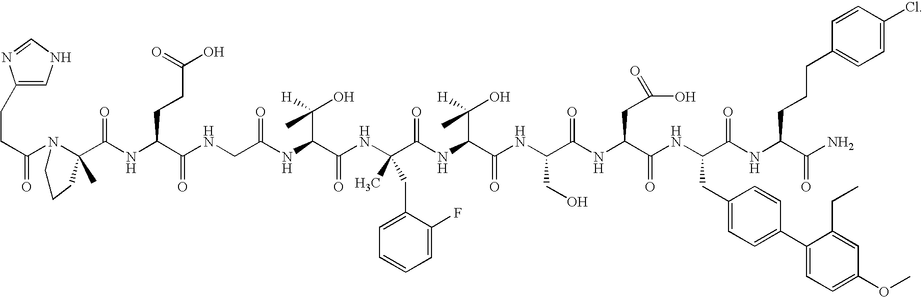

42. An isolated polypeptide of claim 1, wherein said isolated polypeptide is selected from the group consisting of:

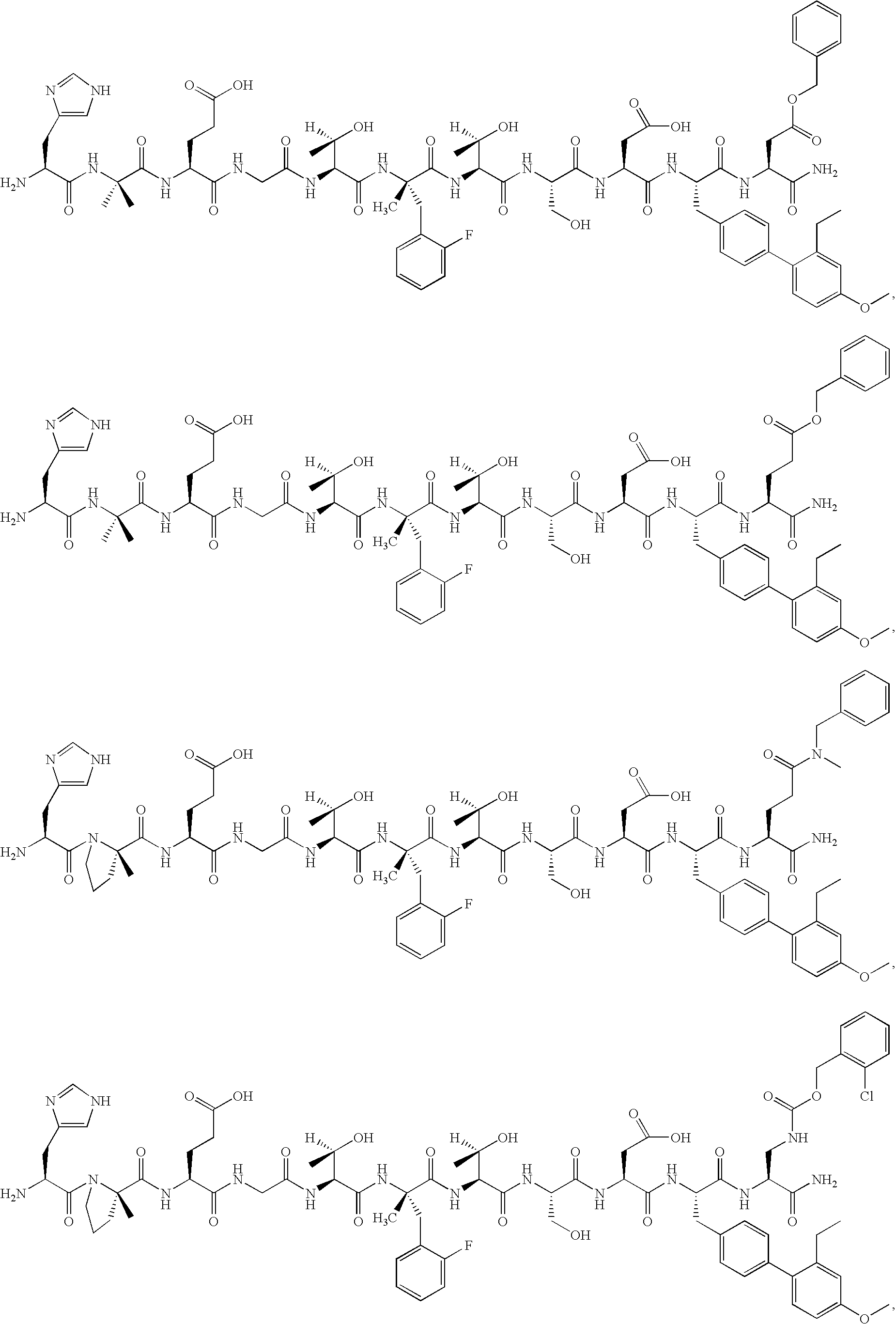

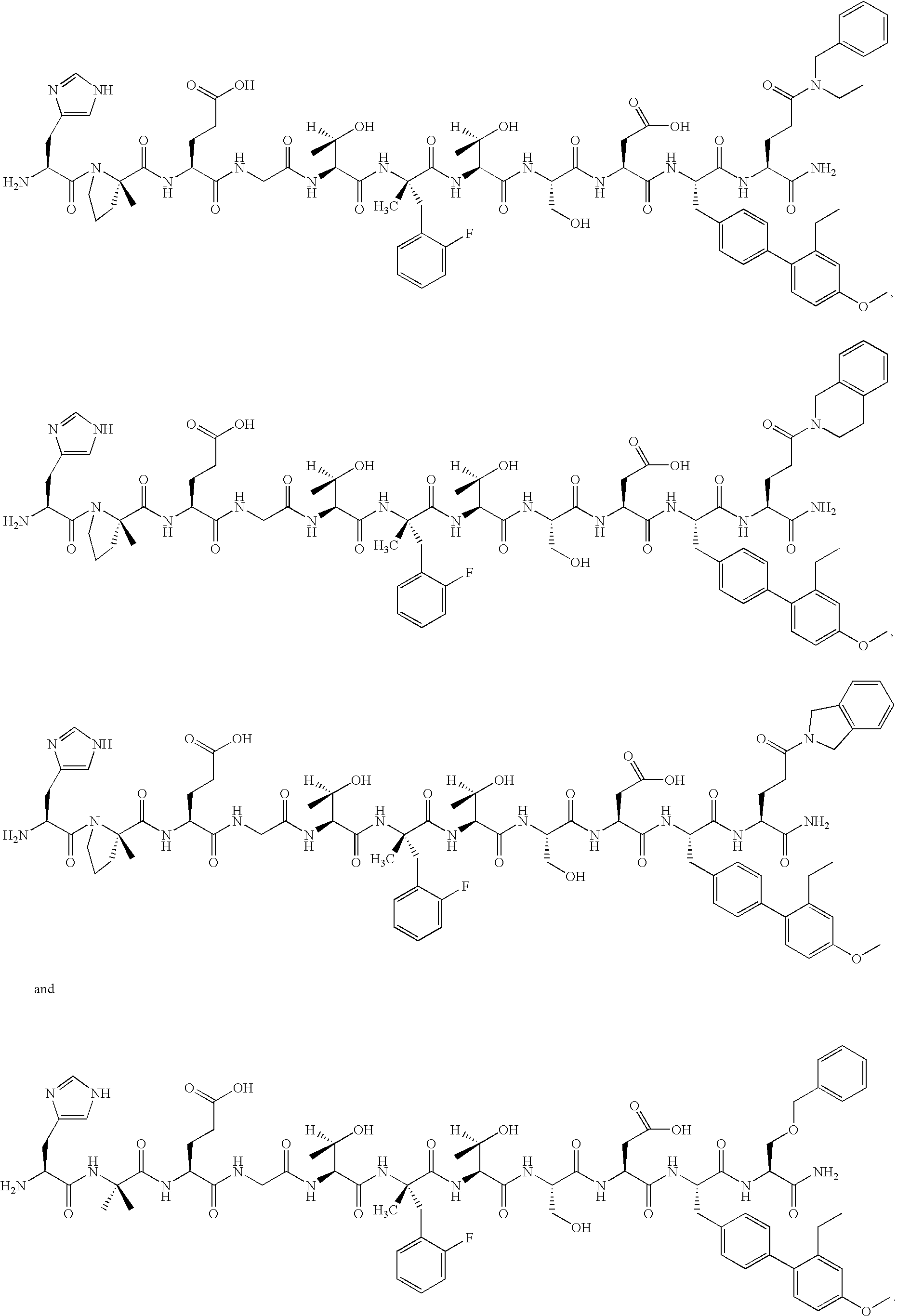

43. An isolated polypeptide of claim 1, wherein said isolated polypeptide is

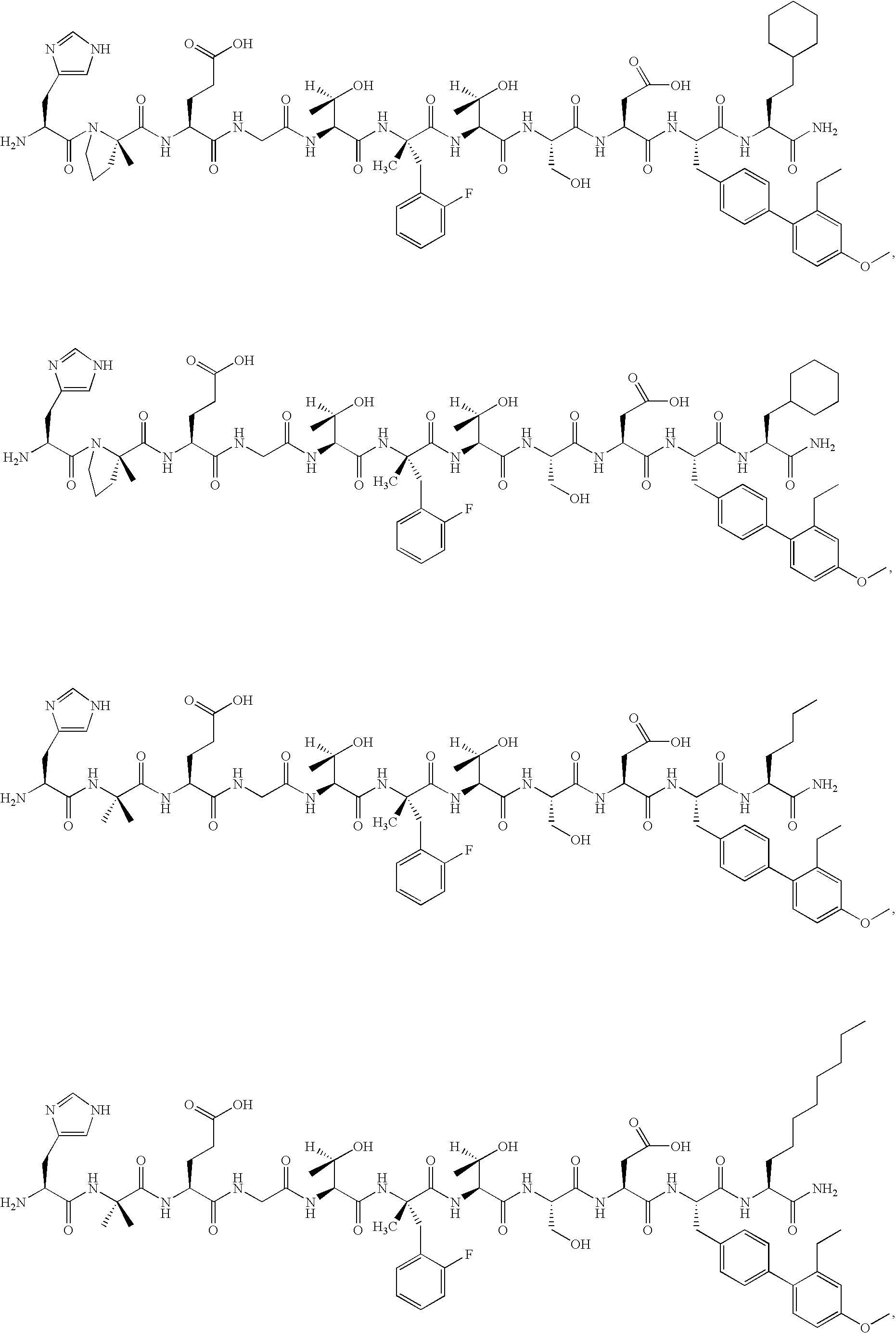

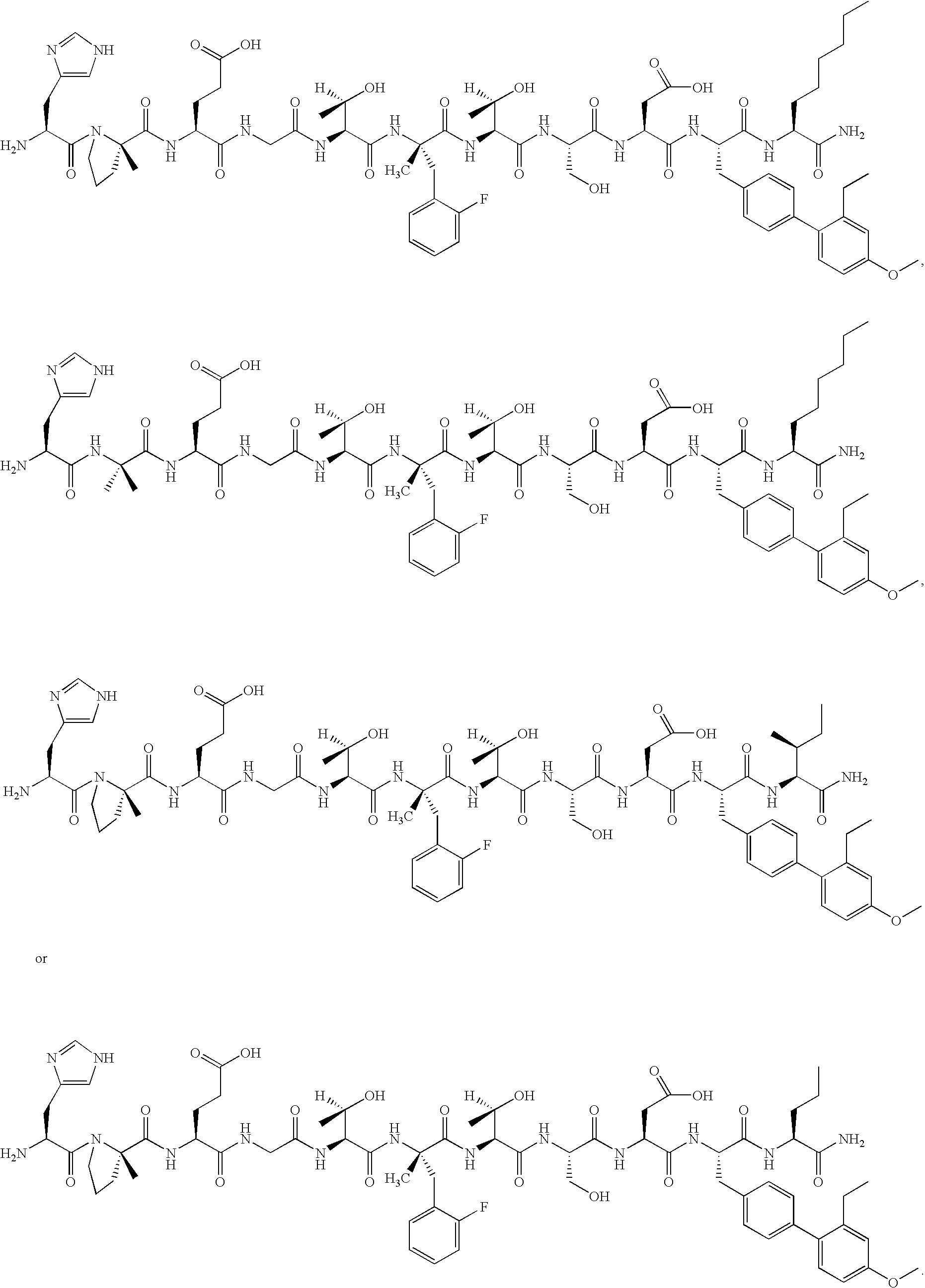

44. A compound of Formula XX: 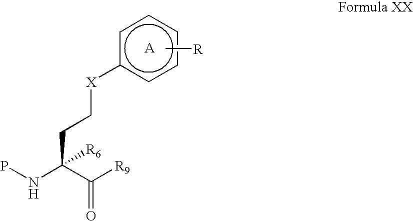
wherein:
P is hydrogen or fluorenylmethyloxycarbonyl (Fmoc) or t-butyloxycarbonyl (t-Boc);
Ring A is selected from the group consisting of aryl and heteroaryl;
R is selected from the group consisting of methyl, ethyl, chloro and fluoro;
R₆ is chosen from the group consisting of hydrogen and methyl;
R₉ is chosen from the group consisting of OH and NH₂; and
X is chosen from the group consisting of CH₂ and O and NH and S.

45. A compound of Formula XXI: 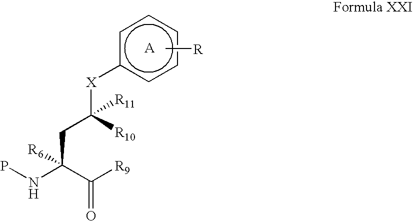
wherein:
P is hydrogen or fluorenylmethyloxycarbonyl (Fmoc) or t-butyloxycarbonyl (t-Boc);
R is selected from the group consisting of methyl, ethyl, chloro, and fluoro,
R₆ is chosen from the group consisting of hydrogen and methyl;
R₉ is chosen from the group consisting of OH and NH₂;
R₁₀ and R₁₁ are each chosen from the group consisting of hydrogen or ethyl or methyl; and
X is chosen from the group consisting of CH₂ and O and NH and S.

46. A pharmaceutical composition comprising an isolated polypeptide of claim 1 or 62 and a pharmaceutically acceptable carrier thereof.

47. A pharmaceutical combination comprising an isolated polypeptide of claim 1 or 62, and at least one therapeutic agent selected from the group consisting of an antidiabetic agent, an anti-obesity agent, an anti-hypertensive agent, an anti-atherosclerotic agent and a lipid-lowering agent.

48. The pharmaceutical combination of claim 47, wherein said antidiabetic agent is selected from the group consisting of a biguanide, a sulfonyl urea, a glucosidase inhibitor, a PPAR γ agonist, a PPAR α/γ dual agonist, an aP2 inhibitor, a DPP4 inhibitor, an insulin sensitizer, a glucagon-like peptide-1 (GLP-1) analog, insulin, and a meglitinide.

49. The pharmaceutical combination of claim 48, wherein said antidiabetic agent is selected from the group consisting of metformin, glyburide, glimepiride, glipyride, glipizide, chlorpropamide, gliclazide, acarbose, miglitol, pioglitazone, troglitazone, rosiglitazone, muraglitazar, insulin, Gl-262570, isaglitazone, JTT-501, N,N-2344, L895645, YM-440, R-119702, AJ9677, repaglinide, nateglinide, KAD1129, AR-HO39242, GW-409544, KRP297, AC2993, LY315902, and NVP-DPP-728A, saxagliptin.

50. The pharmaceutical combination of claim 47, wherein said anti-obesity agent is selected from the group consisting of a beta 3 adrenergic agonist, a lipase inhibitor, a serotonin (and dopamine) reuptake inhibitor, a thyroid receptor beta compound, a CB-1 antagonist and an anorectic agent.

51. The pharmaceutical combination of claim 50, wherein said anti-obesity agent is selected from the group consisting of orlistat, ATL-962, AJ9677, L750355, CP331648, sibutramine, topiramate, axokine, dexamphetamine, phentermine, phenylpropanolamine rimonabant (SR141716A) and mazindol.

52. The pharmaceutical combination of claim 47, wherein said lipid lowering agent is selected from the group consisting of an MTP inhibitor, cholesterol ester transfer protein inhibitor, an HMG CoA reductase inhibitor, a squalene synthetase inhibitor, a fibric acid derivative, an upregulator of LDL receptor activity, a lipoxygenase inhibitor, and an ACAT inhibitor.

53. The pharmaceutical combination of claim 52, wherein said lipid lowering agent is selected from the group consisting of pravastatin, lovastatin, simvastatin, atorvastatin, cerivastatin, fluvastatin, nisvastatin, visastatin, fenofibrate, gemfibrozil, clofibrate, avasimibe, TS-962, MD-700, CP-529414, and LY295427.

54. A method for treating or delaying the progression or onset of diabetes, diabetic retinopathy, diabetic neuropathy, diabetic nephropathy, wound healing, insulin resistance, hyperglycemia, hyperinsulinemia, Syndrome X, diabetic complications, elevated blood levels of free fatty acids or glycerol, hyperlipidemia, obesity, hypertriglyceridemia, atherosclerosis or hypertension, which comprises administering to a mammalian species in need of treatment a therapeutically effective amount of an isolated polypeptide of claim 1 or 62.

55. The method of claim 54, further comprising administering, concurrently or sequentially, a therapeutically effective amount of one or more therapeutic agents selected from the group consisting of an antidiabetic agent, an anti-obesity agent, a anti-hypertensive agent, and an anti-atherosclerotic agent and a lipid-lowering agent.

56. A method for treating or delaying the progression or onset of diabetes, diabetic retinopathy, diabetic neuropathy, diabetic nephropathy, wound healing, insulin resistance, hyperglycemia, hyperinsulinemia, Syndrome X, diabetic complications, elevated blood levels of free fatty acids or glycerol, hyperlipidemia, obesity, hypertriglyceridemia, atherosclerosis or hypertension, which comprises administering to a mammalian species in need of treatment a therapeutically effective amount of a pharmaceutical combination of claim 47.

57. A method of administering a peptide of claim 1 or 62 using a parenteral or non-parenteral formulation.

58. The method of claim 57 wherein said formulation is administered as an immediate release or sustained release formulation.

59. A method for administering a peptide of claim 1 or 62 using a parenteral or non-parenteral formulation, wherein said formulation comprises any one of said compounds as the active ingredient and a pharmaceutically acceptable excipient.

60. A method for administering a peptide of claim 1 or 62 using a parenteral or non-parenteral formulation, wherein said formulation is comprised of said peptide active ingredient and an encapsulated delivery system.

61. An isolated polypeptide of claim 1, wherein said isolated polypeptide is selected from the group consisting of:

| SEQ | | | | | | | | | | | |
|---|---|---|---|---|---|---|---|---|---|---|---|
| ID NO: | Xₐₐ₁ | Xₐₐ₂ | Xₐₐ₃ | Xₐₐ₄ | Xₐₐ₅ | Xₐₐ₆ | Xₐₐ₇ | Xₐₐ₈ | Xₐₐ₉ | Xₐₐ₁₀ | Xₐₐ₁₁—NH₂ |
| 318 | H₃C—O—CO- | (S)-α- | E | G | T | L-α-Me- | T | H | D | Bip(2′- | (S)-2-amino-4-(2- |
| | His | Me-Pro | | | | Phe(2- | | | | Et-4′- | methyl-4- |
| | | | | | | Fluoro) | | | | OMe) | chlorophenoxy) |
| | | | | | | | | | | | butanamide |
| 319 | H₃C—SO₂- | (S)-α- | E | G | T | L-α-Me- | T | S | D | Bip(2′- | (S)-2-amino-4-(2- |
| | His | Me-Pro | | | | Phe(2- | | | | Et-4′- | methyl-4- |
| | | | | | | Fluoro) | | | | OMe) | chlorophenoxy) |
| | | | | | | | | | | | butanamide |
| 320 | H₃C—SO₂- | (S)-α- | E | G | T | L-α-Me- | T | H | D | Bip(2′- | (S)-2-amino-4-(2- |
| | His | Me-Pro | | | | Phe(2- | | | | Et-4′- | methyl-4- |
| | | | | | | Fluoro) | | | | OMe) | chlorophenoxy) |
| | | | | | | | | | | | butanamide |
| 321 | H₃C—O—CO- | (S)-α- | E | G | T | L-α-Me- | T | S | D | Bip(2′- | (S)-2-amino-4-(2- |
| | His | Me-Pro | | | | Phe(2- | | | | Et-4′- | methyl-4- |
| | | | | | | Fluoro) | | | | OMe) | fluorophenoxy) |
| | | | | | | | | | | | butanamide |
| 322 | H₃C—O—CO- | (S)-α- | E | G | T | L-α-Me- | T | H | D | Bip(2′- | (S)-2-amino-4-(2- |
| | His | Me-Pro | | | | Phe(2- | | | | Et-4′- | methyl-4- |
| | | | | | | Fluoro) | | | | OMe) | fluorophenoxy) |
| | | | | | | | | | | | butanamide |
| 323 | H₃C—SO₂- | (S)-α- | E | G | T | L-α-Me- | T | H | D | Bip(2′- | (S)-2-amino-4-(2- |
| | His | Me-Pro | | | | Phe(2- | | | | Et-4′- | methyl-4- |
| | | | | | | Fluoro) | | | | OMe) | fluorophenoxy) |
| | | | | | | | | | | | butanamide; and |
| 324 | (L)-β- | (S)-α- | E | G | T | L-α-Me- | T | S | D | Bip(2′- | (S)-2-amino-4-(2- |
| | Imidazo | Me-Pro | | | | Phe(2- | | | | Et-4′- | methyl-4- |
| | lelactyl | | | | | Fluoro) | | | | OMe) | chlorophenoxy) |
| | | | | | | | | | | | butanamide. |

62. An isolated peptide comprising a core sequence selected from the group consisting of: Thr-Ser-Asp-Bip-Xaa, wherein Xaa is an amino acid comprising a 2-amino-pentamide; Thr-Ser-Asp-Bip-Xaa, wherein Xaa is an amino acid comprising a 2-amino-butanamide; Thr-His-Asp-Bip-Xaa, wherein Xaa is an amino acid comprising a 2-amino-butanamide; Thr-Ser-Asp-Bip-Xaa, wherein Xaa is an amino acid comprising urea; Thr-Ser-Asp-Bip-Xaa, wherein Xaa comprises Glu; Thr-Ser-Asp-Bip-Xaa, wherein Xaa is an amino acid comprising 2-amino-propanoic acid; Thr-Ser-Asp-Bip-Xaa, wherein Xaa is an amino acid comprising a 3-amino-succinamide; Thr-Ser-Asp-Bip-Xaa, wherein Xaa is an amino acid comprising a 2-amino-propanamide; Thr-Ser-Asp-Bip-Xaa, wherein Xaa is an amino acid comprising an ocopropylcarbamate; Thr-Ser-Asp-Bip-Xaa, wherein Xaa is an amino acid comprising an isonicotinamide; Thr-Ser-Asp-Bip-Xaa, wherein Xaa is an amino acid comprising a methylpicolinamide; Thr-Ser-Asp-Bip-Xaa, wherein Xaa is an amino acid further comprising a 1- or 2-amino hexanoic, carboxylic, octanoic, decanoic, butanoic, pentanoic, and enoic acid; and Thr-Ser-Asp-Bip-Xaa, wherein Xaa comprises at least one amino acid coupled to a benzyl group;
wherein said isolated peptide comprising said core sequence binds and activates a GLP-1 receptor.


SEQUENCE LISTING (324 sequences)

SEQ ID NO: 1 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-phenylpentanimide
HXEGTFTSDXX

SEQ ID NO: 2 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(pyridin-2-yl)pentanamide
HXEGTFTSDXX

SEQ ID NO: 3 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(pyridin-3-yl)pentanamide
HXEGTFTSDXX

SEQ ID NO: 4 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(pyrazin-2-yl)pentanamide
HXEGTFTSDXX

SEQ ID NO: 5 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(benzo[d][1,3]dioxol-5-yl) pentanamide
HXEGTFTSDXX

SEQ ID NO: 6 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(3,5-dimethylphenyl) pentanamide
HXEGTFTSDXX

SEQ ID NO: 7 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(2-methylphenyl) pentanamide
HXEGTFTSDXX

SEQ ID NO: 8 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-methylphenyl) pentanamide
HXEGTFTSDXX

SEQ ID NO: 9 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(3-methylphenyl) pentanamide
HPEGTFTSDXX

SEQ ID NO: 10 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-methoxyphenyl) pentanamide
HPEGTFTSDXX

SEQ ID NO: 11 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(5-methylpyridin-2-yl) pentanamide
HPEGTFTSDXX

SEQ ID NO: 12 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-propylphenyl) pentanamide
HPEGTFTSDXX

SEQ ID NO: 13 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(2-ethylphenyl) pentanamide
HPEGTFTSDXX

SEQ ID NO: 14 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-methylpyridin-2-yl) pentanamide
HPEGTFTSDXX

SEQ ID NO: 15 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(2,4-dimethylphenyl) pentanamide)
HPEGTFTSDXX

SEQ ID NO: 16 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(3-methoxyphenyl) pentanamide
HPEGTFTSDXX

SEQ ID NO: 17 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-phenylpentanamide
HPEGTFTSDXX

SEQ ID NO: 18 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-biphenyl) pentanamide
HPEGTFTSDXX

SEQ ID NO: 19 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(3-methylpyridin-2-yl) pentanamide
HPEGTFTSDXX

SEQ ID NO: 20 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(pyridin-4-yl) pentanamide
HPEGTFTSDXX

SEQ ID NO: 21 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(2,3-dimethylphenyl) pentanamide
HPEGTFTSDXX

SEQ ID NO: 22 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(quinolin-6-yl) pentanamide
HPEGTFTSDXX

SEQ ID NO: 23 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(naphthalen-2-yl) pentanamide
HPEGTFTSDXX

SEQ ID NO: 24 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(6-methylpyridin-2-yl) pentanamide
HPEGTFTSDXX

SEQ ID NO: 25 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(2,3-dihydrobenzo[b][1,4]dioxin-6-yl) pentanamide
HPEGTFTSDXX

SEQ ID NO: 26 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(2,6-dimethylpyridin-3-yl) pentanamide
HPEGTFTSDXX

SEQ ID NO: 27 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-chlorophenyl) pentanamide
HPEGTFTSDXX

SEQ ID NO: 28 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(3-chlorophenyl) pentanamide
HPEGTFTSDXX

SEQ ID NO: 29 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-(dimethylamino)phenyl) pentanamide
HPEGTFTSDXX

SEQ ID NO: 30 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(2-chlorophenyl) pentanamide
HPEGTFTSDXX

SEQ ID NO: 31 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-(methylsulfonyl)phenyl) pentanamide
HPEGTFTSDXX

SEQ ID NO: 32 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(quinolin-5-yl) pentanamide
HPEGTFTSDXX

SEQ ID NO: 33 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-(trifluoromethyl)phenyl) pentanamide
HPEGTFTSDXX

SEQ ID NO: 34 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-fluorophenyl) pentanamide
HPEGTFTSDXX

SEQ ID NO: 35 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(3-biphenyl) pentanamide
HPEGTFTSDXX

SEQ ID NO: 36 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is 4-phenylbutan-1-amine
HPEGTFTSDXX

SEQ ID NO: 37 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(2,3,5,6-tetramethylphenyl) pentanamide
HPEGTFTSDXX

SEQ ID NO: 38 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-isopropylphenyl) pentanamide
HPEGTFTSDXX

SEQ ID NO: 39 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(3-biphenyl) pentanamide
HPEGTFTSDXX

SEQ ID NO: 40 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-(dimethylamino)phenyl) pentanamide
HPEGTFTSDXX

SEQ ID NO: 41 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(3-chlorophenyl) pentanamide
HPEGTFTSDXX

SEQ ID NO: 42 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-fluorophenyl) pentanamide
HPEGTFTSDXX

SEQ ID NO: 43 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(2-chlorophenyl) pentanamide
HPEGTFTSDXX

SEQ ID NO: 44 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-(trifluoromethyl)phenyl) pentanamide
HPEGTFTSDXX

SEQ ID NO: 45 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-isopropylphenyl) pentanamide
HPEGTFTSDXX

SEQ ID NO: 46 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-(methylsulfonyl)phenyl) pentanamide
HPEGTFTSDXX

SEQ ID NO: 47 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(quinolin-5-yl) pentanamide
HPEGTFTSDXX

SEQ ID NO: 48 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(2,3,5,6-tetramethylphenyl) pentanamide
HPEGTFTSDXX

SEQ ID NO: 49 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(pyridin-2-yl) pentanamide
HPEGTFTSDXX

SEQ ID NO: 50 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(pyridin-3-yl) pentanamide
HPEGTFTSDXX

SEQ ID NO: 51 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(pyrazin-2-yl) pentanamide
HPEGTFTSDXX

SEQ ID NO: 52 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(benzo[d][1,3]dioxol-5-yl) pentanamide
HPEGTFTSDXX

SEQ ID NO: 53 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(3,5-dimethylphenyl) pentanamide
HPEGTFTSDXX

SEQ ID NO: 54 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-o-tolylpentanamide
HPEGTFTSDXX

SEQ ID NO: 55 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(6-methoxypyridin-2-yl) pentanamide
HPEGTFTSDXX

SEQ ID NO: 56 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(2,3-dihydrobenzofuran-5-yl) pentanamide
HPEGTFTSDXX

SEQ ID NO: 57 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-(4,5-diamino-5-oxopentyl) isonicotinamide
HPEGTFTSDXX

SEQ ID NO: 58 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (R)-2-amino-4-(2-chlorophenoxy) butanamide
HXEGTFTSDXX

SEQ ID NO: 59 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-chlorophenoxy) butanamide
HXEGTFTSDXX

SEQ ID NO: 60 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-chlorophenoxy) butanamide
HPEGTFTSDXX

SEQ ID NO: 61 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-phenoxybutanamide
HXEGTFTSDXX

SEQ ID NO: 62 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-phenoxybutanamide
HPEGTFTSDXX

SEQ ID NO: 63 - protein, 11 aa, Artificial Sequence
  Synthetic Peptide
  MISC_FEATURE (2)..(2): X is aminoisobutyric acid
  MISC_FEATURE (10)..(10): X is biphenylalanine(2′-Et-4′-OMe)
  MISC_FEATURE (11)..(11): X is (S)-2-amino-4-(2,4-dimethylphenoxy) butanamide
HXEGTFTSDXX

SEQ ID NO: 64 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2,4-dimethylphenoxy) butanamide
HPEGTFTSDXX

SEQ ID NO: 65 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methylphenoxy) butanamide
HXEGTFTSDXX

SEQ ID NO: 66 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methylphenoxy) butanamide
HPEGTFTSDXX

SEQ ID NO: 67 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-methylphenoxy) butanamide
HXEGTFTSDXX

SEQ ID NO: 68 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-methoxyphenoxy) butanamide
HPEGTFTSDXX

SEQ ID NO: 69 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-methylphenoxy) butanamide
HXEGTFTSDXX

SEQ ID NO: 70 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-methylphenoxy) butanamide
HPEGTFTSDXX

SEQ ID NO: 71 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(5-methylpyridin-2-yloxy) butanamide
HXEGTFTSDXX

SEQ ID NO: 72 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(5-methylpyridin-2-yloxy) butanamide
HPEGTFTSDXX

SEQ ID NO: 73 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-fluorophenoxy) butanamide
HXEGTFTSDXX

SEQ ID NO: 74 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-fluorophenoxy) butanamide
HPEGTFTSDXX

SEQ ID NO: 75 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-fluorophenoxy) butanamide
HPEGTFTSDXX

SEQ ID NO: 76 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2,4-dichlorophenoxy) butanamide
HPEGTFTSDXX

SEQ ID NO: 77 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2,3-dimethylphenoxy) butanamide
HPEGTFTSDXX

SEQ ID NO: 78 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(3-fluorophenoxy) butanamide
HPEGTFTSDXX

SEQ ID NO: 79 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(3-chlorophenoxy) butanamide
HPEGTFTSDXX

SEQ ID NO: 80 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(3-methoxyphenoxy) butanamide
HPEGTFTSDXX

SEQ ID NO: 81 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(3-methylphenoxy) butanamide
HPEGTFTSDXX

SEQ ID NO: 82 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(3,4-dimethylphenoxy) butanamide
HPEGTFTSDXX

SEQ ID NO: 83 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-phenylphenoxy) butanamide
HXEGTFTSDXX

SEQ ID NO: 84 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2,3-dihydro-1H-inden-5-yloxy) butanamide
HPEGTFTSDXX

SEQ ID NO: 85 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-chloro-2-methylphenoxy) -N-methylbutanamide
HPEGTFTSDXX

SEQ ID NO: 86 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (R)-2-amino-4- (4-(1H-1,2,4-triazol-1-yl-phenoxy)butanamide
HPEGTFTSDXX

SEQ ID NO: 87 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-CN-phenoxy) butanamide
HPEGTFTSDXX

SEQ ID NO: 88 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-MeO-phenoxy) butanamide
HPEGTFTSDXX

SEQ ID NO: 89 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-phenylphenoxy) butanamide
HPEGTFTSDXX

SEQ ID NO: 90 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-CF3-phenoxy) butanamide
HPEGTFTSDXX

SEQ ID NO: 91 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(3-acetamido-phenoxy) butanamide
HPEGTFTSDXX

SEQ ID NO: 92 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(3-dimethylamino-phenoxy) butanamide
HPEGTFTSDXX

SEQ ID NO: 93 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(3-phenylphenoxy) butanamide
HPEGTFTSDXX

SEQ ID NO: 94 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-CL-2-Me-phenoxy) butanamide
HPEGTFTSDXX

SEQ ID NO: 95 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-phenoxy-phenoxy) butanamide
HPEGTFTSDXX

SEQ ID NO: 96 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-CF3-phenoxy) butanamide
HPEGTFTSDXX

SEQ ID NO: 97 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-isopropyl-phenoxy) butanamide
HPEGTFTSDXX

SEQ ID NO: 98 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(1-naphthoxy) butanamide
HPEGTFTSDXX

SEQ ID NO: 99 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-naphthoxy) butanamide
HPEGTFTSDXX

SEQ ID NO: 100 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is 2-amino-4-(4-(imidazol-1-yl)-phenoxy) butanamide
HPEGTFTSDXX

SEQ ID NO: 101 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(benzo[d][1,3]dioxol-5-yl) pentanamide
HPEGTFTSDXX

SEQ ID NO: 102 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(3-dimethylamino-phenoxy) butanamide
HPEGTFTSDXX

SEQ ID NO: 103 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is 2-amino-4-(quinolin-6-yloxy) butanamide
HPEGTFTSDXX

SEQ ID NO: 104 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(3-trifluoromethoxy-phenoxy) butanamide
HPEGTFTSDXX

SEQ ID NO: 105 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is 2-amino-4-(4-methylpyridin-2-yloxy) butanamide
HPEGTFTSDXX

SEQ ID NO: 106 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is 2(S)-amino-4-(2-(1H-prazol-3-yl-phenoxy) butanamide
HPEGTFTSDXX

SEQ ID NO: 107 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is 2(S)-amino-4- (4-(1H-1,2,4-triazol-1-yl-phenoxy)butanamide
HPEGTFTSDXX

SEQ ID NO: 108 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-4-(3,4-diamino-4-oxobutoxy) benzamide
HPEGTFTSDXX

SEQ ID NO: 109 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-phenoxybutanamide
HPEGTFTSDXX

SEQ ID NO: 110 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-fluorophenoxy) butanamide
HPEGTFTSDXX

SEQ ID NO: 111 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-phenylphenoxy) butanamide
HPEGTFTSDXX

SEQ ID NO: 112 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-trifluoromethylphenoxy) butanamide
HPEGTFTSDXX

SEQ ID NO: 113 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methylphenoxy) butanamide
HPEGTFTSDXX

SEQ ID NO: 114 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2,4-dimethylphenoxy) butanamide
HPEGTFTSDXX

SEQ ID NO: 115 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(3-acetamidophenoxy) butanamide
HPEGTFTSDXX

SEQ ID NO: 116 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methyl-4-chlorophenoxy) butanamide
HPEGTFTSDXX

SEQ ID NO: 117 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-phenoxyphenoxy) butanamide
HPEGTFTSDXX

SEQ ID NO: 118 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methoxyphenoxy) butanamide
HPEGTFTSDXX

SEQ ID NO: 119 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-trifluoromethylphenoxy) butanamide
HPEGTFTSDXX

SEQ ID NO: 120 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4- (2,3-dihydro-1H-inden-5-yloxy)butanamide
HPEGTFTSDXX

SEQ ID NO: 121 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(1-naphthoxy)butanamide
HPEGTFTSDXX

SEQ ID NO: 122 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-naphthoxy)butanamide
HPEGTFTSDXX

SEQ ID NO: 123 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4- (benzo[d][1,3]dioxol-5-yloxy)butanamide
HPEGTFTSDXX

SEQ ID NO: 124 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(quinolin-6-yloxy)butanamide
HPEGTFTSDXX

SEQ ID NO: 125 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-phenylpentanamide
HPEGTFTSDXX

SEQ ID NO: 126 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(3-chlorophenyl)pentanamide
HPEGTFTSDXX

SEQ ID NO: 127 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(benzo[d][1,3]dioxol-5-yl) pentanamide
HPEGTFTSDXX

SEQ ID NO: 128 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(3,5-dimethylphenyl) pentanamide
HPEGTFTSDXX

SEQ ID NO: 129 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(3-methoxyphenyl)pentanamide
HPEGTFTSDXX

SEQ ID NO: 130 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(naphthalen-2-yl)pentanamide
HPEGTFTSDXX

SEQ ID NO: 131 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(6-methylpyridin-2-yl) pentanamide
HPEGTFTSDXX

SEQ ID NO: 132 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (R)-2-amino-5-(4-chloro-2-methylphenyl) pentanamide
HPEGTFTSDXX

SEQ ID NO: 133 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-chloro-2-methylphenoxy) -N-methylbutanamide
HPEGTFTSDXX

SEQ ID NO: 134 - protein, 11 aa, Artificial Sequence
  Synthetic Peptide
  MISC_FEATURE (1)..(1): His is des-amino-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (R)-2-amino-4-(4-chloro-2-methylphenoxy) -N-methylbutanamide
HPEGTFTSDXX

SEQ ID NO: 135 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-phenylpentanoyl-NH-Me
HPEGTFTSDXX

SEQ ID NO: 136 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-phenoxybutanamide
HPEGTFTSDXX

SEQ ID NO: 137 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-fluorophenoxy)butanamide
HPEGTFTSDXX

SEQ ID NO: 138 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2,4-dichlorophenoxy) butanamide
HPEGTFTSDXX

SEQ ID NO: 139 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-phenylphenoxy)butanamide
HPEGTFTSDXX

SEQ ID NO: 140 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-trifluoromethylphenoxy) butanamide
HPEGTFTSDXX

SEQ ID NO: 141 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methylphenoxy)butanamide
HPEGTFTSDXX

SEQ ID NO: 142 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2,3-dimethylphenoxy) butanamide
HPEGTFTSDXX

SEQ ID NO: 143 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(3-acetamidophenoxy) butanamide
HPEGTFTSDXX

SEQ ID NO: 144 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(3-phenylphenoxy)butanamide
HPEGTFTSDXX

SEQ ID NO: 145 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methyl-4-chlorophenoxy) butanamide
HPEGTFTSDXX

SEQ ID NO: 146 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methyl-4-phenoxyphenoxy) butanamide
HPEGTFTSDXX

SEQ ID NO: 147 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methyl-4-methoxyphenoxy) butanamide
HPEGTFTSDXX

SEQ ID NO: 148 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4- (2-methyl-4-trifluoromethylphenoxy)butanamide
HPEGTFTSDXX

SEQ ID NO: 149 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4- (2,3-dihydro-1H-inden-5-yloxy)butanamide
HPEGTFTSDXX

SEQ ID NO: 150 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(1-naphthoxy)butanamide
HPEGTFTSDXX

SEQ ID NO: 151 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-naphthoxy)butanamide
HPEGTFTSDXX

SEQ ID NO: 152 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4- (benzo[d][1,3]dioxol-5-yloxy)butanamide
HPEGTFTSDXX

SEQ ID NO: 153 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(quinolin-4-yloxy)butanamide
HPEGTFTSDXX

SEQ ID NO: 154 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(quinolin-6-yloxy)butanamide
HPEGTFTSDXX

SEQ ID NO: 155 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-phenylpentanamide
HPEGTFTSDXX

SEQ ID NO: 156 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(4-chlorophenyl)pentanamide
HPEGTFTSDXX

SEQ ID NO: 157 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(3-chlorophenyl)pentanamide
HPEGTFTSDXX

SEQ ID NO: 158 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(benzo[d][1,3]dioxol-5-yl) pentanamide
HPEGTFTSDXX

SEQ ID NO: 159 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(3,5-dimethylphenyl) pentanamide
HPEGTFTSDXX

SEQ ID NO: 160 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(o-tolyl)pentanamide
HPEGTFTSDXX

SEQ ID NO: 161 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(3-methoxyphenyl)pentanamide
HPEGTFTSDXX

SEQ ID NO: 162 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(naphthalen-2-yl)pentanamide
HPEGTFTSDXX

SEQ ID NO: 163 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(6-methylpyridin-2-yl) pentanamide
HPEGTFTSDXX

SEQ ID NO: 164 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(quinolin-6-yl)pentanamide
HPEGTFTSDXX

SEQ ID NO: 165 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-(2,3-dimethylphenyl) pentanamide
HPEGTFTSDXX

SEQ ID NO: 166 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is 2(R)-amino-4-(2-Me-4-Cl-phenoxy)butanamide
HPEGTFTSDXX

SEQ ID NO: 167 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(4-chloro-2-methylphenoxy) -N-methylbutanamide
HPEGTFTSDXX

SEQ ID NO: 168 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (R)-2-amino-4-(4-chloro-2-methylphenoxy) -N-methylbutanamide
HPEGTFTSDXX

SEQ ID NO: 169 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (D-Ser(OBz))-NH2
HXEGTFTSDXX

SEQ ID NO: 170 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-1-(2,3-diamino-3-oxopropyl) -3-o-tolylurea
HXEGTFTSDXX

SEQ ID NO: 171 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-1-(2,3-diamino-3-oxopropyl) -3-phenylurea
HXEGTFTSDXX

SEQ ID NO: 172 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-1-benzyl-3-(2,3-diamino-3-oxopropyl) urea
HPEGTFTSDXX

SEQ ID NO: 173 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-1-phenyl-3-(2,3-diamino-3-oxopropyl) urea
HPEGTFTSDXX

SEQ ID NO: 174 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(2-o-tolylacetamido) propanamide
HXEGTFTSDXX

SEQ ID NO: 175 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(2-phenylacetamido) propanamide
HXEGTFTSDXX

SEQ ID NO: 176 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-oxo-4-(piperidin-1-yl) butanamide
HXEGTFTSDXX

SEQ ID NO: 177 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-chlorobenzyl 2,3-diamino-3- oxopropylcarbamate
HPEGTFTSDXX

SEQ ID NO: 178 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-3-acetamido-2-aminopropanamide
HXEGTFTSDXX

SEQ ID NO: 179 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-3-amino-N1-(pyridin-2-ylmethyl) succinamide
HXEGTFTSDXX

SEQ ID NO: 180 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-3-amino-N1-2-methylbenzylsuccinamide
HXEGTFTSDXX

SEQ ID NO: 181 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-3-amino-N1-benzylsuccinamide
HXEGTFTSDXX

SEQ ID NO: 182 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-3-amino-N1-isobutylsuccinamide
HXEGTFTSDXX

SEQ ID NO: 183 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-benzyl 2,3-diamino-3- oxopropylcarbamate
HPEGTFTSDXX

SEQ ID NO: 184 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-N-(2,3-diamino-3-oxopropyl)-2- methylbenzamide
HXEGTFTSDXX

SEQ ID NO: 185 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-N-(2,3-diamino-3-oxopropyl)-3- methylbenzamide
HXEGTFTSDXX

SEQ ID NO: 186 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-N-(2,3-diamino-3-oxopropyl)-2- methylpicolinamide
HXEGTFTSDXX

SEQ ID NO: 187 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-N-(2,3-diamino-3-oxopropyl)-4- methylbenzamide
HXEGTFTSDXX

SEQ ID NO: 188 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-N-(2,3-diamino-3-oxopropyl)benzamide
HXEGTFTSDXX

SEQ ID NO: 189 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-N-(2,3-diamino-3-oxopropyl) isonicotinamide
HXEGTFTSDXX

SEQ ID NO: 190 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(1-(2-aminoethyl)piperidine)-NH2
HXEGTFTSDXX

SEQ ID NO: 191 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(1,2,3,4-tetrahydroisoquinoline)-NH2
HPEGTFTSDXX

SEQ ID NO: 192 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(2-(2-aminoethyl)pyridine)-NH2
HXEGTFTSDXX

SEQ ID NO: 193 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(2-fluorobenzylamine)-NH2
HXEGTFTSDXX

SEQ ID NO: 194 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(2-methylbenzylamine)-NH2
HXEGTFTSDXX

SEQ ID NO: 195 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(3-methoxybenzylamine)-NH2
HXEGTFTSDXX

SEQ ID NO: 196 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(3-methoxy-N-methylbenzylamine)-NH2
HPEGTFTSDXX

SEQ ID NO: 197 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(4-CF3-benzylamine)-NH2
HXEGTFTSDXX

SEQ ID NO: 198 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(benzylamine)-NH2
HXEGTFTSDXX

SEQ ID NO: 199 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(dibenzylamine)-NH2
HPEGTFTSDXX

SEQ ID NO: 200 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(isobutylamine)-NH2
HXEGTFTSDXX

SEQ ID NO: 201 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(isoindoline)-NH2
HPEGTFTSDXX

SEQ ID NO: 202 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(N,N-dimethylethylenediamine)-NH2
HXEGTFTSDXX

SEQ ID NO: 203 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(n-butylamine)-NH2
HXEGTFTSDXX

SEQ ID NO: 204 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(N-ethylbenzylamine)-NH2
HPEGTFTSDXX

SEQ ID NO: 205 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(N-methylbenzylamine)-NH2
HPEGTFTSDXX

SEQ ID NO: 206 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(OBz)-NH2
HXEGTFTSDXX

SEQ ID NO: 207 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(OBz)-NH2
HPEGTFTSDXX

SEQ ID NO: 208 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(phenethylamine)-NH2
HXEGTFTSDXX

SEQ ID NO: 209 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Glu(piperidine)-NH2
HXEGTFTSDXX

SEQ ID NO: 210 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Ser(Bzl)-NH2
HXEGTFTSDXX

SEQ ID NO: 211 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is Thr(OBz)-NH2
HXEGTFTSDXX

SEQ ID NO: 212 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is L-Asp(OBz)-NH2
HXEGTFTSDXX

SEQ ID NO: 213 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-(benzamidomethyl)phenyl) propanoic acid
HXEGTFTSDXX

SEQ ID NO: 214 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-(phenylsulfonamidomethyl) phenyl)propanoic acid
HXEGTFTSDXX

SEQ ID NO: 215 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-(methylsulfonamidomethyl) phenyl)propanoic acid
HXEGTFTSDXX

SEQ ID NO: 216 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((2,3-dimethoxybenzamido)methyl)phenyl)propanoic acid
HXEGTFTSDXX

SEQ ID NO: 217 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((2,4-difluorobenzamido)methyl)phenyl)propanoic acid
HXEGTFTSDXX

SEQ ID NO: 218 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((2,4-dimethoxybenzamido)methyl)phenyl)propanoic acid
HXEGTFTSDXX

SEQ ID NO: 219 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((2,5-dimethoxybenzamido)methyl)phenyl)propanoic acid
HXEGTFTSDXX

SEQ ID NO: 220 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((2,6-dichlorophenylsulfonamido)methyl)phenyl)pr opanoic acid
HXEGTFTSDXX

SEQ ID NO: 221 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((2-chlorobenzamido) methyl)phenyl)propanoic acid
HXEGTFTSDXX

SEQ ID NO: 222 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((2-methoxybenzamido)methyl)phenyl)propanoic acid
HXEGTFTSDXX

SEQ ID NO: 223 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((2-methylbenzamido)methyl)phenyl)propanoic acid
HXEGTFTSDXX

SEQ ID NO: 224 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((2-methylphenylsulfonamido)methyl)phenyl)propan oic acid
HXEGTFTSDXX

SEQ ID NO: 225 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3,4-dichlorophenylsulfonamido)methyl)phenyl)pr opanoic acid
HXEGTFTSDXX

SEQ ID NO: 226 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3,4-dimethoxybenzamido)methyl)phenyl)propanoic acid
HXEGTFTSDXX

SEQ ID NO: 227 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((benzo[d][1,3]dioxole-5-carboxamido)methyl)phen yl)propanoic acid
HXEGTFTSDXX

SEQ ID NO: 228 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3,5-dichlorophenylsulfonamido)methyl)phenyl)pr opanoic acid
HXEGTFTSDXX

SEQ ID NO: 229 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3,5-dimethoxybenzamido)methyl)phenyl)propanoic acid
HXEGTFTSDXX

SEQ ID NO: 230 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3,5-dimethylisoxazole-4-sulfonamido)methyl)phe nyl)propanoic acid
HXEGTFTSDXX

SEQ ID NO: 231 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3-trifluoromethyl)benzamido)methyl)phenyl)prop anoic acid
HXEGTFTSDXX

SEQ ID NO: 232 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((3-chlorobenzamido)methyl)phenyl)propanoic acid
HXEGTFTSDXX

SEQ ID NO: 233 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3-chlorophenylsulfonamido)methyl)phenyl)propan oic acid
HXEGTFTSDXX

SEQ ID NO: 234 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((3-cyanobenzamido)methyl)phenyl)propanoic acid
HXEGTFTSDXX

SEQ ID NO: 235 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((3-ethoxybenzamido)methyl)phenyl)propanoic acid
HXEGTFTSDXX

SEQ ID NO: 236 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((3-fluorobenzamido)methyl)phenyl)propanoic acid
HXEGTFTSDXX

SEQ ID NO: 237 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3-isoprophylbenzamido)methyl)phenyl)propanoic acid
HXEGTFTSDXX

SEQ ID NO: 238 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3-methoxy-4-methylbenzamido)methyl)phenyl)prop anoic acid
HXEGTFTSDXX

SEQ ID NO: 239 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3-methoxybenzamido)methyl)phenyl)propanoic acid
HXEGTFTSDXX

SEQ ID NO: 240 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3-methoxyphenylsulfonamido)methyl)phenyl)propa noic acid
HXEGTFTSDXX

SEQ ID NO: 241 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((3-methylbenzamido)methyl)phenyl)propanoic acid
HXEGTFTSDXX

SEQ ID NO: 242 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3-trifluoromethoxy)benzamido)methyl)phenyl)pro panoic acid
HXEGTFTSDXX

SEQ ID NO: 243 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((3-phenylbenzamido)methyl)phenyl)propanoic acid
HXEGTFTSDXX

SEQ ID NO: 244 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(nicotinamidomethyl)phenyl)propanoic acid
HXEGTFTSDXX

SEQ ID NO: 245 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((4-butylbenzamido)methyl)phenyl)propanoic acid
HXEGTFTSDXX

SEQ ID NO: 246 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((4-chlorophenylsulfonamido)methyl)phenyl)propan oic acid
HXEGTFTSDXX

SEQ ID NO: 247 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((4-cyclohexylbenzamido)methyl)phenyl)propanoic acid
HXEGTFTSDXX

SEQ ID NO: 248 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((4-methoxybenzamido)methyl)phenyl)propanoic acid
HXEGTFTSDXX

SEQ ID NO: 249 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((4-methylphenylsulfonamido)methyl)phenyl)propan oic acid
HXEGTFTSDXX

SEQ ID NO: 250 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((4-methylbenzamido)methyl)phenyl)propanoic acid
HXEGTFTSDXX

SEQ ID NO: 251 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((4-benzylbenzamido)methyl)phenyl)propanoic acid
HXEGTFTSDXX

SEQ ID NO: 252 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((4-phenylphenylsulfonamido)methyl)phenyl)propan oic acid
HXEGTFTSDXX

SEQ ID NO: 253 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((4-phenylphenylsulfonamido)methyl)phenyl)propan oic acid
HXEGTFTSDXX

SEQ ID NO: 254 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-3-(4-(acetamidomethyl)phenyl)-2- aminopropanoic acid
HXEGTFTSDXX

SEQ ID NO: 255 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-(cyclohexanecarboxamidomethyl)phenyl)propanoic acid
HXEGTFTSDXX

SEQ ID NO: 256 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3-methylbutanamido)methyl)phenyl)propanoic acide)
HXEGTFTSDXX

SEQ ID NO: 257 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-(2,4-dimethoxybenzamido) phenyl)propanoic acid
HXEGTFTSDXX

SEQ ID NO: 258 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((2,6-dichlorophenylsulfonamido)phenyl)propanoic acid
HXEGTFTSDXX

SEQ ID NO: 259 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(2-chlorobenzamido)phenyl)propanoic acid
HXEGTFTSDXX

SEQ ID NO: 260 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(2-methylbenzamido)phenyl)propanoic acid
HXEGTFTSDXX

SEQ ID NO: 261 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-(3,4-dichlorophenylsulfonamido)phenyl)propanoic acid
HXEGTFTSDXX

SEQ ID NO: 262 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(3,4-dimethoxybenzamido)phenyl)propanoic acid
HXEGTFTSDXX

SEQ ID NO: 263 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-(3,5-dichlorophenylsulfonamido)phenyl)propanoic acid
HXEGTFTSDXX

SEQ ID NO: 264 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-(3,5-dimethylisoxazole-4-sulfonamido)phenyl)prop anoic acid
HXEGTFTSDXX

SEQ ID NO: 265 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(3-chlorophenylsulfonamido)phenyl)propanoic acid
HXEGTFTSDXX

SEQ ID NO: 266 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(3-methylbenzamido)phenyl)propanoic acid
HXEGTFTSDXX

SEQ ID NO: 267 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-(nicotinamido)phenyl) propanoic acid
HXEGTFTSDXX

SEQ ID NO: 268 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(4-methylphenylsulfonamido)phenyl)propanoic acid
HXEGTFTSDXX

SEQ ID NO: 269 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(3-phenylbenzamido)phenyl)propanoic acid
HXEGTFTSDXX

SEQ ID NO: 270 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(4-biphenylsulfonamido)phenyl)propanoic acid
HXEGTFTSDXX

SEQ ID NO: 271 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(phenylsulfonamido)phenyl)propanoic acid
HXEGTFTSDXX

SEQ ID NO: 272 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(methylsulfonamido)phenyl)propanoic acid
HXEGTFTSDXX

SEQ ID NO: 273 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(2,4-difluorobenzamido)phenyl)propanoic acid
HXEGTFTSDXX

SEQ ID NO: 274 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(2-chlorobenzamido)phenyl)propanoic acid
HXEGTFTSDXX

SEQ ID NO: 275 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(2-methoxybenzamido)phenyl)propanoic acid
HXEGTFTSDXX

SEQ ID NO: 276 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(2-methylphenylsulfonamido)phenyl)propanoic acid
HXEGTFTSDXX

SEQ ID NO: 277 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(3-methoxybenzamido)phenyl)propanoic acid
HXEGTFTSDXX

SEQ ID NO: 278 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-(3-methoxyphenylsulfonamido)phenyl)propanoic acid
HXEGTFTSDXX

SEQ ID NO: 279 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-(4-chlorophenylsulfonamido)phenyl)propanoic acid
HXEGTFTSDXX

SEQ ID NO: 280 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-(4-methoxyphenylsulfonamido)phenyl)propanoic acid
HXEGTFTSDXX

SEQ ID NO: 281 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((2,3-dimethoxybenzamido)methyl)phenyl)propanoic acid
HPEGTFTSDXX

SEQ ID NO: 282 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((2,5-dimethoxybenzamido)methyl)phenyl)propanoic acid
HPEGTFTSDXX

SEQ ID NO: 283 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((benzo[d][1,3]dioxole-5-carboxamido)methyl)phen yl)propanoic acid
HPEGTFTSDXX

SEQ ID NO: 284 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3,5-dimethoxybenzamido)methyl)phenyl)propanoic acid
HPEGTFTSDXX

SEQ ID NO: 285 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3-trifluoromethyl)benzamido)methyl)phenyl)prop anoic acid
HPEGTFTSDXX

SEQ ID NO: 286 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((3-cyanobenzamido)methyl)phenyl)propanoic acid
HPEGTFTSDXX

SEQ ID NO: 287 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((3-ethoxybenzamido)methyl)phenyl)propanoic acid
HPEGTFTSDXX

SEQ ID NO: 288 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((3-fluorobenzamido)methyl)phenyl)propanoic acid
HPEGTFTSDXX

SEQ ID NO: 289 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3-methoxy-4-methylbenzamido)methyl)phenyl)prop anoic acid
HPEGTFTSDXX

SEQ ID NO: 290 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((3-methylbenzamido)methyl)phenyl)propanoic acid
HPEGTFTSDXX

SEQ ID NO: 291 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3-trifluoromethoxy)benzamido)methyl)phenyl)pro panoic acid
HPEGTFTSDXX

SEQ ID NO: 292 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((3-phenylbenzamido)methyl)phenyl)propanoic acid
HPEGTFTSDXX

SEQ ID NO: 293 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((4-butylbenzamido)methyl)phenyl)propanoic acid
HPEGTFTSDXX

SEQ ID NO: 294 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((4-cyclohexylbenzamido)methyl)phenyl)propanoic acid
HPEGTFTSDXX

SEQ ID NO: 295 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((4-methoxybenzamido)methyl)phenyl)propanoic acid
HPEGTFTSDXX

SEQ ID NO: 296 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3- (4-((4-benzylbenzamido)methyl)phenyl)propanoic acid
HPEGTFTSDXX

SEQ ID NO: 297 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-3-(4-(acetamidomethyl)phenyl)-2- aminopropanoic acid
HPEGTFTSDXX

SEQ ID NO: 298 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(4-((3-methylbutanamido)methyl)phenyl)propanoic acid
HPEGTFTSDXX

SEQ ID NO: 299 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-5-methylhexanoic acid
HXEGTFTSDXX

SEQ ID NO: 300 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is 1-aminocyclopentanecarboxylic acid
HXEGTFTSDXX

SEQ ID NO: 301 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-aminooctanoic acid
HXEGTFTSDXX

SEQ ID NO: 302 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-aminodecanoic acid
HXEGTFTSDXX

SEQ ID NO: 303 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-aminohexanoic acid
HXEGTFTSDXX

SEQ ID NO: 304 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-methoxybutanoic acid
HXEGTFTSDXX

SEQ ID NO: 305 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-aminooctanoic acid
HPEGTFTSDXX

SEQ ID NO: 306 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-butoxypropanoic acid
HXEGTFTSDXX

SEQ ID NO: 307 - protein, 11 aa, Artificial Sequence
  Synthetic Peptide
  MISC_FEATURE (2)..(2): X is aminoisobutyric acid
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(2-methoxyethoxy)propanoic acid
HXEGTFTSDXX

SEQ ID NO: 308 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-butoxypropanoic acid
HPEGTFTSDXX

SEQ ID NO: 309 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-(2-methoxyethoxy)propanoic acid
HPEGTFTSDXX

SEQ ID NO: 310 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-3-cyclohexylpropanoic acid
HPEGTFTSDXX

SEQ ID NO: 311 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-cyclohexylbutanoic acid
HPEGTFTSDXX

SEQ ID NO: 312 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (R)-2-amino-3-cyclohexylpropanoic acid
HPEGTFTSDXX

SEQ ID NO: 313 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (2S,3S)-2-amino-3-methylpentanoic acid
HPEGTFTSDXX

SEQ ID NO: 314 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-methylpent-4-enoic acid
HPEGTFTSDXX

SEQ ID NO: 315 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-aminopentanoic acid
HPEGTFTSDXX

SEQ ID NO: 316 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (R)-2-aminopentanoic acid
HPEGTFTSDXX

SEQ ID NO: 317 - protein, 11 aa, Artificial Sequence
  Synthetic Peptide
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (R)-2-aminooctanoic acid
HPEGTFTSDXX

SEQ ID NO: 318 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methyl-4-chlorophenoxy) butanamide
HPEGTFTHDXX

SEQ ID NO: 319 - protein, 11 aa, Artificial Sequence
  Synthetic Peptide
  MISC_FEATURE (1)..(1): His is H3C-SO2-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methyl-4-chlorophenoxy) butanamide
HPEGTFTSDXX

SEQ ID NO: 320 - protein, 11 aa, Artificial Sequence
  Synthetic Peptide
  MISC_FEATURE (1)..(1): His is H3C-SO2-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methyl-4-chlorophenoxy) butanamide
HPEGTFTHDXX

SEQ ID NO: 321 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methyl-4-fluorophenoxy) butanamide
HPEGTFTSDXX

SEQ ID NO: 322 - protein, 11 aa, Artificial Sequence
  Synthetic Peptide
  MISC_FEATURE (1)..(1): His is H3C-O-CO-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methyl-4-fluorophenoxy) butanamide
HPEGTFTHDXX

SEQ ID NO: 323 - protein, 11 aa, Artificial Sequence
  Synthetic Peptide
  MISC_FEATURE (1)..(1): His is H3C-SO2-His
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methyl-4-fluorophenoxy) butanamide
HPEGTFTHDXX

SEQ ID NO: 324 - protein, 11 aa, Artificial Sequence
  Synthetic Peptide
  MISC_FEATURE (1)..(1): X is (L)-beta-Imidazolelactyl
  MISC_FEATURE (2)..(2): Pro is (S)-alpha-Me-Pro
  MISC_FEATURE (6)..(6): Phe is L-alpha-Me-Phe(2-Fluoro)
  MISC_FEATURE (10)..(10): X is biphenylalanine(2′-Et-4′-OMe)
  MISC_FEATURE (11)..(11): X is (S)-2-amino-4-(2-methyl-4-chlorophenoxy) butanamide
XPEGTFTSDXX